(19) United States
(12) Patent Application Publication
(10) Pub. No.: US 2024/0368661 A1
(43) Pub. Date: Nov. 7, 2024

(54) COMPOSITIONS AND METHODS FOR PRODUCING REBAUDIOSIDE D

(71) Applicant: Arzeda Corp., Seattle, WA (US)

(72) Inventors: Kyle Eugene ROBERTS, San Marcos, CA (US); Alexandre ZANGHELLINI, Ithaca, NY (US); Daniela GRABS, Seattle, WA (US); Niklas Dalgas KRISTIANSEN, Redmond, WA (US); James J. HAVRANEK, Seattle, WA (US); Yih-En Andrew BAN, Shoreline, WA (US); Ashwini DEVKOTA, Shoreline, WA (US); Mark NANCE, Sammamish, WA (US)

(21) Appl. No.: 18/546,881

(22) PCT Filed: Feb. 17, 2022
(86) PCT No.: PCT/US2022/016820

§ 371 Date: Aug. 17, 2023

Related U.S. Application Data
(60) Provisional application No. 63/150,515, filed on Feb. 17, 2021.

(51) Int. Cl. C12P 19/56; C12N 9/10

(52) U.S. Cl. CPC C12P 19/56; C12N 9/1062; C12Y 204/01013

(57) ABSTRACT

The present disclosure provides enzymes and a method to use those enzymes to transfer a sugar moiety to a substrate steviol glycoside. Specifically, designed beta-1,2-glycosyltransferases and sucrose synthases are used in a one-pot reaction to convert stevioside and Reb A into Reb E and Reb D.

### CROSS-REFERENCE TO RELATED APPLICATIONS

This application claims the benefit of U.S. Provisional Patent Application No. 63/150,515, filed Feb. 17, 2021, the content of which is herein incorporated by reference in its entirety.

### INCORPORATION OF THE SEQUENCE LISTING

The contents of the text file submitted electronically herewith are incorporated herein by reference in their entirety: A computer readable format copy of the Sequence Listing (filename: ARZE_034_01WO_SeqList_ST25.txt, date recorded: Feb. 17, 2022, file size ˜6.84 megabytes).

### FIELD OF THE DISCLOSURE

The present disclosure relates to enzymes and biocatalytic processes for producing steviol glycosides. The present disclosure particularly relates to use of glycosyltransferases that can transfer a glucose moiety from an ADP-glucose sugar donor to steviol glycosides.

### BACKGROUND

Excess sugar consumption has been linked to worldwide health epidemics including diabetes and heart disease. Healthcare systems incur exorbitant costs associated with treating these diseases. Replacing added sugar in food with a low calorie, high-intensity sweetener would have significant health and economic impact.

The species Stevia rebaudiana is commonly grown for its sweet leaves, which have traditionally been used as a sweetener. Stevia extract is 200-300 times sweeter than sugar and is used commercially as a high intensity sweetener. The main glycoside components of stevia leaf are steviosides and rebaudiosides. Over ten different steviol glycosides are present in appreciable quantities in the leaf. The principal sweetening compounds are stevioside and rebaudioside A. Rebaudioside A (Reb A) is considered a higher value compared to stevioside because of its increased sweetness and decreased bitterness.

The sweetness and bitterness profile of rebaudioside D (Reb D) is improved compared to Reb A, but Reb D is present at very low quantities in the stevia leaf. Reb D can be made by the addition of a single glucose molecule to Reb A. Native glycosyltransferases that make Reb D use UDP-glucose as the glucose source for transferring to Reb A.

### BRIEF SUMMARY

The present disclosure provides enzymes, particularly non-natural enzymes, and methods to use those enzymes to transfer a sugar moiety to a substrate steviol glycoside (also referred to herein as a “SG”). Specifically, a beta-1,2-glycosyltransferase (also referred to herein as a “B12GT”) and sucrose synthase (also referred to herein as a “SuSy”) are used in a one-pot reaction to convert stevioside and Reb A into rebaudioside E (Reb E) and Reb D, respectively.

In contrast to native glycosyltransferases, the disclosure provides glycosyltransferase polypeptides that can utilize ADP-glucose as the sugar donor to convert Reb A to Reb D. The disclosure provides glycosyltransferase polypeptides that comprise an amino acid sequence that is at least 60%, at least 65%, at least 70%, at least 75%, at least 80%, at least 85%, at least 90%, at least 95%, at least 98%, at least 99%, or 100% identical to an amino acid sequence selected from the group consisting of SEQ ID NOs: 6-882 and 1333-1466. The glycosyltransferase polypeptide may comprise, or consist of, an amino acid sequence selected from the group consisting of SEQ ID NOs: 6-882 and 1333-1466. The polypeptides may comprise one or more peptide tags used for solubility, expression and/or purification; for example, a polyhistidine tag of between 4 and 10 histidine residues, and preferably 6 histidine residues. Other suitable tags include, but are not limited to, glutathione S-transferase (GST), FLAG, maltose binding protein (MBP), calmodulin binding peptide (CBP), and Myc tag. Suitable linkers include, but are not limited to, polypeptides composed of glycine and serine, such as GSGS, polyglycine linkers, EAAAK repeats, and sequences containing cleavage sites for enzymes such as factor Xa, enterokinase, and thrombin.

Nucleotide sugar donors, including both UDP-glucose and ADP-glucose, are expensive co-substrates and add significant costs to any process that utilizes the compounds. Sucrose synthases (SuSy; EC 2.4.1.13) catalyze the chemical reaction of nucleotide diphosphate (NDP) and sucrose to form NDP-glucose and fructose. Therefore, sucrose synthases can be used to convert an NDP into an NDP-glucose required by B12GTs (an exemplary glycosyltransferase). The disclosure provides SuSy polypeptides that comprise an amino acid sequence that is at least 60%, at least 65%, at least 70%, at least 75%, at least 80%, at least 85%, at least 90%, at least 95%, at least 98%, at least 99%, or 100% identical to an amino acid sequence selected from the group consisting of SEQ ID NOs: 890-1227 and 1231-1332. Specifically, the disclosed sucrose synthases can convert ADP into the ADP-glucose cofactor required by the disclosed B12GTs.

The disclosure additionally provides a method to utilize a SuSy ADP-glucose recycling system combined with a B12GT polypeptide in a one-pot reaction to convert Reb A and/or stevioside into Reb D and Reb E, respectively. In some embodiments, the method comprises contacting a stevia leaf extract purified to contain greater than 50% Reb A (RA50), ADP, and sucrose with a B1,2 glycosyltransferase and sucrose synthase to make Reb D and/or Reb E.

### BRIEF DESCRIPTION OF THE DRAWINGS

The accompanying drawings are included to provide a further understanding of the disclosure. The drawings illustrate embodiments of the disclosure and together with the description, serve to explain the principles of the embodiments of the disclosure.

FIG. 1 shows the conversion (glycosylation) of rebaudioside A (Reb A) to rebaudioside D (Reb D).

FIG. 2 shows the measured Reb A to Reb D activity of three native UDP-glucose B12GTs when using either ADP-glucose or GDP-glucose as the sugar donor.

FIG. 3 shows the measured ability of native sucrose synthases to convert ADP to ADP-glucose (top), GDP to GDP-glucose (middle), and UDP to UDP-glucose (bottom).

FIG. 4 (top) shows the measured conversion of Reb A to Reb D to Reb M2 in a one-pot reaction of the B12GT pA10143 and one of seven native sucrose synthases. FIG. 4 (bottom) shows the measured conversion of Reb A to Reb D in a one-pot reaction of the B12GT pA12549 and one of seven native sucrose synthases.

FIG. 5 shows the top designs of pA10143 from an active-site site saturation mutagenesis library. The parent enzyme, pA10143, is shown in gray.

FIG. 6 shows the measured Reb A to Reb D conversion for all enzymes from the pA10143 active-site SSM library by mutated residue.

FIG. 7 shows the LCMS chromatogram of the reaction product produced from a scaled-up one-pot reaction of pA21841 and pA29798.

FIG. 8 shows the LCMS chromatogram of the reaction product produced from a scaled-up one-pot reaction of pA21841 and pA29646.

FIG. 9 shows an SDS-PAGE gel of designed B12GTs purified from Pichia pastoris expression.

FIG. 10A shows an SDS-PAGE gel of two designed B12GTs purified from a 1 L Pichia pastoris fermentation (order from left to right: pA29798 (B12GT-1), ladder, pA32946 (B12GT-2)). FIG. 10B shows SDS-PAGE gels of two designed SuSys purified from a 1 L Pichia pastoris fermentation (order from left to right: ladder, pA34103 (SuSy-1), ladder, pA32691 (SuSy-2)).

### DETAILED DESCRIPTION

The present disclosure provides enzymes and biocatalytic processes for preparing a composition comprising a target steviol glycoside by contacting a starting composition comprising a substrate steviol glycoside, sucrose, and NDP with an NDP-glucosyltransferase polypeptide and a sucrose synthase, thereby producing a composition comprising a target steviol glycoside comprising one or more additional glucose units than the substrate steviol glycoside.

As used herein, “biocatalysis” or “biocatalytic” refers to the use of natural catalysts, such as protein enzymes, to perform chemical transformations on organic compounds. Biocatalysis is alternatively known as biotransformation or biosynthesis. Both isolated and whole cell biocatalysis methods are known in the art. Biocatalyst protein enzymes can be naturally occurring or recombinant proteins.

As used herein, the term “steviol glycoside(s)” refers to a glycoside of steviol, including, but not limited to, naturally occurring steviol glycosides, e.g. steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, synthetic steviol glycosides, e.g. enzymatically glucosylated steviol glycosides and combinations thereof.

As used herein, “starting composition” refers to any composition (generally an aqueous solution) containing one or more steviol glycosides, where the one or more steviol glycosides serve as the substrate for the biotransformation.

As used herein, the terms “polynucleotide” or “nucleic acid” are used interchangeably, unless indicated by context, and is used to refer to a polymeric form of nucleotides of any length, either ribonucleotides or deoxyribonucleotides, typically DNA.

As used herein, “expression” refers to either or both steps, depending on context, of the two-step process by which polynucleotides are transcribed into mRNA and the transcribed mRNA is subsequently translated into polypeptides.

“Under transcriptional control” means that transcription of a polynucleotide, usually a DNA sequence, depends on its being operatively linked to an element that promotes transcription.

“Operatively linked” means that the polynucleotide elements are arranged in a manner that allows them to function in a cell; typically to produce polypeptides in the cell; for example, the disclosure provides promoters operatively linked to the downstream sequences encoding polypeptides.

The term “encode” refers to the ability of a polynucleotide to produce an mRNA or a polypeptide if it can be transcribed to produce the mRNA and then translated to produce the polypeptide or a fragment thereof. In each case, the polynucleotide is referred to as encoding the mRNA and encoding the polypeptide. The antisense strand is the complement of such a nucleic acid, and the encoding sequence can be deduced therefrom. Similarly, a “coding sequence” refers to a region of a nucleic acid that encodes an mRNA or a polypeptide.

The term “promoter” as used herein refers to a control sequence that is a portion of a polynucleotide sequence that controls the initiation and rate of transcription of a coding sequence. An “enhancer” is a regulatory element that increases the expression of a target sequence. A “promoter/enhancer” is a polynucleotide with sequences that provide both promoter and enhancer functions.

The regulatory elements, e.g. enhancers and promoters, may be “homologous” or “heterologous.” A “homologous” regulatory element is one which is naturally linked with a given polynucleotide in the genome; for example, it may be the promoter found natively in the organism upstream of the encoded polypeptide. A “heterologous” regulatory element is one which is placed in juxtaposition to a polynucleotide by means of recombinant molecular biological techniques but is not a combination found in nature. Often, promoters, enhancers and other regulatory elements are heterologous so as to facilitate expression of a polypeptide in a host cell other than one in which a polypeptide naturally occurs. Thus, “heterologous expression”, as used herein, refers to producing an mRNA and/or a polypeptide in a host cell, such as a microorganism, where the polynucleotide is not found naturally or one or more regulatory elements are not naturally found operably linked to the polynucleotide in the host cell.

The term “polypeptide” is used here to refer to a molecule of two or more subunits of amino acids linked by peptide bonds. Typically, though not always, the polypeptides contain several hundred amino acids; for example, about 400 to about 800 amino acids.

A “plasmid” is a DNA molecule that is typically separate from and capable of replicating independently of the chromosomal DNA. In many cases, it is circular and double-stranded. It is known in the art that while plasmid vectors often exist as extrachromosomal circular DNA molecules, plasmid vectors may also be designed to be stably integrated into a host chromosome either randomly or in a targeted manner. Many plasmids are commercially available for varied uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Typically, the polypeptides disclosed herein are expressed from plasmids.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein the terms “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “microbes” of lists and figures present in the disclosure. This characterization can refer to not only the identified taxonomic genera but also the identified taxonomic species, as well as the various novel and newly identified or designed strains of any organism in said tables or figures. The same characterization holds true for the recitation of these terms in other parts of the Specification, such as in the Examples.

When referring to a nucleic acid sequence or protein sequence, the term “identity” is used to denote similarity between two sequences. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl.

Acid Res. 12, 387-395 (1984), or by inspection. Another suitable algorithm is the BLAST algorithm, described in Altschul et al., J Mol. Biol. 215, 403-410, (1990) and Karlin et al., Proc. Natl. Acad. Sci. USA 90, 5873-5787 (1993). An exemplary BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the sequence and composition of the particular database against which the sequence of interest is being searched; however, the values may be adjusted to increase sensitivity. Another algorithm is gapped BLAST as reported by Altschul et al, (1997) Nucleic Acids Res. 25, 3389-3402. Other algorithms may be described herein.

The present disclosure provides non-natural, engineered B-1,2-ADP glycosyltransferases (B12GTs) that can use an ADP-glucose sugar donor to convert stevioside to Reb E and Reb A to Reb D. In a particular embodiment, the glucosyltransferase polypeptide is one of SEQ ID NOs: 6-882 and 1333-1466. In another embodiment, the glucosyltransferase polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or 100% identical to one of SEQ ID NOs: 6-882 and 1333-1466.

In bioinformatics, several methods have been developed to find and determine related polypeptide sequences. For example, percent sequence identity, position-specific scoring matrices (PSSMs) and hidden Markov models (HMMs) are all commonly employed to find sequences that are similar to a given query sequence. Percent sequence identity calculates the number of amino acids that are shared between two sequences. Percent sequence identity is calculated in the context of a given alignment between two sequences. Percentage identity may be calculated using the alignment program Clustal Omega (available at /www.ebi.ac.uk/Tools/msa/clustalo/) with default settings. The default transition matrix is Gonnet, gap opening penalty is 6 bits, and gap extension is 1 bit. Clustal Omega uses the HHalign algorithm and its default settings as its core alignment engine. The algorithm is described in Söding, J. (2005) ‘Protein homology detection by HMM-HMM comparison’. Bioinformatics 21, 951-960.

Position-specific scoring matrices (PSSMs) are a concise way to represent many related sequences. PSSMs are often generated using multiple sequence alignments. The sequence search tool PSI-BLAST generates PSSMs and uses them to search for related polypeptide sequences. A PSSM used to score polypeptide sequences is a matrix (i.e. table) composed of 21 columns by N rows, where N is the length of the related sequences. Each row corresponds to a position within the polypeptide sequence and each column represents a different amino acid (or gap) that the residue position can take on. Each entry in the PSSM represents a score for the specific amino acid at the specific position within the polypeptide sequence. A sequence can be scored with a PSSM by first aligning the sequence to a reference sequence, and then calculating the following sum: S_{PSSM}=Σᵢ₌₁^{N} PSSM (i, aaᵢ), where i is the sequence position and aaᵢ is the amino acid at position i. Related polypeptide sequences will all have high PSSM scores, while unrelated sequences will yield low scores.

The present disclosure also provides non-natural, engineered sucrose synthases (SuSys) that can use a sucrose sugar donor to convert ADP to ADP-glucose. In a particular embodiment, the SuSy polypeptide is one of SEQ ID NOs: 890-1227 and 1231-1332. In another embodiment, the SuSy polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or 100% identical to one of SEQ ID NOs: 890-1227 and 1231-1332.

In some embodiments, the glucosyltransferase and/or sucrose synthase polypeptides are prepared by expression in a host microorganism. Suitable host microorganisms include, but are not limited to, E. coli, Saccharomyces sp., Aspergillus sp., Pichia sp., Bacillus sp. In a particular embodiment, the glucosyltransferase and sucrose synthase are expressed in E. coli. In a particular embodiment, the glucosyltransferase and sucrose synthase are expressed in Pichia pastoris.

The B12GT and/or SuSy polypeptide can be provided in any suitable form, including free, immobilized, or as a whole cell system. The degree of purity of the glucosyltransferase polypeptide may vary, e.g., it may be provided as a crude, semi-purified, or purified enzyme preparation(s). In one embodiment, the glycosyltransferase polypeptide is free. In another embodiment, the glycosyltransferase polypeptide is immobilized to a solid support, for example on an inorganic or organic support. In some embodiments, the solid support is derivatized cellulose, glass, ceramic, methacrylate, styrene, acrylic, a metal oxide, or a membrane. In some embodiments, the glucosyltransferase polypeptide is immobilized to the solid support by covalent attachment, adsorption, cross-linking, entrapment, or encapsulation.

In yet another embodiment, the B12GT and/or SuSy polypeptide is provided in the form of a whole cell system, for example as a living fermentative microbial cell, or as dead and stabilized microbial cell, or in the form of a cell lysate.

The present disclosure provides a biocatalytic process for the preparation of a composition comprising a target steviol glycoside from a starting composition comprising a substrate steviol glycoside, wherein the target steviol glycoside comprises one or more additional glucose units than the substrate steviol glycoside. The biocatalytic process comprises contacting a B12GT and a SuSy with a starting composition comprising one or more steviol glycosides, a non-UDP nucleotide diphosphate, and sucrose. In another embodiment, the biocatalytic process comprises contacting an engineered B12GT and a SuSy with a starting composition comprising one or more steviol glycosides, a non-UDP nucleotide diphosphate, and sucrose. In another embodiment, the biocatalytic process comprises contacting an engineered B12GT and an engineered SuSy with a starting composition comprising one or more steviol glycosides, a non-UDP nucleotide diphosphate, and sucrose. In some embodiments, the method comprises contacting RA50, ADP, and sucrose with an engineered B1,2 glycosyltransferase and a sucrose synthase to make Reb D and Reb E.

In one embodiment, the B12GT polypeptide is one of SEQ ID NOs: 1-882 and 1333-1466. In another embodiment, the glucosyltransferase polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or 100% identical to one of SEQ ID NOs: 1-882. In another embodiment, the glucosyltransferase polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or 100% identical to one of SEQ ID NOs: 6-882 and 1333-1466. Preferably, the catalytic domain in the B12GT polypeptide contains residues corresponding to H at position 15, D at position 114, D at position 357, and Q at position 358, numbered according to SEQ ID NO: 5.

In one embodiment, the sucrose synthase is any polypeptide with sucrose synthase activity. In another embodiment, the sucrose synthase is derived from an organism from the Bacteria domain. In another embodiment, the sucrose synthase is derived from an organism from the Plantae kingdom. In another embodiment, the sucrose synthase is derived from an organism from the Plantae kingdom. In another embodiment, the sucrose synthase is derived from an organism from the proteobacteria, deferribacteres, or cyanobacteria phylum. In another embodiment, the sucrose synthase is derived from the species Acidithiobacillus caldus, Nitrosomonas europaea, Denitrovibrio acetiphilus, Thermosynechococcus elongatus, Oryza sativa, Arabidopsis thaliana, or Coffea arabica. In one embodiment, the sucrose synthase is one of SEQ ID NOs: 883-1227 and 1231-1332. In another embodiment, the sucrose synthase is an engineered sucrose synthase with a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to one of SEQ ID NOs: 883-1227. In another embodiment, the sucrose synthase is an engineered sucrose synthase with a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to one of SEQ ID NOs: 890-1227 and 1231-1332. Preferably, the catalytic domain in the SuSy polypeptide contains residues corresponding to H at position 425, R at position 567, K at position 572, and E at position 663, numbered according to SEQ ID NO: 885 or residues corresponding to H at position 436, R at position 578, K at position 583, and E at position 674, numbered according to SEQ ID NO: 888.

In some embodiments, the glucosyltransferase and/or sucrose synthase polypeptides are prepared by expression in a host microorganism. Suitable host microorganisms include, but are not limited to, E. coli, Saccharomyces sp., Aspergillus sp., Pichia sp., Bacillus sp. In a particular embodiment, the glucosyltransferase and sucrose synthase are expressed in E. coli. In another embodiment, the glucosyltransferase and sucrose synthase are expressed in Pichia pastoris. In another embodiment, the glucosyltransferase and/or sucrose synthase polypeptides are prepared by cell-free expression.

The B12GT and sucrose synthase polypeptides can be provided in any suitable form, including free, immobilized, or as a whole cell system. The degree of purity of the polypeptides may vary, e.g., they may be provided as a crude, semi-purified, or purified enzyme preparation(s). In one embodiment, the B12GT and/or SuSy polypeptide is free. In another embodiment, the B12GT and/or SuSy polypeptide is immobilized to a solid support, for example on an inorganic or organic support. In some embodiments, the solid support is derivatized cellulose, glass, ceramic, methacrylate, styrene, acrylic, a metal oxide, or a membrane. In some embodiments, the B12GT and/or SuSy polypeptide is immobilized to the solid support by covalent attachment, adsorption, cross-linking, entrapment, or encapsulation.

In yet another embodiment, the B12GT and/or SuSy polypeptide is provided in the form of a whole cell system, for example as a living fermentative microbial cell, or as dead and stabilized microbial cell, or in the form of a cell lysate.

The steviol glycoside component(s) of the starting composition serves as a substrate(s) for the production of the target steviol glycoside(s), as described herein. The target steviol glycoside target(s) differs chemically from its corresponding substrate steviol glycoside(s) by the addition of one or more glucose units.

The starting steviol glycoside composition can contain at least one substrate steviol glycoside. In an embodiment, the substrate steviol glycoside is selected from the group consisting of steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof. In another embodiment, the starting steviol glycoside composition is composed of stevioside and Reb A. In another embodiment, the starting steviol glycoside composition is composed of stevioside. In yet another embodiment, the starting steviol glycoside composition is composed of Reb A.

The starting steviol glycoside composition may be synthetic or purified (partially or entirely), commercially available or prepared. One example of a starting composition useful in the method of the present disclosure is an extract obtained from purification of Stevia rebaudiana plant material (e.g. leaves). Another example of a starting composition is a commercially available stevia extract brought into solution with a solvent. Yet another example of a starting composition is a commercially available mixture of steviol glycosides brought into solution with a solvent. Other suitable starting compositions include by-products of processes to isolate and purify steviol glycosides.

In one embodiment, the starting composition comprises a purified substrate steviol glycoside. For example, the starting composition may comprise greater than about 50%, greater than about 60%, greater than about 70%, greater than about 80%, greater than about 85%, greater than about 90%, greater than about 91%, greater than about 92%, greater than about 93%, greater than about 94%, greater than about 95%, greater than about 96%, greater than about 97%, greater than about 98%, greater than about 99%, or greater than about 99.6% of one or more substrate steviol glycosides by weight on an anhydrous basis.

In another embodiment, the starting composition comprises a partially purified substrate steviol glycoside composition. For example, the starting composition contains greater than about 0.5%, greater than about 1%, greater than about 2%, greater than about 3%, greater than about 4%, greater than about 5%, greater than about 10%, greater than about 20%, greater than about 30%, greater than about 40%, or greater than about 50%, of one or more substrate steviol glycosides by weight on an anhydrous basis.

In another embodiment, the substrate steviol glycoside is purified rebaudioside A, or isomers thereof. In a particular embodiment, the substrate steviol glycoside contains greater than 99% rebaudioside A, or isomers thereof, by weight on an anhydrous basis. In another embodiment, the substrate steviol glycoside comprises partially purified rebaudioside A. In a particular embodiment, the substrate steviol glycoside contains greater than about 1%, 5%, 10%, 20%, 30%, 40%, 50%, 60%, 70%, 80% or 90% rebaudioside A by weight on an anhydrous basis.

In yet another embodiment, the substrate steviol glycoside comprises purified stevioside, or isomers thereof. In a particular embodiment, the substrate steviol glycoside contains greater than 99% stevioside, or isomers thereof, by weight on an anhydrous basis. In another embodiment, the substrate steviol glycoside comprises partially purified stevioside. In a particular embodiment, the substrate steviol glycoside contains greater than about 10%, 20%, 30%, 40%, 50%, 60%, 70%, 80% or 90% stevioside by weight on an anhydrous basis.

In yet another embodiment, the substrate steviol glycoside is a combination of stevioside and rebaudioside A. In a particular embodiment, the substrate steviol glycoside contains greater than about 5% stevioside and greater than about 5% Reb A, greater than about 10% stevioside and greater than about 10% Reb A, greater than about 20% stevioside and greater than about 20% Reb A, greater than about 30% stevioside and greater than about 30% Reb A, greater than about 40% stevioside and greater than about 40% Reb A, greater than about 45% stevioside and greater than about 45% Reb A, greater than about 40% stevioside and greater than about 50% Reb A, greater than about 30% stevioside and greater than about 60% Reb A, greater than about 20% stevioside and greater than about 70% Reb A, greater than about 10% stevioside and greater than about 80% Reb A, greater than about 5% stevioside and greater than about 90% Reb A, greater than about 50% stevioside and greater than about 40% Reb A, greater than about 60% stevioside and greater than about 30% Reb A, greater than about 70% stevioside and greater than about 20% Reb A, greater than about 80% stevioside and greater than about 10% Reb A, or greater than about 90% stevioside and greater than about 5% Reb A by weight on an anhydrous basis.

In still another embodiment, the substrate steviol glycoside is derived from stevia leaf extract. In one embodiment, RA50, stevia leaf extract purified to contain greater than 50% Reb A, is used as the steviol glycoside substrate. In one embodiment, RA50 is used at a concentration between about 1 and 800 mg/mL. In another embodiment, RA50 is used at a concentration of about 100 mg/mL.

The one pot reaction can be carried out with a nucleotide cofactor that can be converted to an NDP-glucose by sucrose synthase. In some embodiments, the nucleotide can be a non-UDP nucleotide (i.e. ADP-glucose, GDP-glucose, CDP-glucose, or TDP-glucose). In another embodiment, the nucleotide is ADP. In a particular embodiment, the one pot reaction can be carried out with ADP at a concentration between about 0.01 and 10 mM, such as, for example, between 0.01 mM and 0.05 mM, between 0.05 mM and 0.1 mM, between 0.1 mM and 0.5 mM, between 0.5 mM and 1 mM, between 1 mM and 5 mM, or between 5 mM and 10 mM. In a particular embodiment, ADP is used at a concentration of 0.5 mM.

The one pot reaction can be carried out with a sucrose concentration between about 10 mM and 2M, such as, for example, greater than 10 mM, greater than 50 mM, greater than 100 mM, greater than 250 mM, greater than 500 mM, greater than 1 M, greater than 1.5 M and greater than 2 M. In a particular embodiment, sucrose is used at a concentration of 250 mM.

In one embodiment, the reaction is run at any temperature. In another embodiment, the one-pot reaction is run at a temperature between about 10° C. and 80° C. Such as, for example, between 10° C. to 20° C., between 20° C. to 30° C., between 30° C. to 40° C., between 40° C. to 50° C., between 50° C. to 60° C., between 60° C. to 70° C., between 70° C. to 80° C. or 80° C. In a particular embodiment, the one-pot reaction is carried out at 60° C.

The reaction medium for conversion is generally aqueous, e.g., purified water, buffer, or a combination thereof. In a particular embodiment, the reaction medium is a buffer. Suitable buffers include, but are not limited to, acetate buffer, citrate buffer, HEPES, and phosphate buffer. In a particular embodiment, the reaction medium is phosphate buffer. The reaction medium can have a pH between about 4 and 10. In a particular embodiment, the reaction medium has a pH of 6. The reaction medium can also be, alternatively, an organic solvent.

The step of contacting the starting composition with the glycosyltransferase and sucrose synthase polypeptides can be carried out in a duration of time between about 1 hour and 1 week, such as, for example, between 30 minutes and 1 hours, between 1 hour and 4 hours, between 4 hours and 6 hours, between 6 hours and 12 hours, between 12 hours and 24 hours, between 1 day and 2 days, between 2 days and 3 days, 3 days and 4 days, between 4 days and 5 days, between 6 days and 7 days. In a particular embodiment, the reaction is carried out for 24 hours.

The reaction can be monitored by suitable method including, but not limited to, HPLC, LCMS, TLC, IR or NMR.

The target steviol glycoside can be any steviol glycoside. In one embodiment, the target steviol glycoside is steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, a rebaudioside with 7 covalently attached glucose units (e.g. rebaudioside M plus 1 glucose unit), a synthetic steviol glycoside, an isomer thereof, and/or a steviol glycoside composition. In another embodiment, the target steviol glycoside is rebaudioside E, or isomers thereof. In another embodiment, the target steviol glycoside is rebaudioside D, or isomers thereof. In still another embodiment, the target steviol glycosides are Reb D and Reb E.

In one embodiment, the conversion of Reb A to Reb D and/or Reb D isomer(s) is at least about 2% complete, as determined by any of the methods mentioned above. In a particular embodiment, the conversion of Reb A to Reb D and/or Reb D isomer(s) is at least about 10% complete, at least about 20% complete, at least about 30% complete, at least about 40% complete, at least about 50% complete, at least about 60% complete, at least about 70% complete, at least about 80% complete, or at least about 90% complete. In a particular embodiment, the conversion of reb A to reb D and/or rebD isomer(s) is at least about 95% complete. In some embodiments, at least about 5%, 10%, 20%, 30%, 40%, 50%, 60%, 70%, 80%, or 90% of the Reb A in the starting composition is converted to Reb D and/or Reb D isomer(s).

In one embodiment, the conversion of stevioside to Reb E and/or Reb E isomer(s) is at least about 2% complete, as determined by any of the methods mentioned above. In a particular embodiment, the conversion of stevioside to Reb E and/or Reb E isomer(s) is at least about 10% complete, at least about 20% complete, at least about 30% complete, at least about 40% complete, at least about 50% complete, at least about 60% complete, at least about 70% complete, at least about 80% complete, or at least about 90% complete. In a particular embodiment, the conversion of stevioside to Reb E and/or Reb E isomer(s) is at least about 95% complete. In some embodiments, at least about 5%, 10%, 20%, 30%, 40%, 50%, 60%, 70%, 80%, or 90% of the stevioside in the starting composition is converted to Reb E and/or Reb E isomer(s).

The target steviol glycoside(s) can be in any polymorphic or amorphous form, including hydrates, solvates, anhydrous or combinations thereof.

Optionally, the method of the present disclosure further comprises separating the target steviol glycoside from the target composition. The target steviol glycoside(s) can be separated by any suitable method, such as, for example, crystallization, separation by membranes, centrifugation, extraction, chromatographic separation or a combination of such methods.

In one embodiment, the separation of target steviol glycosides produces a composition comprising greater than about 80% by weight of the target steviol glycoside(s) on an anhydrous basis, i.e., a highly purified steviol glycoside composition. In another embodiment, separation produces a composition comprising greater than about 0.5%, greater than about 1%, greater than about 2%, greater than about 3%, greater than about 4%, greater than about 5%, greater than about 10%, greater than about 20%, greater than about 30%, greater than about 40%, greater than about 50%, greater than about 60%, greater than about 70%, greater than about 80%, greater than about 85%, greater than about 90%, greater than about 91%, greater than about 92%, greater than about 93%, greater than about 94%, greater than about 95%, greater than about 96%, greater than about 97%, greater than about 98%, greater than about 99%, or greater than about 99.6% by weight of the target steviol glycosides. In particular embodiments, the composition comprises greater than about 95% by weight of the target steviol glycoside(s). Purified target steviol glycosides can be used in consumable products as a sweetener.

Suitable consumer products include, but are not limited to, food, beverages, pharmaceutical compositions, tobacco products, nutraceutical compositions, oral hygiene compositions, and cosmetic compositions.

Plasmids containing nucleic acids encoding enzymes having SEQ ID NOS: 1-1227 and 1231-1466 are described in the Table 1 below.

TABLE 1

| SEQ ID | Plasmid |
|---|---|
| NO | ID |
| 1 | pA10132 |
| 2 | pA10143 |
| 3 | pA12549 |
| 4 | pA12550 |
| 5 | pA28422 |
| 6 | pA30328 |
| 7 | pA30329 |
| 8 | pA30331 |
| 9 | pA30332 |
| 10 | pA30333 |
| 11 | pA30334 |
| 12 | pA30335 |
| 13 | pA30337 |
| 14 | pA30338 |
| 15 | pA30339 |
| 16 | pA30340 |
| 17 | pA30341 |
| 18 | pA30342 |
| 19 | pA30343 |
| 20 | pA30345 |
| 21 | pA30347 |
| 22 | pA30348 |
| 23 | pA30350 |
| 24 | pA30351 |
| 25 | pA30352 |
| 26 | pA30354 |
| 27 | pA30358 |
| 28 | pA30362 |
| 29 | pA30463 |
| 30 | pA30468 |
| 31 | pA30471 |
| 32 | pA30472 |
| 33 | pA30473 |
| 34 | pA30557 |
| 35 | pA30565 |
| 36 | pA30568 |
| 37 | pA30569 |
| 38 | pA30570 |
| 39 | pA30571 |
| 40 | pA30785 |
| 41 | pA30787 |
| 42 | pA30790 |
| 43 | pA30791 |
| 44 | pA30793 |
| 45 | pA30796 |
| 46 | pA30797 |
| 47 | pA30798 |
| 48 | pA30799 |
| 49 | pA30802 |
| 50 | pA30803 |
| 51 | pA30805 |
| 52 | pA30806 |
| 53 | pA30808 |
| 54 | pA30811 |
| 55 | pA30812 |
| 56 | pA30814 |
| 57 | pA30817 |
| 58 | pA30820 |
| 59 | pA30879 |
| 60 | pA30880 |
| 61 | pA30882 |
| 62 | pA30883 |
| 63 | pA30884 |
| 64 | pA30885 |
| 65 | pA30886 |
| 66 | pA30887 |
| 67 | pA30888 |
| 68 | pA30892 |
| 69 | pA30894 |
| 70 | pA30895 |
| 71 | pA30935 |
| 72 | pA30936 |
| 73 | pA30937 |
| 74 | pA30938 |
| 75 | pA30939 |
| 76 | pA30940 |
| 77 | pA30941 |
| 78 | pA30942 |
| 79 | pA30943 |
| 80 | pA30944 |
| 81 | pA30945 |
| 82 | pA30946 |
| 83 | pA30947 |
| 84 | pA30948 |
| 85 | pA30949 |
| 86 | pA30951 |
| 87 | pA30952 |
| 88 | pA30953 |
| 89 | pA30997 |
| 90 | pA30998 |
| 91 | pA31001 |
| 92 | pA31003 |
| 93 | pA31004 |
| 94 | pA31005 |
| 95 | pA31006 |
| 96 | pA31009 |
| 97 | pA31011 |
| 98 | pA31012 |
| 99 | pA31014 |
| 100 | pA31016 |
| 101 | pA31017 |
| 102 | pA31018 |
| 103 | pA31020 |
| 104 | pA31022 |
| 105 | pA31024 |
| 106 | pA31026 |
| 107 | pA31029 |
| 108 | pA31068 |
| 109 | pA31069 |
| 110 | pA31070 |
| 111 | pA31071 |
| 112 | pA31073 |
| 113 | pA31074 |
| 114 | pA31075 |
| 115 | pA31076 |
| 116 | pA31078 |
| 117 | pA31080 |
| 118 | pA31081 |
| 119 | pA31086 |
| 120 | pA31125 |
| 121 | pA31126 |
| 122 | pA31127 |
| 123 | pA31128 |
| 124 | pA31129 |
| 125 | pA31130 |
| 126 | pA31134 |
| 127 | pA31135 |
| 128 | pA31136 |
| 129 | pA31138 |
| 130 | pA31142 |
| 131 | pA31163 |
| 132 | pA31167 |
| 133 | pA31169 |
| 134 | pA31172 |
| 135 | pA31173 |
| 136 | pA31174 |
| 137 | pA31175 |
| 138 | pA31177 |
| 139 | pA31178 |
| 140 | pA31179 |
| 141 | pA31181 |
| 142 | pA31258 |
| 143 | pA31259 |
| 144 | pA31261 |
| 145 | pA31262 |
| 146 | pA31263 |
| 147 | pA31264 |
| 148 | pA31265 |
| 149 | pA31267 |
| 150 | pA31268 |
| 151 | pA31273 |
| 152 | pA31274 |
| 153 | pA31276 |
| 154 | pA31278 |
| 155 | pA31279 |
| 156 | pA31280 |
| 157 | pA31282 |
| 158 | pA31285 |
| 159 | pA31286 |
| 160 | pA31287 |
| 161 | pA31288 |
| 162 | pA31289 |
| 163 | pA31290 |
| 164 | pA31291 |
| 165 | pA31294 |
| 166 | pA31297 |
| 167 | pA31298 |
| 168 | pA31300 |
| 169 | pA31302 |
| 170 | pA31303 |
| 171 | pA31304 |
| 172 | pA31305 |
| 173 | pA31306 |
| 174 | pA31307 |
| 175 | pA31308 |
| 176 | pA31309 |
| 177 | pA31310 |
| 178 | pA31311 |
| 179 | pA31313 |
| 180 | pA31336 |
| 181 | pA31345 |
| 182 | pA31346 |
| 183 | pA31347 |
| 184 | pA31350 |
| 185 | pA31352 |
| 186 | pA31353 |
| 187 | pA31354 |
| 188 | pA31355 |
| 189 | pA31356 |
| 190 | pA31358 |
| 191 | pA31360 |
| 192 | pA31362 |
| 193 | pA31364 |
| 194 | pA31366 |
| 195 | pA31367 |
| 196 | pA31368 |
| 197 | pA31369 |
| 198 | pA31505 |
| 199 | pA31506 |
| 200 | pA31507 |
| 201 | pA31508 |
| 202 | pA31509 |
| 203 | pA31510 |
| 204 | pA31511 |
| 205 | pA31512 |
| 206 | pA31513 |
| 207 | pA31515 |
| 208 | pA31516 |
| 209 | pA31517 |
| 210 | pA31518 |
| 211 | pA31519 |
| 212 | pA31520 |
| 213 | pA31521 |
| 214 | pA31522 |
| 215 | pA31523 |
| 216 | pA31545 |
| 217 | pA31548 |
| 218 | pA31549 |
| 219 | pA31558 |
| 220 | pA31559 |
| 221 | pA31560 |
| 222 | pA31561 |
| 223 | pA29916 |
| 224 | pA29917 |
| 225 | pA29918 |
| 226 | pA29919 |
| 227 | pA29920 |
| 228 | pA29921 |
| 229 | pA29922 |
| 230 | pA29923 |
| 231 | pA29924 |
| 232 | pA29925 |
| 233 | pA29926 |
| 234 | pA29927 |
| 235 | pA29928 |
| 236 | pA29929 |
| 237 | pA29930 |
| 238 | pA29931 |
| 239 | pA29932 |
| 240 | pA29933 |
| 241 | pA29934 |
| 242 | pA29935 |
| 243 | pA29937 |
| 244 | pA29938 |
| 245 | pA29939 |
| 246 | pA29941 |
| 247 | pA29942 |
| 248 | pA29943 |
| 249 | pA29944 |
| 250 | pA29945 |
| 251 | pA29946 |
| 252 | pA29947 |
| 253 | pA29948 |
| 254 | pA29949 |
| 255 | pA29950 |
| 256 | pA29951 |
| 257 | pA29952 |
| 258 | pA29953 |
| 259 | pA29954 |
| 260 | pA29955 |
| 261 | pA29956 |
| 262 | pA29957 |
| 263 | pA29958 |
| 264 | pA29959 |
| 265 | pA29960 |
| 266 | pA29961 |
| 267 | pA29962 |
| 268 | pA29963 |
| 269 | pA29964 |
| 270 | pA29965 |
| 271 | pA29966 |
| 272 | pA29967 |
| 273 | pA29968 |
| 274 | pA29969 |
| 275 | pA29970 |
| 276 | pA29971 |
| 277 | pA29972 |
| 278 | pA29973 |
| 279 | pA29976 |
| 280 | pA29977 |
| 281 | pA29978 |
| 282 | pA29979 |
| 283 | pA29980 |
| 284 | pA29981 |
| 285 | pA29982 |
| 286 | pA29983 |
| 287 | pA29984 |
| 288 | pA29985 |
| 289 | pA29986 |
| 290 | pA29987 |
| 291 | pA29989 |
| 292 | pA29990 |
| 293 | pA29991 |
| 294 | pA29992 |
| 295 | pA29993 |
| 296 | pA29994 |
| 297 | pA29995 |
| 298 | pA29996 |
| 299 | pA29997 |
| 300 | pA29998 |
| 301 | pA30000 |
| 302 | pA30001 |
| 303 | pA30002 |
| 304 | pA30003 |
| 305 | pA30004 |
| 306 | pA30005 |
| 307 | pA30006 |
| 308 | pA30007 |
| 309 | pA30008 |
| 310 | pA30009 |
| 311 | pA30010 |
| 312 | pA30011 |
| 313 | pA30012 |
| 314 | pA30013 |
| 315 | pA30014 |
| 316 | pA29705 |
| 317 | pA29706 |
| 318 | pA29707 |
| 319 | pA29708 |
| 320 | pA29709 |
| 321 | pA29710 |
| 322 | pA29711 |
| 323 | pA29712 |
| 324 | pA29713 |
| 325 | pA29714 |
| 326 | pA29715 |
| 327 | pA29716 |
| 328 | pA29717 |
| 329 | pA29718 |
| 330 | pA29719 |
| 331 | pA29720 |
| 332 | pA29721 |
| 333 | pA29722 |
| 334 | pA29723 |
| 335 | pA29724 |
| 336 | pA29725 |
| 337 | pA29726 |
| 338 | pA29727 |
| 339 | pA29728 |
| 340 | pA29729 |
| 341 | pA29730 |
| 342 | pA29731 |
| 343 | pA29732 |
| 344 | pA29733 |
| 345 | pA29734 |
| 346 | pA29735 |
| 347 | pA29736 |
| 348 | pA29737 |
| 349 | pA29738 |
| 350 | pA29740 |
| 351 | pA29741 |
| 352 | pA29742 |
| 353 | pA29743 |
| 354 | pA29744 |
| 355 | pA29745 |
| 356 | pA29746 |
| 357 | pA29747 |
| 358 | pA29748 |
| 359 | pA29749 |
| 360 | pA29750 |
| 361 | pA29751 |
| 362 | pA29752 |
| 363 | pA29753 |
| 364 | pA29754 |
| 365 | pA29755 |
| 366 | pA29756 |
| 367 | pA29757 |
| 368 | pA29758 |
| 369 | pA29759 |
| 370 | pA29760 |
| 371 | pA29761 |
| 372 | pA29762 |
| 373 | pA29763 |
| 374 | pA29764 |
| 375 | pA29765 |
| 376 | pA32567 |
| 377 | pA32568 |
| 378 | pA32569 |
| 379 | pA32570 |
| 380 | pA32571 |
| 381 | pA32572 |
| 382 | pA32573 |
| 383 | pA32574 |
| 384 | pA32576 |
| 385 | pA33083 |
| 386 | pA33084 |
| 387 | pA33085 |
| 388 | pA33086 |
| 389 | pA33087 |
| 390 | pA33088 |
| 391 | pA33089 |
| 392 | pA33090 |
| 393 | pA33091 |
| 394 | pA33092 |
| 395 | pA33094 |
| 396 | pA33095 |
| 397 | pA33096 |
| 398 | pA33097 |
| 399 | pA33098 |
| 400 | pA33099 |
| 401 | pA33100 |
| 402 | pA33101 |
| 403 | pA33102 |
| 404 | pA33103 |
| 405 | pA33104 |
| 406 | pA33105 |
| 407 | pA33106 |
| 408 | pA33107 |
| 409 | pA33108 |
| 410 | pA33109 |
| 411 | pA33110 |
| 412 | pA33111 |
| 413 | pA33112 |
| 414 | pA33114 |
| 415 | pA33115 |
| 416 | pA33116 |
| 417 | pA33117 |
| 418 | pA33118 |
| 419 | pA33119 |
| 420 | pA33120 |
| 421 | pA33122 |
| 422 | pA33123 |
| 423 | pA33124 |
| 424 | pA33125 |
| 425 | pA33129 |
| 426 | pA33130 |
| 427 | pA33131 |
| 428 | pA33132 |
| 429 | pA33133 |
| 430 | pA33134 |
| 431 | pA33135 |
| 432 | pA33136 |
| 433 | pA33137 |
| 434 | pA33138 |
| 435 | pA33139 |
| 436 | pA33140 |
| 437 | pA33141 |
| 438 | pA33142 |
| 439 | pA33143 |
| 440 | pA33144 |
| 441 | pA33145 |
| 442 | pA33146 |
| 443 | pA33147 |
| 444 | pA33148 |
| 445 | pA33149 |
| 446 | pA33150 |
| 447 | pA33151 |
| 448 | pA33152 |
| 449 | pA33153 |
| 450 | pA33154 |
| 451 | pA33159 |
| 452 | pA33160 |
| 453 | pA33162 |
| 454 | pA33163 |
| 455 | pA33164 |
| 456 | pA33165 |
| 457 | pA33166 |
| 458 | pA33167 |
| 459 | pA33168 |
| 460 | pA32557 |
| 461 | pA32558 |
| 462 | pA32559 |
| 463 | pA32560 |
| 464 | pA32562 |
| 465 | pA32563 |
| 466 | pA32565 |
| 467 | pA32566 |
| 468 | pA33169 |
| 469 | pA33171 |
| 470 | pA33172 |
| 471 | pA33173 |
| 472 | pA33174 |
| 473 | pA33175 |
| 474 | pA33176 |
| 475 | pA33177 |
| 476 | pA33178 |
| 477 | pA33179 |
| 478 | pA33180 |
| 479 | pA33181 |
| 480 | pA33182 |
| 481 | pA33184 |
| 482 | pA33185 |
| 483 | pA33186 |
| 484 | pA33188 |
| 485 | pA33189 |
| 486 | pA33190 |
| 487 | pA33191 |
| 488 | pA33192 |
| 489 | pA33193 |
| 490 | pA33194 |
| 491 | pA33195 |
| 492 | pA33196 |
| 493 | pA33197 |
| 494 | pA33198 |
| 495 | pA33199 |
| 496 | pA33200 |
| 497 | pA33201 |
| 498 | pA33202 |
| 499 | pA33203 |
| 500 | pA33204 |
| 501 | pA33205 |
| 502 | pA33206 |
| 503 | pA33207 |
| 504 | pA33208 |
| 505 | pA33209 |
| 506 | pA33210 |
| 507 | pA33211 |
| 508 | pA33212 |
| 509 | pA33213 |
| 510 | pA33214 |
| 511 | pA33215 |
| 512 | pA33216 |
| 513 | pA33217 |
| 514 | pA33218 |
| 515 | pA33219 |
| 516 | pA33220 |
| 517 | pA33221 |
| 518 | pA33222 |
| 519 | pA33223 |
| 520 | pA33224 |
| 521 | pA33225 |
| 522 | pA33226 |
| 523 | pA33227 |
| 524 | pA33228 |
| 525 | pA33229 |
| 526 | pA33230 |
| 527 | pA33231 |
| 528 | pA33232 |
| 529 | pA33233 |
| 530 | pA33234 |
| 531 | pA33235 |
| 532 | pA33236 |
| 533 | pA33237 |
| 534 | pA33238 |
| 535 | pA29767 |
| 536 | pA29768 |
| 537 | pA29769 |
| 538 | pA29770 |
| 539 | pA29771 |
| 540 | pA29772 |
| 541 | pA29773 |
| 542 | pA29774 |
| 543 | pA29775 |
| 544 | pA29776 |
| 545 | pA29777 |
| 546 | pA29778 |
| 547 | pA29779 |
| 548 | pA29780 |
| 549 | pA29781 |
| 550 | pA29782 |
| 551 | pA29783 |
| 552 | pA29784 |
| 553 | pA29785 |
| 554 | pA29786 |
| 555 | pA29787 |
| 556 | pA29788 |
| 557 | pA29789 |
| 558 | pA29790 |
| 559 | pA29791 |
| 560 | pA29792 |
| 561 | pA29793 |
| 562 | pA29794 |
| 563 | pA29795 |
| 564 | pA29796 |
| 565 | pA29797 |
| 566 | pA29798 |
| 567 | pA29799 |
| 568 | pA29800 |
| 569 | pA29801 |
| 570 | pA29802 |
| 571 | pA29803 |
| 572 | pA29804 |
| 573 | pA29805 |
| 574 | pA29806 |
| 575 | pA29807 |
| 576 | pA29808 |
| 577 | pA29809 |
| 578 | pA29810 |
| 579 | pA29811 |
| 580 | pA29812 |
| 581 | pA29813 |
| 582 | pA29814 |
| 583 | pA29815 |
| 584 | pA29816 |
| 585 | pA29817 |
| 586 | pA29818 |
| 587 | pA29615 |
| 588 | pA29616 |
| 589 | pA29617 |
| 590 | pA29618 |
| 591 | pA29619 |
| 592 | pA29620 |
| 593 | pA29621 |
| 594 | pA29622 |
| 595 | pA29623 |
| 596 | pA29624 |
| 597 | pA29625 |
| 598 | pA29626 |
| 599 | pA29627 |
| 600 | pA29628 |
| 601 | pA29629 |
| 602 | pA29630 |
| 603 | pA29631 |
| 604 | pA29632 |
| 605 | pA29633 |
| 606 | pA29634 |
| 607 | pA29635 |
| 608 | pA29636 |
| 609 | pA29637 |
| 610 | pA29638 |
| 611 | pA29639 |
| 612 | pA29640 |
| 613 | pA29641 |
| 614 | pA29642 |
| 615 | pA29643 |
| 616 | pA29645 |
| 617 | pA29646 |
| 618 | pA29647 |
| 619 | pA29648 |
| 620 | pA29649 |
| 621 | pA29650 |
| 622 | pA29651 |
| 623 | pA29652 |
| 624 | pA29653 |
| 625 | pA29654 |
| 626 | pA29655 |
| 627 | pA29656 |
| 628 | pA29657 |
| 629 | pA29658 |
| 630 | pA29659 |
| 631 | pA29660 |
| 632 | pA29661 |
| 633 | pA29662 |
| 634 | pA29664 |
| 635 | pA29665 |
| 636 | pA29666 |
| 637 | pA29667 |
| 638 | pA29668 |
| 639 | pA29669 |
| 640 | pA29671 |
| 641 | pA29672 |
| 642 | pA29673 |
| 643 | pA29675 |
| 644 | pA29676 |
| 645 | pA29677 |
| 646 | pA29678 |
| 647 | pA29679 |
| 648 | pA29680 |
| 649 | pA29681 |
| 650 | pA29682 |
| 651 | pA29683 |
| 652 | pA29684 |
| 653 | pA29685 |
| 654 | pA29686 |
| 655 | pA29687 |
| 656 | pA29688 |
| 657 | pA29689 |
| 658 | pA29690 |
| 659 | pA29692 |
| 660 | pA29693 |
| 661 | pA29694 |
| 662 | pA29695 |
| 663 | pA29696 |
| 664 | pA29697 |
| 665 | pA29698 |
| 666 | pA29699 |
| 667 | pA29700 |
| 668 | pA29701 |
| 669 | pA29702 |
| 670 | pA29703 |
| 671 | pA29704 |
| 672 | pA30015 |
| 673 | pA30016 |
| 674 | pA30017 |
| 675 | pA30018 |
| 676 | pA30019 |
| 677 | pA30020 |
| 678 | pA30021 |
| 679 | pA30022 |
| 680 | pA30023 |
| 681 | pA30024 |
| 682 | pA30025 |
| 683 | pA30026 |
| 684 | pA30027 |
| 685 | pA30028 |
| 686 | pA30029 |
| 687 | pA30030 |
| 688 | pA30031 |
| 689 | pA30032 |
| 690 | pA30033 |
| 691 | pA30034 |
| 692 | pA30035 |
| 693 | pA30036 |
| 694 | pA30037 |
| 695 | pA30038 |
| 696 | pA30039 |
| 697 | pA30040 |
| 698 | pA30041 |
| 699 | pA30042 |
| 700 | pA30044 |
| 701 | pA30045 |
| 702 | pA30046 |
| 703 | pA30047 |
| 704 | pA30048 |
| 705 | pA30049 |
| 706 | pA30050 |
| 707 | pA32939 |
| 708 | pA32940 |
| 709 | pA32941 |
| 710 | pA32942 |
| 711 | pA32943 |
| 712 | pA32944 |
| 713 | pA32945 |
| 714 | pA32946 |
| 715 | pA32947 |
| 716 | pA32948 |
| 717 | pA32949 |
| 718 | pA32950 |
| 719 | pA32951 |
| 720 | pA32952 |
| 721 | pA32953 |
| 722 | pA32954 |
| 723 | pA32955 |
| 724 | pA32956 |
| 725 | pA32957 |
| 726 | pA32958 |
| 727 | pA32959 |
| 728 | pA32960 |
| 729 | pA32962 |
| 730 | pA32963 |
| 731 | pA32965 |
| 732 | pA32966 |
| 733 | pA32967 |
| 734 | pA32968 |
| 735 | pA32969 |
| 736 | pA32970 |
| 737 | pA32971 |
| 738 | pA32972 |
| 739 | pA32973 |
| 740 | pA32977 |
| 741 | pA32978 |
| 742 | pA32979 |
| 743 | pA32980 |
| 744 | pA32981 |
| 745 | pA32982 |
| 746 | pA32983 |
| 747 | pA32984 |
| 748 | pA32985 |
| 749 | pA32987 |
| 750 | pA32988 |
| 751 | pA32989 |
| 752 | pA32990 |
| 753 | pA32991 |
| 754 | pA32992 |
| 755 | pA32993 |
| 756 | pA32994 |
| 757 | pA32995 |
| 758 | pA32996 |
| 759 | pA32997 |
| 760 | pA32998 |
| 761 | pA32999 |
| 762 | pA33000 |
| 763 | pA33001 |
| 764 | pA33002 |
| 765 | pA33004 |
| 766 | pA33005 |
| 767 | pA33006 |
| 768 | pA33007 |
| 769 | pA33008 |
| 770 | pA33009 |
| 771 | pA33010 |
| 772 | pA33011 |
| 773 | pA33012 |
| 774 | pA33014 |
| 775 | pA33015 |
| 776 | pA33016 |
| 777 | pA33017 |
| 778 | pA33018 |
| 779 | pA33019 |
| 780 | pA33020 |
| 781 | pA33021 |
| 782 | pA33023 |
| 783 | pA33024 |
| 784 | pA33025 |
| 785 | pA33027 |
| 786 | pA33028 |
| 787 | pA33029 |
| 788 | pA33030 |
| 789 | pA33031 |
| 790 | pA33032 |
| 791 | pA33033 |
| 792 | pA33035 |
| 793 | pA33036 |
| 794 | pA33037 |
| 795 | pA33038 |
| 796 | pA33040 |
| 797 | pA33041 |
| 798 | pA33042 |
| 799 | pA33043 |
| 800 | pA33044 |
| 801 | pA33046 |
| 802 | pA33047 |
| 803 | pA33048 |
| 804 | pA33051 |
| 805 | pA33053 |
| 806 | pA33054 |
| 807 | pA33056 |
| 808 | pA33057 |
| 809 | pA33058 |
| 810 | pA33059 |
| 811 | pA33060 |
| 812 | pA33061 |
| 813 | pA33063 |
| 814 | pA33065 |
| 815 | pA33066 |
| 816 | pA33067 |
| 817 | pA33068 |
| 818 | pA33069 |
| 819 | pA33070 |
| 820 | pA33071 |
| 821 | pA33074 |
| 822 | pA33075 |
| 823 | pA33076 |
| 824 | pA33077 |
| 825 | pA33078 |
| 826 | pA33079 |
| 827 | pA33080 |
| 828 | pA33081 |
| 829 | pA33082 |
| 830 | pA29819 |
| 831 | pA29820 |
| 832 | pA29821 |
| 833 | pA29822 |
| 834 | pA29823 |
| 835 | pA29824 |
| 836 | pA29825 |
| 837 | pA29826 |
| 838 | pA29827 |
| 839 | pA29828 |
| 840 | pA29829 |
| 841 | pA29830 |
| 842 | pA29831 |
| 843 | pA29832 |
| 844 | pA29833 |
| 845 | pA29834 |
| 846 | pA29835 |
| 847 | pA29836 |
| 848 | pA29837 |
| 849 | pA29838 |
| 850 | pA29839 |
| 851 | pA29840 |
| 852 | pA29841 |
| 853 | pA29842 |
| 854 | pA29843 |
| 855 | pA29844 |
| 856 | pA29845 |
| 857 | pA29846 |
| 858 | pA29847 |
| 859 | pA29848 |
| 860 | pA29849 |
| 861 | pA29850 |
| 862 | pA29851 |
| 863 | pA29852 |
| 864 | pA29853 |
| 865 | pA29854 |
| 866 | pA29855 |
| 867 | pA29856 |
| 868 | pA29857 |
| 869 | pA29858 |
| 870 | pA29859 |
| 871 | pA29860 |
| 872 | pA29861 |
| 873 | pA29862 |
| 874 | pA29863 |
| 875 | pA29864 |
| 876 | pA29865 |
| 877 | pA29866 |
| 878 | pA29867 |
| 879 | pA29868 |
| 880 | pA29869 |
| 881 | pA29870 |
| 882 | pA29871 |
| 883 | pA10142 |
| 884 | pA12546 |
| 885 | pA21838 |
| 886 | pA21839 |
| 887 | pA21840 |
| 888 | pA21841 |
| 889 | pA21842 |
| 890 | pA33240 |
| 891 | pA33241 |
| 892 | pA33242 |
| 893 | pA33244 |
| 894 | pA33246 |
| 895 | pA33247 |
| 896 | pA33248 |
| 897 | pA33249 |
| 898 | pA33250 |
| 899 | pA33252 |
| 900 | pA33253 |
| 901 | pA33254 |
| 902 | pA33255 |
| 903 | pA33258 |
| 904 | pA33259 |
| 905 | pA33260 |
| 906 | pA33261 |
| 907 | pA33262 |
| 908 | pA33263 |
| 909 | pA33264 |
| 910 | pA33267 |
| 911 | pA33269 |
| 912 | pA33270 |
| 913 | pA33271 |
| 914 | pA33272 |
| 915 | pA33273 |
| 916 | pA33274 |
| 917 | pA33276 |
| 918 | pA33278 |
| 919 | pA33279 |
| 920 | pA33280 |
| 921 | pA33281 |
| 922 | pA33282 |
| 923 | pA33285 |
| 924 | pA33286 |
| 925 | pA32582 |
| 926 | pA32586 |
| 927 | pA32589 |
| 928 | pA32591 |
| 929 | pA32594 |
| 930 | pA32595 |
| 931 | pA32596 |
| 932 | pA32600 |
| 933 | pA32601 |
| 934 | pA32602 |
| 935 | pA32603 |
| 936 | pA32605 |
| 937 | pA32606 |
| 938 | pA32608 |
| 939 | pA32609 |
| 940 | pA32614 |
| 941 | pA32616 |
| 942 | pA32620 |
| 943 | pA32622 |
| 944 | pA32623 |
| 945 | pA32625 |
| 946 | pA32629 |
| 947 | pA32639 |
| 948 | pA32650 |
| 949 | pA32651 |
| 950 | pA32652 |
| 951 | pA32655 |
| 952 | pA32657 |
| 953 | pA32659 |
| 954 | pA32661 |
| 955 | pA32662 |
| 956 | pA32663 |
| 957 | pA32664 |
| 958 | pA32665 |
| 959 | pA32666 |
| 960 | pA32667 |
| 961 | pA32668 |
| 962 | pA32669 |
| 963 | pA32670 |
| 964 | pA32671 |
| 965 | pA32672 |
| 966 | pA32675 |
| 967 | pA32678 |
| 968 | pA32680 |
| 969 | pA32682 |
| 970 | pA32684 |
| 971 | pA32686 |
| 972 | pA32688 |
| 973 | pA32690 |
| 974 | pA32692 |
| 975 | pA32696 |
| 976 | pA32697 |
| 977 | pA32698 |
| 978 | pA32700 |
| 979 | pA32701 |
| 980 | pA32702 |
| 981 | pA32704 |
| 982 | pA32706 |
| 983 | pA32708 |
| 984 | pA32712 |
| 985 | pA32715 |
| 986 | pA32716 |
| 987 | pA32717 |
| 988 | pA32719 |
| 989 | pA32721 |
| 990 | pA32723 |
| 991 | pA32731 |
| 992 | pA32732 |
| 993 | pA32734 |
| 994 | pA32736 |
| 995 | pA32737 |
| 996 | pA32739 |
| 997 | pA32748 |
| 998 | pA32750 |
| 999 | pA32754 |
| 1000 | pA32758 |
| 1001 | pA32767 |
| 1002 | pA32770 |
| 1003 | pA32779 |
| 1004 | pA32781 |
| 1005 | pA32787 |
| 1006 | pA32790 |
| 1007 | pA32793 |
| 1008 | pA32796 |
| 1009 | pA32797 |
| 1010 | pA32799 |
| 1011 | pA32800 |
| 1012 | pA32801 |
| 1013 | pA32802 |
| 1014 | pA32804 |
| 1015 | pA32806 |
| 1016 | pA32808 |
| 1017 | pA32816 |
| 1018 | pA32819 |
| 1019 | pA32820 |
| 1020 | pA32821 |
| 1021 | pA32824 |
| 1022 | pA32825 |
| 1023 | pA32826 |
| 1024 | pA32828 |
| 1025 | pA32831 |
| 1026 | pA32834 |
| 1027 | pA32835 |
| 1028 | pA32837 |
| 1029 | pA32842 |
| 1030 | pA32844 |
| 1031 | pA32845 |
| 1032 | pA32847 |
| 1033 | pA32848 |
| 1034 | pA32854 |
| 1035 | pA32855 |
| 1036 | pA32858 |
| 1037 | pA32863 |
| 1038 | pA32864 |
| 1039 | pA32865 |
| 1040 | pA32866 |
| 1041 | pA32868 |
| 1042 | pA32876 |
| 1043 | pA32880 |
| 1044 | pA32916 |
| 1045 | pA32919 |
| 1046 | pA32924 |
| 1047 | pA32926 |
| 1048 | pA32934 |
| 1049 | pA32632 |
| 1050 | pA32634 |
| 1051 | pA32638 |
| 1052 | pA32646 |
| 1053 | pA32653 |
| 1054 | pA32722 |
| 1055 | pA32724 |
| 1056 | pA32727 |
| 1057 | pA32729 |
| 1058 | pA32735 |
| 1059 | pA32738 |
| 1060 | pA32742 |
| 1061 | pA32743 |
| 1062 | pA32746 |
| 1063 | pA32747 |
| 1064 | pA32749 |
| 1065 | pA32751 |
| 1066 | pA32752 |
| 1067 | pA32757 |
| 1068 | pA32760 |
| 1069 | pA32761 |
| 1070 | pA32763 |
| 1071 | pA32765 |
| 1072 | pA32769 |
| 1073 | pA32775 |
| 1074 | pA32780 |
| 1075 | pA32782 |
| 1076 | pA32785 |
| 1077 | pA32791 |
| 1078 | pA32869 |
| 1079 | pA32871 |
| 1080 | pA32875 |
| 1081 | pA32879 |
| 1082 | pA32881 |
| 1083 | pA32882 |
| 1084 | pA32884 |
| 1085 | pA32885 |
| 1086 | pA32886 |
| 1087 | pA32888 |
| 1088 | pA32892 |
| 1089 | pA32894 |
| 1090 | pA32895 |
| 1091 | pA32902 |
| 1092 | pA32903 |
| 1093 | pA32904 |
| 1094 | pA32906 |
| 1095 | pA32908 |
| 1096 | pA32912 |
| 1097 | pA32913 |
| 1098 | pA32914 |
| 1099 | pA32917 |
| 1100 | pA32918 |
| 1101 | pA32922 |
| 1102 | pA32923 |
| 1103 | pA32930 |
| 1104 | pA32932 |
| 1105 | pA32579 |
| 1106 | pA32581 |
| 1107 | pA32583 |
| 1108 | pA32585 |
| 1109 | pA32587 |
| 1110 | pA32597 |
| 1111 | pA32599 |
| 1112 | pA32610 |
| 1113 | pA32613 |
| 1114 | pA32617 |
| 1115 | pA32619 |
| 1116 | pA32621 |
| 1117 | pA32627 |
| 1118 | pA32631 |
| 1119 | pA32633 |
| 1120 | pA32637 |
| 1121 | pA32641 |
| 1122 | pA32647 |
| 1123 | pA32656 |
| 1124 | pA32658 |
| 1125 | pA32673 |
| 1126 | pA32677 |
| 1127 | pA32679 |
| 1128 | pA32681 |
| 1129 | pA32683 |
| 1130 | pA32685 |
| 1131 | pA32689 |
| 1132 | pA32691 |
| 1133 | pA32693 |
| 1134 | pA32695 |
| 1135 | pA32711 |
| 1136 | pA32725 |
| 1137 | pA32740 |
| 1138 | pA32744 |
| 1139 | pA32764 |
| 1140 | pA32768 |
| 1141 | pA32772 |
| 1142 | pA32773 |
| 1143 | pA32774 |
| 1144 | pA32776 |
| 1145 | pA32777 |
| 1146 | pA32778 |
| 1147 | pA32795 |
| 1148 | pA32803 |
| 1149 | pA32805 |
| 1150 | pA32807 |
| 1151 | pA32809 |
| 1152 | pA32813 |
| 1153 | pA32815 |
| 1154 | pA32817 |
| 1155 | pA32827 |
| 1156 | pA32832 |
| 1157 | pA32833 |
| 1158 | pA32839 |
| 1159 | pA32841 |
| 1160 | pA32843 |
| 1161 | pA32849 |
| 1162 | pA32851 |
| 1163 | pA32853 |
| 1164 | pA32857 |
| 1165 | pA32859 |
| 1166 | pA32867 |
| 1167 | pA32872 |
| 1168 | pA32873 |
| 1169 | pA32887 |
| 1170 | pA32889 |
| 1171 | pA32891 |
| 1172 | pA32897 |
| 1173 | pA32901 |
| 1174 | pA32907 |
| 1175 | pA32911 |
| 1176 | pA32915 |
| 1177 | pA32920 |
| 1178 | pA32927 |
| 1179 | pA32929 |
| 1180 | pA32931 |
| 1181 | pA34086 |
| 1182 | pA34087 |
| 1183 | pA34088 |
| 1184 | pA34089 |
| 1185 | pA34090 |
| 1186 | pA34091 |
| 1187 | pA34092 |
| 1188 | pA34094 |
| 1189 | pA34095 |
| 1190 | pA34096 |
| 1191 | pA34097 |
| 1192 | pA34098 |
| 1193 | pA34099 |
| 1194 | pA34100 |
| 1195 | pA34101 |
| 1196 | pA34102 |
| 1197 | pA34103 |
| 1198 | pA34104 |
| 1199 | pA34105 |
| 1200 | pA34106 |
| 1201 | pA34107 |
| 1202 | pA34109 |
| 1203 | pA34110 |
| 1204 | pA34111 |
| 1205 | pA34112 |
| 1206 | pA34113 |
| 1207 | pA34115 |
| 1208 | pA34116 |
| 1209 | pA34117 |
| 1210 | pA34118 |
| 1211 | pA34119 |
| 1212 | pA34120 |
| 1213 | pA34121 |
| 1214 | pA34122 |
| 1215 | pA34123 |
| 1216 | pA34124 |
| 1217 | pA34125 |
| 1218 | pA34126 |
| 1219 | pA34127 |
| 1220 | pA34128 |
| 1221 | pA34129 |
| 1222 | pA34130 |
| 1223 | pA34131 |
| 1224 | pA34132 |
| 1225 | pA34133 |
| 1226 | pA34134 |
| 1227 | pA34135 |
| 1231 | pA35407 |
| 1232 | pA35408 |
| 1233 | pA35409 |
| 1234 | pA35410 |
| 1235 | pA35411 |
| 1236 | pA35412 |
| 1237 | pA35413 |
| 1238 | pA35414 |
| 1239 | pA35415 |
| 1240 | pA35416 |
| 1241 | pA35417 |
| 1242 | pA35418 |
| 1243 | pA35419 |
| 1244 | pA35420 |
| 1245 | pA35421 |
| 1246 | pA35422 |
| 1247 | pA35423 |
| 1248 | pA35424 |
| 1249 | pA35425 |
| 1250 | pA35426 |
| 1251 | pA35427 |
| 1252 | pA35428 |
| 1253 | pA35429 |
| 1254 | pA35430 |
| 1255 | pA35431 |
| 1256 | pA35432 |
| 1257 | pA35434 |
| 1258 | pA35435 |
| 1259 | pA35436 |
| 1260 | pA35437 |
| 1261 | pA35438 |
| 1262 | pA35439 |
| 1263 | pA35440 |
| 1264 | pA35441 |
| 1265 | pA35442 |
| 1266 | pA35443 |
| 1267 | pA35444 |
| 1268 | pA35872 |
| 1269 | pA35873 |
| 1270 | pA35874 |
| 1271 | pA35875 |
| 1272 | pA35876 |
| 1273 | pA35877 |
| 1274 | pA35878 |
| 1275 | pA35879 |
| 1276 | pA35880 |
| 1277 | pA35881 |
| 1278 | pA35882 |
| 1279 | pA35884 |
| 1280 | pA35886 |
| 1281 | pA35887 |
| 1282 | pA35888 |
| 1283 | pA35889 |
| 1284 | pA35890 |
| 1285 | pA35891 |
| 1286 | pA35892 |
| 1287 | pA35893 |
| 1288 | pA35895 |
| 1289 | pA35896 |
| 1290 | pA35897 |
| 1291 | pA35898 |
| 1292 | pA35899 |
| 1293 | pA35901 |
| 1294 | pA35902 |
| 1295 | pA35903 |
| 1296 | pA35904 |
| 1297 | pA35905 |
| 1298 | pA35906 |
| 1299 | pA35907 |
| 1300 | pA35908 |
| 1301 | pA35909 |
| 1302 | pA35910 |
| 1303 | pA35911 |
| 1304 | pA35912 |
| 1305 | pA35913 |
| 1306 | pA35914 |
| 1307 | pA35915 |
| 1308 | pA35916 |
| 1309 | pA35917 |
| 1310 | pA35919 |
| 1311 | pA35921 |
| 1312 | pA35922 |
| 1313 | pA35923 |
| 1314 | pA35924 |
| 1315 | pA35925 |
| 1316 | pA35926 |
| 1317 | pA35927 |
| 1318 | pA35928 |
| 1319 | pA35929 |
| 1320 | pA35930 |
| 1321 | pA35931 |
| 1322 | pA35932 |
| 1323 | pA35933 |
| 1324 | pA35935 |
| 1325 | pA35936 |
| 1326 | pA35937 |
| 1327 | pA35938 |
| 1328 | pA35939 |
| 1329 | pA35940 |
| 1330 | pA35941 |
| 1331 | pA35942 |
| 1332 | pA35943 |
| 1333 | pA35481 |
| 1334 | pA35482 |
| 1335 | pA35483 |
| 1336 | pA35484 |
| 1337 | pA35485 |
| 1338 | pA35486 |
| 1339 | pA35487 |
| 1340 | pA35488 |
| 1341 | pA35489 |
| 1342 | pA35490 |
| 1343 | pA35491 |
| 1344 | pA35492 |
| 1345 | pA35493 |
| 1346 | pA35494 |
| 1347 | pA35495 |
| 1348 | pA35496 |
| 1349 | pA35497 |
| 1350 | pA35498 |
| 1351 | pA35499 |
| 1352 | pA35500 |
| 1353 | pA35501 |
| 1354 | pA35502 |
| 1355 | pA35503 |
| 1356 | pA35504 |
| 1357 | pA35505 |
| 1358 | pA35506 |
| 1359 | pA35507 |
| 1360 | pA35508 |
| 1361 | pA35509 |
| 1362 | pA35510 |
| 1363 | pA35511 |
| 1364 | pA35512 |
| 1365 | pA35513 |
| 1366 | pA35514 |
| 1367 | pA35515 |
| 1368 | pA35516 |
| 1369 | pA35517 |
| 1370 | pA35518 |
| 1371 | pA35519 |
| 1372 | pA35520 |
| 1373 | pA35521 |
| 1374 | pA35522 |
| 1375 | pA35523 |
| 1376 | pA35524 |
| 1377 | pA35525 |
| 1378 | pA35526 |
| 1379 | pA35527 |
| 1380 | pA35528 |
| 1381 | pA35529 |
| 1382 | pA35530 |
| 1383 | pA35531 |
| 1384 | pA35532 |
| 1385 | pA35533 |
| 1386 | pA35534 |
| 1387 | pA35535 |
| 1388 | pA35536 |
| 1389 | pA35537 |
| 1390 | pA35538 |
| 1391 | pA35539 |
| 1392 | pA35540 |
| 1393 | pA35541 |
| 1394 | pA35542 |
| 1395 | pA35543 |
| 1396 | pA35544 |
| 1397 | pA35545 |
| 1398 | pA35546 |
| 1399 | pA35547 |
| 1400 | pA35548 |
| 1401 | pA35549 |
| 1402 | pA35550 |
| 1403 | pA35551 |
| 1404 | pA35552 |
| 1405 | pA35553 |
| 1406 | pA35554 |
| 1407 | pA35555 |
| 1408 | pA35556 |
| 1409 | pA35557 |
| 1410 | pA35558 |
| 1411 | pA35559 |
| 1412 | pA35560 |
| 1413 | pA35561 |
| 1414 | pA35562 |
| 1415 | pA35563 |
| 1416 | pA35564 |
| 1417 | pA35565 |
| 1418 | pA35566 |
| 1419 | pA35567 |
| 1420 | pA35568 |
| 1421 | pA35569 |
| 1422 | pA35570 |
| 1423 | pA35571 |
| 1424 | pA35572 |
| 1425 | pA35573 |
| 1426 | pA35574 |
| 1427 | pA35575 |
| 1428 | pA35576 |
| 1429 | pA35577 |
| 1430 | pA35578 |
| 1431 | pA35579 |
| 1432 | pA35580 |
| 1433 | pA35581 |
| 1434 | pA35582 |
| 1435 | pA35583 |
| 1436 | pA35584 |
| 1437 | pA35585 |
| 1438 | pA35586 |
| 1439 | pA35587 |
| 1440 | pA35588 |
| 1441 | pA35589 |
| 1442 | pA35590 |
| 1443 | pA35591 |
| 1444 | pA35592 |
| 1445 | pA35593 |
| 1446 | pA35594 |
| 1447 | pA35595 |
| 1448 | pA35596 |
| 1449 | pA35597 |
| 1450 | pA35598 |
| 1451 | pA35599 |
| 1452 | pA35600 |
| 1453 | pA35601 |
| 1454 | pA35602 |
| 1455 | pA35603 |
| 1456 | pA35604 |
| 1457 | pA35605 |
| 1458 | pA35606 |
| 1459 | pA35607 |
| 1460 | pA35608 |
| 1461 | pA35609 |
| 1462 | pA35610 |
| 1463 | pA35611 |
| 1464 | pA35612 |
| 1465 | pA35613 |
| 1466 | pA35614 |

### EXAMPLES

### Example 1: In Vivo Production of Native Beta-1,2-Glycosyltransferases (B12GTs)

Polynucleotides encoding the amino acid sequences for known beta-1,2-UDP-glycosyl-transferases from five different organisms (Table 1.1) were synthesized (Twist Bioscience) and inserted into the pARZ4 expression vector. Polynucleotides were either ordered as full-length genes or ordered as gene fragments and then assembled using Gibson assembly. The recombinant vectors were used in a heat shock method to transform E. coli HMS174 (DE3) (Novagen), thereby preparing recombinant microorganisms.

Each transformed recombinant microorganism was inoculated to 1 ml LB-kanamycin medium, cultured by shaking at 37° C. overnight. The culture was inoculated to 5 ml TB-kanamycin medium and grown for 2 hours at 37° C., followed by 25° C. for 1 hour. The culture was induced with 50 uL 50 mM IPTG and grown overnight. Finally, the culture was centrifuged at top-speed for 5 minutes and stored at −80° C.

TABLE 1.1
Native Beta-1,2-UDP-Glycosyltransferases

| PlasmidID | Organism |
|---|---|
| pA10132 | Oryza sativa subsp. japonica (Rice) |
| pA10143 | Solanum lycopersicum (Tomato) |
| pA12549 | Lycium barbarum (Barbary matrimony-vine) |
| pA12550 | Solanum lycopersicum (Tomato) |
| pA28422 | Solanum tuberosum (Potato) |

### Example 2: Purification of Beta-1,2-Glycosyltransferases (B12GTs)

The microorganisms created in Example I were dissolved in a lysis buffer (lysozyme, DNAseI, Bugbuster, 300 mL 20 mM HEPES pH 7.5, 500 mM NaCl, and 20 mM Imidazole). Two to three glass beads were added to each well and were disrupted by shaking at 25° C. and 220 rpm for 30 minutes. The disrupted liquid was centrifuged at 2200×g for 6-10 minutes. The obtained supernatant was loaded onto a Ni-NTA plate and shaken for 10 minutes at room temperature. The plate was centrifuged for 4 minutes at 100×g followed by two washes of 500 uL binding buffer (300 mL 20 mM HEPES pH 7.5, 500 mM NaCl, 20 mM Imidazole) and two-minute centrifugation (500×g). The proteins were eluted with 150 uL elution buffer (15 mL 20 mM HEPES pH 7.5, 500 mM NaCl, 500 mM Imidazole) and shaken for 1 minute at 0.25 maximum shaking speed followed by centrifugation for 2 minutes at 500×g. The recovered protein was desalted into a buffer solution for enzyme activity evaluation (50 mM HEPES pH 7.5, 50 mM NaCl).

### Example 3: Measure Beta-1,2-Glycosyltransferase (B12GT) Activity with ADP-Glucose and GDP-Glucose

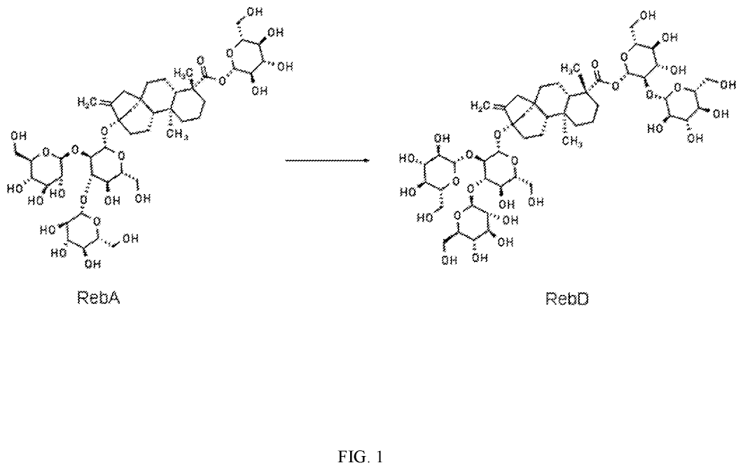

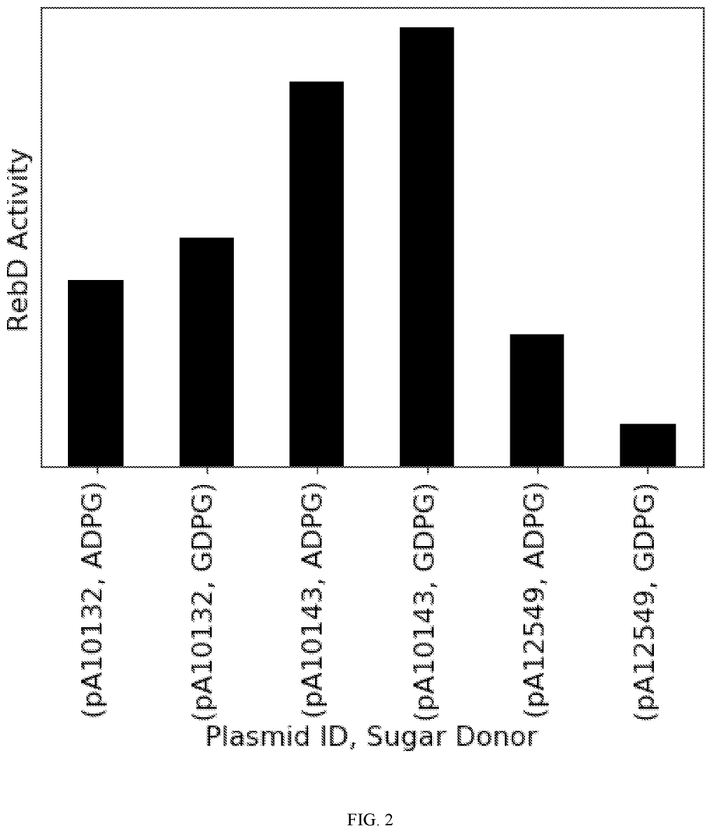

The wild-type beta-1,2-UDP-glycosyltransferases, pA10132, pA10143 and pA12549, were assayed for activity with ADP-glucose and GDP-glucose. Purified protein was reacted with 0.5 mM RA99 (99% Pure Reb A), 2 mM NDP-glucose (ADP-glucose or GDP-glucose) in 50 mM MOPS pH 7.8 buffer for 72 hours at 30° C. Conversion of Reb A to Reb D, as schematized in FIG. 1, was monitored by liquid chromatography-mass spectrometry (LCMS) using an Agilent 6470 QQQ mass spectrometer (column: Waters ACQUITY UPLC HSS T3 Column, 100 mm×2.1 mm). The QQQ was run with multi reaction monitoring (MS/MS) to accurately quantitate steviol glycosides of interest. All three wild-type B12GTs had minimal Reb A to Reb D activity when using ADP-glucose or GDP-glucose as the reaction sugar donor (FIG. 2).

### Example 4: In Vivo Production of Native Sucrose Synthases

Polynucleotides encoding the amino acid sequences for sucrose synthases (SuSys) from seven different organisms (Table 2) were synthesized (Twist Bioscience) and inserted into the pARZ4 expression vector. Polynucleotides were either ordered as full-length genes or ordered as gene fragments and then assembled using Gibson assembly. The recombinant vectors were used in a heat shock method to transform E. coli NEBT7EL (New England Biolabs), thereby preparing recombinant microorganisms.

Each transformed recombinant microorganism was inoculated to 1 ml LB-kanamycin medium, cultured by shaking at 37° C. overnight. The culture was inoculated to 5 ml TB-kanamycin medium and grown for 2 hours at 37° C., followed by 25° C. for 1 hour. The culture was induced with 50 uL 50 mM IPTG and grown overnight. Finally, the culture was centrifuged at top-speed for 5 minutes and stored at −80° C.

TABLE 2
Wildtype Sucrose Synthase Sequences

| PlasmidID | Organism |
|---|---|
| pA10142 | Arabidopsis thaliana |
| pA12546 | Coffea arabica |
| pA21838 | Acidithiobacillus caldus |
| pA21839 | Nitrosomonas europaea |
| pA21840 | Denitrovibrio acetiphilus |
| pA21841 | Thermosynechococcus elongatus |
| pA21842 | Oryza sativa subsp. japonica (Rice) |

### Example 5: Purification of Native Sucrose Synthases

The microorganisms created in Example 4 were dissolved in a lysis buffer (lysozyme, DNAseI, Bugbuster, 300 mL 20 mM HEPES pH 7.5, 500 mM NaCl, and 20 mM Imidazole). Two to three glass beads were added to each well and were disrupted by shaking at 25° C. and 220 rpm for 30 minutes. The disrupted liquid was centrifuged at 2200×g for 6-10 minutes. The obtained supernatant was loaded onto a Ni-NTA plate and shaken for 10 minutes at room temperature. The plate was centrifuged for 4 minutes at 100×g followed by two washes of 500 uL binding buffer (300 mL 20 mM HEPES pH 7.5, 500 mM NaCl, 20 mM Imidazole) and two-minute centrifugation (500×g). The proteins were eluted with 150 uL elution buffer (15 mL 20 mM HEPES pH 7.5, 500 mM NaCl, 500 mM Imidazole) and shaken for 1 minute at 0.25 maximum shaking speed followed by centrifugation for 2 minutes at 500×g. The recovered protein was desalted into a buffer solution for enzyme activity evaluation (50 mM MOPS pH 6.5, 50 mM NaCl).

### Example 6: Measure Sucrose Synthase Activity with UDP, GDP and ADP

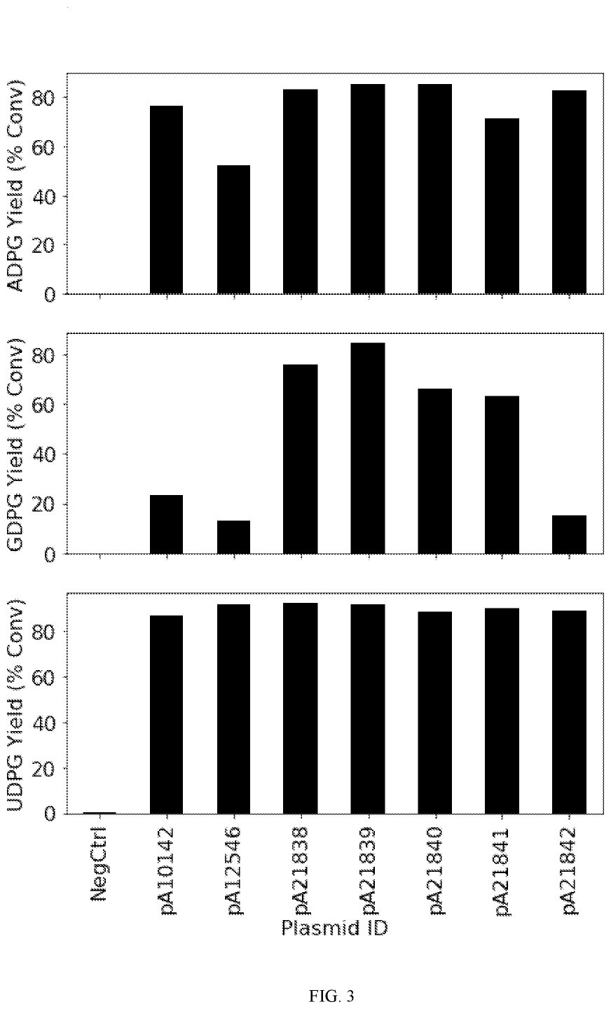

Purified enzyme from Example 5 was reacted with 50 mM sucrose and 5 mM nucleotide (ADP, GDP or UDP) in 50 mM MOPS buffer (pH 6.5) and 50 mM NaCl for 24 hours at 60° C. Conversion of NDP to NDP-glucose was monitored by liquid chromatography-mass spectrometry (LCMS) using an Agilent 6545 QTOF mass spectrometer (column: Agilient HILIC-OH 150×2.1 mm). The wild-type sucrose synthases were active on all three nucleotides (FIG. 3).

### Example 7: Conversion of Reb A to Reb D in a One-Pot Reaction

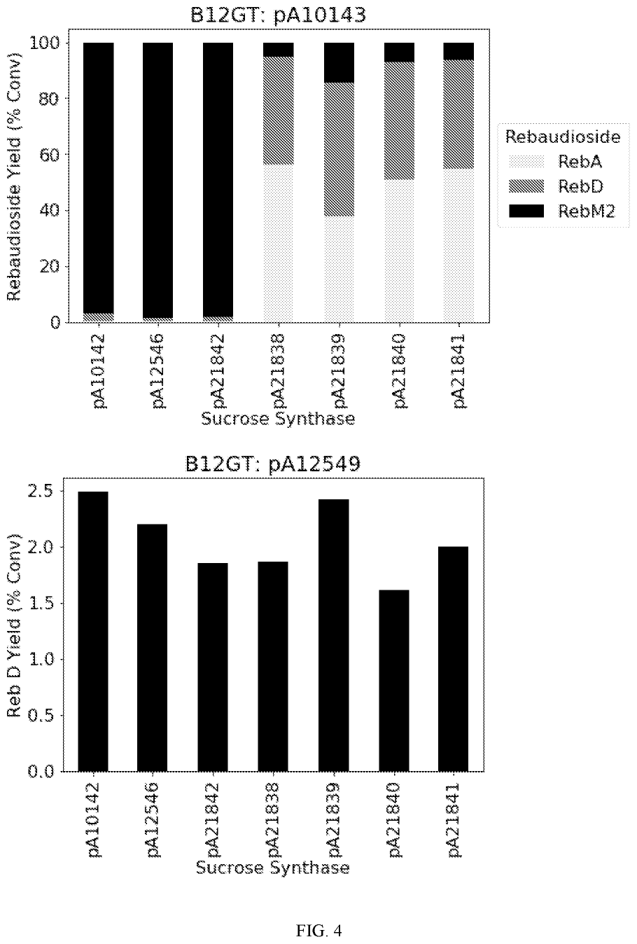

One-pot reactions containing a B12GT and a SuSy were conducted to demonstrate the ability to convert Reb A to Reb D using ADP-glucose generated by the SuSy. Purified B12GT (pA10143 (FIG. 4 (top)) or pA12549 (FIG. 4 (bottom))) and purified SuSy (pA10142, pA12546, pA21838, pA21839, pA21840, pA21841, or pA21842) were reacted with 0.5 mg/mL RA99, 50 mM Sucrose, and 5 mM ADP in 50 mM pH 6.5 MOPS buffer, 3.0 mM MgCl₂ and 50 mM NaCl for 24 hours at 30° C. Product rebaudiosides were monitored by LCMS similar to Example 3. All one-pot reactions were able to generate Reb D from Reb A (FIG. 4). The one-pot reactions containing pA10143 further converted the generated Reb D to Reb M2.

### Example 8: Improved Activity of pA10143 by Site-Saturation Mutagenesis

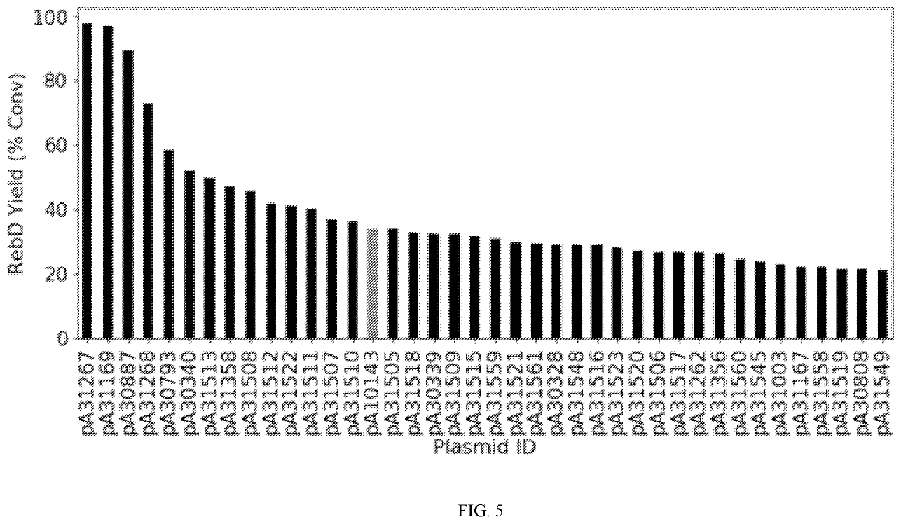

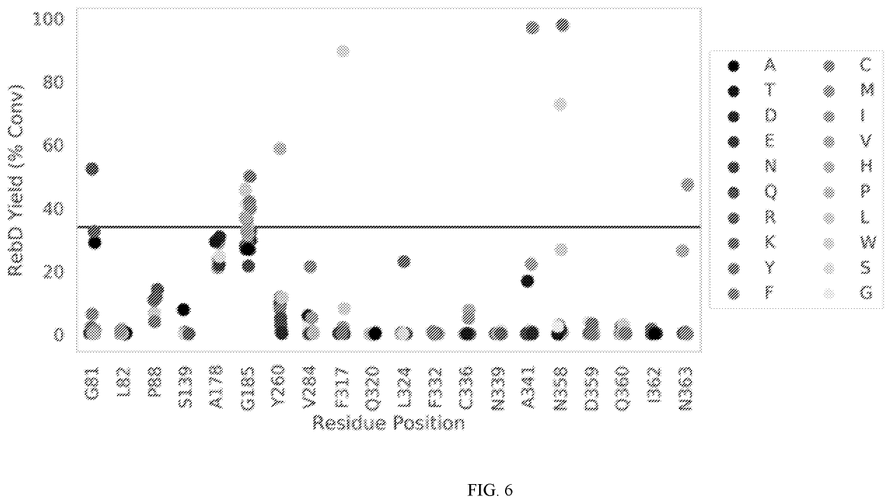

Homology models of the B12GT encoded by pA10143 were generated and used to identify the active site residues of the protein. The following twenty active site residue positions were chosen for site-saturation mutagenesis: 81, 82, 88, 139, 178, 185, 260, 284, 317, 320, 324, 332, 336, 339, 341, 358, 359, 360, 362, 363. Gibson assembly using bridging oligos was used to create 217 single point mutant variants of pA10143 (SEQ ID NOs: 6-222). Each B12GT variant was expressed and purified as in Example 2. Each B12GT variant was assayed in a one-pot reaction with the SuSy, pA10142. The purified B12GT and SuSy were reacted with 4 mg/mL RA50, 40 mM Sucrose, and 1 mM ADP in 50 mM pH 7 phosphate buffer, 3.0 mM MgCl₂ and 50 mM NaCl for 24 hours at 30° C. Product rebaudiosides were monitored by LCMS similar to Example 3. Several variants showed improved activity compared to the parent pA10143 (FIG. 5). Active site positions 358, 341 and 317 had the greatest improvements in activity (FIG. 6).

### Example 9: Improved Activity of pA10143 by Computational Design

Homology models of the B12GT encoded by pA10143 were used as input to computational designs to improve pA10143. Computational designs were conducted to improve the stability and expression of the B12GT. Ninety-three computational designs were chosen for experimental validation (SEQ ID NOs: 223-315). Expression plasmids for the computational designs were built as in Example 1. Each B12GT variant was expressed and purified as in Example 2. Each B12GT variant was assayed in a one-pot reaction with the SuSy, pA10142. The purified B12GT and SuSy were reacted with 4 mg/mL RA50, 40 mM Sucrose, and 1 mM ADP in 50 mM pH 7 phosphate buffer, 3.0 mM MgCl₂ and 50 mM NaCl for 24 hours at 30° C. Product rebaudiosides were monitored by LCMS similar to Example 3. Several variants showed improved expression and/or Reb D conversion compared to the parent pA10143 (18% conversion, 39 uM purified protein; Table 3).

TABLE 3
Top Computational Designs of pA10143

| | | Purified |
|---|---|---|
| ID | % Conversion | Conc (uM) |
| pA29938 | 60.5 | 79.2 |
| pA29923 | 44.5 | 24.6 |
| pA29955 | 34.5 | 48 |
| pA29979 | 34.4 | 56.4 |
| pA29977 | 25.9 | 68 |
| pA29983 | 25.7 | 65.3 |
| pA29920 | 25.3 | 21.3 |
| pA29932 | 25 | 74.1 |
| pA29934 | 22 | 54.6 |
| pA29939 | 21.8 | 38.5 |
| pA29953 | 18.4 | 42.7 |
| pA29918 | 16.1 | 119.5 |
| pA29984 | 14 | 32.6 |
| pA29967 | 13.3 | 38.2 |
| pA30006 | 12.7 | 10 |
| pA29957 | 11.2 | 22.5 |
| pA29963 | 10.4 | 23.2 |
| pA29944 | 9.5 | 64.5 |
| pA29961 | 9.1 | 28 |
| pA29916 | 8 | 35.5 |
| pA29949 | 7.9 | 36.4 |
| pA29966 | 7.9 | 22.8 |
| pA29951 | 7.8 | 45.6 |
| pA30008 | 7.7 | 7.6 |
| pA29921 | 7.4 | 15.2 |
| pA29981 | 7.1 | 39.8 |
| pA29973 | 6.7 | 33.2 |
| pA29995 | 5.2 | 61.9 |
| pA29985 | 5 | 65.1 |
| pA29969 | 4.8 | 22.6 |
| pA29950 | 4.8 | 46.2 |
| pA29931 | 4.7 | 22.5 |
| pA29968 | 4.6 | 51.1 |
| pA29933 | 4.6 | 8.6 |
| pA30007 | 4.6 | 8 |
| pA29917 | 4.5 | 26.6 |
| pA29992 | 4.4 | 37.7 |
| pA29978 | 4.4 | 44.6 |
| pA29960 | 4.3 | 19 |
| pA29986 | 4.3 | 17.5 |
| pA29919 | 3.8 | 14.3 |
| pA29942 | 3.7 | 43.3 |
| pA29954 | 3.6 | 10.8 |
| pA29970 | 3.5 | 13.4 |
| pA29996 | 3.4 | 39.8 |
| pA29922 | 2.9 | 13.2 |
| pA29990 | 2.7 | 35.5 |
| pA29926 | 2.5 | 81 |
| pA29991 | 2.4 | 52.8 |
| pA29964 | 2.3 | 14 |
| pA29987 | 2.2 | 67.6 |
| pA29937 | 2.1 | 10.4 |
| pA29925 | 1.5 | 21.9 |
| pA30003 | 1.5 | 30.4 |
| pA29952 | 1.4 | 9.1 |
| pA29941 | 1.4 | 9.8 |
| pA29993 | 1.3 | 55.7 |
| pA29971 | 1.3 | 11.1 |
| pA29998 | 1.2 | 32.3 |
| pA29935 | 1.2 | 14.6 |
| pA30005 | 1.2 | 6.5 |
| pA30001 | 1 | 37.6 |
| pA29945 | 1 | 7.7 |
| pA29962 | 1 | 6.3 |
| pA29948 | 1 | 9.8 |
| pA29980 | 0.9 | 14 |
| pA29959 | 0.9 | 38.5 |
| pA29976 | 0.8 | 17.8 |
| pA29927 | 0.7 | 12 |
| pA29989 | 0.7 | 35.3 |
| pA29946 | 0.6 | 9.8 |
| pA29928 | 0.6 | 39.5 |
| pA29947 | 0.6 | 8.9 |
| pA29924 | 0.4 | 130.4 |
| pA29972 | 0.4 | 56.6 |
| pA30009 | 0.3 | 6.4 |
| pA29982 | 0.3 | 33 |

Computational designs of pA10143 were also conducted to improve the stability and expression of the B12GT using coevolutionary information. Sixty computational designs were chosen for experimental validation (SEQ ID NOs: 316-375). Expression plasmids for the computational designs were built as in Example 1. Each B12GT variant was expressed and purified as in Example 2. Each B12GT variant was assayed in a one-pot reaction with the SuSy, pA10142. The purified B12GT and SuSy were reacted with 4 mg/mL RA50, 40 mM Sucrose, and 1 mM ADP in 50 mM pH 7 phosphate buffer, 3.0 mM MgCl₂ and 50 mM NaCl for 24 hours at 30° C. Product rebaudiosides were monitored by LCMS similar to Example 3. Computational design variants showed improved expression and/or Reb D conversion compared to the parent pA10143 (4.7% conversion, 35 uM purified protein; Table 4).

TABLE 4
Top Computational Designs of pA10143

| | | Purified |
|---|---|---|
| ID | % Conversion | Conc (uM) |
| pA29711 | 6.7 | 45.4 |
| pA29743 | 5.9 | 18.6 |
| pA29733 | 5.6 | 27.8 |
| pA29734 | 5.1 | 5.4 |
| pA29713 | 3 | 20 |
| pA29717 | 2.3 | 41.6 |
| pA29729 | 2.1 | 12.4 |
| pA29745 | 2.1 | 40.2 |
| pA29741 | 1.6 | 27.7 |
| pA29742 | 1.5 | 7.5 |
| pA29723 | 1.3 | 30.4 |
| pA29763 | 1.3 | 24.7 |
| pA29719 | 1.1 | 29.3 |
| pA29747 | 0.9 | 34.4 |
| pA29735 | 0.9 | 21.1 |
| pA29765 | 0.8 | 23.7 |
| pA29740 | 0.8 | 12.5 |
| pA29707 | 0.7 | 36.8 |
| pA29751 | 0.7 | 28.3 |
| pA29720 | 0.7 | 23.2 |
| pA29705 | 0.7 | 38 |
| pA29715 | 0.7 | 30 |
| pA29760 | 0.7 | 18.2 |
| pA29721 | 0.6 | 11.3 |
| pA29757 | 0.5 | 27.6 |
| pA29749 | 0.5 | 16.1 |
| pA29709 | 0.5 | 45.5 |
| pA29710 | 0.4 | 6.4 |
| pA29759 | 0.4 | 24.5 |
| pA29712 | 0.4 | 4.5 |
| pA29764 | 0.4 | 0.6 |
| pA29744 | 0.4 | 26 |
| pA29736 | 0.4 | 24.8 |
| pA29727 | 0.4 | 21.2 |
| pA29737 | 0.4 | 31.1 |
| pA29738 | 0.4 | 9.6 |
| pA29725 | 0.4 | 19.8 |
| pA29728 | 0.3 | 17.6 |
| pA29731 | 0.3 | 3.2 |
| pA29752 | 0.3 | 26.6 |
| pA29718 | 0.3 | 3.4 |
| pA29753 | 0.3 | 36.6 |
| pA29746 | 0.2 | 3.6 |
| pA29730 | 0.2 | 1.9 |
| pA29750 | 0.2 | 1.6 |
| pA29748 | 0.2 | 2.7 |
| pA29722 | 0.2 | ND |
| pA29756 | 0.1 | 1.7 |
| pA29724 | 0.1 | ND |
| pA29761 | 0.1 | 35.3 |
| pA29708 | 0.1 | 1.7 |
| pA29758 | 0.1 | 6.9 |
| pA29732 | 0.1 | ND |

Computational designs of pA10143 were also conducted to combine active site mutations. Nine computational designs were chosen for experimental validation (SEQ ID NOs: 376-384). Expression plasmids for the computational designs were built as in Example 1. Each B12GT variant was expressed and purified as in Example 2. Each B12GT variant was assayed in a one-pot reaction with the SuSy, pA10142. The purified B12GT and SuSy were reacted with 4 mg/mL RA50, 40 mM Sucrose, and 1 mM ADP in 50 mM pH 7 phosphate buffer, 3.0 mM MgCl₂ and 50 mM NaCl for 24 hours at 30° C. Product rebaudiosides were monitored by LCMS similar to Example 3. The top design, pA32576, showed activity comparable to the parent pA10143 (21% conversion; Table 5).

TABLE 5
Top Computational Designs of pA10143

| | | Purified |
|---|---|---|
| ID | % Conversion | Conc (uM) |
| pA32576 | 20.1 | 63.1 |
| pA32568 | 0.9 | 20 |
| pA32572 | 0.7 | 21.8 |
| pA32569 | 0.7 | 25.2 |
| pA32571 | 0.6 | 18.7 |
| pA32570 | 0.6 | 24.5 |
| pA32573 | 0.5 | 24.3 |
| pA32567 | 0.5 | 21.7 |
| pA32574 | 0.3 | 14.7 |

Computational designs of pA10143 were also conducted to combine additional mutations. Seventy-five computational designs were chosen for experimental validation (SEQ ID NOs: 385-459). Expression plasmids for the computational designs were built as in Example 1. Each B12GT variant was expressed and purified as in Example 2. Each B12GT variant was assayed in a one-pot reaction with the SuSy, pA10142. The purified B12GT and SuSy were reacted with 4 mg/mL RA50, 40 mM Sucrose, and 1 mM ADP in 50 mM pH 7 phosphate buffer, 3.0 mM MgCl₂ and 50 mM NaCl for 24 hours at 30° C. Product rebaudiosides were monitored by LCMS similar to Example 3. Computational design variants showed Reb A to Reb D conversion (Table 6).

TABLE 6
Top Computational Designs of pA10143

| | | Purified |
|---|---|---|
| ID | % Conversion | Conc (uM) |
| pA33106 | 3.9 | 9.4 |
| pA33091 | 2.7 | 11.8 |
| pA33123 | 2.2 | 10.2 |
| pA33137 | 1.1 | 57.5 |
| pA33154 | 0.7 | 15.8 |
| pA33149 | 0.6 | 14.7 |
| pA33088 | 0.6 | 13.4 |
| pA33086 | 0.5 | 17.3 |
| pA33110 | 0.5 | 19.7 |
| pA33083 | 0.2 | 21.9 |
| pA33085 | 0.1 | 15.3 |
| pA33165 | 0.1 | 17.2 |

### Example 10: Improved Activity of pA12549 by Computational Design

Homology models of the B12GT encoded by pA12549 were used as input to computational designs to improve pA12549. Computational designs of pA12549 were conducted to combine active site mutations known to be beneficial in homologous B12GTs. Eight computational designs were chosen for experimental validation (SEQ ID NOs: 460-467). Expression plasmids for the computational designs were built as in Example 1. Each B12GT variant was expressed and purified as in Example 2. Each B12GT variant was assayed in a one-pot reaction with the SuSy, pA10142. The purified B12GT and SuSy were reacted with 0.5 mg/mL RA99, 10 mM Sucrose, and 1 mM ADP in 50 mM pH 7 phosphate buffer, 3.0 mM MgCl₂ and 50 mM NaCl for 24 hours at 30° C. Product rebaudiosides were monitored by LCMS similar to Example 3. Several variants showed improved expression and/or Reb D conversion compared to the parent pA12549 (Table 7).

TABLE 7
Top pA12549 Computational Designs

| | | Purified |
|---|---|---|
| ID | % Conversion | Conc (uM) |
| pA32563 | 12.7 | 9.2 |
| pA32562 | 8 | 6.8 |
| pA32565 | 6.5 | 38.9 |
| pA32560 | 4.6 | 10.1 |
| pA32566 | 4.5 | 11.2 |
| pA32558 | 3.6 | 11.6 |
| pA32559 | 1.7 | 12.1 |

Computational designs of pA12549 were also conducted to combine mutations known to be beneficial in homologous B12GTs. Sixty-seven computational designs were chosen for experimental validation (SEQ ID NOs: 468-534). Expression plasmids for the computational designs were built as in Example 1. Each B12GT variant was expressed and purified as in Example 2. Each B12GT variant was assayed in a one-pot reaction with the SuSy, pA10142. The purified B12GT and SuSy were reacted with 0.5 mg/mL RA99, 10 mM Sucrose, and 1 mM ADP in 50 mM pH 7 phosphate buffer and 50 mM NaCl for 24 hours at 30° C. Product rebaudiosides were monitored by LCMS similar to Example 3. One computational design variant showed an improvement in Reb A to Reb D conversion (Table 8).

TABLE 8
Top pA12549 Computational Designs

| | | Purified |
|---|---|---|
| ID | % Conversion | Conc (uM) |
| pA33202 | 25.2 | 11.4 |
| pA33194 | 6.6 | 16.7 |
| pA33196 | 5.3 | 14.1 |
| pA33236 | 4.8 | 14.1 |
| pA33200 | 4.2 | 8.8 |
| pA33223 | 3 | 6.1 |
| pA33177 | 1.9 | 9.9 |
| pA33217 | 1.8 | 22.7 |
| pA33205 | 1.7 | 10.6 |
| pA33229 | 1.3 | 11 |
| pA33176 | 1 | 16.7 |
| pA33235 | 0.9 | 14.4 |
| pA33174 | 0.5 | 15.2 |
| pA33185 | 0.5 | 15.3 |
| pA33171 | 0.5 | 15.8 |
| pA33173 | 0.5 | 9.5 |
| pA33220 | 0.4 | 7.8 |
| pA33204 | 0.4 | 11.3 |
| pA33180 | 0.3 | 10.5 |
| pA33186 | 0.3 | 5.9 |
| pA33172 | 0.3 | 13.7 |
| pA33179 | 0.3 | 11.7 |
| pA33175 | 0.2 | 10.8 |
| pA33182 | 0.2 | 15.1 |
| pA33232 | 0.2 | 18 |

### Example 11: Improved Activity and Expression of the SUS1 from Arabidopsis thaliana by Computational Design

The crystal structure of SUS1 from Arabidopsis thaliana was used as input to computational designs to improve pA10142. Computational designs were conducted to improve the stability and expression of the SuSy. Thirty-five computational designs were chosen for experimental validation (SEQ ID NOs: 890-924). Expression plasmids for the computational designs were built as in Example 1. Each SuSy variant was expressed and purified as in Example 2. Each SuSy variant was assayed in a one-pot reaction with the B12GT, pA10143. The purified B12GT and SuSy were reacted with 4 mg/mL RA50, 40 mM Sucrose, and 1 mM ADP in 50 mM pH 7 phosphate buffer and 50 mM NaCl for 24 hours at 30° C. Product rebaudiosides were monitored by LCMS similar to Example 3. Several variants showed improved expression and/or Reb D conversion compared to the parent pA10142. The top designs showed up to a 2-fold improvement in yield and 3-fold improvement in expression (43% conversion, 8 uM purified protein; Table 9).

TABLE 9
Top Computational Designs of pA10142

| | | Purified |
|---|---|---|
| ID | % Conversion | Conc (uM) |
| pA33271 | 79.8 | 23.2 |
| pA33264 | 79.7 | 11.3 |
| pA33276 | 79 | 17.5 |
| pA33273 | 78.1 | 11.2 |
| pA33272 | 77.9 | 8.8 |
| pA33279 | 77.6 | 24.6 |
| pA33281 | 76.6 | 18.8 |
| pA33248 | 76.4 | 32.9 |
| pA33263 | 76.3 | 19.8 |
| pA33285 | 75.9 | 17.2 |
| pA33247 | 75.4 | 26.6 |
| pA33261 | 74.5 | 12.1 |
| pA33240 | 74 | 20.5 |
| pA33262 | 74 | 21 |
| pA33260 | 72.7 | 8.8 |
| pA33259 | 71 | 23.7 |
| pA33280 | 70.2 | 26.1 |
| pA33267 | 70 | 20 |
| pA33282 | 70 | 21.7 |
| pA33269 | 68.3 | 12.5 |
| pA33242 | 65.5 | 17.6 |
| pA33254 | 64.8 | 15.9 |
| pA33241 | 62.8 | 22.8 |
| pA33274 | 57.8 | 8.6 |
| pA33270 | 57.1 | 12.2 |
| pA33244 | 45.7 | 26 |
| pA33278 | 44.8 | 9.4 |
| pA33258 | 42.3 | 25.1 |
| pA33286 | 38.1 | 13 |
| pA33249 | 26.4 | 16 |
| pA33246 | 20.8 | 24.5 |
| pA33250 | 16.8 | 15.1 |
| pA33253 | 10.6 | 2.8 |
| pA33255 | 1.6 | 11.2 |
| pA33252 | 0.3 | 4.5 |

### Example 12: Computational Design of ADP-Glucose Dependent B12GTs

Structural models of a B12GT variant of pA28422 were generated and used as the starting point for computational designs. Computational designs were conducted to improve the stability and expression of the B12GT. Fifty-two computational designs were chosen for experimental validation (SEQ ID NOs: 535-586). Expression plasmids for the computational designs were built as in Example 1. Each B12GT variant was expressed and purified as in Example 2. Each B12GT variant was assayed in a one-pot reaction with a SuSy variant of pA21838. The purified B12GT and SuSy were reacted with 100 mg/mL RA50, 250 mM Sucrose, and 0.5 mM ADP in 50 mM pH 6 phosphate buffer, 3 mM MgCl₂ and 50 mM NaCl for 24 hours at 60° C. Product rebaudiosides were monitored by LCMS similar to Example 3. Several designed enzymes expressed well and were active for Reb A to Reb D conversion (Table 10).

TABLE 10
Top B12GT Computational Designs

| | | Purified |
|---|---|---|
| ID | % Conversion | Conc (uM) |
| pA29798 | 76.1 | 57.3 |
| pA29781 | 67.1 | 51.9 |
| pA29807 | 64.9 | 17.9 |
| pA29795 | 61.7 | 64.8 |
| pA29796 | 57.8 | 74.3 |
| pA29771 | 54.3 | 49.3 |
| pA29775 | 53.6 | 67 |
| pA29770 | 45 | 61 |
| pA29808 | 43.4 | 95.1 |
| pA29774 | 42.8 | 86.8 |
| pA29784 | 38.5 | 44.1 |
| pA29767 | 33.8 | 37 |
| pA29801 | 16.9 | 74.7 |
| pA29779 | 13 | 39.6 |
| pA29773 | 12 | 49.7 |
| pA29800 | 9.4 | 95.8 |
| pA29799 | 9.3 | 39.7 |
| pA29789 | 8.7 | 75.8 |
| pA29804 | 7.9 | 88.1 |
| pA29797 | 7.4 | 69.3 |
| pA29806 | 7 | 77.9 |
| pA29769 | 6.4 | 37.9 |
| pA29787 | 5.2 | 68.6 |
| pA29805 | 4.8 | 38.4 |
| pA29817 | 3.3 | 37.3 |
| pA29790 | 3.2 | 77.5 |
| pA29802 | 3.1 | 35.8 |
| pA29792 | 3 | 71.7 |
| pA29816 | 2.2 | 30.9 |
| pA29782 | 2.1 | 58.1 |
| pA29809 | 1.9 | 56.5 |
| pA29780 | 1.6 | 57.3 |
| pA29783 | 1.3 | 41.3 |
| pA29811 | 1 | 77.4 |
| pA29813 | 1 | 37.3 |
| pA29794 | 0.7 | 32.8 |
| pA29785 | 0.6 | 19.6 |
| pA29814 | 0.4 | 21.4 |
| pA29791 | 0.4 | 17.5 |
| pA29788 | 0.4 | 7.7 |
| pA29812 | 0.1 | 44.6 |
| pA29776 | 0.1 | 37.8 |

Computational designs were also conducted to improve the stability and expression of the B12GT using coevolutionary information. Eighty-five computational designs were chosen for experimental validation (SEQ ID NOs: 587-671). Expression plasmids for the computational designs were built as in Example 1. Each B12GT variant was expressed and purified as in Example 2. Each B12GT variant was assayed in a one-pot reaction with a SuSy variant of pA21838. The purified B12GT and SuSy were reacted with 100 mg/mL RA50, 250 mM Sucrose, and 0.5 mM ADP in 50 mM pH 6 phosphate buffer, 3 mM MgCl₂ and 50 mM NaCl for 24 hours at 60° C. Product rebaudiosides were monitored by LCMS similar to Example 3. Several designed enzymes expressed well and were active for Reb A to Reb D conversion (Table 11).

TABLE 11
Top B12GT Computational Designs

| | | Purified |
|---|---|---|
| ID | % Conversion | Conc (uM) |
| pA29620 | 92.6 | 93.7 |
| pA29619 | 91.8 | 29.7 |
| pA29665 | 90.5 | 142.7 |
| pA29621 | 88.7 | 62 |
| pA29661 | 87 | 29.4 |
| pA29618 | 82.5 | 77.7 |
| pA29660 | 56 | 7.6 |
| pA29615 | 54.4 | 191.9 |
| pA29616 | 53.8 | 123 |
| pA29646 | 40.4 | 60.8 |
| pA29617 | 34.7 | 150.1 |
| pA29642 | 32.3 | 71.3 |
| pA29636 | 24.9 | 28.1 |
| pA29662 | 22.7 | 12.4 |
| pA29639 | 21.9 | 49.3 |
| pA29669 | 21.4 | 60.3 |
| pA29625 | 21.2 | 11.4 |
| pA29632 | 20.9 | 94.1 |
| pA29676 | 18.7 | 74.5 |
| pA29664 | 18.5 | 19.7 |
| pA29640 | 16.7 | 37.6 |
| pA29683 | 13.2 | 19.9 |
| pA29672 | 12.9 | 19 |
| pA29624 | 12.7 | 66.8 |
| pA29638 | 12.2 | 29.4 |
| pA29671 | 11.8 | 12.6 |
| pA29675 | 10.9 | 23 |
| pA29650 | 9.9 | 18.9 |
| pA29677 | 9.2 | 48.8 |
| pA29657 | 8.5 | 11.8 |
| pA29668 | 8.5 | 74.8 |
| pA29666 | 8.1 | 108 |
| pA29630 | 7.7 | 20.7 |
| pA29652 | 7.1 | 11.2 |
| pA29623 | 7 | 25.3 |
| pA29667 | 6.7 | 67.9 |
| pA29684 | 6.5 | 105.2 |
| pA29678 | 6.4 | 15 |
| pA29654 | 6.1 | 22.2 |
| pA29685 | 5.8 | 91.7 |
| pA29634 | 5.2 | 47.7 |
| pA29627 | 4.9 | 26.6 |
| pA29682 | 4.9 | 82.9 |
| pA29622 | 4.8 | 68.4 |
| pA29659 | 3.9 | 31.1 |
| pA29635 | 3.9 | 29.1 |
| pA29648 | 3.5 | 10.1 |
| pA29633 | 3.4 | 9.7 |
| pA29673 | 3.3 | 50.6 |
| pA29626 | 3.2 | 26.5 |
| pA29680 | 3 | 18.6 |
| pA29679 | 1.9 | 15.2 |
| pA29681 | 1.9 | 106.5 |
| pA29631 | 1.6 | 34.9 |
| pA29637 | 1.6 | 77.7 |
| pA29687 | 0.9 | 44 |
| pA29695 | 0.7 | 56.4 |
| pA29690 | 0.7 | 36.3 |
| pA29686 | 0.4 | 24.8 |
| pA29689 | 0.4 | 52.7 |
| pA29628 | 0.4 | 5.2 |
| pA29688 | 0.4 | 11 |
| pA29629 | 0.3 | 102.1 |
| pA29696 | 0.2 | 31 |
| pA29703 | 0.2 | 29 |
| pA29698 | 0.1 | 43.9 |

Computational designs were also conducted to improve the stability and expression of the B12GT by redesigning and repacking buried protein cores. Thirty-five computational designs were chosen for experimental validation (SEQ ID NOs: 672-706). Expression plasmids for the computational designs were built as in Example 1. Each B12GT variant was expressed and purified as in Example 2. Each B12GT variant was assayed in a one-pot reaction with a SuSy variant of pA21838. The purified B12GT and SuSy were reacted with 100 mg/mL RA50, 250 mM Sucrose, and 0.5 mM ADP in 50 mM pH 6 phosphate buffer, 3 mM MgCl₂ and 50 mM NaCl for 24 hours at 60° C. Product rebaudiosides were monitored by LCMS similar to Example 3. Several designed enzymes expressed well and were active for Reb A to Reb D conversion (Table 12).

TABLE 12
Top B12GT Computational Designs

| | | Purified |
|---|---|---|
| ID | % Conversion | Conc (uM) |
| pA30048 | 86.4 | 96.8 |
| pA30047 | 50.9 | 106.7 |
| pA30027 | 48.7 | 168 |
| pA30050 | 35.9 | 185.3 |
| pA30037 | 26.7 | 95.9 |
| pA30019 | 24.4 | 93.4 |
| pA30034 | 23.5 | 69.8 |
| pA30028 | 14.1 | 229.9 |
| pA30031 | 13.8 | 9.6 |
| pA30036 | 13.3 | 107.1 |
| pA30017 | 12.9 | 68.2 |
| pA30040 | 8.5 | 48.2 |
| pA30049 | 8.3 | 209.7 |
| pA30032 | 8.3 | 7.6 |
| pA30039 | 7.3 | 92.1 |
| pA30042 | 4.8 | 60.4 |
| pA30029 | 4.5 | 141 |
| pA30035 | 3.6 | 21.6 |
| pA30026 | 3 | 158.6 |
| pA30030 | 2.8 | 280.3 |
| pA30041 | 2.2 | 69.1 |
| pA30025 | 1.9 | 74.8 |
| pA30045 | 1.9 | 70 |
| pA30046 | 1.4 | 103.1 |
| pA30018 | 1.2 | 38.5 |
| pA30038 | 1.1 | 94.4 |
| pA30015 | 0.8 | 13.3 |
| pA30020 | 0.7 | 69.8 |
| pA30023 | 0.7 | 128.2 |
| pA30016 | 0.3 | 16.3 |
| pA30033 | 0.2 | 16.8 |

Computational designs were also conducted to improve the B12GT by combining mutations known to be beneficial in homologous B12GTs. Fifty-nine computational designs were chosen for experimental validation (SEQ ID NOs: 707-765). Expression plasmids for the computational designs were built as in Example 1. Each B12GT variant was expressed and purified as in Example 2. Each B12GT variant was assayed in a one-pot reaction with a SuSy variant of pA21838. The purified B12GT and SuSy were reacted with 100 mg/mL RA50, 250 mM Sucrose, and 0.5 mM ADP in 50 mM pH 6 phosphate buffer and 50 mM NaCl for 24 hours at 60° C. Product rebaudiosides were monitored by LCMS similar to Example 3. Several designed enzymes expressed well and were active for Reb A to Reb D conversion (

Table 13). To distinguish between the top designs, they were re-assayed at lower protein concentrations (Table 14).

TABLE 13
Top B12GT Computational Designs

| | | Purified |
|---|---|---|
| ID | % Conversion | Conc (uM) |
| pA32962 | 92.3 | 95.6 |
| pA32940 | 91.9 | 122.6 |
| pA32945 | 91.3 | 74.5 |
| pA32947 | 91.2 | 80 |
| pA32952 | 90.9 | 83.3 |
| pA32941 | 90.8 | 114.3 |
| pA32946 | 90.7 | 75.8 |
| pA32943 | 90.3 | 113.7 |
| pA32942 | 89.4 | 120.4 |
| pA32939 | 85.7 | 115.5 |
| pA32944 | 77.4 | 31.6 |
| pA32970 | 33.4 | 56.9 |
| pA32960 | 30.3 | 12.6 |
| pA32956 | 30.1 | 44.1 |
| pA32951 | 27.5 | 81.2 |
| pA32966 | 26.2 | 31.6 |
| pA32967 | 12.6 | 64.9 |
| pA32954 | 8 | 111 |
| pA32950 | 6.6 | 48.2 |
| pA32949 | 6.5 | 16.7 |
| pA32978 | 5.1 | 33.9 |
| pA32971 | 4.1 | 19.6 |
| pA32988 | 3.9 | 18.7 |
| pA33004 | 3.4 | 15.1 |
| pA32987 | 3.3 | 17.6 |
| pA32981 | 3.1 | 32.8 |
| pA32958 | 2.7 | 26.6 |
| pA32959 | 2.2 | 20.2 |
| pA32963 | 1.8 | 13.4 |
| pA32953 | 1.7 | 97 |
| pA32973 | 1.3 | 64 |
| pA32948 | 1.2 | 10.9 |
| pA32965 | 1.2 | 7.6 |
| pA32957 | 0.8 | 14.7 |
| pA32955 | 0.7 | 19.1 |
| pA32998 | 0.7 | 30 |
| pA33001 | 0.7 | 12.8 |
| pA32982 | 0.3 | 16 |
| pA32997 | 0.2 | 32.2 |
| pA33002 | 0.2 | 14.2 |

TABLE 14
Top B12GT Computational Designs

| | | Purified |
|---|---|---|
| ID | % Conversion | Conc (uM) |
| pA32947 | 82 | 89 |
| pA32941 | 81.4 | 154 |
| pA32940 | 78.3 | 120 |
| pA32946 | 77.8 | 85.5 |
| pA32962 | 60 | 96.1 |
| pA32945 | 59.4 | 65.7 |
| pA32943 | 55.3 | 102.9 |
| pA32939 | 42 | 150.1 |
| pA32942 | 40.5 | 96 |
| pA32952 | 39.6 | 120.2 |
| pA32944 | 28.5 | 31.4 |
| pA32960 | 17.3 | 9.4 |
| pA32966 | 15.5 | 52.8 |
| pA32951 | 13 | 104.5 |
| pA32970 | 11.3 | 44.3 |
| pA32956 | 10.1 | 33.5 |
| pA32967 | 6.4 | 56.9 |
| pA32949 | 5.8 | 9.5 |
| pA32950 | 4.6 | 45.5 |
| pA32954 | 3.3 | 115.2 |

### Example 13: Computational Design of ADP-Glucose Dependent B12GTs

Structural models of a second B12GT variant of pA28422 were generated and used as the starting point for computational designs. Computational designs were conducted to improve the B12GT by combining mutations known to be beneficial in homologous B12GTs. Sixty-four computational designs were chosen for experimental validation (SEQ ID NOs: 766-829). Expression plasmids for the computational designs were built as in Example 1. Each B12GT variant was expressed and purified as in Example 2. Each B12GT variant was assayed in a one-pot reaction with a SuSy variant of pA21838. The purified B12GT and SuSy were reacted with 100 mg/mL RA50, 250 mM Sucrose, and 0.5 mM ADP in 50 mM pH 6 phosphate buffer and 50 mM NaCl for 24 hours at 60° C. Product rebaudiosides were monitored by LCMS similar to Example 3. Several designed enzymes expressed well and were active for Reb A to Reb D conversion (Table 15). To distinguish between the top designs, they were re-assayed at lower protein concentrations (Table 16).

TABLE 15
Top B12GT Computational Designs

| | | Purified |
|---|---|---|
| ID | % Conversion | Conc (uM) |
| pA33014 | 91 | 55.4 |
| pA33009 | 90.8 | 39.3 |
| pA33008 | 90.1 | 74.5 |
| pA33016 | 89 | 64 |
| pA33020 | 57.3 | 72.7 |
| pA33007 | 52.7 | 31.9 |
| pA33012 | 44.3 | 51.6 |
| pA33019 | 40.2 | 143.3 |
| pA33011 | 24.9 | 93.2 |
| pA33043 | 20.5 | 59.7 |
| pA33018 | 16 | 79.5 |
| pA33005 | 14.3 | 26.9 |
| pA33056 | 13.3 | 14.4 |
| pA33015 | 9.4 | 29.2 |
| pA33030 | 5.2 | 48.4 |
| pA33006 | 4.9 | 37.5 |
| pA33038 | 4.3 | 40.1 |
| pA33010 | 3.1 | 49.7 |
| pA33029 | 1.9 | 95.4 |
| pA33065 | 1.8 | 32.1 |
| pA33017 | 1 | 64.8 |
| pA33025 | 0.8 | 29.3 |
| pA33057 | 0.8 | 25.5 |
| pA33031 | 0.4 | 19 |
| pA33077 | 0.4 | 38.2 |
| pA33069 | 0.3 | 25 |
| pA33070 | 0.2 | 32 |
| pA33078 | 0.1 | 37.5 |
| pA33035 | 0.1 | 18.8 |
| pA33068 | 0.1 | 46.5 |

TABLE 16
Top B12GT Computational Designs

| ID | % Conversion | Purified Conc (uM) |
|---|---|---|
| pA33008 | 88.7 | 37.4 |
| pA33009 | 85.4 | 28.3 |
| pA33016 | 71.5 | 25.4 |
| pA33007 | 49.5 | 10.2 |
| pA33014 | 41.7 | 54.1 |
| pA33020 | 28.3 | 77.3 |
| pA33005 | 27.9 | 16.2 |
| pA33012 | 21.7 | 55 |
| pA33019 | 19.9 | 120.2 |
| pA33056 | 16.9 | 3.7 |
| pA33011 | 15 | 58.3 |
| pA33043 | 14.7 | 19.3 |
| pA33018 | 10.4 | 52 |
| pA33015 | 6.2 | 11.5 |
| pA33006 | 3.9 | 15.1 |
| pA33030 | 3 | 33.8 |

### Example 14: Representing Successful B12GTs Designs with a PSSM

The successful B12GT designs from Example 12 and Example 13 were used to generate a PSSM (Table 17). The PSSM is a concise way to represent the successful designs and related sequences. Sequences that have a PSSM score greater than 266.7 are considered related to the active computational designs described in Example 12 and Example 13. To score a sequence with the PSSM, it must first be aligned with the representative sequence Seq ID No: 5. For example, the following successful designs, pA29646, pA32946, pA29642, pA29798, have the following PSSM scores: 287.2, 288.0, 279.2, 276.8, while the wild-type B12GT pA28422, has a PSSM score of only 257.4.

TABLE 17
Position Specific Scoring Matrix (PSSM) of Successful B12GT Designs

| Seq | Amino Acid | | | | | | | | | | | | | | | | | | | | |
|---|---|---|---|---|---|---|---|---|---|---|---|---|---|---|---|---|---|---|---|---|---|
| Pos | A | R | N | D | C | Q | E | G | H | I | L | K | M | F | P | S | T | W | Y | V | — |
| 1 | 0 | 1 | 0 | 1 | 0 | 0.6 | 0 | 0 | 0 | 0 | 0 | 0 | 0 | 0 | 1 | 1 | 1 | 0 | 0 | 0 | 0 |
| 2 | 0 | 0 | 1 | 0 | 0 | 0 | 0 | 0 | 0 | 0 | 1 | 0 | 0 | 0 | 0 | 1 | 0.6 | 0 | 0 | 0 | 0 |
| 3 | 0 | 0 | 0 | 0 | 0 | 1 | 0 | 0 | 0 | 0 | 0.6 | 0 | 0 | 1 | 0 | 0 | 0 | 0 | 0 | 0 | 0 |
| 4 | 0 | 0.6 | 0 | 0 | 0 | 1 | 0 | 0 | 0 | 0 | 0 | 0 | 0 | 0 | 0 | 0 | 1 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 6 | 1 | 0 | 0 | 0 | 0 | 0 | 0 | 0 | 0 | 0 | 0.6 | 0 | 0 | 0 | 0 | 0 | 0.6 | 0 | 0 | 1 | 0 |
| 7 | 0 | 0 | 0 | 0 | 0 | 0 | 0 | 0 | 0 | 0 | 0 | 0 | 0.6 | 0 | 0 | 0 | 0 | 0 | 0 | 0 | 0 |
| 8 | 0 | 0 | 0 | 0 | 0 | 0 | 0 | 0 | 0 | 0 | 1 | 0 | 0 | 0.6 | 0 | 0 | 0 | 0 | 0 | 1 | 0 |
| 9 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 10 | 0 | 0 | 0 | 0 | 0 | 0 | 0 | 0 | 0 | 0 | 0 | 0 | 0 | 0 | 0 | 0 | 0 | 0.6 | 0 | 0 | 0 |
| 11 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 12 | 0.6 | 0 | 0 | 0 | 0 | 0 | 0 | 0.6 | 0 | 0 | 0 | 0 | 0 | 0 | 0 | 0 | 0 | 0 | 0 | 0 | 0 |
| 13 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 14 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 15 | 0 | 0 | 0 | 0 | 0 | 0 | 0 | 0 | 0.6 | 0 | 0 | 0 | 0 | 0 | 0 | 0 | 0 | 0 | 0 | 0 | 0 |
| 16 | 0 | 0 | 0 | 0 | 0 | 0 | 0 | 0 | 0 | 1 | 0 | 0 | 0 | 0 | 0 | 0 | 0 | 0 | 0 | 0.6 | 0 |
| 17 | 0 | 0 | 0.6 | 0 | 0 | 0 | 0 | 0 | 0 | 0 | 0 | 0 | 0 | 0 | 0 | 0 | 0 | 0 | 0 | 0 | 0 |
| 18 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 19 | 0 | 0 | 0 | 0 | 0 | 0 | 0 | 0 | 0 | 0 | 0 | 0 | 0 | 0.6 | 0 | 0 | 0 | 0 | 1 | 0 | 0 |
| 20 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 21 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 22 | 0 | 0 | 0 | 0 | 0 | 0 | 0 | 0 | 0 | 0.6 | 1 | 0 | 0 | 0 | 0 | 0 | 0 | 0 | 0 | 1 | 0 |
| 23 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 24 | 0 | 1 | 0 | 0 | 0 | 0 | 0 | 0 | 0 | 0 | 0 | 0.6 | 0 | 0 | 0 | 0 | 0 | 0 | 0 | 0 | 0 |
| 25 | 0 | 0 | 0 | 0 | 0 | 0.6 | 0 | 0 | 0 | 0 | 0 | 1 | 0 | 0 | 0 | 0 | 0 | 0 | 0 | 0 | 0 |
| 26 | 0 | 0 | 0 | 0 | 0 | 0 | 0 | 0 | 0 | 0 | 0.6 | 0 | 1 | 0 | 0 | 0 | 0 | 0 | 0 | 0 | 0 |
| 27 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 28 | 0 | 0 | 0 | 0.6 | 0 | 0 | 0 | 0 | 0 | 0 | 0 | 1 | 0 | 0 | 0 | 0 | 0 | 0 | 0 | 0 | 0 |
| 29 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 30 | 0 | 0 | 1 | 0 | 0 | 0 | 0 | 0.6 | 0 | 0 | 0 | 0 | 0 | 0 | 0 | 0 | 0 | 0 | 0 | 0 | 0 |
| 31 | 0 | 0 | 0 | 0 | 0 | 0 | 0 | 0 | 0 | 0 | 0 | 0 | 1 | 0.6 | 0 | 0 | 0 | 0 | 0 | 0 | 0 |
| 32 | 0 | 0 | 0 | 0 | 0 | 0 | 0 | 0 | 1 | 1 | 1 | 0 | 0 | 0 | 0 | 1 | 0 | 0 | 1 | 0.6 | 0 |
| 33 | 0 | 0 | 0 | 0 | 0 | 0 | 0 | 0 | 0 | 0.6 | 0 | 0 | 0 | 0 | 0 | 0 | 0 | 0 | 0 | 1 | 0 |
| 34 | 0 | 0 | 0 | 0 | 0 | 0 | 0 | 0 | 1 | 0 | 0 | 0 | 0 | 0 | 0 | 0 | 0 | 0 | 0.6 | 0 | 0 |
| 35 | 0 | 0 | 0 | 0 | 0 | 0 | 0 | 0 | 0 | 0 | 0.6 | 0 | 1 | 0 | 0 | 0 | 0 | 0 | 0 | 1 | 0 |
| 36 | 1 | 0 | 0 | 0 | 1 | 0 | 0 | 0 | 0 | 0 | 1 | 0 | 0 | 0 | 0 | 0 | 0 | 0 | 0 | 0.6 | 0 |
| 37 | 0 | 0 | 1 | 0 | 0 | 0 | 0 | 0 | 0 | 0 | 0 | 0 | 0 | 0 | 0 | 0.6 | 0 | 0 | 0 | 0 | 0 |
| 38 | 0 | 0 | 0 | 0 | 0 | 0 | 0 | 0 | 0 | 0 | 0 | 0 | 0 | 0 | 0 | 0 | 0.6 | 0 | 0 | 0 | 0 |
| 39 | 0.6 | 0 | 0 | 0 | 0 | 0 | 0 | 0 | 0 | 0 | 0 | 1 | 1 | 0 | 1 | 0 | 0 | 0 | 0 | 0 | 0 |
| 40 | 0 | 0 | 0 | 0 | 0 | 0 | 0 | 0 | 0 | 0.6 | 0 | 0 | 0 | 0 | 0 | 0 | 0 | 0 | 0 | 1 | 0 |
| 41 | 0 | 0 | 0.6 | 0 | 0 | 1 | 0 | 0 | 0 | 0 | 0 | 0 | 0 | 0 | 0 | 0 | 0 | 0 | 0 | 1 | 0 |
| 42 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 43 | 0 | 0 | 1 | 0 | 0 | 0 | 0.6 | 0 | 0 | 0 | 0 | 1 | 0 | 0 | 0 | 1 | 0 | 0 | 0 | 0 | 0 |
| 44 | 0 | 0 | 0 | 0 | 0 | 0 | 0 | 0 | 0 | 0 | 1 | 0 | 0.6 | 0 | 0 | 0.6 | 0 | 0 | 0 | 0 | 0 |
| 45 | 1 | 0 | 0 | 0 | 0 | 0 | 0 | 0 | 0 | 0.6 | 1 | 0 | 0 | 0 | 0 | 0 | 1 | 0 | 0 | 0 | 0 |
| 46 | 0 | 1 | 0 | 0 | 0 | 0 | 0 | 0 | 0 | 0 | 0 | 0.6 | 0 | 0 | 0 | 0 | 0 | 0 | 0 | 0 | 0 |
| 47 | 0 | 0 | 0 | 0 | 0 | 0 | 0 | 1 | 1 | 0 | 0 | 0.6 | 0 | 0 | 0 | 0 | 0 | 0 | 0 | 0 | 0 |
| 48 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 49 | 0 | 0 | 0 | 0 | 0 | 0 | 0 | 0 | 0 | 0.6 | 0 | 0 | 0 | 0 | 0 | 0 | 0 | 0 | 0 | 0 | 0 |
| 50 | 0 | 0 | 0 | 0 | 0 | 0 | 0 | 0 | 0 | 0 | 0 | 0 | 0 | 0 | 0.6 | 0 | 1 | 0 | 0 | 0 | 0 |
| 51 | 0 | 0 | 1 | 0 | 0 | 0 | 0.6 | 0 | 0 | 0 | 0 | 1 | 0 | 0 | 0 | 0 | 0 | 0 | 1 | 0 | 0 |
| 52 | 1 | 0 | 0 | 0 | 0 | 0 | 0 | 0 | 0 | 0 | 0 | 0.6 | 0 | 0 | 0 | 0 | 0 | 0 | 0 | 0 | 0 |
| 53 | 0 | 0 | 0 | 0 | 0 | 0 | 0 | 0 | 0 | 0 | 0 | 0 | 0 | 0 | 0 | 0 | 0 | 0 | 0.6 | 0 | 0 |
| 54 | 1 | 0 | 0 | 0 | 0 | 1 | 0 | 0 | 0 | 0 | 1 | 0 | 0 | 0 | 0 | 0.6 | 0 | 0 | 0 | 1 | 0 |
| 55 | 0 | 0 | 0.6 | 1 | 0 | 1 | 1 | 0 | 0 | 0 | 1 | 0 | 0 | 0 | 0 | 1 | 0 | 0 | 0 | 0 | 0 |
| 56 | 0 | 0 | 0 | 0 | 0 | 0 | 0 | 0 | 0 | 0 | 1 | 0 | 0 | 0 | 0 | 0.6 | 0 | 0 | 0 | 0 | 0 |
| 57 | 0 | 0 | 0 | 0 | 0 | 0 | 0 | 0 | 0 | 0.6 | 0 | 0 | 0 | 0 | 0 | 0 | 0 | 0 | 0 | 0 | 0 |
| 58 | 0 | 0 | 0 | 0 | 0 | 1 | 1 | 0 | 0.6 | 1 | 0 | 0 | 0 | 0 | 0 | 0 | 0 | 0 | 0 | 0 | 0 |
| 59 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 60 | 0 | 0 | 0 | 0 | 0 | 0 | 0 | 0 | 0 | 1 | 0 | 0 | 0 | 0 | 0 | 0 | 0 | 0 | 0 | 0.6 | 0 |
| 61 | 0 | 0 | 0 | 0 | 0 | 0 | 0.6 | 0 | 0 | 0 | 0 | 0 | 0 | 0 | 0 | 0 | 1 | 0 | 0 | 0 | 0 |
| 62 | 0 | 0 | 0 | 0 | 0 | 0 | 0 | 0 | 0 | 0 | 0.6 | 0 | 0 | 0 | 0 | 1 | 0 | 0 | 1 | 1 | 0 |
| 63 | 0.6 | 0.6 | 0 | 0 | 0 | 1 | 0 | 0 | 1 | 0 | 0 | 0 | 0 | 0 | 0 | 1 | 0 | 0 | 0 | 0 | 0 |
| 64 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 65 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 66 | 0 | 0 | 0 | 0 | 0 | 1 | 0.6 | 0 | 0 | 0 | 1 | 0 | 0 | 0 | 0 | 0 | 0 | 0 | 0 | 0 | 0 |
| 67 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 68 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 69 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 70 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 71 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 72 | 0 | 0 | 0 | 0 | 0 | 0 | 0 | 0 | 0 | 0 | 0 | 0 | 0 | 0 | 0.6 | 1 | 0 | 0 | 0 | 0 | 0 |
| 73 | 0 | 0 | 0 | 0 | 0 | 0 | 0 | 0 | 0.6 | 0 | 0 | 0 | 0 | 0 | 0 | 0 | 0 | 0 | 1 | 0 | 0 |
| 74 | 0 | 0 | 0 | 0 | 0 | 0 | 0 | 0 | 0 | 0 | 1 | 0 | 0 | 0 | 0 | 0 | 0 | 0 | 0.6 | 0 | 0 |
| 75 | 0 | 0 | 0 | 0 | 0 | 0 | 0 | 0 | 0.6 | 0 | 0 | 0 | 0 | 0 | 0 | 0 | 0 | 1 | 0 | 0 | 0 |
| 76 | 0 | 0 | 0 | 0 | 0 | 0 | 0 | 0 | 0 | 0 | 0 | 0 | 0 | 0 | 0 | 0 | 0.6 | 0 | 0 | 0 | 0 |
| 77 | 0 | 0 | 0 | 0 | 0 | 0 | 0 | 0 | 0 | 0 | 0 | 0 | 0 | 0 | 0 | 0 | 0.6 | 0 | 0 | 0 | 0 |
| 78 | 0 | 0 | 0.6 | 0 | 0 | 0 | 0 | 0 | 0 | 0 | 0 | 0 | 0 | 0 | 0 | 0 | 0 | 0 | 0 | 0 | 0 |
| 79 | 1 | 0 | 0 | 0 | 0 | 0 | 0 | 0.6 | 0 | 0 | 0 | 0 | 0 | 0 | 0 | 0 | 0 | 0 | 0 | 0 | 0 |
| 80 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 81 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 82 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 83 | 0 | 0 | 0 | 0 | 0 | 0 | 0 | 0 | 0.6 | 0 | 0 | 0 | 0 | 0 | 0 | 0 | 0 | 0 | 0 | 0 | 0 |
| 84 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 85 | 0 | 0 | 0.6 | 0 | 0 | 0 | 0 | 0 | 0 | 0 | 0 | 0 | 0 | 0 | 0 | 0 | 0 | 0 | 0 | 0 | 0 |
| 86 | 0 | 0 | 0 | 0 | 0 | 0 | 0 | 0.6 | 0 | 0 | 0 | 0.6 | 0 | 0 | 0 | 0 | 0 | 0 | 0 | 0 | 0 |
| 87 | 0 | 1 | 0 | 0 | 0 | 0 | 0 | 0 | 0 | 1 | 1 | 0 | 0 | 0 | 0 | 0 | 0.6 | 0 | 0 | 1 | 0 |
| 88 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 89 | 0 | 1 | 0 | 0 | 0 | 1 | 0 | 0 | 0.6 | 1 | 0 | 1 | 0 | 0 | 0 | 0 | 0 | 0 | 0 | 0 | 0 |
| 90 | 0 | 1 | 0 | 0 | 0 | 1 | 0 | 0 | 0 | 0 | 0 | 0.6 | 0 | 0 | 0 | 0 | 0 | 0 | 0 | 0 | 0 |
| 91 | 0.6 | 0 | 0 | 0 | 0 | 0 | 0 | 0 | 0 | 0 | 1 | 0 | 0 | 0 | 0 | 0 | 0 | 0 | 0 | 0 | 0 |
| 92 | 0 | 0 | 0 | 0 | 0 | 0 | 0 | 0 | 0 | 0 | 0.6 | 0 | 0 | 0 | 0 | 0 | 0 | 0 | 0 | 1 | 0 |
| 93 | 0 | 1 | 0 | 0 | 0 | 1 | 0 | 0 | 0 | 0 | 0 | 0.6 | 0 | 0 | 0 | 0 | 0 | 0 | 0 | 0 | 0 |
| 94 | 0 | 0 | 0 | 0 | 0 | 0 | 0 | 0 | 0 | 0 | 1 | 0 | 0.6 | 0 | 0 | 0 | 0 | 0 | 0 | 0 | 0 |
| 95 | 1 | 0 | 0 | 0 | 0 | 0 | 0 | 0 | 0 | 0 | 0 | 0 | 0 | 0 | 0 | 0.6 | 0 | 0 | 0 | 0 | 0 |
| 96 | 0.6 | 1 | 0 | 0 | 0 | 1 | 0 | 0 | 0 | 0 | 0 | 0 | 0 | 0 | 0 | 0 | 0 | 0 | 0 | 0 | 0 |
| 97 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 98 | 0 | 0 | 0.6 | 0 | 0 | 0 | 1 | 0 | 0 | 0 | 0 | 0 | 0 | 0 | 0 | 0 | 1 | 0 | 0 | 0 | 0 |
| 99 | 0 | 0 | 0 | 0 | 0 | 0 | 0 | 0 | 0 | 1 | 1 | 0 | 0 | 0.6 | 0 | 0 | 0 | 0 | 0 | 1 | 0 |
| 100 | 0 | 0 | 0 | 0 | 0 | 0 | 0 | 0 | 0 | 0 | 0 | 0 | 0 | 0 | 0 | 0.6 | 0 | 0 | 0 | 0 | 0 |
| 101 | 0 | 0 | 0 | 0 | 0 | 0 | 0 | 0 | 0 | 0 | 0 | 0.6 | 0 | 0 | 0 | 0 | 0 | 0 | 0 | 0 | 0 |
| 102 | 0 | 0 | 0 | 0 | 0 | 1 | 0 | 0 | 0 | 0.6 | 1 | 0 | 0 | 0 | 0 | 0 | 1 | 0 | 0 | 1 | 0 |
| 103 | 0 | 0 | 0 | 0 | 0 | 0 | 0 | 0 | 0 | 1 | 0.6 | 0 | 0 | 0 | 0 | 0 | 0 | 0 | 0 | 1 | 0 |
| 104 | 0 | 1 | 0 | 0 | 0 | 0.6 | 1 | 0 | 0 | 0 | 0 | 1 | 0 | 0 | 0 | 1 | 0 | 0 | 0 | 0 | 0 |
| 105 | 1 | 0 | 0.6 | 1 | 0 | 0 | 0 | 0 | 0 | 0 | 0 | 0 | 0 | 0 | 0 | 0 | 1 | 0 | 0 | 0 | 0 |
| 106 | 0 | 0 | 0 | 0 | 0 | 0 | 0 | 0 | 0 | 0.6 | 0.6 | 0 | 0 | 0 | 0 | 0 | 0 | 1 | 0 | 0 | 0 |
| 107 | 0 | 0 | 1 | 0 | 0 | 0 | 0 | 0 | 0 | 0 | 0 | 0.6 | 0 | 0 | 0 | 0 | 0 | 0 | 0 | 0 | 0 |
| 108 | 1 | 0 | 0 | 0 | 0 | 0 | 0 | 0 | 0 | 0 | 0 | 0 | 0 | 0 | 0.6 | 1 | 0 | 0 | 0 | 1 | 0 |
| 109 | 1 | 0 | 0 | 0.6 | 0 | 0 | 0 | 0 | 0 | 0 | 0 | 0 | 0 | 0 | 0 | 1 | 1 | 0 | 0 | 0 | 0 |
| 110 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 111 | 0 | 0 | 0 | 0 | 0 | 0 | 0 | 0 | 0 | 1 | 1 | 0 | 0 | 0 | 0 | 0 | 0 | 0 | 0 | 0.6 | 0 |
| 112 | 0 | 0 | 0 | 0 | 0 | 0 | 0 | 0 | 0 | 1 | 0.6 | 0 | 0 | 0 | 0 | 0 | 0 | 0 | 0 | 1 | 0 |
| 113 | 0 | 0 | 0 | 0 | 0 | 0 | 0 | 0 | 0 | 0 | 1 | 0 | 0 | 1 | 0 | 0 | 0 | 0 | 0.6 | 1 | 0 |
| 114 | 0 | 0 | 0 | 0.6 | 0 | 0 | 0 | 0 | 0 | 0 | 0 | 0 | 0 | 0 | 0 | 0 | 0 | 0 | 0 | 0 | 0 |
| 115 | 0 | 0 | 0 | 0 | 0 | 0 | 0 | 0 | 0 | 1 | 1 | 0 | 1 | 0.6 | 0 | 0 | 0 | 0 | 0 | 0 | 0 |
| 116 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 117 | 1 | 0 | 0 | 0 | 0 | 0.6 | 0 | 0 | 0 | 1 | 1 | 0 | 0 | 0 | 0 | 0 | 0 | 0 | 0 | 0.6 | 0 |
| 118 | 0 | 0 | 0 | 0 | 0 | 1 | 0 | 0 | 0 | 0 | 0 | 0 | 0 | 0 | 0.6 | 0 | 0 | 0 | 0 | 0 | 0 |
| 119 | 0 | 0 | 0 | 0 | 0 | 0 | 0 | 0 | 0 | 0 | 0 | 0 | 0 | 0 | 0 | 0 | 0 | 0.6 | 0 | 0 | 0 |
| 120 | 0.6 | 0 | 0 | 0 | 0 | 0 | 0 | 0 | 0 | 0 | 1 | 0 | 0 | 0 | 0 | 0 | 0 | 0 | 0 | 0 | 0 |
| 121 | 0 | 0 | 0 | 0 | 0 | 0 | 0.6 | 0 | 0 | 0 | 0 | 0 | 0 | 0 | 0 | 1 | 0 | 0 | 0 | 0 | 0 |
| 122 | 0 | 0 | 0 | 0 | 0 | 0 | 0 | 0 | 0 | 0 | 0 | 0.6 | 0 | 0 | 0 | 0 | 0 | 0 | 0 | 0 | 0 |
| 123 | 0 | 0 | 0 | 0 | 0 | 0 | 0 | 0 | 0 | 1 | 1 | 0 | 0 | 0 | 0 | 1 | 0 | 0 | 0 | 0.6 | 0 |
| 124 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 125 | 0 | 0 | 1 | 0 | 0 | 0 | 0 | 0 | 0 | 0 | 0.6 | 1 | 0 | 0 | 0 | 0 | 0 | 0 | 0 | 0 | 0 |
| 126 | 0 | 0 | 0 | 1 | 0 | 0 | 0.6 | 0 | 0 | 0 | 0 | 0 | 0 | 0 | 0 | 1 | 0 | 0 | 0 | 0 | 0 |
| 127 | 0 | 1 | 0 | 0 | 0 | 0.6 | 0 | 0 | 0 | 0 | 1 | 0 | 0 | 0 | 0 | 0 | 0 | 0 | 0 | 0 | 0 |
| 128 | 0 | 0 | 1 | 0 | 0 | 0 | 0 | 0.6 | 0 | 0 | 0 | 0 | 0 | 0 | 0 | 0 | 0 | 0 | 0 | 0 | 0 |
| 129 | 0 | 0 | 0 | 0 | 0 | 0 | 0 | 0 | 0 | 0.6 | 0 | 0 | 0 | 0 | 0 | 0 | 0 | 0 | 0 | 1 | 0 |
| 130 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 131 | 0.6 | 0 | 0 | 0 | 0 | 0 | 0 | 1 | 0 | 0 | 0 | 0 | 0 | 0 | 0 | 1 | 0 | 0 | 0 | 1 | 0 |
| 132 | 0 | 0 | 0 | 0 | 0 | 0 | 0 | 0 | 0 | 1 | 0 | 0 | 0 | 0 | 0 | 0 | 0 | 0 | 0 | 0.6 | 0 |
| 133 | 0 | 1 | 0 | 0 | 0 | 0 | 0 | 0 | 0 | 0 | 0 | 1 | 0 | 0 | 0.6 | 0 | 0 | 0 | 0 | 0 | 0 |
| 134 | 0 | 0 | 0 | 0 | 0 | 0 | 0 | 0 | 0 | 0 | 0.6 | 0 | 0 | 1 | 0 | 0 | 0 | 0 | 0 | 0 | 0 |
| 135 | 1 | 0 | 0 | 0 | 0 | 0 | 0 | 0 | 0 | 1 | 0.6 | 0 | 0 | 0 | 0 | 0 | 0 | 1 | 0 | 0 | 0 |
| 136 | 0 | 0 | 0 | 0 | 0 | 0 | 0 | 0 | 0 | 0 | 0 | 0 | 0 | 0 | 0 | 0 | 0.6 | 0 | 0 | 0 | 0 |
| 137 | 0 | 0 | 0 | 0 | 0 | 0 | 0 | 0 | 0 | 0 | 0 | 0 | 0 | 1 | 0 | 0.6 | 0 | 0 | 0 | 0 | 0 |
| 138 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 139 | 0.6 | 0 | 0 | 0 | 0 | 0 | 0 | 0 | 0 | 0 | 1 | 0 | 0 | 0 | 0 | 0 | 0 | 0 | 0 | 1 | 0 |
| 140 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 141 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 142 | 0 | 0 | 0 | 0 | 0 | 0 | 0 | 0 | 0 | 0 | 1 | 0 | 0 | 0.6 | 0 | 0 | 0 | 1 | 0 | 0 | 0 |
| 143 | 1 | 0 | 0 | 0 | 0 | 0 | 0 | 0 | 0 | 0 | 0 | 0 | 0 | 0 | 0 | 0.6 | 0 | 0 | 0 | 0 | 0 |
| 144 | 0 | 0 | 0 | 0 | 0 | 0 | 0 | 0 | 0 | 0 | 0 | 0 | 0 | 0 | 0 | 0 | 0 | 0 | 0.6 | 0 | 0 |
| 145 | 0 | 0 | 0 | 0 | 0 | 0 | 0 | 0 | 0 | 1 | 1 | 0 | 0 | 0.6 | 0 | 0 | 0 | 0 | 0 | 0 | 0 |
| 146 | 0 | 0 | 0 | 0 | 0 | 0 | 0 | 0 | 0 | 0 | 1 | 0 | 1 | 0.6 | 0 | 0 | 0 | 1 | 0 | 1 | 0 |
| 147 | 0 | 0 | 0.6 | 0 | 0 | 1 | 1 | 0 | 1 | 0 | 0 | 0 | 0 | 0 | 0 | 0 | 0 | 0 | 0 | 0 | 0 |
| 148 | 0 | 0 | 0 | 0 | 0 | 0 | 0 | 0 | 1 | 0 | 0 | 0 | 0 | 0.6 | 0 | 0 | 0 | 0 | 0 | 0 | 0 |
| 149 | 0 | 0 | 0 | 0 | 0 | 0 | 0 | 0 | 0 | 0 | 0.6 | 0 | 0 | 0 | 0 | 0 | 0 | 0 | 0 | 1 | 0 |
| 150 | 0 | 1 | 0 | 1 | 0 | 0 | 0 | 0 | 0 | 0 | 0 | 0.6 | 0 | 0 | 0 | 0 | 1 | 0 | 0 | 0 | 0 |
| 151 | 0 | 0.6 | 1 | 0 | 0 | 0 | 0 | 0 | 0 | 0 | 0 | 1 | 0 | 0 | 0 | 0 | 0 | 0 | 0 | 0 | 0 |
| 152 | 0 | 0 | 0 | 0 | 0 | 0 | 0 | 0 | 0 | 0 | 1 | 0 | 0 | 0 | 0.6 | 0 | 0 | 0 | 0 | 0 | 0 |
| 153 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 154 | 0 | 0 | 1 | 0 | 0 | 0 | 0.6 | 0 | 0.6 | 0 | 0 | 0 | 0 | 0 | 0 | 0 | 0 | 0 | 0 | 1 | 0 |
| 155 | 0 | 0 | 0 | 0 | 0 | 0 | 0.6 | 0 | 0 | 0 | 0 | 0 | 0 | 0 | 1 | 0 | 0 | 0 | 0 | 0 | 0 |
| 156 | 0 | 0 | 0 | 0 | 0 | 0 | 0 | 0 | 0 | 0 | 0 | 0 | 0 | 0.6 | 0 | 0 | 0 | 0 | 0 | 0 | 0 |
| 157 | 0 | 0 | 0 | 0 | 0 | 0 | 0 | 0 | 0 | 0 | 1 | 0 | 0 | 0 | 0.6 | 0 | 0 | 0 | 0 | 0 | 0 |
| 158 | 0 | 0 | 0 | 0 | 0 | 0 | 0 | 0 | 0 | 0 | 0 | 0 | 0 | 0.6 | 0 | 0 | 0 | 0 | 0 | 0 | 0 |
| 159 | 0 | 0 | 0 | 0 | 0 | 0 | 0.6 | 0 | 0 | 0 | 0 | 0 | 0 | 0 | 0.6 | 0 | 0 | 0 | 0 | 0 | 0 |
| 160 | 0.6 | 0 | 0 | 0 | 0 | 0 | 1 | 0 | 0 | 0 | 0 | 0 | 0 | 0 | 0 | 0 | 0 | 0 | 0 | 0 | 0 |
| 161 | 0 | 0 | 0 | 0 | 0 | 0 | 0 | 0 | 0 | 0.6 | 0 | 0 | 0 | 0 | 0 | 0 | 0 | 0 | 0 | 0 | 0 |
| 162 | 0 | 0.6 | 0 | 1 | 0 | 0 | 1 | 0 | 1 | 0 | 0 | 1 | 1 | 0 | 0 | 1 | 0 | 0 | 1 | 0 | 0 |
| 163 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 164 | 0 | 1 | 0 | 0 | 0 | 0 | 0 | 0 | 0 | 0 | 0 | 0 | 0 | 0 | 0 | 0.6 | 0 | 0 | 0 | 0 | 0 |
| 165 | 0 | 0 | 0 | 0 | 0 | 0 | 1 | 0 | 0 | 0 | 0 | 0.6 | 0 | 0 | 0 | 0 | 0 | 0 | 0 | 0 | 0 |
| 166 | 0 | 0.6 | 0 | 0 | 0 | 0 | 0 | 0 | 0 | 1 | 1 | 0 | 0 | 0 | 0 | 0 | 0 | 1 | 1 | 0 | 0 |
| 167 | 1 | 0 | 0 | 0 | 0 | 0 | 0.6 | 0 | 0 | 0 | 0 | 0 | 0 | 0 | 0 | 0 | 0 | 0 | 0 | 0 | 0 |
| 168 | 0 | 0 | 0 | 0 | 0 | 0.6 | 0 | 0 | 0 | 0 | 1 | 1 | 0 | 0 | 0 | 0 | 0 | 0 | 0 | 0 | 0 |
| 169 | 1 | 0 | 0 | 0.6 | 0 | 1 | 0 | 0 | 0 | 0 | 0 | 0 | 0 | 0 | 0 | 0 | 0 | 0 | 0 | 1 | 0 |
| 170 | 0 | 0 | 0 | 0 | 0 | 0 | 0 | 0 | 0 | 0 | 0 | 0.6 | 0 | 0 | 0 | 0 | 0 | 1 | 0 | 0 | 0 |
| 171 | 0 | 0 | 0 | 0 | 0 | 0 | 0 | 0 | 1 | 0 | 1 | 0 | 0.6 | 0 | 0 | 0 | 0 | 0 | 0 | 0 | 0 |
| 172 | 0 | 0.6 | 0 | 0 | 0 | 0 | 0 | 0 | 0 | 0 | 0 | 0 | 0 | 0 | 0 | 0 | 0 | 0 | 1 | 0 | 0 |
| 173 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 174 | 1 | 0 | 0 | 0 | 0 | 0 | 0 | 0 | 0 | 0 | 1 | 0 | 0.6 | 0 | 0 | 0 | 0 | 0 | 0 | 0 | 0 |
| 175 | 0 | 0 | 0 | 0 | 0 | 0 | 0 | 0 | 0 | 0 | 0 | 0 | 1 | 0.6 | 0 | 0 | 0 | 0 | 0 | 0 | 0 |
| 176 | 0 | 0 | 0 | 0 | 0 | 0 | 1 | 0.6 | 0 | 0 | 0 | 0 | 0 | 0 | 0 | 0 | 0 | 0 | 0 | 0 | 0 |
| 177 | 0 | 1 | 0 | 0 | 0 | 0 | 0 | 0 | 0 | 0 | 0 | 1 | 0 | 0 | 0 | 0 | 0.6 | 0 | 0 | 0 | 0 |
| 178 | 1 | 0 | 0 | 0 | 0 | 1 | 0.6 | 1 | 0 | 0 | 0 | 0 | 0 | 0 | 0 | 0 | 0 | 0 | 0 | 1 | 0 |
| 179 | 0 | 0 | 0 | 0 | 0 | 0 | 0 | 1 | 0 | 0 | 0 | 0 | 0 | 0 | 0.6 | 0 | 0 | 0 | 0 | 0 | 0 |
| 180 | 0 | 0 | 0 | 1 | 0 | 0 | 0 | 0 | 0 | 0 | 0 | 1 | 0 | 0 | 0.6 | 0 | 0.6 | 0 | 0 | 0 | 0 |
| 181 | 0 | 0 | 0 | 1 | 0 | 0 | 0.6 | 0 | 0 | 0 | 0 | 0 | 0 | 0 | 0 | 0 | 0 | 0 | 0 | 0 | 0 |
| 182 | 0 | 1 | 0 | 1 | 0 | 0 | 0.6 | 0 | 0 | 0 | 1 | 1 | 0 | 0 | 0 | 0 | 0 | 0 | 0 | 0 | 0 |
| 183 | 0 | 0 | 0 | 0.6 | 0 | 0 | 0 | 0 | 0 | 0 | 0 | 0 | 0 | 1 | 0 | 0 | 0 | 0 | 0 | 0 | 0 |
| 184 | 0 | 0 | 0 | 0 | 0 | 0 | 0 | 0 | 0 | 0 | 0 | 0 | 0 | 0.6 | 0 | 0 | 0 | 0 | 0 | 0 | 0 |
| 185 | 0 | 0 | 0 | 0 | 0 | 0 | 0 | 0 | 0 | 0 | 0.6 | 0 | 0 | 1 | 0 | 0 | 0 | 0 | 0 | 0 | 0 |
| 186 | 0.6 | 0 | 0 | 0 | 0 | 0 | 0 | 0 | 0 | 0 | 0 | 0 | 0 | 0 | 0 | 0 | 0 | 0 | 0 | 1 | 0 |
| 187 | 0 | 0 | 0 | 1 | 0 | 0 | 1 | 0 | 0 | 0 | 0 | 1 | 0 | 0 | 0.6 | 0 | 0 | 0 | 0 | 1 | 0 |
| 188 | 0.6 | 0 | 0 | 0 | 0 | 0 | 0 | 0.6 | 0 | 0 | 0 | 0 | 0 | 1 | 0 | 0 | 0 | 0 | 0 | 0 | 0 |
| 189 | 0 | 1 | 0.6 | 0 | 0 | 0.6 | 0 | 0 | 0 | 0 | 0 | 0 | 0 | 0 | 1 | 1 | 0 | 0 | 0 | 0 | 0 |
| 190 | 0.6 | 0 | 1 | 0 | 1 | 0 | 0 | 0 | 0 | 0 | 0 | 1 | 1 | 0 | 0 | 1 | 1 | 0 | 0 | 0 | 0 |
| 191 | 1 | 0 | 0 | 0 | 0 | 1 | 0 | 0.6 | 0 | 0 | 0 | 1 | 0 | 0 | 0 | 0 | 0 | 0 | 0 | 0 | 0 |
| 192 | 1 | 0 | 0 | 0 | 0 | 0 | 0 | 0 | 0 | 0.6 | 0 | 0 | 0 | 0 | 0 | 0 | 0 | 0 | 1 | 0 | 0 |
| 193 | 0 | 0 | 0 | 0 | 0 | 0 | 0 | 0 | 0 | 1 | 1 | 0 | 0.6 | 0 | 0 | 0 | 1 | 0 | 0 | 1 | 0 |
| 194 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 195 | 0 | 0 | 0 | 0 | 0 | 0 | 0 | 0 | 0 | 1 | 0 | 0 | 0.6 | 0 | 0 | 0 | 0 | 0 | 0 | 1 | 0 |
| 196 | 0 | 0 | 0 | 0 | 0.6 | 0 | 0 | 0 | 0 | 0 | 0 | 0 | 1 | 0 | 0 | 0 | 0 | 0 | 0 | 0 | 0 |
| 197 | 0 | 0 | 0 | 0 | 0 | 0 | 0 | 0 | 0 | 0 | 0 | 0 | 0 | 0 | 0 | 1 | 0.6 | 0 | 0 | 0 | 0 |
| 198 | 0 | 0 | 0 | 0 | 0 | 0 | 0 | 0 | 0 | 0 | 0 | 0 | 0 | 0 | 0 | 0.6 | 0 | 0 | 0 | 0 | 0 |
| 199 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 200 | 1 | 0 | 0 | 0 | 0 | 0 | 1 | 0 | 0 | 1 | 0 | 0 | 0 | 0 | 0 | 0 | 0 | 0 | 0 | 0.6 | 0 |
| 201 | 0 | 0 | 0 | 0 | 0 | 0 | 0 | 0 | 0 | 0.6 | 1 | 0 | 0 | 0 | 0 | 0 | 0 | 0 | 0 | 0 | 0 |
| 202 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 203 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 204 | 0 | 0 | 0 | 0 | 0 | 0 | 0 | 0 | 0 | 0 | 0 | 0.6 | 0 | 0 | 0 | 0 | 0 | 0 | 0 | 0 | 0 |
| 205 | 0 | 0 | 0 | 0 | 0 | 0 | 0 | 0 | 0 | 0 | 0 | 0 | 0 | 0 | 0 | 0 | 0 | 0 | 0.6 | 0 | 0 |
| 206 | 0 | 0 | 0 | 0 | 0 | 0 | 0 | 0 | 0 | 1 | 0.6 | 0 | 1 | 0 | 0 | 0 | 0 | 0 | 0 | 0 | 0 |
| 207 | 0 | 0 | 0 | 0.6 | 0 | 0 | 0 | 0 | 0 | 0 | 0 | 0 | 0 | 0 | 0 | 0 | 0 | 0 | 0 | 0 | 0 |
| 208 | 0 | 0 | 0 | 0 | 0 | 0 | 0 | 0 | 0 | 0 | 0 | 0 | 0 | 0 | 0 | 0 | 0 | 0 | 0.6 | 0 | 0 |
| 209 | 0 | 0 | 0 | 0 | 0.6 | 0 | 0 | 0 | 0 | 0 | 1 | 0 | 0 | 0 | 0 | 0 | 0 | 0 | 0 | 0 | 0 |
| 210 | 1 | 0 | 0 | 0 | 0 | 1 | 0 | 0 | 0 | 0 | 0 | 0 | 1 | 0 | 0 | 1 | 0.6 | 0 | 0 | 0 | 0 |
| 211 | 0 | 0 | 0 | 0 | 0 | 0 | 0.6 | 0 | 0 | 0 | 0 | 1 | 0 | 0 | 0 | 0 | 1 | 0 | 0 | 0 | 0 |
| 212 | 0 | 0 | 0 | 0 | 0 | 0 | 0 | 0 | 0 | 0 | 0.6 | 0 | 0 | 0 | 0 | 0 | 0 | 1 | 0 | 0 | 0 |
| 213 | 0 | 0 | 0 | 0 | 0 | 0 | 0 | 0 | 0 | 0 | 0 | 0 | 1 | 0 | 0 | 1 | 0.6 | 0 | 0 | 0 | 0 |
| 214 | 0 | 0 | 0.6 | 0 | 0 | 0 | 0 | 1 | 0 | 0 | 0 | 0 | 0 | 0 | 0 | 0 | 0 | 0 | 0 | 0 | 0 |
| 215 | 0 | 1 | 0 | 0 | 0 | 0 | 0 | 0 | 0 | 1 | 1 | 1 | 0 | 0 | 0 | 0 | 0 | 1 | 0 | 0.6 | 0 |
| 216 | 0 | 0 | 0 | 0 | 0 | 1 | 0 | 0 | 0 | 0 | 0 | 0.6 | 0 | 0 | 0 | 0 | 0 | 0 | 0 | 0 | 0 |
| 217 | 0 | 0 | 0 | 0 | 0 | 0 | 0 | 0 | 0 | 1 | 0 | 0 | 0 | 0 | 0 | 0 | 0 | 0 | 0 | 0.6 | 0 |
| 218 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 219 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 220 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 221 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 222 | 1 | 0 | 0 | 0 | 0 | 0 | 0 | 0 | 0 | 0 | 0 | 0 | 0 | 0 | 0.6 | 0 | 0 | 0 | 0 | 0 | 0 |
| 223 | 0 | 0 | 0 | 0 | 0 | 0 | 0 | 0 | 0 | 0 | 0 | 0 | 0 | 0 | 0.6 | 1 | 1 | 0 | 0 | 0 | 0 |
| 224 | 0 | 0 | 0 | 0 | 0 | 0 | 0 | 0 | 0 | 0 | 0 | 0 | 0 | 0.6 | 0 | 0 | 0 | 0 | 1 | 1 | 0 |
| 225 | 0 | 0 | 0 | 0 | 0 | 0.6 | 0 | 0 | 0 | 0 | 1 | 0 | 0 | 0 | 0 | 0 | 0 | 0 | 0 | 0 | 0 |
| 226 | 0 | 0 | 0 | 0.6 | 0 | 0 | 0 | 0 | 0 | 0 | 0 | 0 | 0 | 0 | 0 | 0 | 1 | 0 | 0 | 1 | 0 |
| 227 | 1 | 0 | 0 | 0 | 0 | 0 | 0 | 0 | 0 | 0 | 1 | 0 | 0 | 0 | 0.6 | 0 | 0 | 0 | 0 | 0 | 0 |
| 228 | 0 | 0 | 1 | 0 | 0 | 0 | 0 | 0 | 0 | 0 | 0.6 | 0 | 0 | 0 | 0 | 0 | 1 | 0 | 0 | 1 | 0 |
| 229 | 0 | 0 | 0 | 0 | 0 | 0 | 0 | 0 | 0 | 0 | 0 | 0 | 0 | 1 | 1 | 0 | 0.6 | 0 | 1 | 0 | 0 |
| 230 | 0 | 0 | 1 | 1 | 0 | 0 | 0.6 | 0 | 0 | 0 | 1 | 0 | 0 | 0 | 0 | 1 | 0 | 0 | 0 | 0 | 0 |
| 231 | 0 | 0 | 0 | 0.6 | 0 | 0 | 0 | 0 | 0 | 0 | 0 | 0 | 0 | 0 | 0 | 0 | 0 | 0 | 0 | 0 | 0 |
| 232 | 1 | 0 | 1 | 1 | 0 | 0 | 1 | 0 | 0 | 0.6 | 1 | 0 | 0 | 0 | 0 | 0 | 0 | 0 | 0 | 0 | 0 |
| 233 | 0 | 1 | 0 | 0.6 | 0 | 0 | 0 | 1 | 0 | 0 | 0 | 0 | 0 | 0 | 0 | 1 | 0 | 0 | 1 | 0 | 0 |
| 234 | 0 | 0 | 0 | 0.6 | 0 | 0 | 0 | 0 | 0 | 0 | 0 | 0 | 0 | 0 | 0 | 0 | 0 | 0 | 0 | 0 | 0 |
| 235 | 0 | 0 | 0 | 0 | 0 | 0 | 0 | 0 | 0 | 1 | 0 | 1 | 1 | 0 | 0.6 | 1 | 0 | 0 | 0 | 1 | 0 |
| 236 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 237 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 238 | 0 | 0 | 0 | 0 | 0 | 0 | 0 | 0 | 0 | 0.6 | 0 | 0 | 0.6 | 0 | 0 | 0 | 0 | 0 | 0 | 0 | 0 |
| 239 | 0 | 1 | 0 | 0.6 | 0 | 0 | 0 | 0 | 0 | 0 | 0 | 1 | 0 | 0 | 0 | 0 | 0 | 0 | 0 | 0 | 0 |
| 240 | 0 | 0 | 0 | 0 | 0 | 0 | 0 | 0 | 0 | 0 | 0 | 0 | 0 | 0 | 0 | 0 | 0 | 0.6 | 0 | 0 | 0 |
| 241 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 242 | 0 | 0 | 0 | 0.6 | 0 | 0 | 0 | 1 | 0 | 0 | 0 | 0 | 0 | 0 | 0 | 0 | 0 | 0 | 0 | 0 | 0 |
| 243 | 0 | 0 | 0 | 0 | 0 | 0 | 0 | 0 | 0 | 0 | 0 | 1 | 0 | 0 | 0 | 0 | 0.6 | 0 | 0 | 0 | 0 |
| 244 | 0 | 0 | 0 | 0 | 0 | 0 | 0 | 0 | 0 | 0 | 0 | 0.6 | 0 | 0 | 0 | 0 | 0 | 0 | 0 | 0 | 0 |
| 245 | 0 | 0 | 0 | 1 | 0 | 0 | 0 | 0 | 0 | 0 | 0 | 0 | 0 | 0 | 0.6 | 0 | 0 | 0 | 0 | 0 | 0 |
| 246 | 0 | 0 | 0 | 0 | 0 | 0 | 0.6 | 0 | 0 | 0 | 1 | 0 | 0 | 0 | 1 | 0 | 1 | 0 | 0 | 0 | 0 |
| 247 | 0 | 0 | 0.6 | 0 | 0 | 0 | 0 | 0 | 0.6 | 0 | 0 | 0 | 0 | 0 | 0 | 0 | 0 | 0 | 0 | 0 | 0 |
| 248 | 1 | 0 | 0 | 0 | 0 | 0 | 0 | 0 | 0 | 0 | 0 | 0 | 0 | 0 | 0 | 0.6 | 0 | 0 | 0 | 0 | 0 |
| 249 | 0 | 0 | 0 | 0 | 0 | 0 | 0 | 0 | 0 | 0 | 0 | 0 | 0 | 0 | 0 | 0 | 1 | 0 | 0 | 0.6 | 0 |
| 250 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 251 | 0 | 0 | 0 | 0 | 0 | 0 | 0 | 0 | 0 | 0 | 0 | 0 | 0 | 1 | 0 | 0 | 0 | 0 | 0.6 | 0 | 0 |
| 252 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 253 | 0 | 0 | 0 | 0 | 0 | 0 | 0 | 0 | 0 | 0 | 0 | 0 | 0 | 0 | 0 | 0.6 | 0 | 0 | 0 | 0 | 0 |
| 254 | 0 | 0 | 0 | 0 | 0 | 0 | 0 | 0 | 0 | 0 | 0 | 0 | 0 | 0.6 | 0 | 0 | 0 | 0 | 0 | 0 | 0 |
| 255 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 256 | 0 | 0 | 0 | 0 | 0 | 0 | 0 | 0 | 0 | 0 | 0 | 0 | 0 | 0 | 0 | 0.6 | 0 | 0 | 0 | 0 | 0 |
| 257 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 258 | 0.6 | 0 | 0 | 0 | 0 | 0 | 0 | 0 | 0 | 0 | 0 | 0 | 0 | 0 | 0 | 0 | 0 | 0 | 0.6 | 0 | 0 |
| 259 | 0 | 0 | 0 | 0 | 0 | 0 | 0 | 0 | 0 | 0 | 0 | 0 | 0 | 0.6 | 0 | 0 | 0 | 0 | 0 | 0 | 0 |
| 260 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 261 | 0 | 0 | 0 | 0 | 0 | 0 | 0 | 0 | 0 | 0 | 0 | 0 | 0 | 0 | 0 | 0.6 | 0 | 0 | 0 | 0 | 0 |
| 262 | 0 | 0.6 | 0 | 0 | 0 | 0 | 0 | 0 | 0 | 0 | 0 | 1 | 0 | 0 | 0 | 0 | 0 | 0 | 0 | 0 | 0 |
| 263 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 264 | 0 | 0 | 0 | 0.6 | 0 | 1 | 0 | 0 | 0 | 0 | 0 | 0 | 0 | 0 | 0 | 0 | 0 | 0 | 0 | 0 | 0 |
| 265 | 0 | 0 | 0 | 0 | 0 | 0 | 0 | 0 | 0 | 0 | 1 | 0 | 0.6 | 0 | 0 | 0 | 0 | 0 | 0 | 0 | 0 |
| 266 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 267 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 268 | 0 | 0 | 0 | 0 | 0 | 0 | 0 | 0 | 0 | 1 | 1 | 0 | 0 | 0 | 0 | 0 | 0 | 0 | 0 | 0.6 | 0 |
| 269 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 270 | 0 | 0 | 0 | 0 | 0 | 0 | 0 | 0 | 1 | 0 | 0 | 0 | 0 | 0.6 | 0 | 0 | 0 | 1 | 0 | 0 | 0 |
| 271 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 272 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 273 | 0 | 0 | 0 | 0 | 0 | 0 | 0.6 | 0 | 0 | 0 | 0 | 0 | 0 | 0 | 0 | 0 | 0 | 0 | 0 | 1 | 0 |
| 274 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 275 | 0 | 0 | 0 | 0 | 0 | 0 | 0 | 0 | 0 | 0 | 0 | 0 | 0 | 0 | 0 | 0.6 | 0 | 0 | 0 | 0 | 0 |
| 276 | 0 | 0 | 0.6 | 0 | 0 | 0 | 0 | 0.6 | 0 | 0 | 0 | 0 | 0 | 0 | 0 | 0 | 0 | 0 | 0 | 0 | 0 |
| 277 | 1 | 0 | 0 | 0 | 0 | 0 | 0 | 0 | 0 | 1 | 0 | 0 | 0 | 0 | 0 | 1 | 0 | 0 | 0 | 0.6 | 0 |
| 278 | 0 | 0 | 0.6 | 0 | 0 | 0 | 0 | 0 | 0 | 0 | 0 | 0 | 0 | 0 | 0 | 0 | 0 | 0 | 0 | 0 | 0 |
| 279 | 1 | 0 | 0 | 0 | 0 | 0 | 0 | 0 | 0 | 0 | 0 | 0 | 0 | 0.6 | 0 | 0 | 0 | 0 | 0 | 0 | 0 |
| 280 | 0 | 0 | 0 | 0 | 0 | 0 | 0 | 0 | 0 | 0.6 | 0 | 0 | 0 | 0 | 0 | 0 | 0 | 0 | 0 | 0 | 0 |
| 281 | 0 | 0 | 0 | 0 | 0 | 0 | 0 | 0 | 0 | 1 | 0 | 0 | 0 | 0 | 0 | 0 | 0 | 0.6 | 0 | 0 | 0 |
| 282 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 283 | 0.6 | 0 | 0 | 0 | 0 | 0 | 0 | 0 | 0 | 0 | 0 | 0 | 0 | 0 | 0 | 0 | 0 | 0 | 0 | 1 | 0 |
| 284 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 285 | 0 | 0 | 0 | 0 | 0 | 0 | 0 | 0 | 0 | 0 | 0 | 0 | 0 | 0.6 | 0 | 0 | 0 | 0 | 0 | 0 | 0 |
| 286 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 287 | 0 | 0 | 0 | 0 | 0 | 0 | 0 | 0 | 0 | 0 | 0 | 0.6 | 0 | 0 | 0 | 0 | 0 | 0 | 0 | 0 | 0 |
| 288 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 289 | 1 | 1 | 0 | 0 | 0 | 0 | 0.6 | 0 | 0 | 0 | 0 | 0 | 0 | 0 | 0 | 0 | 0 | 0 | 0 | 1 | 0 |
| 290 | 0 | 1 | 0 | 0 | 0 | 0 | 0.6 | 0 | 0 | 0 | 0 | 1 | 0 | 0 | 0 | 0 | 0 | 0 | 0 | 0 | 0 |
| 291 | 1 | 1 | 0 | 0 | 0 | 0.6 | 0 | 0 | 0 | 1 | 1 | 0 | 0 | 0 | 0 | 0 | 0 | 0 | 0 | 1 | 0 |
| 292 | 0 | 0.6 | 0.6 | 0 | 0 | 0 | 0 | 0 | 1 | 0 | 1 | 0 | 0 | 0 | 0 | 0 | 1 | 0 | 0 | 0 | 0 |
| 293 | 1 | 0 | 0 | 0 | 0 | 0 | 0 | 0 | 0 | 0 | 0.6 | 0 | 0 | 0 | 0 | 0 | 0 | 0 | 0 | 0 | 0 |
| 294 | 0 | 0 | 0 | 0 | 0 | 0 | 0.6 | 0 | 0 | 1 | 0 | 0 | 0 | 0 | 0 | 0 | 0 | 0 | 0 | 0 | 0 |
| 295 | 0 | 0 | 0 | 0.6 | 0 | 0 | 1 | 0 | 0 | 0 | 0 | 0 | 0 | 0 | 0 | 0 | 0 | 0 | 0 | 0 | 0 |
| 296 | 1 | 0 | 0 | 0 | 0 | 0 | 0 | 0 | 0 | 0 | 0 | 0 | 0 | 0 | 0 | 0 | 0 | 0 | 0 | 0.6 | 0 |
| 297 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 298 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 299 | 0 | 1 | 0 | 0 | 0 | 0 | 0 | 0 | 0 | 0 | 0 | 0.6 | 0 | 0 | 1 | 1 | 0 | 0 | 0 | 0 | 0 |
| 300 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 301 | 0 | 0 | 0 | 0 | 0 | 0 | 0 | 0 | 0 | 0 | 0 | 0 | 0 | 0.6 | 0 | 0 | 1 | 0 | 0 | 0 | 0 |
| 302 | 0 | 0 | 0 | 0 | 0 | 0 | 0 | 0 | 0 | 0 | 0.6 | 0 | 0 | 0 | 0 | 1 | 0 | 0 | 0 | 0 | 0 |
| 303 | 0 | 0 | 0 | 1 | 0 | 0 | 0.6 | 0 | 0 | 0 | 0 | 0 | 0 | 0 | 0 | 0 | 0 | 0 | 0 | 0 | 0 |
| 304 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 305 | 0 | 0 | 0 | 0 | 0 | 0 | 0 | 1 | 0 | 1 | 0 | 0 | 0 | 0 | 0 | 0 | 0 | 0 | 0 | 0.6 | 0 |
| 306 | 0 | 1 | 0 | 0 | 0 | 0 | 0 | 0.6 | 0 | 0 | 0 | 0 | 0 | 0 | 0 | 0 | 0 | 0 | 0 | 0 | 0 |
| 307 | 0 | 0 | 0 | 0.6 | 0 | 0 | 1 | 0 | 0 | 0 | 0 | 0 | 0 | 0 | 0 | 0 | 0 | 0 | 0 | 0 | 0 |
| 308 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 309 | 1 | 0 | 0 | 0 | 0 | 0 | 0 | 0.6 | 0 | 0 | 0 | 0 | 0 | 0 | 0 | 0 | 0 | 0 | 0 | 0 | 0 |
| 310 | 0 | 0.6 | 0 | 0 | 0 | 0 | 0 | 0 | 0 | 0 | 0 | 0 | 1 | 0 | 0 | 0 | 0 | 0 | 1 | 0 | 0 |
| 311 | 0 | 0 | 0 | 0 | 0 | 0 | 0 | 0 | 0 | 0 | 0 | 0 | 0 | 0 | 0 | 0 | 0 | 1 | 0 | 0.6 | 0 |
| 312 | 0 | 1 | 0 | 0 | 0 | 0 | 0 | 0 | 0 | 0 | 0.6 | 0 | 0 | 0 | 0 | 0 | 0 | 0 | 0 | 0 | 0 |
| 313 | 0 | 0 | 0 | 0.6 | 0 | 0 | 0 | 0 | 0 | 0 | 0 | 0 | 0 | 0 | 1 | 0 | 1 | 0 | 0 | 0 | 0 |
| 314 | 0 | 0 | 0 | 0 | 0 | 0 | 0 | 0 | 0.6 | 0 | 0 | 0 | 0 | 0 | 0 | 0 | 0 | 0 | 0 | 0 | 0 |
| 315 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 316 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 317 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 318 | 0 | 0 | 0 | 0 | 0 | 0.6 | 0 | 0 | 0 | 0 | 0 | 0 | 0 | 0 | 0 | 0 | 0 | 0 | 0 | 0 | 0 |
| 319 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 320 | 0 | 0 | 0 | 0 | 1 | 0 | 0 | 0 | 0.6 | 0 | 0 | 0 | 0 | 0 | 0 | 0 | 0 | 0 | 0 | 0 | 0 |
| 321 | 0 | 0 | 0 | 0 | 0 | 0 | 0 | 0 | 0 | 0.6 | 0 | 0 | 0 | 0 | 0 | 0 | 0 | 0 | 0 | 0 | 0 |
| 322 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 323 | 0 | 0 | 0.6 | 0 | 0 | 0 | 0 | 0 | 0 | 0 | 0 | 1 | 0 | 0 | 0 | 1 | 0 | 0 | 0 | 0 | 0 |
| 324 | 0 | 0 | 0 | 0 | 0 | 0 | 0 | 0 | 0.6 | 0 | 0 | 0 | 0 | 0 | 0 | 0 | 0 | 0 | 0 | 0 | 0 |
| 325 | 0 | 0 | 0 | 0 | 0 | 0 | 0 | 0 | 0 | 0 | 0 | 1 | 0 | 0 | 0.6 | 1 | 0 | 0 | 0 | 0 | 0 |
| 326 | 1 | 0 | 0 | 0 | 0 | 0 | 0 | 0 | 0 | 0 | 0 | 0 | 0 | 0 | 0 | 0.6 | 0 | 0 | 0 | 0 | 0 |
| 327 | 0 | 0 | 0 | 0 | 0 | 0 | 0 | 0 | 0 | 1 | 0 | 0 | 0 | 0 | 0 | 0 | 0.6 | 0 | 0 | 1 | 0 |
| 328 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 329 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 330 | 0 | 0 | 0 | 0 | 0 | 0 | 0 | 0 | 0 | 0 | 0 | 0 | 0 | 0.6 | 0 | 0 | 0 | 0 | 0 | 0 | 0 |
| 331 | 0 | 0 | 0 | 0 | 0 | 0 | 0 | 0 | 0 | 0.6 | 0 | 0 | 1 | 0 | 0 | 0 | 0 | 0 | 0 | 1 | 0 |
| 332 | 0 | 0 | 0 | 0 | 0 | 0 | 0 | 0 | 0 | 0 | 0 | 0 | 0 | 0 | 0 | 0.6 | 0 | 0 | 0 | 0 | 0 |
| 333 | 0 | 0 | 0 | 0 | 0 | 0 | 0 | 0 | 0.6 | 0 | 0 | 0 | 0 | 0 | 0 | 0 | 0 | 0 | 0 | 0 | 0 |
| 334 | 0 | 0 | 0 | 0 | 0.6 | 0 | 0 | 0 | 0 | 0 | 0 | 0 | 0 | 0 | 0 | 0 | 0 | 0 | 0 | 0 | 0 |
| 335 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 336 | 0 | 0 | 0 | 0 | 0 | 0 | 0 | 0 | 0 | 0 | 0 | 0 | 0 | 0 | 0 | 0 | 0 | 0.6 | 0 | 0 | 0 |
| 337 | 0 | 0 | 0.6 | 0 | 0 | 0 | 0 | 0 | 0 | 0 | 0 | 0 | 0 | 0 | 0 | 0 | 0 | 0 | 0 | 0 | 0 |
| 338 | 0 | 0 | 0 | 0 | 0 | 0 | 0 | 0 | 0 | 0 | 0 | 0 | 0 | 0 | 0 | 0.6 | 0 | 0 | 0 | 0 | 0 |
| 339 | 0 | 0 | 0 | 0 | 0 | 0 | 0 | 0 | 0 | 1 | 0 | 0 | 0 | 0 | 0 | 0 | 0 | 1 | 0 | 0.6 | 0 |
| 340 | 0 | 0 | 0 | 0 | 0 | 0 | 0 | 0 | 0 | 0 | 1 | 0 | 0.6 | 0 | 0 | 0 | 0 | 0 | 0 | 0 | 0 |
| 341 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 342 | 1 | 0 | 0 | 0 | 0 | 0 | 0 | 0 | 0 | 0 | 0 | 0 | 0 | 0 | 0 | 0.6 | 0 | 0 | 0 | 0 | 0 |
| 343 | 0 | 0 | 0 | 0 | 0 | 0 | 0 | 0 | 0 | 0.6 | 1 | 0 | 0 | 0 | 0 | 0 | 0 | 0 | 0 | 0 | 0 |
| 344 | 0 | 0 | 0 | 0.6 | 0 | 0 | 0 | 0 | 1 | 0 | 0 | 0 | 0 | 0 | 0 | 0 | 0 | 0 | 1 | 0 | 0 |
| 345 | 0 | 0 | 0 | 0 | 0 | 0 | 0 | 0 | 0 | 0 | 0 | 0 | 0 | 0.6 | 0 | 0 | 0 | 0 | 0 | 0 | 0 |
| 346 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 347 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 348 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 349 | 0 | 0 | 0 | 0 | 0 | 0 | 0 | 0 | 0 | 0.6 | 0 | 0 | 0 | 0 | 0 | 0 | 0 | 0 | 0 | 0 | 0 |
| 350 | 0 | 0 | 0 | 0 | 0 | 0 | 0 | 0 | 0 | 0.6 | 0 | 0 | 0 | 0 | 0 | 0 | 0 | 0 | 0 | 0 | 0 |
| 351 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 352 | 0 | 0 | 0 | 0 | 0 | 0 | 0 | 0 | 0 | 0 | 0 | 0 | 0.6 | 0 | 0 | 0 | 0 | 0 | 0 | 0 | 0 |
| 353 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 354 | 0 | 0 | 0 | 0 | 0 | 0 | 0 | 0 | 0 | 0 | 0 | 0 | 0.6 | 0 | 0 | 0 | 0 | 0 | 0 | 0 | 0 |
| 355 | 0 | 0 | 0 | 0 | 0 | 0.6 | 0 | 0 | 0 | 0 | 0 | 0 | 0 | 0 | 0 | 0 | 0 | 0 | 0 | 0 | 0 |
| 356 | 0 | 0 | 0 | 0 | 0 | 0 | 0 | 0 | 0 | 0 | 0 | 0 | 0 | 0 | 0 | 0 | 0 | 0.6 | 0 | 0 | 0 |
| 357 | 0 | 0 | 0 | 0.6 | 0 | 0 | 0 | 0 | 0 | 0 | 0 | 0 | 0 | 0 | 0 | 0 | 0 | 0 | 0 | 0 | 0 |
| 358 | 0 | 0 | 0 | 0 | 0 | 0.6 | 0 | 0 | 0 | 0 | 0 | 0 | 0 | 0 | 0 | 0 | 0 | 0 | 0 | 0 | 0 |
| 359 | 0 | 0 | 0 | 0 | 0 | 0 | 0 | 0 | 0 | 0 | 0 | 0 | 0 | 0 | 0.6 | 0 | 0 | 0 | 0 | 0 | 0 |
| 360 | 0 | 0 | 0 | 0 | 0 | 0 | 0 | 0 | 0 | 0.6 | 0 | 0 | 0 | 0 | 0 | 0 | 0 | 0 | 0 | 0 | 0 |
| 361 | 0 | 0 | 0.6 | 0 | 0 | 0 | 0 | 0 | 0 | 0 | 0 | 0 | 0 | 0 | 0 | 0 | 0 | 0 | 0 | 0 | 0 |
| 362 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 363 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 364 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 365 | 0 | 0 | 0 | 0 | 0 | 0 | 0 | 0 | 0 | 1 | 0.6 | 0 | 1 | 0 | 0 | 0 | 0 | 0 | 0 | 1 | 0 |
| 366 | 0 | 1 | 1 | 0 | 0 | 0 | 0 | 0 | 0 | 0 | 0 | 0 | 0 | 0 | 0 | 0 | 0 | 0 | 0 | 0.6 | 0 |
| 367 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 368 | 0 | 0 | 0 | 0 | 0 | 0 | 0 | 0 | 0 | 0 | 0.6 | 0 | 1 | 0 | 0 | 0 | 0 | 1 | 0 | 0 | 0 |
| 369 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 370 | 0 | 0 | 0 | 0 | 0 | 0 | 0 | 0 | 0 | 1 | 0 | 0 | 0 | 0 | 0 | 0 | 0 | 0 | 0 | 0.6 | 0 |
| 371 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 372 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 373 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 374 | 0 | 0 | 0 | 0 | 0 | 0 | 0 | 0 | 0 | 0.6 | 0 | 0 | 0 | 0 | 0 | 0 | 0 | 0 | 0 | 1 | 0 |
| 375 | 0 | 1 | 0 | 0 | 0 | 0 | 0 | 0 | 0 | 0 | 0 | 0 | 0 | 0 | 0.6 | 1 | 0 | 0 | 0 | 0.6 | 0 |
| 376 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 377 | 0 | 1 | 0 | 0.6 | 0 | 1 | 0 | 0 | 0 | 0 | 0 | 0 | 0 | 0 | 0 | 0 | 0 | 0 | 0 | 0 | 0 |
| 378 | 0 | 0 | 0 | 1 | 0 | 0 | 0.6 | 0 | 0 | 0 | 0 | 0 | 0 | 0 | 0 | 0 | 0 | 0 | 0 | 0 | 0 |
| 379 | 0 | 0 | 1 | 0.6 | 0 | 0 | 1 | 0 | 0 | 0 | 1 | 0 | 0 | 0 | 0 | 0 | 0 | 0 | 0 | 0 | 0 |
| 380 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 381 | 0 | 0.6 | 0 | 0 | 0 | 0 | 0 | 0 | 0 | 0 | 0 | 1 | 0 | 0 | 0 | 1 | 0 | 0 | 0 | 0 | 0 |
| 382 | 0 | 0 | 0 | 0 | 0 | 0 | 0 | 0 | 0 | 0.6 | 0 | 0 | 0 | 0 | 0 | 0 | 0 | 0 | 0 | 0.6 | 0 |
| 383 | 0 | 0 | 0 | 0 | 0 | 0 | 0 | 0 | 0.6 | 0 | 0 | 0 | 0 | 0 | 1 | 0 | 0 | 0 | 0 | 0 | 0 |
| 384 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 385 | 0.6 | 0 | 1 | 1 | 0 | 0 | 1 | 0.6 | 0 | 0 | 0 | 0 | 0 | 0 | 0 | 0 | 0 | 0 | 0 | 0 | 0 |
| 386 | 1 | 0 | 0 | 0 | 0 | 0 | 0.6 | 0 | 0 | 0 | 0 | 0 | 0 | 0 | 0 | 0 | 0 | 0 | 0 | 0 | 0 |
| 387 | 0 | 0 | 0 | 0 | 0 | 0 | 0 | 0 | 0 | 0.6 | 0 | 0 | 0 | 0 | 0 | 0 | 0 | 0 | 0 | 0 | 0 |
| 388 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 389 | 0 | 0.6 | 0 | 0 | 0 | 0.6 | 1 | 0 | 0 | 0 | 0 | 1 | 0 | 0 | 0 | 0 | 0 | 0 | 0 | 0 | 0 |
| 390 | 0 | 0 | 0 | 0 | 1 | 0 | 0 | 0 | 0 | 0 | 0 | 0 | 0 | 0 | 0 | 0 | 1 | 0 | 0 | 0.6 | 0 |
| 391 | 0 | 0 | 0 | 0 | 0 | 0 | 0 | 0 | 0 | 1 | 0.6 | 0 | 0 | 0 | 0 | 0 | 0 | 0 | 0 | 1 | 0 |
| 392 | 0 | 1 | 0 | 0 | 0 | 0 | 0 | 0 | 0 | 0 | 0 | 0.6 | 0 | 0 | 0 | 0 | 0 | 0 | 0 | 0 | 0 |
| 393 | 0 | 0 | 0 | 0.6 | 0 | 0 | 1 | 0 | 0 | 0 | 0 | 0 | 0 | 0 | 0 | 1 | 0 | 0 | 0 | 0 | 0 |
| 394 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 395 | 0 | 0 | 0 | 0 | 0 | 0 | 0 | 0 | 0 | 0.6 | 0 | 0 | 1 | 0 | 0 | 0 | 0 | 0 | 0 | 1 | 0 |
| 396 | 0 | 0 | 1 | 0 | 1 | 0 | 0 | 1 | 0 | 0 | 0 | 0 | 0 | 1 | 0 | 0.6 | 1 | 0 | 0 | 1 | 0 |
| 397 | 0 | 0 | 0 | 0 | 0 | 0 | 1 | 0.6 | 0 | 0 | 0 | 0 | 0 | 0 | 0 | 0 | 0 | 0 | 0 | 0 | 0 |
| 398 | 1 | 0 | 0 | 0 | 0 | 0 | 0 | 0 | 0 | 0 | 0 | 1 | 0 | 0 | 0.6 | 0 | 0 | 0 | 0 | 0 | 0 |
| 399 | 0 | 0 | 0 | 0 | 0 | 0 | 0 | 0 | 0 | 1 | 1 | 0 | 0 | 0 | 0 | 0 | 0.6 | 0 | 0 | 0 | 0 |
| 400 | 0 | 0 | 0 | 0 | 0 | 0 | 0 | 0.6 | 0 | 0 | 0 | 0 | 0 | 0 | 0 | 0 | 0 | 0 | 0 | 0 | 0 |
| 401 | 0 | 0 | 0 | 0 | 0 | 1 | 0.6 | 0 | 0 | 0 | 0 | 0 | 0 | 0 | 0 | 0 | 0 | 0 | 0 | 0 | 0 |
| 402 | 0 | 0 | 1 | 0 | 0 | 0 | 1 | 0 | 0 | 0.6 | 0 | 1 | 0 | 0 | 0 | 0 | 0 | 0 | 0 | 0 | 0 |
| 403 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 404 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 405 | 0.6 | 1 | 0 | 0 | 0 | 0 | 0 | 0 | 1 | 0 | 0 | 1 | 0 | 0 | 0 | 0 | 0 | 0 | 0 | 0 | 0 |
| 406 | 0 | 0 | 1 | 0 | 0 | 0 | 0 | 0 | 0 | 0 | 0 | 0.6 | 0 | 0 | 0 | 0 | 0 | 0 | 0 | 0 | 0 |
| 407 | 0 | 0 | 0 | 0 | 0 | 0 | 0 | 0 | 0 | 0 | 0 | 0 | 0 | 0 | 0 | 0 | 0 | 0 | 0 | 0.6 | 0 |
| 408 | 1 | 0.6 | 0 | 0 | 0 | 0 | 0 | 0 | 0 | 0 | 0 | 1 | 0 | 0 | 0 | 0 | 0 | 0 | 0 | 0 | 0 |
| 409 | 0 | 0 | 0 | 0.6 | 0 | 0 | 1 | 0 | 0 | 0 | 0 | 0 | 0 | 0 | 0 | 0 | 0 | 0 | 0 | 0 | 0 |
| 410 | 0 | 0 | 0 | 0 | 0 | 0 | 0 | 0 | 0 | 0.6 | 1 | 0 | 0 | 0 | 0 | 0 | 0 | 0 | 0 | 0 | 0 |
| 411 | 1 | 0 | 0 | 0 | 0 | 0 | 0 | 1 | 0 | 0 | 0 | 0 | 0 | 0 | 0 | 0.6 | 0 | 0 | 0 | 0 | 0 |
| 412 | 0.6 | 1 | 0 | 0 | 0 | 0 | 1 | 0 | 0 | 0 | 1 | 0.6 | 0 | 0 | 0 | 0 | 1 | 0 | 0 | 0 | 0 |
| 413 | 0 | 0.6 | 1 | 0 | 0 | 0 | 0 | 0 | 0 | 0 | 0 | 1 | 0 | 0 | 0 | 0 | 0 | 0 | 0 | 0 | 0 |
| 414 | 0 | 0 | 0 | 0 | 0 | 0 | 0 | 0 | 0 | 0 | 0.6 | 0 | 0 | 0 | 0 | 0 | 0 | 1 | 0 | 0 | 0 |
| 415 | 0 | 0.6 | 0 | 0 | 0 | 0 | 0 | 0 | 0 | 0 | 0 | 1 | 0 | 0 | 0 | 0 | 0 | 0 | 0 | 0 | 0 |
| 416 | 0.6 | 1 | 0 | 0 | 0 | 0 | 1 | 0 | 0 | 0 | 1 | 0 | 0 | 0 | 0 | 1 | 0 | 0 | 0 | 0 | 0 |
| 417 | 1 | 0.6 | 0 | 0 | 0 | 0 | 0 | 0 | 0 | 1 | 0 | 1 | 0 | 0 | 0 | 0 | 1 | 0 | 0 | 0 | 0 |
| 418 | 0 | 0.6 | 0 | 0 | 0 | 0 | 0 | 0 | 0 | 0 | 0 | 0 | 0 | 0 | 0 | 0 | 0 | 0 | 0 | 0 | 0 |
| 419 | 0 | 0 | 1 | 1 | 0 | 0 | 0.6 | 1 | 0 | 0 | 0 | 1 | 0 | 0 | 0 | 0 | 0 | 0 | 0 | 0 | 0 |
| 420 | 0 | 0 | 0 | 0 | 0 | 0 | 0.6 | 0 | 0 | 0 | 0 | 0 | 0 | 0 | 1 | 0 | 0 | 0 | 0 | 1 | 0 |
| 421 | 0 | 0 | 0 | 0 | 0 | 0 | 0.6 | 0 | 0 | 0 | 0 | 0 | 0 | 0 | 0 | 0 | 0 | 0 | 0 | 0 | 0 |
| 422 | 0 | 0 | 0 | 0 | 0 | 0 | 0 | 0 | 0 | 0 | 0 | 0 | 0.6 | 0 | 0 | 0 | 0 | 0 | 0 | 0 | 0 |
| 423 | 0 | 0 | 0.6 | 1 | 0 | 1 | 0 | 1 | 0 | 0 | 0 | 0 | 0 | 0 | 0 | 0 | 1 | 0 | 0 | 0 | 0 |
| 424 | 0.6 | 0 | 0 | 0 | 0 | 0 | 0 | 0 | 0 | 1 | 0 | 1 | 0 | 0 | 0 | 0 | 0 | 0 | 0 | 0 | 0 |
| 425 | 0.6 | 0 | 0 | 0 | 0 | 0 | 0 | 0 | 0 | 0 | 1 | 0 | 0 | 0 | 0 | 0 | 0 | 0 | 0 | 0 | 0 |
| 426 | 0.6 | 0 | 0 | 0 | 0 | 0 | 0 | 0 | 0 | 0 | 0 | 0 | 1 | 0 | 0 | 0 | 0 | 0 | 0 | 1 | 0 |
| 427 | 0 | 0 | 0 | 0 | 0 | 0 | 0.6 | 0 | 0 | 0 | 1 | 0 | 0 | 0 | 0 | 0 | 0 | 0 | 0 | 0 | 0 |
| 428 | 1 | 0 | 0 | 0 | 0 | 0 | 0.6 | 0 | 0 | 0 | 0 | 0 | 0 | 0 | 0 | 0 | 0 | 0 | 0 | 0 | 0 |
| 429 | 0 | 0 | 0 | 0 | 0 | 0 | 0 | 0 | 0 | 0 | 0.6 | 0 | 0 | 1 | 0 | 0 | 0 | 0 | 1 | 0 | 0 |
| 430 | 0 | 0 | 0 | 0 | 0 | 0 | 0 | 0 | 0 | 0.6 | 1 | 1 | 1 | 0 | 0 | 0 | 0 | 0 | 0 | 0 | 0 |
| 431 | 1 | 1 | 0 | 0 | 0 | 0.6 | 0 | 0 | 0 | 0 | 1 | 1 | 0 | 0 | 0 | 1 | 0 | 0 | 0 | 0 | 0 |
| 432 | 0 | 0 | 0 | 0 | 0 | 0 | 0 | 0 | 0 | 0 | 0.6 | 0 | 0 | 0 | 0 | 0 | 0 | 0 | 0 | 0 | 0 |
| 433 | 0 | 0 | 0 | 0 | 0.6 | 0 | 0 | 1 | 1 | 0 | 0 | 0 | 0 | 0 | 0 | 0 | 0 | 0 | 0 | 0 | 0 |
| 434 | 0 | 0.6 | 0 | 0 | 0 | 1 | 0 | 0 | 1 | 0 | 0 | 1 | 0 | 1 | 1 | 0 | 1 | 0 | 1 | 1 | 0 |
| 435 | 0 | 0 | 0.6 | 0 | 0 | 0 | 0 | 0 | 1 | 0 | 0 | 1 | 0 | 0 | 0 | 0 | 0 | 0 | 0 | 0 | 0 |
| 436 | 0 | 0.6 | 0 | 0 | 0 | 0 | 0 | 0 | 0 | 0 | 1 | 0 | 0 | 0 | 0 | 0 | 0 | 0 | 0 | 0 | 0 |
| 437 | 1 | 1 | 0.6 | 0 | 0 | 0 | 0 | 0 | 0 | 0 | 1 | 0 | 0 | 0 | 0 | 1 | 0 | 0 | 0 | 0 | 0 |
| 438 | 0.6 | 0 | 0 | 0 | 0 | 0 | 0 | 0 | 0 | 0 | 0 | 0 | 0 | 0 | 0 | 0 | 0 | 0 | 0 | 0 | 0 |
| 439 | 1 | 0 | 0 | 0 | 0 | 0 | 0 | 0 | 0 | 0 | 1 | 1 | 0 | 1 | 0 | 1 | 1 | 0 | 0.6 | 0 | 0 |
| 440 | 0 | 0 | 1 | 0 | 0 | 0 | 0 | 0 | 0 | 0 | 0 | 0.6 | 0 | 0 | 0 | 0 | 0 | 0 | 0 | 1 | 0 |

### Example 15: Computational Design of ADP-Glucose Dependent B12GTs

Improved B12GTs were designed by using the designed B12GTs from Example 12 and Example 13 as starting scaffolds for further design rounds. Computational design methods were used to improve the stability and expression of seven B12GTs from Example 12 and Example 13. One hundred thirty-four computational designs were chosen for experimental validation (SEQ ID NOs: 1333-1466). Expression plasmids for the computational designs were built as in Example 1. Each B12GT variant was expressed and purified as in Example 2. Each B12GT variant was assayed in a one-pot reaction with a SuSy variant of pA21838. The purified B12GT and SuSy were reacted with 100 mg/mL RA50, 250 mM Sucrose, and 0.5 mM ADP in 50 mM pH 6 phosphate buffer and 50 mM NaCl for 24 hours at 60° C. Product rebaudiosides were monitored by LCMS similar to Example 3. Several designed enzymes expressed well and were active for Reb A to Reb D conversion (Table 18).

TABLE 18
Computational Designs of Improved B12GTs

| ID | % Conversion | Purified Conc (uM) |
|---|---|---|
| pA35535 | 42.8 | 38.1 |
| pA35532 | 38.9 | 27.1 |
| pA35534 | 37.9 | 44.6 |
| pA35594 | 36 | 12.7 |
| pA35536 | 35.5 | 65.5 |
| pA35568 | 33.5 | 25 |
| pA35531 | 33 | 20.7 |
| pA35574 | 32.5 | 42.4 |
| pA35526 | 31.8 | 13.6 |
| pA35565 | 31.8 | 28.5 |
| pA35576 | 30 | 13.5 |
| pA35564 | 28.9 | 23.8 |
| pA35575 | 25.6 | 22.3 |
| pA35525 | 22.7 | 10.5 |
| pA35511 | 22.4 | 42.9 |
| pA35562 | 20.6 | 43.2 |
| pA35508 | 19.2 | 92.7 |
| pA35561 | 18.3 | 37.3 |
| pA35503 | 18.1 | 86.4 |
| pA35533 | 17.6 | 56.9 |
| pA35522 | 17.1 | 13.2 |
| pA35530 | 16.9 | 38.6 |
| pA35613 | 16.5 | 11.8 |
| pA35515 | 13.4 | 66.4 |
| pA35507 | 12.1 | 76.3 |
| pA35510 | 11.9 | 33.2 |
| pA35516 | 11.8 | 57.7 |
| pA35506 | 11.5 | 63.3 |
| pA35513 | 11.1 | 48.3 |
| pA35514 | 9.8 | 65.8 |
| pA35592 | 8.5 | 18.4 |
| pA35563 | 8.2 | 32.9 |
| pA35593 | 8.1 | 6 |
| pA35567 | 7.2 | 49.1 |
| pA35614 | 6.5 | 9 |
| pA35501 | 6.3 | 69.1 |
| pA35496 | 6.1 | 36.6 |
| pA35598 | 5.9 | 8.4 |
| pA35502 | 5.9 | 69.7 |
| pA35493 | 5.6 | 40.5 |
| pA35570 | 5.6 | 25.5 |
| pA35497 | 5.4 | 27.3 |
| pA35509 | 5.4 | 53.2 |
| pA35495 | 5.4 | 40.9 |
| pA35499 | 5.2 | 67.2 |
| pA35517 | 5.1 | 6.4 |
| pA35569 | 5 | 47 |
| pA35505 | 4.8 | 97.3 |
| pA35528 | 4.7 | 14.1 |
| pA35490 | 4.6 | 68.8 |
| pA35603 | 4.5 | 10.1 |
| pA35487 | 4.4 | 44.9 |
| pA35498 | 4.4 | 31.1 |
| pA35584 | 4.3 | 17.6 |
| pA35566 | 4.3 | 47.6 |
| pA35492 | 4.2 | 55 |
| pA35527 | 4.1 | 11.8 |
| pA35518 | 3.9 | 5.7 |
| pA35520 | 3.8 | 2.6 |
| pA35529 | 3.7 | 13.5 |
| pA35504 | 3.6 | 50.1 |
| pA35484 | 3.6 | 13.9 |
| pA35489 | 3.6 | 36.2 |
| pA35548 | 3.6 | 5.4 |
| pA35571 | 3.5 | 27.6 |
| pA35524 | 3.5 | 3.1 |
| pA35488 | 3.5 | 36 |
| pA35483 | 3.5 | 14.8 |
| pA35543 | 3.5 | 2.9 |
| pA35541 | 3.5 | 3.6 |
| pA35482 | 3.5 | 16.2 |
| pA35500 | 3.4 | 46.5 |
| pA35546 | 3.4 | 2.2 |
| pA35545 | 3.4 | 4.1 |
| pA35494 | 3.4 | 44.3 |
| pA35519 | 3.4 | 3.1 |
| pA35491 | 3.3 | 44.5 |
| pA35604 | 3.3 | 6.9 |
| pA35521 | 3.3 | 4.4 |
| pA35542 | 3.3 | 3.9 |
| pA35486 | 3.2 | 31.2 |
| pA35547 | 3.2 | 2.1 |
| pA35544 | 3.1 | 4.2 |
| pA35523 | 3.1 | 1.8 |
| pA35481 | 3.1 | 18.4 |
| pA35537 | 3 | 0.8 |
| pA35485 | 3 | 31.3 |
| pA35538 | 3 | 2.1 |
| pA35540 | 2.8 | 3.2 |
| pA35559 | 2.7 | 14.6 |
| pA35539 | 2.7 | 3.9 |
| pA35557 | 2.7 | 25 |
| pA35581 | 1.5 | 24.3 |
| pA35560 | 1.4 | 40.5 |
| pA35512 | 1.1 | 24 |
| pA35588 | 1.1 | 16.5 |
| pA35580 | 1.1 | 6.8 |
| pA35589 | 0.9 | 2.8 |
| pA35591 | 0.8 | 16.6 |
| pA35587 | 0.6 | 10 |
| pA35556 | 0.5 | 16.5 |
| pA35602 | 0.4 | 3.8 |
| pA35586 | 0.4 | 13.2 |
| pA35590 | 0.4 | 16.2 |
| pA35553 | 0.4 | 9.9 |
| pA35555 | 0.2 | 15.9 |
| pA35558 | 0.2 | 30.4 |

### Example 16: Computational Design of ADP-Glucose Dependent B12GTs

Structural models of a third B12GT variant of pA28422 were generated and used as the starting point for computational designs. Computational designs were conducted to improve the stability and expression of the B12GT. Fifty-three computational designs were chosen for experimental validation (SEQ ID NOs: 830-882). Expression plasmids for the computational designs were built as in Example 1. Each B12GT variant was expressed and purified as in Example 2. Each B12GT variant was assayed in a one-pot reaction with a SuSy variant of pA21838. The purified B12GT and SuSy were reacted with 10 mg/mL RA50, 100 mM Sucrose, and 0.5 mM ADP in 50 mM pH 6 phosphate buffer, 3 mM MgCl₂ and 50 mM NaCl for 24 hours at 60° C. Product rebaudiosides were monitored by LCMS similar to Example 3. Several designed enzymes expressed well and were active for Reb A to Reb D conversion (Table 19).

TABLE 19
Top B12GT Computational Designs

| ID | % Conversion | Purified Conc (uM) |
|---|---|---|
| pA29846 | 21 | 11.3 |
| pA29856 | 20 | 27.6 |
| pA29850 | 19.6 | 28.9 |
| pA29870 | 13.6 | 18.2 |
| pA29845 | 13.5 | 20.1 |
| pA29823 | 10.6 | 0.6 |
| pA29847 | 9.7 | 12.1 |
| pA29822 | 8.7 | 20.7 |
| pA29837 | 8.4 | −3.1 |
| pA29848 | 7.6 | 54 |
| pA29829 | 7 | 9.1 |
| pA29821 | 6.4 | 5.7 |
| pA29828 | 6.1 | 3.8 |
| pA29859 | 5.3 | 12.6 |
| pA29853 | 4.7 | 29.8 |
| pA29824 | 4.3 | 10.5 |
| pA29854 | 3.9 | 16.9 |
| pA29852 | 3.7 | |
| pA29836 | 3.4 | 0.5 |
| pA29839 | 3.1 | 2 |
| pA29864 | 2.9 | 75.3 |
| pA29860 | 2.8 | 44.5 |
| pA29831 | 0.7 | 20.3 |

### Example 17: Computational Design of ADP Dependent Sucrose Synthases

Structural models of two SuSy variants of pA21838 were generated and used as the starting point for computational designs. The design strategies used to design ADPG-dependent B12GTs were used to design improved ADP dependent sucrose synthases. Two hundred and fifty-six computational designs were chosen for experimental validation (SEQ ID NOs: 925-1180). Expression plasmids for the computational designs were built as in Example 4. Each SuSy variant was expressed and purified as in Example 5. Each SuSy variant was assayed in a one-pot reaction with a B12GT variant of pA28422. The purified B12GT and SuSy were reacted with 100 mg/mL RA50, 250 mM Sucrose, and 0.5 mM ADP in 50 mM pH 6 phosphate buffer, 3 mM MgCl₂ and 50 mM NaCl for 24 hours at 60° C. Product rebaudiosides were monitored by LCMS similar to Example 3. The relative expression and Reb A to Reb D conversion of the designed enzymes, SEQ ID: 925-1048, are shown in Table 20. The relative expression and Reb A to Reb D conversion of the designed enzymes, SEQ ID: 1049-1104, are shown in Table 21. The top hits from the previous two experiments were re-evaluated with more relevant protein concentrations (Table 22). Finally, the relative expression and Reb A to Reb D conversion of the designed enzymes, SEQ ID: 1105-1180 are shown in Table 23.

TABLE 20
Top SuSy Computational Designs

| ID | % Conversion | Purified Conc (uM) |
|---|---|---|
| pA32819 | 11.9 | 15.5 |
| pA32719 | 11.7 | 22.2 |
| pA32712 | 11 | 13.6 |
| pA32667 | 10.5 | 22.7 |
| pA32602 | 9.9 | 19 |
| pA32854 | 9.9 | 26.9 |
| pA32924 | 9.9 | 18 |
| pA32858 | 9.6 | 30.2 |
| pA32732 | 9.6 | 31.1 |
| pA32652 | 9.6 | 23.5 |
| pA32582 | 9.5 | 32.3 |
| pA32692 | 9.5 | 35.4 |
| pA32657 | 9.2 | 13.2 |
| pA32734 | 9.2 | 26.3 |
| pA32821 | 9.1 | 25.3 |
| pA32704 | 9.1 | 20.9 |
| pA32601 | 9 | 15 |
| pA32706 | 8.8 | 28 |
| pA32721 | 8.6 | 20.6 |
| pA32614 | 8.5 | 23.8 |
| pA32684 | 8.5 | 26.7 |
| pA32736 | 8.3 | 33.2 |
| pA32609 | 8.3 | 30.3 |
| pA32926 | 8.3 | 19.5 |
| pA32639 | 8.2 | 13.1 |
| pA32655 | 8.1 | 64.4 |
| pA32696 | 8.1 | 30.3 |
| pA32855 | 8 | 30.2 |
| pA32704 | 7.9 | 19.4 |
| pA32820 | 7.9 | 20.1 |
| pA32651 | 7.9 | 8.4 |
| pA32819 | 7.7 | 10.6 |
| pA32697 | 7.7 | 11.2 |
| pA32602 | 7.6 | 19.4 |
| pA32723 | 7.5 | 59 |
| pA32625 | 7.5 | 27.1 |
| pA32678 | 7.4 | 51.5 |
| pA32622 | 7.3 | 15 |
| pA32731 | 7 | 5.4 |
| pA32629 | 6.9 | 11.8 |
| pA32700 | 6.7 | 24.4 |
| pA32608 | 6.6 | 26.4 |
| pA32708 | 6.4 | 9.3 |
| pA32686 | 6.2 | 15 |
| pA32698 | 6 | 28.6 |
| pA32589 | 5.9 | 35.5 |
| pA32680 | 5.8 | 22.3 |
| pA32586 | 5.7 | 22.8 |
| pA32700 | 5.7 | 28.6 |
| pA32606 | 5.6 | 24.6 |
| pA32620 | 5.6 | 15.6 |
| pA32596 | 5.5 | 54.2 |
| pA32616 | 5.4 | 39 |
| pA32696 | 5.4 | 66.8 |
| pA32600 | 4.8 | 29.4 |
| pA32669 | 4.7 | 56.5 |
| pA32682 | 4.6 | 33.2 |
| pA32845 | 4.2 | 17.1 |
| pA32868 | 4.1 | 9.6 |
| pA32715 | 4.1 | 27.5 |
| pA32650 | 3.8 | 29.7 |
| pA32620 | 3.6 | 16.4 |
| pA32690 | 3.6 | 15 |
| pA32716 | 3.5 | 37.9 |
| pA32702 | 3.5 | 13.2 |
| pA32863 | 3.4 | 10 |
| pA32605 | 3.2 | 29.1 |
| pA32595 | 2.9 | 41.2 |
| pA32847 | 2.6 | 8.8 |
| pA32594 | 2.5 | 23.5 |
| pA32670 | 2.4 | 24.7 |
| pA32675 | 2.2 | 28.8 |
| pA32880 | 2.1 | 11.5 |
| pA32837 | 2.1 | 19.6 |
| pA32702 | 2.1 | 12.4 |
| pA32688 | 2 | 22.8 |
| pA32591 | 1.9 | 47.5 |
| pA32662 | 1.9 | 23.6 |
| pA32701 | 1.7 | 21.9 |
| pA32758 | 1.5 | 19.7 |
| pA32603 | 1.5 | 57.3 |
| pA32671 | 1.2 | 15.6 |
| pA32671 | 1.1 | 22.4 |
| pA32842 | 0.9 | 5.5 |
| pA32781 | 0.9 | 10 |
| pA32934 | 0.9 | 15.3 |
| pA32831 | 0.9 | 7.6 |
| pA32793 | 0.8 | 7.4 |
| pA32662 | 0.8 | 15.2 |
| pA32701 | 0.8 | 23.3 |
| pA32717 | 0.8 | 30.7 |
| pA32748 | 0.8 | 28.3 |
| pA32672 | 0.7 | 32.3 |
| pA32826 | 0.7 | 2 |
| pA32816 | 0.7 | 13.6 |
| pA32664 | 0.7 | 9.7 |
| pA32754 | 0.6 | 22.6 |
| pA32666 | 0.6 | 46 |
| pA32770 | 0.5 | 11.7 |
| pA32779 | 0.5 | 8.3 |
| pA32844 | 0.5 | 6.4 |
| pA32665 | 0.4 | 21 |
| pA32659 | 0.4 | 25.1 |
| pA32799 | 0.3 | 33.5 |
| pA32790 | 0.2 | 22.6 |
| pA32787 | 0.2 | 14.3 |
| pA32623 | 0.2 | 13.8 |
| pA32866 | 0.2 | 29.3 |
| pA32767 | 0.2 | 23 |
| pA32828 | 0.2 | 9 |
| pA32876 | 0.2 | 5.4 |
| pA32797 | 0.1 | 9.6 |

TABLE 21
Top SuSy Computational Designs

| ID | % Conversion | Purified Conc (uM) |
|---|---|---|
| pA32930 | 89.9 | 2.7 |
| pA32894 | 87.3 | 4.8 |
| pA32892 | 86 | 9.6 |
| pA32729 | 83.3 | 3.1 |
| pA32732 | 80.2 | 3.3 |
| pA32869 | 78.3 | 11.8 |
| pA32734 | 77.3 | 3.4 |
| pA32646 | 69.8 | 5.4 |
| pA32735 | 53.9 | 4 |
| pA32652 | 49.8 | 2.8 |
| pA32923 | 47.8 | 5.5 |
| pA32724 | 40.5 | 2.8 |
| pA32722 | 36.8 | 1.3 |
| pA32723 | 36.4 | 2.7 |
| pA32727 | 30.5 | 1.8 |
| pA32653 | 26.1 | 3.4 |
| pA32888 | 25.3 | 3.9 |
| pA32650 | 24.3 | 3.1 |
| pA32868 | 21.7 | 2.6 |
| pA32632 | 20.2 | 3 |
| pA32634 | 19.2 | 2.7 |
| pA32922 | 18 | 1.7 |
| pA32765 | 12.3 | 2.8 |
| pA32895 | 12.3 | 2.8 |
| pA32884 | 10.9 | 4.3 |
| pA32880 | 10.6 | 3 |
| pA32638 | 10.2 | 3.3 |
| pA32751 | 10.2 | 1.2 |
| pA32903 | 9.5 | 3.3 |
| pA32769 | 8.9 | 1.6 |
| pA32871 | 8.5 | 2.8 |
| pA32770 | 7.9 | 1.3 |
| pA32780 | 7.9 | 1.9 |
| pA32754 | 7.2 | 1.6 |
| pA32752 | 6.6 | 3.8 |
| pA32760 | 6.4 | 1.5 |
| pA32775 | 6.3 | 2.5 |
| pA32904 | 6.2 | 3.2 |
| pA32761 | 6.2 | 2.2 |
| pA32793 | 6 | 2.1 |
| pA32881 | 5.7 | 1.4 |
| pA32902 | 5.5 | 3.2 |
| pA32779 | 5.5 | 1.7 |
| pA32623 | 5.4 | 0.8 |
| pA32918 | 5.2 | 4.8 |
| pA32758 | 5.2 | 1.3 |
| pA32746 | 5 | 3.8 |
| pA32750 | 4.6 | 2.1 |
| pA32882 | 4.6 | 1.9 |
| pA32747 | 4.5 | 5.1 |
| pA32876 | 4.2 | 1.2 |
| pA32782 | 4.1 | 1.4 |
| pA32767 | 4 | 2.2 |
| pA32785 | 3.9 | 2.1 |
| pA32749 | 3.9 | 3.1 |
| pA32914 | 3.7 | 2.2 |
| pA32875 | 3.7 | 2.1 |
| pA32917 | 3.4 | 2 |
| pA32763 | 3.2 | 2.1 |
| pA32737 | 3.2 | 1.8 |
| pA32886 | 3.2 | 2.4 |
| pA32757 | 3.2 | 2.3 |
| pA32743 | 2.8 | 3.6 |
| pA32932 | 2.8 | 2.8 |
| pA32885 | 2.8 | 2.6 |
| pA32865 | 2.7 | 1.3 |
| pA32791 | 2.7 | 2.4 |
| pA32913 | 2.6 | 2.3 |
| pA32742 | 2.5 | 1.4 |
| pA32879 | 2.4 | 1 |
| pA32738 | 2.1 | 3.7 |
| pA32916 | 1.9 | 0.9 |
| pA32912 | 1.7 | 0.8 |
| pA32906 | 1.7 | 3.1 |

TABLE 22
Top SuSy Computational Designs

| ID | % Conversion | Purified Conc (uM) |
|---|---|---|
| pA32821 | 57.6 | 39.4 |
| pA32888 | 54.8 | 40.1 |
| pA32854 | 52.3 | 28.7 |
| pA32858 | 51.8 | 55.2 |
| pA32855 | 47.6 | 28.8 |
| pA32868 | 46.4 | 35.4 |
| pA32924 | 45.7 | 25.2 |
| pA32930 | 45.7 | 44 |
| pA32926 | 42.6 | 31.7 |
| pA32692 | 41.7 | 28.5 |
| pA32684 | 40.8 | 31.1 |
| pA32819 | 40.7 | 19.5 |
| pA32820 | 40 | 24.7 |
| pA32731 | 37.9 | 67.2 |
| pA32655 | 37.5 | 39.4 |
| pA32894 | 37 | 48.2 |
| pA32646 | 36.9 | 28.8 |
| pA32892 | 35.9 | 50.8 |
| pA32629 | 35.4 | 12.5 |
| pA32625 | 34.5 | 11.4 |
| pA32869 | 30.9 | 54.8 |
| pA32732 | 30.7 | 83.5 |
| pA32923 | 28.7 | 40.4 |
| pA32706 | 28.7 | 41.3 |
| pA32719 | 26.9 | 47.4 |
| pA32729 | 26.6 | 87.8 |
| pA32697 | 26.4 | 13.5 |
| pA32652 | 26.3 | 37.2 |
| pA32704 | 23.9 | 29.1 |
| pA32696 | 23.3 | 56.5 |
| pA32712 | 22.1 | 22.6 |
| pA32650 | 21 | 34.9 |
| pA32639 | 19.8 | 14.7 |
| pA32722 | 18.5 | 44.1 |
| pA32734 | 18.1 | 66 |
| pA32724 | 17.7 | 25.7 |
| pA32727 | 17.7 | 27 |
| pA32922 | 17.3 | 25.2 |
| pA32715 | 17.1 | 31.7 |
| pA32845 | 16.9 | 7.4 |
| pA32651 | 16.8 | 15.4 |
| pA32620 | 16.1 | 19.3 |
| pA32602 | 16 | 37.9 |
| pA32735 | 15.5 | 76.1 |
| pA32667 | 15 | 44.6 |
| pA32736 | 14 | 59.7 |
| pA32622 | 14 | 29.6 |
| pA32708 | 13.7 | 9.7 |
| pA32721 | 13.4 | 49.5 |
| pA32723 | 12.8 | 53.7 |
| pA32698 | 11.2 | 23.8 |
| pA32680 | 10.5 | 18.6 |
| pA32601 | 10.5 | 44.7 |
| pA32586 | 8 | 45.5 |
| pA32682 | 7.2 | 50.4 |
| pA32582 | 7.1 | 55.4 |
| pA32609 | 6.7 | 49.5 |
| pA32600 | 6.6 | 23.1 |
| pA32608 | 6.2 | 41.2 |
| pA32700 | 6 | 47.4 |
| pA32686 | 5.7 | 23.3 |
| pA32669 | 5.5 | 52.3 |
| pA32606 | 5.5 | 33.4 |
| pA32589 | 4.9 | 74.3 |
| pA32616 | 4.6 | 53.3 |
| pA32678 | 4 | 70.8 |
| pA32614 | 3.9 | 71 |
| pA32657 | 3.6 | 18.5 |
| pA32596 | 2.8 | 100.6 |

TABLE 23
Top SuSy Computational Designs

| ID | % Conversion | Purified Conc (uM) |
|---|---|---|
| pA32929 | 86.6 | 7.5 |
| pA32891 | 86.3 | 8.3 |
| pA32853 | 86.2 | 13.7 |
| pA32587 | 86 | 26.3 |
| pA32887 | 85 | 17 |
| pA32725 | 81.7 | 24.1 |
| pA32889 | 79.2 | 7.2 |
| pA32599 | 78.5 | 16.1 |
| pA32633 | 78 | 10.8 |
| pA32927 | 68.1 | 8.5 |
| pA32691 | 58.5 | 17.9 |
| pA32658 | 49.4 | 19.7 |
| pA32683 | 48.3 | 18.4 |
| pA32931 | 27.7 | 11.2 |
| pA32597 | 24.8 | 33 |
| pA32579 | 23.5 | 20.5 |
| pA32679 | 21.7 | 13.3 |
| pA32621 | 21.7 | 3.7 |
| pA32581 | 21.3 | 19.5 |
| pA32695 | 17.2 | 40.2 |
| pA32647 | 15.8 | 5.9 |
| pA32637 | 14.8 | 18.9 |
| pA32873 | 11 | 10.9 |
| pA32585 | 10.9 | 13.5 |
| pA32631 | 10.4 | 5.4 |
| pA32859 | 7.7 | 12.6 |
| pA32619 | 6.6 | 5.9 |
| pA32689 | 6.2 | 10.9 |
| pA32617 | 4.9 | 3.8 |
| pA32613 | 4.7 | 25.6 |
| pA32641 | 4 | 24 |
| pA32768 | 3.6 | 10.9 |
| pA32774 | 2.9 | 5.9 |
| pA32778 | 2.8 | 4.2 |
| pA32773 | 2.5 | 5.4 |
| pA32673 | 1.9 | 13.4 |
| pA32843 | 1.7 | 3.8 |
| pA32583 | 1.3 | 22.1 |
| pA32897 | 1.1 | 3.1 |
| pA32610 | 1 | 10.2 |
| pA32776 | 0.7 | 5.3 |
| pA32849 | 0.6 | 10.7 |
| pA32685 | 0.5 | 37.1 |
| pA32839 | 0.5 | 2.2 |

### Example 18: Representing Successful SuSy Designs with a PSSM

The successful SuSy designs from Example 17 were used to create a PSSM (Table 24). The PSSM is a concise way to represent the successful designs and related sequences. Sequences the have a PSSM score greater than 556 are considered related to the active computational designs described in Example 17. To score a sequence with the generated PSSM, it must first be aligned with the representative sequence pA21838 (Seq ID No: 885). For example, the following successful designs, pA32853, pA32891, pA32892, pA32929, have the following PSSM scores: 557.2, 558.1, 558.1, 557.8, while the wild-type SuSy pA21838, has a PSSM score of only 536.3.

TABLE 24
Position Specific Scoring Matrix (PSSM) of Successful SuSy Designs

| Seq | Amino Acid | | | | | | | | | | | | | | | | | | | | |
|---|---|---|---|---|---|---|---|---|---|---|---|---|---|---|---|---|---|---|---|---|---|
| Pos | A | R | N | D | C | Q | E | G | H | I | L | K | M | F | P | S | T | W | Y | V | — |
| 1 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 2 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 3 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 4 | 1 | 1 | 0 | 0 | 0 | 0 | 0.7 | 0 | 0 | 0 | 0 | 0 | 0 | 0 | 0 | 1 | 0 | 0 | 0 | 0.7 | 0 |
| 5 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 6 | 0 | 0.7 | 0 | 1 | 0 | 0 | 0 | 0 | 0 | 0 | 0 | 0 | 0 | 0 | 0 | 0 | 0 | 0 | 0 | 0 | 0 |
| 7 | 0 | 0 | 0 | 0 | 0 | 0.7 | 1 | 0 | 0 | 0 | 0 | 0 | 0 | 0 | 0 | 0 | 0 | 0 | 0 | 0 | 0 |
| 8 | 0 | 0 | 0 | 0 | 0 | 0.7 | 0 | 0 | 0 | 0 | 0 | 0 | 0 | 1 | 0 | 0 | 0 | 0 | 0 | 0 | 0 |
| 9 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 10 | 1 | 1 | 0 | 0 | 0 | 1 | 0 | 0 | 0 | 0 | 0.7 | 1 | 0 | 0 | 0 | 1 | 0 | 0 | 0 | 0 | 0 |
| 11 | 0 | 0 | 0 | 0.7 | 0 | 1 | 1 | 0 | 0 | 0 | 0 | 0 | 0 | 0 | 0 | 1 | 0 | 0 | 0 | 0 | 0 |
| 12 | 0 | 0 | 1 | 0 | 0 | 0 | 0 | 0 | 1 | 0 | 0 | 0 | 0 | 0 | 0 | 0.7 | 0 | 0 | 0 | 0 | 0 |
| 13 | 0 | 1 | 0 | 0 | 0 | 0 | 0 | 0 | 0 | 0 | 0 | 0 | 0 | 0 | 0.7 | 0 | 0 | 0 | 0 | 0 | 0 |
| 14 | 0 | 0.7 | 1 | 1 | 0 | 0 | 1 | 0 | 0 | 0 | 0 | 0 | 0 | 0 | 0 | 0 | 0 | 0 | 0 | 0 | 0 |
| 15 | 1 | 0 | 0 | 1 | 0 | 1 | 1 | 0 | 0 | 0 | 0 | 0 | 0 | 0 | 0 | 0.7 | 1 | 0 | 0 | 0 | 0 |
| 16 | 0 | 0 | 0 | 0 | 0 | 0 | 0 | 0 | 0 | 0 | 1 | 0 | 0 | 0 | 0 | 0 | 0 | 0.7 | 0 | 1 | 0 |
| 17 | 0 | 0.7 | 0 | 0 | 0 | 0 | 0 | 0 | 1 | 0 | 0 | 0 | 0 | 0 | 0 | 0 | 0 | 0 | 1 | 0 | 0 |
| 18 | 0.7 | 1 | 0 | 0 | 0 | 0 | 0 | 0 | 0 | 0 | 1 | 0 | 0 | 0 | 0 | 0 | 0 | 0 | 0 | 0 | 0 |
| 19 | 0 | 0 | 0 | 0 | 0 | 0 | 0 | 0 | 0 | 0 | 1 | 0 | 0 | 0.7 | 0 | 0 | 0 | 0 | 0 | 0 | 0 |
| 20 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 21 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 22 | 0 | 1 | 0 | 0 | 0 | 0 | 0 | 0 | 0.7 | 0 | 0 | 0 | 0 | 0 | 0 | 0 | 0 | 0 | 0 | 0 | 0 |
| 23 | 0 | 0 | 0 | 0 | 0 | 0 | 0 | 0 | 0 | 0 | 0.7 | 0 | 0 | 0 | 0 | 0 | 0 | 0 | 1 | 0 | 0 |
| 24 | 0 | 0 | 0 | 0 | 0 | 0 | 0 | 0 | 0 | 0 | 1 | 0 | 0 | 0 | 0 | 0 | 0 | 0 | 0 | 0.7 | 0 |
| 25 | 0.7 | 0 | 0 | 0 | 0 | 0 | 0 | 1 | 0 | 0 | 0 | 0 | 0 | 0 | 0 | 0 | 0 | 0 | 0 | 0 | 0 |
| 26 | 0 | 0 | 0 | 0 | 0 | 1 | 0 | 0 | 0 | 0 | 1 | 0 | 0 | 0 | 0 | 0.7 | 0 | 0 | 0 | 0 | 0 |
| 27 | 0 | 0 | 0 | 0 | 0 | 0.7 | 0 | 1 | 0 | 0 | 0 | 0 | 0 | 0 | 0 | 0 | 0 | 0 | 0 | 0 | 0 |
| 28 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 29 | 0 | 0 | 0 | 0.7 | 0 | 0 | 0 | 1 | 0 | 0 | 0 | 0 | 0 | 0 | 1 | 0 | 1 | 0 | 0 | 0 | 0 |
| 30 | 0 | 0 | 0 | 0 | 0 | 0 | 0 | 0 | 0 | 1 | 1 | 0 | 1 | 1 | 0 | 0.7 | 0 | 0 | 0 | 0 | 0 |
| 31 | 0 | 0 | 0 | 0 | 0 | 0 | 0 | 0 | 0 | 0 | 1 | 0 | 0 | 0 | 0 | 0 | 0 | 0.7 | 0 | 0 | 0 |
| 32 | 0 | 0 | 0 | 0 | 0 | 1 | 0 | 0 | 0 | 0 | 0.7 | 0 | 0 | 0 | 0 | 0 | 0 | 0 | 0 | 0 | 0 |
| 33 | 0 | 1 | 0 | 0 | 0 | 0 | 0 | 0 | 0.7 | 0 | 0 | 0 | 0 | 0 | 0 | 0 | 0 | 0 | 0 | 0 | 0 |
| 34 | 0 | 0 | 0 | 0 | 0 | 0 | 0 | 0 | 1 | 0 | 0 | 0 | 0 | 0 | 0 | 1 | 0.7 | 0 | 0 | 0 | 0 |
| 35 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 36 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 37 | 0 | 0 | 0 | 0 | 0 | 0.7 | 0 | 0 | 0 | 0 | 0 | 0 | 0 | 0 | 0 | 0 | 0 | 1 | 0 | 0 | 0 |
| 38 | 0 | 0.7 | 0 | 1 | 0 | 0 | 0 | 0 | 0.7 | 0 | 0 | 0 | 0 | 0 | 0 | 0 | 0 | 0 | 0 | 0 | 0 |
| 39 | 0.7 | 0 | 0 | 0 | 0 | 0 | 1 | 0 | 0 | 0 | 0 | 0 | 0 | 0 | 0 | 0 | 0 | 0 | 0 | 0 | 0 |
| 40 | 0 | 0 | 0 | 0 | 0.7 | 0 | 0 | 0 | 0 | 0 | 1 | 0 | 0 | 1 | 0 | 0 | 0 | 0 | 0 | 0 | 0 |
| 41 | 1 | 0 | 0 | 1 | 0 | 0 | 1 | 0 | 0 | 0 | 1 | 0.7 | 0 | 0 | 0 | 0 | 0 | 0 | 0 | 0 | 0 |
| 42 | 1 | 1 | 1 | 1 | 0 | 0 | 1 | 0 | 0 | 0 | 0 | 0 | 0 | 0 | 0 | 0 | 0.7 | 0 | 0 | 0 | 0 |
| 43 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 44 | 0 | 0.7 | 0 | 0 | 1 | 0 | 0 | 0 | 0 | 0 | 0 | 0 | 0 | 0 | 0 | 0 | 0 | 0 | 1 | 0 | 0 |
| 45 | 1 | 0 | 0 | 0 | 0 | 0 | 0.7 | 0 | 0 | 0 | 0 | 0 | 0 | 0 | 0 | 0 | 0 | 0 | 0 | 0 | 0 |
| 46 | 0 | 0 | 0 | 0 | 0 | 0.7 | 1 | 0 | 0 | 0 | 0 | 0 | 0 | 0 | 1 | 0 | 0 | 0 | 0 | 0 | 0 |
| 47 | 0 | 0 | 0 | 1 | 0 | 1 | 1 | 0 | 0 | 0 | 0 | 0 | 0 | 0 | 0.7 | 0 | 0 | 0 | 0 | 0 | 0 |
| 48 | 0 | 0 | 0 | 1 | 0 | 0 | 0 | 1 | 0 | 0 | 0 | 0 | 0 | 0 | 0.7 | 0 | 0 | 0 | 0 | 0 | 0 |
| 49 | 0 | 0 | 1 | 0 | 0 | 0 | 0.7 | 0 | 0 | 0 | 0 | 0 | 0 | 0 | 0 | 0 | 0 | 0 | 0 | 0 | 0 |
| 50 | 0 | 0 | 0 | 0 | 0 | 0 | 1 | 0.7 | 0 | 0 | 0 | 0 | 0 | 0 | 0 | 0 | 0 | 0 | 0 | 0 | 0 |
| 51 | 0 | 0 | 0 | 0 | 0 | 0 | 1 | 0 | 0 | 0 | 0 | 0 | 0 | 0 | 0 | 1 | 0 | 0 | 0.7 | 0 | 0 |
| 52 | 1 | 0 | 0 | 0 | 0 | 0 | 1 | 1 | 0 | 0 | 1 | 0 | 0 | 0 | 0.7 | 0 | 0 | 0 | 0 | 0 | 0 |
| 53 | 0 | 0 | 0 | 0 | 0 | 0 | 0.7 | 0 | 0 | 0 | 1 | 0 | 0 | 0 | 0 | 0 | 0 | 0 | 0 | 0 | 0 |
| 54 | 1 | 0 | 0 | 0.7 | 0 | 0 | 0 | 1 | 0 | 0 | 1 | 1 | 1 | 0 | 0 | 0 | 1 | 0 | 0 | 0 | 0 |
| 55 | 0 | 0 | 0 | 1 | 0 | 0 | 0 | 1 | 0 | 0.7 | 0 | 0 | 0 | 0 | 0 | 1 | 1 | 0 | 0 | 0 | 0 |
| 56 | 0 | 0 | 0 | 0 | 0 | 0 | 0 | 0.7 | 0 | 0 | 0 | 0 | 0 | 0 | 0 | 1 | 0 | 0 | 0 | 0 | 0 |
| 57 | 1 | 0 | 0 | 0 | 0 | 0 | 0 | 0 | 0 | 0 | 0 | 0 | 0 | 0 | 1 | 0 | 0 | 0.7 | 0 | 1 | 0 |
| 58 | 0 | 0 | 0 | 0 | 0 | 0 | 0 | 0 | 0 | 0 | 0.7 | 0 | 0 | 1 | 0 | 0 | 0 | 0 | 0 | 0 | 0 |
| 59 | 0.7 | 0 | 0 | 0 | 0 | 0 | 1 | 1 | 0 | 0 | 0 | 0 | 0 | 0 | 0 | 0 | 0 | 0 | 0 | 0 | 0 |
| 60 | 0 | 1 | 0 | 0.7 | 0 | 1 | 1 | 0 | 0 | 0 | 0 | 0 | 0 | 0 | 0 | 0 | 0 | 0 | 0 | 0 | 0 |
| 61 | 1 | 0 | 0 | 0 | 0 | 0 | 0 | 0 | 0 | 0 | 1 | 0 | 0 | 0.7 | 0 | 0 | 0 | 0 | 0 | 1 | 0 |
| 62 | 0 | 0 | 0 | 0 | 0 | 0 | 0 | 0 | 0 | 0.7 | 0 | 0 | 0 | 0 | 0 | 0 | 0 | 0 | 0 | 0.7 | 0 |
| 63 | 0.7 | 1 | 0 | 0 | 0 | 1 | 1 | 0 | 0 | 0 | 0 | 1 | 0 | 0 | 0 | 0 | 0 | 0 | 0 | 0 | 0 |
| 64 | 1 | 1 | 0 | 0 | 0 | 0 | 0 | 0 | 0.7 | 0 | 0 | 1 | 0 | 0 | 0 | 0 | 0 | 0 | 0 | 0 | 0 |
| 65 | 1 | 0 | 0 | 0 | 0.7 | 0 | 0 | 0 | 0 | 0 | 0 | 0 | 0 | 0 | 0 | 0 | 0.7 | 0 | 0 | 0 | 0 |
| 66 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 67 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 68 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 69 | 1 | 0 | 0 | 0 | 0 | 0 | 0 | 0 | 0 | 0.7 | 0 | 0 | 0 | 0 | 0 | 0 | 0 | 0 | 0 | 1 | 0 |
| 70 | 0 | 0 | 0 | 0 | 0 | 0 | 0 | 0 | 0 | 1 | 1 | 0 | 0 | 0.7 | 0 | 0 | 0 | 0 | 0 | 1 | 0 |
| 71 | 0 | 0.7 | 0 | 1 | 0 | 0 | 0 | 0 | 1 | 0 | 1 | 0 | 0 | 0 | 0 | 0 | 0 | 0 | 0 | 0 | 0 |
| 72 | 1 | 0 | 0 | 0.7 | 0 | 0 | 0 | 0 | 0 | 0 | 0 | 0 | 0 | 0 | 0 | 0 | 0 | 0 | 0 | 0 | 0 |
| 73 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 74 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 75 | 1 | 0 | 0 | 0 | 0 | 0 | 0 | 0 | 0 | 1 | 0 | 0 | 0.7 | 1 | 0 | 0 | 0 | 0 | 0 | 1 | 0 |
| 76 | 0 | 0 | 0 | 0 | 1 | 0 | 0 | 0 | 0 | 0 | 0 | 0 | 0 | 0 | 0 | 0 | 0 | 0 | 1 | 0.7 | 0 |
| 77 | 0 | 0 | 0 | 0 | 0 | 0 | 0 | 0 | 0 | 0 | 1 | 0 | 0 | 0.7 | 0 | 0 | 0 | 0 | 0 | 0 | 0 |
| 78 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 79 | 0 | 0 | 0 | 0 | 0 | 0 | 0 | 0 | 0 | 1 | 1 | 0 | 0 | 0 | 0 | 0 | 0 | 0.7 | 0 | 1 | 0 |
| 80 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 81 | 0 | 0 | 0 | 0 | 0 | 0 | 1 | 0 | 0 | 0 | 0.7 | 0 | 0 | 0 | 1 | 0 | 0 | 0 | 0 | 0 | 0 |
| 82 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 83 | 0 | 0 | 0 | 0 | 0 | 0 | 0 | 0 | 0 | 0 | 0 | 0 | 0 | 0 | 0.7 | 0 | 0 | 0 | 0 | 1 | 0 |
| 84 | 1 | 0 | 0 | 0 | 0 | 0 | 0 | 0.7 | 0 | 0 | 0 | 0 | 0 | 0 | 0 | 0 | 0 | 0 | 0 | 0 | 0 |
| 85 | 0 | 1 | 0 | 0 | 0 | 0 | 0 | 0 | 0 | 0 | 0 | 0 | 0 | 0 | 0 | 0 | 0 | 0 | 0 | 0.7 | 0 |
| 86 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 87 | 0 | 1 | 0 | 0 | 0 | 0 | 0.7 | 0 | 0 | 0 | 0 | 0 | 0 | 0 | 0 | 0 | 0 | 0 | 1 | 0 | 0 |
| 88 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 89 | 0 | 0 | 0 | 0 | 0 | 0 | 0 | 0 | 0 | 0 | 1 | 0 | 0 | 0 | 0 | 0 | 0 | 0 | 1 | 0.7 | 0 |
| 90 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 91 | 0 | 0 | 0 | 0 | 0 | 0 | 0 | 0 | 0 | 0.7 | 0 | 0 | 0 | 1 | 0 | 0 | 0 | 0 | 0 | 0 | 0 |
| 92 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 93 | 0 | 0 | 0 | 0 | 0 | 0 | 0 | 0 | 0 | 1 | 1 | 0 | 0 | 0 | 0 | 0 | 0 | 0 | 0 | 0.7 | 0 |
| 94 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 95 | 0 | 0 | 0 | 1 | 0 | 0.7 | 0 | 0 | 0 | 0 | 0 | 0 | 0 | 0 | 0 | 0 | 1 | 0 | 0 | 0 | 0 |
| 96 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 97 | 0.7 | 0 | 0 | 1 | 0 | 0 | 1 | 0 | 0 | 0 | 0 | 0 | 0 | 0 | 0 | 0 | 1 | 0 | 0 | 1 | 0 |
| 98 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 99 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 100 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 101 | 0 | 0 | 0 | 0 | 0 | 0 | 0 | 0 | 0 | 1 | 0.7 | 0 | 0 | 0 | 0 | 0 | 0 | 0 | 0 | 1 | 0 |
| 102 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 103 | 0 | 0 | 0 | 0 | 0 | 0 | 0 | 0 | 0 | 0 | 0 | 0 | 0 | 0 | 0 | 0 | 0.7 | 0 | 0 | 1 | 0 |
| 104 | 0 | 0 | 0 | 0.7 | 0 | 0 | 1 | 0 | 0 | 0 | 0 | 0 | 0 | 0 | 0 | 1 | 0 | 0 | 0 | 0 | 0 |
| 105 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 106 | 0 | 0 | 0 | 0 | 0 | 0 | 0 | 0 | 0 | 0 | 0 | 0 | 0 | 1 | 0 | 0 | 0 | 0 | 0.7 | 0 | 0 |
| 107 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 108 | 1 | 0 | 0 | 0 | 0 | 0.7 | 0 | 0 | 0 | 0 | 0 | 0 | 0 | 0 | 0 | 0 | 0 | 0 | 0 | 0 | 0 |
| 109 | 0.7 | 0 | 0 | 0 | 0 | 0 | 0 | 0 | 0 | 0 | 0 | 0 | 0 | 1 | 0 | 0 | 0 | 0 | 0 | 0 | 0 |
| 110 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 111 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 112 | 0 | 1 | 0 | 0 | 0 | 0.7 | 1 | 10 | 0 | 0 | 0 | 0 | 0 | 0 | 0 | 0 | 0 | 0 | 0 | 0 | 0 |
| 113 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 114 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 115 | 0 | 0 | 1 | 1 | 0 | 0 | 0 | 0.7 | 0 | 0 | 0 | 1 | 0 | 0 | 0 | 0 | 1 | 0 | 0 | 0 | 0 |
| 116 | 0 | 0 | 0 | 0 | 0 | 0 | 0 | 1 | 0 | 0 | 0.7 | 0 | 0 | 0 | 0 | 0 | 0 | 0 | 0 | 0 | 0 |
| 117 | 1 | 0 | 1 | 1 | 0 | 0 | 1 | 0.7 | 0 | 0 | 0 | 0 | 0 | 0 | 0 | 0 | 0 | 0 | 0 | 0 | 0 |
| 118 | 0.7 | 0 | 0 | 0 | 0 | 0 | 0 | 1 | 1 | 0 | 0 | 0 | 0 | 0 | 1 | 1 | 0 | 0 | 0 | 0 | 0 |
| 119 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 120 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 121 | 0 | 0 | 1 | 1 | 0 | 0 | 0.7 | 0 | 0 | 0 | 0 | 0 | 0 | 0 | 1 | 0 | 0 | 0 | 0 | 0 | 0 |
| 122 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 123 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 124 | 0 | 0 | 0 | 0 | 0 | 0 | 0 | 0 | 0 | 0 | 0.7 | 0 | 0 | 0 | 0 | 0 | 1 | 1 | 0 | 0 | 0 |
| 125 | 0 | 0 | 0 | 0 | 0 | 0 | 1 | 0 | 0 | 0 | 0 | 1 | 0 | 0 | 0 | 0 | 0.7 | 0 | 0 | 0 | 0 |
| 126 | 0 | 0 | 0 | 0 | 0 | 0 | 0 | 0 | 0 | 1 | 0 | 0 | 0 | 0 | 0 | 0 | 0 | 1 | 0 | 0.7 | 0 |
| 127 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 128 | 0 | 0 | 0 | 0 | 0 | 0 | 0 | 0 | 0 | 0 | 1 | 0 | 0 | 0.7 | 0 | 0 | 0 | 1 | 0 | 0 | 0 |
| 129 | 0 | 0 | 0 | 0 | 0 | 0 | 0.7 | 1 | 0 | 0 | 0 | 0 | 0 | 0 | 0 | 0 | 0 | 0 | 0 | 0 | 0 |
| 130 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 131 | 0 | 0 | 0 | 0 | 0 | 0 | 0 | 0 | 0 | 0 | 1 | 0 | 0 | 0.7 | 0 | 0 | 0 | 0 | 0 | 0 | 0 |
| 132 | 0 | 0.7 | 1 | 0 | 0 | 0 | 0 | 0 | 0 | 0 | 0 | 0 | 0 | 0 | 0 | 0 | 0 | 0 | 0 | 0 | 0 |
| 133 | 0 | 1 | 0 | 0 | 0 | 0 | 0 | 0 | 0 | 0 | 0 | 0 | 0 | 0 | 0.7 | 0 | 0 | 0 | 0 | 0 | 0 |
| 134 | 0 | 0 | 1 | 0 | 0 | 0 | 1 | 1 | 1 | 0 | 0 | 0 | 0 | 0 | 0 | 1 | 1 | 0 | 0 | 0.7 | 0 |
| 135 | 0 | 0 | 0 | 0 | 0 | 0 | 0 | 0 | 0 | 0 | 0 | 0 | 0 | 1 | 0 | 0.7 | 0 | 0 | 0 | 1 | 0 |
| 136 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 137 | 0 | 0.7 | 0 | 0 | 0 | 0 | 0 | 0 | 0 | 0 | 0 | 1 | 0 | 0 | 0 | 0 | 0 | 0 | 0 | 0 | 0 |
| 138 | 0 | 0 | 0 | 0 | 0 | 0 | 0 | 0 | 0 | 0 | 0.7 | 0 | 1 | 0 | 0 | 0 | 0 | 0 | 0 | 0 | 0 |
| 139 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 140 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 141 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 142 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 143 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 144 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 145 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 146 | 0 | 1 | 1 | 0.7 | 0 | 0 | 0 | 0 | 0 | 0 | 0 | 0 | 0 | 0 | 0 | 0 | 0 | 0 | 0 | 0 | 0 |
| 147 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 148 | 0 | 0 | 0 | 0 | 0 | 0 | 0 | 0 | 0 | 0 | 0.7 | 0 | 0 | 0 | 0 | 0 | 0 | 0 | 0 | 1 | 0 |
| 149 | 0 | 0 | 0 | 0 | 0 | 1 | 1 | 0 | 0 | 0 | 0 | 0 | 0 | 0 | 0 | 0 | 0.7 | 0 | 0 | 0 | 0 |
| 150 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 151 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 152 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 153 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 154 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 155 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 156 | 0.7 | 0 | 0 | 0 | 0 | 0 | 0 | 0 | 0 | 0 | 0 | 0 | 0 | 0 | 0 | 1 | 0 | 0 | 0 | 0 | 0 |
| 157 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 158 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 159 | 0 | 0 | 0 | 0 | 0 | 0 | 0 | 0 | 0 | 0.7 | 1 | 0 | 1 | 0 | 0 | 0 | 0 | 0 | 0 | 0 | 0 |
| 160 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 161 | 0 | 0 | 0 | 0 | 0 | 1 | 0 | 1 | 0 | 0 | 0 | 0 | 0 | 0 | 0 | 0 | 0.7 | 0 | 0 | 0 | 0 |
| 162 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 163 | 0 | 0 | 0 | 0 | 0 | 0 | 0 | 0 | 0 | 0 | 0.7 | 0 | 0 | 0 | 0 | 0 | 1 | 0 | 0 | 0 | 0 |
| 164 | 0.7 | 0 | 0 | 0 | 0 | 0 | 0 | 1 | 0 | 0 | 0 | 1 | 0 | 0 | 0 | 0 | 0 | 0 | 0 | 0 | 0 |
| 165 | 0.7 | 1 | 0 | 1 | 0 | 1 | 1 | 1 | 0 | 0 | 0 | 1 | 0 | 0 | 0 | 0 | 0 | 0 | 0 | 0 | 0 |
| 166 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 167 | 0 | 0.7 | 0 | 1 | 0 | 0 | 0 | 0 | 0 | 0 | 1 | 0 | 1 | 0 | 0 | 0 | 0 | 0 | 0 | 0 | 0 |
| 168 | 0 | 0 | 0 | 1 | 0 | 1 | 1 | 0 | 0 | 0 | 0 | 0 | 0 | 0 | 0 | 0.7 | 0 | 0 | 1 | 0 | 0 |
| 169 | 0.7 | 1 | 0 | 0 | 0 | 0.7 | 0 | 0 | 0 | 0 | 0 | 1 | 0 | 0 | 0 | 0 | 0 | 0 | 0 | 0 | 0 |
| 170 | 0 | 0 | 0 | 0 | 0 | 0 | 0 | 0 | 0 | 0.7 | 1 | 0 | 0 | 0 | 0 | 0 | 0 | 0 | 0 | 0 | 0 |
| 171 | 0 | 0 | 0 | 0 | 0 | 0 | 0 | 0 | 0 | 1 | 0.7 | 0 | 0 | 0 | 0 | 0 | 0 | 0 | 0 | 0 | 0 |
| 172 | 0 | 0 | 0 | 1 | 0 | 0 | 0.7 | 0 | 0 | 0 | 0 | 0 | 0 | 0 | 0 | 0 | 0 | 0 | 0 | 0 | 0 |
| 173 | 0 | 0 | 0 | 0 | 0 | 0 | 0 | 0 | 0 | 0 | 0 | 0 | 0 | 0.7 | 0 | 0 | 0 | 1 | 0 | 0 | 0 |
| 174 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 175 | 0 | 1 | 0 | 0 | 0 | 0 | 0 | 0.7 | 0 | 0 | 0 | 0 | 0 | 0 | 0 | 1 | 0 | 0 | 0 | 0 | 0 |
| 176 | 0 | 0 | 0 | 0 | 0 | 0 | 0 | 0 | 0 | 0 | 0.7 | 0 | 0 | 0 | 0 | 0 | 0 | 0 | 0 | 1 | 0 |
| 177 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 178 | 0 | 0.7 | 0 | 0 | 0 | 1 | 0 | 0 | 0 | 0 | 0 | 0 | 0 | 0 | 0 | 0 | 0 | 0 | 0 | 0 | 0 |
| 179 | 0 | 0 | 0 | 0 | 0 | 0 | 0 | 0 | 1 | 1 | 0.7 | 0 | 0 | 0 | 0 | 0 | 0 | 0 | 1 | 0 | 0 |
| 180 | 0 | 1 | 0 | 0.7 | 0 | 0 | 0 | 0 | 0 | 0 | 0 | 0 | 0 | 0 | 0 | 0 | 0 | 0 | 0 | 0 | 0 |
| 181 | 0 | 0 | 0.7 | 0 | 0 | 0 | 0 | 1 | 0 | 0 | 0 | 0 | 0 | 0 | 0 | 0 | 0 | 0 | 0 | 0 | 0 |
| 182 | 0 | 1 | 0 | 0 | 0 | 0.7 | 0 | 0 | 0 | 0 | 0 | 0 | 0 | 0 | 0 | 0 | 0 | 0 | 0 | 0 | 0 |
| 183 | 0 | 0 | 0.7 | 0 | 0 | 1 | 0 | 0 | 1 | 0 | 0 | 0 | 0 | 0 | 0 | 0 | 0 | 0 | 0 | 0 | 0 |
| 184 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 185 | 0 | 0 | 0 | 0 | 0 | 0 | 0 | 1 | 0 | 0 | 0 | 0 | 0.7 | 0 | 0 | 0 | 0 | 0 | 0 | 0 | 0 |
| 186 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 187 | 0 | 0 | 1 | 0 | 0 | 0 | 0 | 0 | 0 | 0 | 0 | 0 | 0 | 0 | 0 | 0.7 | 0 | 0 | 0 | 0 | 0 |
| 188 | 0 | 0 | 0.7 | 1 | 0 | 0 | 1 | 0 | 0 | 0 | 0 | 0 | 0 | 0 | 0 | 0 | 0 | 0 | 0 | 0 | 0 |
| 189 | 0 | 1 | 0 | 0 | 0 | 0 | 0 | 0.7 | 0 | 0 | 0 | 0 | 0 | 0 | 0 | 0 | 0 | 0 | 0 | 0 | 0 |
| 190 | 0 | 0 | 0.7 | 0 | 0 | 0 | 0 | 0 | 0 | 1 | 0 | 0 | 1 | 1 | 0 | 0 | 0 | 0 | 0 | 0 | 0 |
| 191 | 0 | 1 | 0 | 0 | 0 | 1 | 0 | 0 | 0 | 0 | 0 | 1 | 0 | 0 | 0 | 0 | 0.7 | 0 | 0 | 0 | 0 |
| 192 | 0 | 0 | 0 | 0.7 | 0 | 0 | 0 | 0 | 0 | 0 | 0 | 0 | 0 | 0 | 0 | 1 | 0 | 0 | 0 | 0 | 0 |
| 193 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 194 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 195 | 1 | 0 | 0 | 0 | 0 | 0 | 1 | 1 | 0 | 0 | 0 | 0 | 0 | 0 | 0 | 0.7 | 0 | 0 | 0 | 0 | 0 |
| 196 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 197 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 198 | 0 | 1 | 0 | 0 | 0 | 0.7 | 0 | 0 | 0 | 0 | 0 | 0 | 0 | 0 | 0 | 0 | 0 | 0 | 0 | 0 | 0 |
| 199 | 1 | 0 | 0 | 0 | 0 | 0 | 0 | 0 | 0 | 0 | 0 | 0 | 0 | 0 | 0 | 0 | 0.7 | 0 | 0 | 1 | 0 |
| 200 | 0 | 0 | 0 | 0 | 0 | 0 | 1 | 0 | 0 | 1 | 1 | 0 | 1 | 0 | 0 | 0 | 0 | 0 | 0 | 0.7 | 0 |
| 201 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 202 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 203 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 204 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 205 | 0 | 1 | 0 | 0 | 0 | 0 | 0 | 1 | 0 | 0 | 0 | 1 | 0 | 0 | 0 | 0 | 0.7 | 0 | 0 | 0 | 0 |
| 206 | 0 | 0 | 0 | 0 | 0 | 1 | 0 | 0 | 0 | 0 | 0.7 | 0 | 0 | 0 | 0 | 0 | 0 | 0 | 0 | 0 | 0 |
| 207 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 208 | 1 | 0.7 | 0 | 1 | 0 | 0 | 1 | 0 | 0 | 0 | 0 | 0 | 0 | 0 | 1 | 0 | 0 | 0 | 0 | 0 | 0 |
| 209 | 0 | 0 | 0 | 1 | 0 | 0 | 0.7 | 0 | 0 | 0 | 0 | 0 | 0 | 0 | 0 | 0 | 0 | 0 | 0 | 0 | 0 |
| 210 | 1 | 0 | 0 | 0 | 0 | 0 | 0 | 0 | 0 | 0 | 0 | 0 | 0 | 0 | 0 | 0 | 0.7 | 0 | 0 | 0 | 0 |
| 211 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 212 | 0 | 0 | 0 | 0 | 0 | 0 | 0 | 0 | 0 | 0 | 1 | 0 | 0 | 0 | 0 | 0 | 0 | 0.7 | 0 | 0 | 0 |
| 213 | 0.7 | 0 | 0 | 0 | 0 | 0 | 1 | 0 | 0 | 0 | 0 | 0 | 0 | 0 | 0 | 1 | 0 | 0 | 0 | 0 | 0 |
| 214 | 0 | 0 | 0 | 1 | 0 | 0 | 0.7 | 0 | 0 | 0 | 0 | 0 | 0 | 0 | 0 | 0 | 0 | 0 | 0 | 0 | 0 |
| 215 | 0 | 0 | 0 | 0 | 0 | 0 | 0 | 0 | 0 | 0 | 1 | 0 | 0 | 0.7 | 0 | 0 | 0 | 0 | 0 | 1 | 0 |
| 216 | 1 | 0.7 | 0 | 0 | 0 | 1 | 1 | 1 | 0 | 0 | 0 | 0 | 0 | 0 | 0 | 0 | 0 | 0 | 0 | 0 | 0 |
| 217 | 0 | 0 | 0 | 0 | 0 | 0 | 0.7 | 0 | 1 | 0 | 0 | 0 | 0 | 0 | 1 | 0 | 0 | 0 | 0 | 0 | 0 |
| 218 | 1 | 1 | 0 | 0.7 | 0 | 0 | 1 | 0 | 0 | 0 | 0 | 1 | 0 | 0 | 0 | 0 | 1 | 0 | 0 | 0 | 0 |
| 219 | 0 | 0 | 0 | 0 | 0 | 0 | 0 | 0 | 0 | 0 | 1 | 0 | 0.7 | 0 | 0 | 0 | 0 | 0 | 0 | 0 | 0 |
| 220 | 0 | 0.7 | 0 | 0 | 0 | 1 | 0 | 0 | 0 | 0 | 0 | 0 | 0 | 0 | 0 | 0 | 0 | 0 | 0 | 0 | 0 |
| 221 | 0 | 0.7 | 0 | 0 | 0 | 0 | 1 | 0 | 0 | 0 | 0 | 0 | 0 | 0 | 0 | 0 | 0 | 0 | 0 | 0 | 0 |
| 222 | 0 | 0.7 | 0 | 0 | 0 | 0 | 0 | 0 | 0 | 0 | 1 | 0 | 1 | 0 | 0 | 0 | 0 | 1 | 0 | 0 | 0 |
| 223 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 224 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 225 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 226 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 227 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 228 | 0 | 0 | 0 | 0 | 0 | 0 | 0 | 0 | 0 | 0 | 1 | 0 | 0 | 0 | 0 | 0 | 0 | 0.7 | 0 | 0 | 0 |
| 229 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 230 | 0 | 1 | 0.7 | 1 | 0 | 0 | 0 | 0 | 0 | 0 | 0 | 0 | 0 | 0 | 0 | 0 | 0 | 0 | 0 | 0 | 0 |
| 231 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 232 | 0.7 | 0 | 0 | 0 | 0 | 0 | 0 | 0 | 0 | 0 | 0 | 0 | 0 | 0 | 0 | 0 | 0 | 0 | 0 | 1 | 0 |
| 233 | 1 | 0 | 0 | 0 | 0 | 1 | 1 | 0.7 | 0 | 0 | 0 | 0 | 0 | 0 | 0 | 0 | 0 | 0 | 0 | 0 | 0 |
| 234 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 235 | 0 | 0 | 0 | 0 | 0 | 0 | 0 | 0 | 0 | 1 | 0 | 0 | 1 | 0 | 0 | 0 | 0 | 0 | 0 | 0.7 | 0 |
| 236 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 237 | 0 | 0 | 0 | 1 | 0 | 0 | 0.7 | 0 | 0 | 0 | 0 | 0 | 0 | 0 | 0 | 0 | 0 | 0 | 0 | 0 | 0 |
| 238 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 239 | 0 | 0 | 0 | 0 | 0 | 0 | 0 | 0 | 0 | 0 | 1 | 0 | 0.7 | 0 | 0 | 0 | 0 | 0 | 0 | 0 | 0 |
| 240 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 241 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 242 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 243 | 0 | 0 | 0 | 0 | 0 | 0 | 0 | 0 | 0 | 0 | 0 | 0 | 0.7 | 0 | 0 | 0 | 0 | 0 | 0 | 1 | 0 |
| 244 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 245 | 0 | 0 | 0 | 0 | 0 | 0 | 0 | 0 | 0 | 1 | 0.7 | 0 | 0 | 0 | 0 | 0 | 0 | 0 | 0 | 0 | 0 |
| 246 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 247 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 248 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 249 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 250 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 251 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 252 | 0.7 | 1 | 0 | 0 | 0 | 0 | 1 | 1 | 0 | 0 | 0 | 0 | 0 | 0 | 0 | 1 | 0 | 0 | 0 | 0 | 0 |
| 253 | 0.7 | 0 | 1 | 0 | 0 | 0 | 0 | 0 | 0 | 0 | 0 | 0 | 0 | 0 | 0 | 0 | 1 | 0 | 0 | 0 | 0 |
| 254 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 255 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 256 | 0 | 1 | 0 | 0 | 0 | 0 | 1 | 0 | 0 | 0 | 0 | 1 | 0 | 0 | 0 | 0.7 | 1 | 0 | 0 | 0 | 0 |
| 257 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 258 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 259 | 1 | 0 | 0 | 0.7 | 0 | 0 | 0 | 1 | 0 | 0 | 0 | 0 | 0 | 0 | 0 | 0 | 0 | 0 | 0 | 0 | 0 |
| 260 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 261 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 262 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 263 | 0 | 0 | 0 | 0 | 0 | 0 | 0 | 0 | 0 | 0 | 1 | 0 | 0.7 | 0 | 0 | 0 | 0 | 0 | 0 | 0 | 0 |
| 264 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 265 | 0 | 0 | 0 | 0 | 0 | 0 | 0 | 0 | 0 | 0 | 0 | 0 | 0 | 1 | 0 | 0.7 | 0 | 0 | 0 | 0 | 0 |
| 266 | 0 | 0 | 0.7 | 0 | 0 | 0 | 0 | 0 | 0 | 0 | 0 | 0 | 0 | 0 | 0 | 1 | 0 | 0 | 0 | 0 | 0 |
| 267 | 0 | 0 | 0 | 0 | 0 | 0 | 0 | 0 | 0 | 1 | 1 | 0 | 0 | 0 | 0 | 0 | 0 | 0 | 0 | 0.7 | 0 |
| 268 | 1 | 0 | 0 | 0 | 0 | 0 | 0 | 0 | 0 | 1 | 0.7 | 0 | 0 | 0 | 0 | 0 | 1 | 0 | 0 | 1 | 0 |
| 269 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 270 | 0 | 0 | 0 | 0 | 0 | 0 | 0 | 0 | 0 | 1 | 1 | 0 | 0 | 0 | 0 | 0 | 0 | 0 | 0 | 0.7 | 0 |
| 271 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 272 | 0 | 0 | 0 | 0 | 0 | 0 | 0 | 0 | 0 | 0.7 | 0 | 0 | 0 | 0 | 1 | 0 | 0 | 0 | 0 | 1 | 0 |
| 273 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 274 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 275 | 0 | 0 | 0 | 0 | 0 | 0 | 0 | 0 | 0 | 0 | 0 | 0 | 0 | 0 | 0 | 0 | 0 | 0.7 | 1 | 0 | 0 |
| 276 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 277 | 0.7 | 0 | 0 | 0 | 0 | 0 | 0 | 1 | 0 | 0 | 0 | 0 | 0 | 0 | 0 | 0 | 0 | 0 | 0 | 0 | 0 |
| 278 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 279 | 1 | 0 | 0 | 0.7 | 0 | 0 | 0 | 0 | 0 | 0 | 0 | 0 | 0 | 0 | 0 | 1 | 0 | 0 | 0 | 0 | 0 |
| 280 | 0 | 0 | 1 | 0 | 0 | 0 | 0 | 0 | 0 | 0 | 0 | 0.7 | 0 | 0 | 0 | 0 | 0 | 0 | 0 | 0 | 0 |
| 281 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 282 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 283 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 284 | 0 | 0.7 | 0 | 0 | 0 | 0 | 0 | 0 | 0 | 0 | 1 | 0 | 0 | 0 | 0 | 0 | 0 | 0 | 1 | 0 | 0 |
| 285 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 286 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 287 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 288 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 289 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 290 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 291 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 292 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 293 | 0 | 0 | 0 | 0 | 0 | 0 | 0 | 0 | 0 | 0 | 0 | 0 | 0 | 1 | 0 | 0 | 0 | 0 | 0.7 | 0 | 0 |
| 294 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 295 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 296 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 297 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 298 | 0.7 | 0 | 0 | 0 | 0 | 0 | 0 | 0 | 0 | 0 | 0 | 0 | 0 | 0 | 0 | 0 | 0 | 0 | 0 | 1 | 0 |
| 299 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 300 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 301 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 302 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 303 | 0 | 0.7 | 0 | 0 | 0 | 0 | 0 | 0 | 0 | 0 | 0 | 1 | 0 | 0 | 0 | 0 | 0 | 0 | 0 | 0 | 0 |
| 304 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 305 | 0 | 0 | 0 | 0 | 0 | 0 | 0 | 0 | 0 | 1 | 1 | 0 | 0.7 | 0 | 0 | 0 | 0 | 0 | 0 | 0 | 0 |
| 306 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 307 | 0 | 1 | 0.7 | 1 | 0 | 1 | 1 | 0 | 0 | 0 | 0 | 1 | 0 | 0 | 0 | 0 | 0 | 0 | 0 | 0 | 0 |
| 308 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 309 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 310 | 1 | 0.7 | 0 | 0 | 0 | 0 | 0 | 0 | 1 | 0 | 0 | 0 | 0 | 0 | 0 | 0 | 0 | 0 | 1 | 0 | 0 |
| 311 | 0 | 0 | 0 | 0 | 0 | 0.7 | 1 | 0 | 0 | 0 | 0 | 0 | 0 | 0 | 0 | 0 | 0 | 0 | 0 | 0 | 0 |
| 312 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 313 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 314 | 0 | 0 | 0 | 0 | 0 | 0 | 0 | 0 | 0 | 0 | 1 | 0 | 0 | 0 | 0 | 0 | 0 | 0 | 0 | 0.7 | 0 |
| 315 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 316 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 317 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 318 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 319 | 0 | 0.7 | 0 | 0 | 0 | 1 | 0 | 0 | 0 | 0 | 0 | 0 | 0 | 0 | 0 | 0 | 0 | 0 | 0 | 0 | 0 |
| 320 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 321 | 0 | 0 | 0 | 0 | 0 | 0 | 0 | 0 | 0 | 1 | 0.7 | 0 | 0 | 0 | 0 | 0 | 0 | 0 | 0 | 0 | 0 |
| 322 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 323 | 0.7 | 0 | 0 | 0 | 0 | 0 | 0 | 0 | 0 | 1 | 1 | 0 | 0 | 0 | 0 | 0 | 0 | 0 | 0 | 1 | 0 |
| 324 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 325 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 326 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 327 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 328 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 329 | 0 | 0 | 0 | 1 | 0 | 0 | 0.7 | 0 | 0 | 0 | 0 | 0 | 0 | 0 | 0 | 0 | 0 | 0 | 0 | 0 | 0 |
| 330 | 1 | 0 | 0 | 0 | 0 | 0 | 0 | 0 | 0 | 0 | 0 | 0 | 0 | 0 | 0 | 0.7 | 0 | 0 | 0 | 0 | 0 |
| 331 | 0 | 0 | 0 | 0.7 | 0 | 0 | 1 | 1 | 0 | 0 | 0 | 1 | 0 | 0 | 0 | 0 | 0 | 0 | 0 | 0 | 0 |
| 332 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 333 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 334 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 335 | 0 | 0 | 0 | 0 | 0.7 | 0 | 0 | 0 | 0 | 0 | 0 | 0 | 0 | 0 | 0 | 0 | 0 | 0 | 0 | 0 | 0 |
| 336 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 337 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 338 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 339 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 340 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 341 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 342 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 343 | 0 | 0 | 0 | 0 | 0 | 0 | 0 | 0 | 0.7 | 0 | 0 | 0 | 0 | 0 | 0 | 1 | 0 | 0 | 0 | 1 | 0 |
| 344 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 345 | 0.7 | 0 | 0 | 0 | 0 | 0 | 0 | 0 | 0 | 0 | 0 | 0 | 0 | 0 | 0 | 0 | 1 | 0 | 0 | 0 | 0 |
| 346 | 0 | 1 | 0 | 0 | 0 | 0 | 0.7 | 0 | 0 | 0 | 0 | 0 | 0 | 0 | 0 | 0 | 0 | 0 | 0 | 0 | 0 |
| 347 | 0 | 0 | 0.7 | 0 | 0 | 0 | 0 | 0 | 1 | 0 | 0 | 0 | 0 | 0 | 0 | 0 | 0 | 0 | 1 | 0 | 0 |
| 348 | 1 | 0 | 0 | 0 | 0 | 0 | 0 | 0 | 0 | 0 | 0 | 0 | 0 | 0 | 0 | 0 | 0 | 0 | 0 | 0.7 | 0 |
| 349 | 0 | 1 | 0 | 0 | 0 | 0.7 | 0 | 0 | 1 | 0 | 0 | 0 | 0 | 0 | 0 | 0 | 0 | 1 | 0 | 0 | 0 |
| 350 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 351 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 352 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 353 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 354 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 355 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 356 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 357 | 0 | 0 | 1 | 0 | 0 | 0 | 0 | 0 | 0 | 0 | 0 | 0 | 0 | 0 | 0 | 0 | 0 | 0 | 0.7 | 0 | 0 |
| 358 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 359 | 0 | 0 | 0 | 0.7 | 0 | 0 | 0 | 0 | 0 | 0 | 0 | 0 | 0 | 0 | 0 | 1 | 0 | 0 | 0 | 0 | 0 |
| 360 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 361 | 0 | 0.7 | 1 | 0 | 0 | 0 | 1 | 0 | 0 | 0 | 0 | 0 | 0 | 0 | 0 | 0 | 1 | 0 | 0 | 0 | 0 |
| 362 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 363 | 0 | 0 | 0 | 0 | 0 | 0 | 0 | 0 | 0.7 | 1 | 1 | 0 | 0 | 0 | 0 | 0 | 0 | 0 | 0 | 1 | 0 |
| 364 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 365 | 0 | 0 | 0 | 0 | 0 | 1 | 0 | 0 | 0.7 | 0 | 0 | 0 | 0 | 0 | 0 | 0 | 0 | 0 | 0 | 0 | 0 |
| 366 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 367 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 368 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 369 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 370 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 371 | 0 | 1 | 0 | 0 | 0 | 0 | 1 | 0 | 0 | 0 | 0 | 0.7 | 0 | 0 | 0 | 0 | 0 | 0 | 0 | 0 | 0 |
| 372 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 373 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 374 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 375 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 376 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 377 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 378 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 379 | 0 | 0 | 0 | 0 | 0 | 0 | 0 | 0 | 0 | 0 | 0 | 0 | 0 | 1 | 0 | 0 | 0 | 1 | 0.7 | 0 | 0 |
| 380 | 0.7 | 0 | 0 | 0 | 0 | 0 | 0 | 0 | 0 | 0 | 0 | 0 | 0 | 0 | 0 | 0 | 0 | 0 | 0 | 1 | 0 |
| 381 | 0 | 0.7 | 0 | 0 | 0 | 1 | 1 | 0 | 0 | 0 | 1 | 0 | 0 | 0 | 0 | 0.7 | 0 | 0 | 0 | 0 | 0 |
| 382 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 383 | 1 | 0 | 0 | 0 | 0 | 0 | 0 | 0 | 0 | 0 | 0.7 | 0 | 0 | 0 | 0 | 0 | 0 | 0 | 0 | 1 | 0 |
| 384 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 385 | 0 | 0.7 | 0 | 0 | 0 | 0 | 0 | 0 | 0 | 0 | 0 | 1 | 0 | 0 | 0 | 0 | 0 | 0 | 0 | 0 | 0 |
| 386 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 387 | 0 | 0 | 0 | 0 | 0 | 0 | 0 | 0 | 0 | 1 | 1 | 0 | 0 | 0 | 0 | 0 | 0 | 0 | 0 | 0.7 | 0 |
| 388 | 0 | 0 | 0 | 0 | 0 | 0 | 0 | 0 | 0 | 0 | 1 | 0.7 | 0 | 0 | 0 | 0 | 0 | 0 | 0 | 0 | 0 |
| 389 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 390 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 391 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 392 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 393 | 0 | 0 | 0 | 0 | 0 | 0 | 0 | 1 | 0 | 0 | 0 | 0 | 0 | 0 | 0 | 0.7 | 0 | 0 | 0 | 0 | 0 |
| 394 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 395 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 396 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 397 | 1 | 0 | 0 | 0 | 0 | 0 | 0 | 0 | 0 | 0 | 0.7 | 0 | 0 | 0 | 0 | 0 | 0 | 0 | 0 | 1 | 0 |
| 398 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 399 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 400 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 401 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 402 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 403 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 404 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 405 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 406 | 0 | 0 | 0.7 | 0 | 0 | 0 | 0 | 0.7 | 0 | 0 | 0 | 0 | 0 | 0 | 0 | 0 | 0 | 0 | 0 | 0 | 0 |
| 407 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 408 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 409 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 410 | 0 | 0 | 0 | 0 | 0 | 0 | 0 | 0 | 0 | 0 | 0 | 0 | 0 | 0 | 0 | 1 | 0.7 | 0 | 0 | 0 | 0 |
| 411 | 0 | 0 | 0 | 0 | 0 | 0 | 0 | 0 | 0 | 0.7 | 0.7 | 0 | 0 | 0 | 0 | 0 | 0 | 0 | 0 | 0 | 0 |
| 412 | 0 | 0 | 0 | 0 | 0 | 0 | 0 | 0 | 0 | 1 | 0.7 | 0 | 1 | 0 | 0 | 0 | 0 | 0 | 0 | 0 | 0 |
| 413 | 1 | 0 | 0 | 0 | 0 | 0 | 0 | 0 | 0 | 0 | 0 | 0 | 0 | 0 | 0 | 0.7 | 0 | 0 | 0 | 0 | 0 |
| 414 | 0 | 0 | 0 | 0 | 0 | 1 | 0.7 | 0 | 0 | 0 | 0 | 0 | 0 | 0 | 0 | 0 | 0 | 0 | 0 | 0 | 0 |
| 415 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 416 | 0 | 0 | 0 | 0 | 0 | 0 | 0 | 0 | 0 | 0 | 0.7 | 0 | 0 | 0 | 0 | 0 | 0 | 1 | 0 | 0 | 0 |
| 417 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 418 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 419 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 420 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 421 | 0 | 0 | 0 | 0 | 0.7 | 0 | 0 | 0 | 0 | 1 | 0 | 0 | 1 | 0 | 0 | 0 | 1 | 0 | 0 | 1 | 0 |
| 422 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 423 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 424 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 425 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 426 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 427 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 428 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 429 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 430 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 431 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 432 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 433 | 0 | 0 | 0 | 0 | 0 | 0 | 0 | 0 | 0 | 0 | 0.7 | 0 | 0 | 0 | 0.7 | 0 | 0 | 0 | 0 | 0 | 0 |
| 434 | 0 | 0 | 0 | 1 | 0 | 0 | 0 | 0.7 | 0 | 0 | 0 | 0 | 0 | 0 | 0 | 0 | 0 | 0 | 0.7 | 0 | 0 |
| 435 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 436 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 437 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 438 | 0 | 0 | 0 | 0 | 0 | 0 | 0 | 0 | 0.7 | 0 | 0 | 0 | 0 | 0 | 0 | 0 | 0 | 0 | 1 | 0 | 0 |
| 439 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 440 | 0 | 1 | 0 | 0 | 0 | 0 | 0 | 0 | 0 | 0 | 0 | 1 | 0 | 0 | 0.7 | 1 | 0 | 0 | 0 | 0 | 0 |
| 441 | 0 | 1 | 0 | 0.7 | 0 | 0 | 0 | 0 | 0 | 0 | 0.7 | 1 | 0 | 0 | 0 | 0 | 0 | 0 | 0 | 0 | 0 |
| 442 | 0 | 0 | 1 | 0 | 0 | 0 | 0 | 0 | 0.7 | 0 | 0 | 0 | 0 | 1 | 0 | 0 | 0 | 0 | 0.7 | 0 | 0 |
| 443 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 444 | 1 | 0 | 0 | 1 | 0 | 0.7 | 1 | 0 | 0 | 0 | 0 | 0 | 0 | 0 | 1 | 0 | 0 | 0 | 0 | 0 | 0 |
| 445 | 0 | 0 | 0 | 0.7 | 0 | 1 | 0 | 0 | 0 | 0 | 0 | 1 | 0 | 0 | 0 | 0 | 0 | 0 | 0 | 0 | 0 |
| 446 | 0 | 0 | 0 | 0 | 0 | 0 | 0 | 0 | 0.7 | 0 | 0 | 0 | 0 | 0 | 0 | 0 | 0 | 0 | 1 | 0 | 0 |
| 447 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 448 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 449 | 0.7 | 0 | 0 | 0 | 0 | 0 | 0 | 0 | 0 | 0 | 0 | 0 | 0 | 0 | 0 | 1 | 0 | 0 | 0 | 0 | 0 |
| 450 | 0 | 0 | 0 | 0 | 0.7 | 0 | 0 | 0 | 0 | 0 | 0 | 0 | 0 | 0 | 0 | 0 | 0 | 0 | 0 | 0 | 0 |
| 451 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 452 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 453 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 454 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 455 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 456 | 0 | 0 | 0 | 0 | 0 | 0 | 0 | 0 | 0 | 0 | 0.7 | 0 | 0 | 0 | 0 | 0 | 0 | 1 | 0 | 0 | 0 |
| 457 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 458 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 459 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 460 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 461 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 462 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 463 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 464 | 1 | 0 | 0 | 0 | 0 | 0 | 0 | 0 | 0 | 0.7 | 0 | 0 | 0 | 1 | 0 | 0 | 0 | 0 | 0 | 1 | 0 |
| 465 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 466 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 467 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 468 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 469 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 470 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 471 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 472 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 473 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 474 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 475 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 476 | 0 | 0 | 0.7 | 0 | 0 | 0 | 0 | 0 | 0 | 0 | 0 | 0 | 0 | 0 | 0 | 0 | 1 | 0 | 0 | 0 | 0 |
| 477 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 478 | 0 | 0.7 | 1 | 1 | 0 | 0 | 0 | 0 | 1 | 0 | 0 | 0 | 0 | 0 | 0 | 0 | 0 | 0 | 0 | 0 | 0 |
| 479 | 0 | 0 | 0 | 0 | 0 | 0 | 0.7 | 0 | 0 | 0 | 0 | 0 | 0 | 0 | 0 | 1 | 0 | 0 | 0 | 0 | 0 |
| 480 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 481 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 482 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 483 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 484 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 485 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 486 | 0 | 0 | 0 | 0 | 0 | 0 | 0 | 0 | 0.7 | 0 | 0 | 0 | 0 | 0 | 0 | 0 | 0 | 0 | 1 | 0 | 0 |
| 487 | 1 | 0 | 0 | 0 | 0 | 0.7 | 0 | 1 | 0 | 0 | 0 | 0 | 0 | 0 | 0 | 1 | 0 | 0 | 0 | 0 | 0 |
| 488 | 1 | 0 | 0 | 0.7 | 0 | 0 | 0 | 0 | 1 | 0 | 0 | 0 | 0 | 0 | 0 | 1 | 0 | 0 | 0 | 0 | 0 |
| 489 | 0 | 0 | 0 | 0 | 0 | 0 | 0 | 0 | 0 | 0 | 0 | 0 | 0 | 1 | 0 | 0 | 0 | 0 | 0.7 | 0 | 0 |
| 490 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 491 | 0 | 0 | 0 | 0 | 0 | 0 | 0 | 0 | 0 | 0 | 0.7 | 0 | 1 | 0 | 0 | 0 | 0 | 0 | 0 | 0 | 0 |
| 492 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 493 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 494 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 495 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 496 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 497 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 498 | 0 | 0 | 0 | 0 | 0 | 0 | 0.7 | 0 | 0 | 1 | 0 | 0 | 0 | 0 | 0 | 0 | 0 | 0 | 0 | 1 | 0 |
| 499 | 0 | 0 | 0.7 | 0 | 0 | 0 | 0 | 0 | 1 | 0 | 0 | 0 | 0 | 0 | 0 | 0 | 0 | 0 | 0 | 0 | 0 |
| 500 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 501 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 502 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 503 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 504 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 505 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 506 | 0 | 0 | 0 | 0 | 0 | 0 | 0 | 0 | 0 | 0 | 0 | 0 | 0 | 0 | 1 | 0.7 | 0 | 0 | 0 | 0 | 0 |
| 507 | 0 | 1 | 0 | 0 | 0 | 0 | 0 | 0 | 0 | 0 | 0 | 0.7 | 0 | 0 | 0 | 0 | 0 | 0 | 0 | 0 | 0 |
| 508 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 509 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 510 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 511 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 512 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 513 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 514 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 515 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 516 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 517 | 1 | 0 | 0 | 0 | 0 | 0 | 0 | 0 | 0 | 0 | 0 | 0 | 0 | 0 | 0.7 | 0 | 0 | 0 | 0 | 0 | 0 |
| 518 | 0 | 0.7 | 0 | 0 | 0 | 0 | 1 | 0 | 0 | 0 | 0 | 0 | 0 | 0 | 0 | 0.7 | 0 | 0 | 0 | 0 | 0 |
| 519 | 0 | 0 | 0 | 0 | 0 | 0 | 0 | 0 | 0 | 1 | 0 | 0 | 0 | 0 | 0 | 0 | 0.7 | 0 | 0 | 1 | 0 |
| 520 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 521 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 522 | 0 | 0 | 0 | 0 | 0 | 0 | 0 | 0 | 0 | 0 | 0 | 0 | 0 | 0 | 1 | 0.7 | 0 | 0 | 0 | 0 | 0 |
| 523 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 524 | 0.7 | 0 | 0 | 0 | 0 | 0 | 0 | 0 | 0 | 0 | 0 | 0 | 0 | 0 | 0 | 1 | 1 | 0 | 0 | 0 | 0 |
| 525 | 0 | 0.7 | 0 | 1 | 0 | 0 | 1 | 0 | 0 | 0 | 0 | 0 | 0 | 0 | 0 | 0 | 0 | 0 | 0 | 0 | 0 |
| 526 | 0 | 0 | 0 | 0 | 0 | 0 | 0 | 0 | 0.7 | 0 | 0 | 1 | 0 | 0 | 0 | 0 | 0.7 | 0 | 0 | 0 | 0 |
| 527 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 528 | 0 | 1 | 0 | 0 | 0 | 0 | 0.7 | 0 | 0 | 0 | 0 | 1 | 0 | 0 | 0 | 0 | 0 | 0 | 0 | 0 | 0 |
| 529 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 530 | 0 | 0 | 0 | 0 | 0 | 0 | 0 | 0 | 0 | 0 | 1 | 0 | 0 | 0.7 | 0 | 0 | 0 | 0 | 0 | 0 | 0 |
| 531 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 532 | 0 | 0 | 0 | 0 | 0 | 0 | 0 | 1 | 0 | 0 | 0 | 0 | 0 | 0 | 0 | 0.7 | 0 | 0 | 0 | 0 | 0 |
| 533 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 534 | 0 | 0 | 0 | 0 | 0 | 0 | 0 | 0 | 1 | 0 | 0 | 0 | 0 | 0 | 0 | 0 | 0 | 0.7 | 0 | 0 | 0 |
| 535 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 536 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 537 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 538 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 539 | 0 | 1 | 0 | 0 | 0 | 0 | 1 | 0 | 0 | 0 | 0 | 1 | 0 | 0 | 0 | 0.7 | 0 | 0 | 0 | 0 | 0 |
| 540 | 0 | 0 | 0 | 0 | 0 | 0 | 0 | 0 | 0 | 1 | 0.7 | 0 | 1 | 0 | 0 | 0 | 0 | 0 | 0 | 0 | 0 |
| 541 | 0 | 0 | 0 | 0 | 0 | 0 | 0 | 0 | 0 | 1 | 0.7 | 0 | 0 | 0 | 0 | 0 | 0 | 1 | 0 | 1 | 0 |
| 542 | 0 | 0 | 0 | 0 | 0 | 0 | 0 | 0 | 0 | 0 | 0 | 0 | 0 | 0.7 | 0 | 0 | 0 | 0 | 1 | 0 | 0 |
| 543 | 0 | 0 | 0 | 0 | 0 | 0 | 0 | 0.7 | 0 | 0 | 0 | 0 | 0 | 0 | 0 | 1 | 0 | 0 | 0 | 0 | 0 |
| 544 | 0 | 0.7 | 0 | 1 | 0 | 0 | 0 | 1 | 0 | 0 | 0 | 0 | 0 | 0 | 1 | 0 | 0 | 0 | 0 | 0 | 0 |
| 545 | 0 | 0 | 0 | 1 | 0 | 0 | 0.7 | 0 | 0 | 0 | 0 | 0 | 0 | 0 | 0 | 0 | 0 | 0 | 0 | 0 | 0 |
| 546 | 0 | 0 | 0 | 1 | 0 | 1 | 1 | 0 | 0 | 0 | 0 | 0 | 0 | 0 | 0.7 | 0 | 0 | 0 | 0 | 0 | 0 |
| 547 | 0 | 1 | 0 | 0 | 0 | 0 | 0 | 0.7 | 0 | 0 | 0 | 0 | 0 | 0 | 0 | 0 | 0 | 0 | 0 | 0 | 0 |
| 548 | 1 | 0 | 0 | 0 | 0 | 0 | 0 | 1 | 0 | 0 | 0 | 0 | 0 | 0 | 0.7 | 0 | 0 | 0 | 0 | 0 | 0 |
| 549 | 0 | 0 | 0 | 0.7 | 0 | 0 | 1 | 0 | 0 | 0 | 0 | 0 | 0 | 0 | 1 | 0 | 0 | 0 | 0 | 0 | 0 |
| 550 | 1 | 0 | 0 | 0 | 0 | 0 | 0 | 0 | 1 | 0.7 | 0 | 0 | 0 | 0 | 0 | 0 | 0 | 0 | 0 | 0 | 0 |
| 551 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 552 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 553 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 554 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 555 | 1 | 0 | 0 | 0 | 0 | 0 | 0.7 | 0 | 0 | 0 | 0 | 1 | 0 | 0 | 0 | 1 | 0 | 0 | 0 | 0 | 0 |
| 556 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 557 | 0 | 0.7 | 0 | 0 | 0 | 0 | 0 | 0 | 0 | 0 | 0 | 0 | 0 | 0 | 0.7 | 0 | 0 | 0 | 0 | 0 | 0 |
| 558 | 0 | 0 | 0 | 1 | 0 | 0.7 | 0 | 0 | 0 | 0 | 0 | 0 | 0 | 0 | 0 | 1 | 0 | 0 | 0 | 0 | 0 |
| 559 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 560 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 561 | 0 | 0 | 0 | 0 | 0 | 0 | 0 | 0 | 0 | 0.7 | 1 | 0 | 0 | 0 | 0 | 0 | 0 | 0 | 0 | 1 | 0 |
| 562 | 0 | 0 | 0 | 0 | 0 | 0 | 0 | 0 | 0 | 0.7 | 1 | 0 | 0 | 0 | 0 | 0 | 0 | 0 | 0 | 0 | 0 |
| 563 | 0 | 0 | 0 | 0 | 0 | 0 | 0 | 0 | 0 | 0 | 0.7 | 0 | 0 | 1 | 0 | 0 | 0 | 0 | 0 | 0 | 0 |
| 564 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 565 | 0 | 0 | 0 | 0 | 0 | 0 | 0 | 0 | 0 | 1 | 0 | 0 | 0.7 | 0 | 0 | 0 | 0 | 0 | 0 | 0.7 | 0 |
| 566 | 0.7 | 0 | 0 | 0 | 0 | 0 | 0 | 0 | 0 | 0 | 0 | 0 | 1 | 0 | 0 | 1 | 0 | 0 | 0 | 0 | 0 |
| 567 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 568 | 0 | 0 | 0 | 0 | 0 | 0 | 0 | 0 | 0 | 0 | 1 | 0 | 0.7 | 0 | 0 | 0 | 0 | 0 | 0 | 0 | 0 |
| 569 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 570 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 571 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 572 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 573 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 574 | 0 | 0 | 0 | 0 | 0 | 1 | 0 | 0 | 0 | 1 | 0.7 | 0 | 1 | 0 | 0 | 0 | 0 | 0 | 0 | 0 | 0 |
| 575 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 576 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 577 | 0 | 0 | 0 | 0 | 0 | 0 | 0 | 0 | 0 | 0 | 0.7 | 0 | 0 | 0 | 0 | 0 | 0 | 1 | 0 | 0 | 0 |
| 578 | 0.7 | 0 | 0 | 0 | 0 | 0 | 0 | 0 | 0 | 0 | 1 | 0 | 1 | 0 | 0 | 0 | 0 | 0 | 0 | 1 | 0 |
| 579 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 580 | 0 | 0 | 0 | 0 | 0 | 0 | 0 | 0 | 0 | 1 | 0.7 | 0 | 0 | 0 | 0 | 0 | 0 | 1 | 0 | 0 | 0 |
| 581 | 0 | 0 | 0 | 0 | 0 | 0 | 0 | 0 | 0 | 0 | 0 | 0 | 0 | 1 | 0 | 0 | 0 | 0 | 0.7 | 1 | 0 |
| 582 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 583 | 1 | 0.7 | 0 | 0 | 0 | 0 | 0 | 0 | 0 | 0 | 0 | 0 | 0 | 0 | 0 | 0 | 0 | 0 | 0 | 0 | 0 |
| 584 | 0 | 0 | 1 | 0 | 0 | 0 | 0 | 0 | 0 | 0 | 0 | 0 | 0 | 0 | 0 | 0.7 | 0 | 0 | 0 | 0 | 0 |
| 585 | 0.7 | 0 | 1 | 0 | 0 | 0 | 1 | 0 | 0 | 0 | 0 | 0 | 0 | 0 | 1 | 0.7 | 0 | 0 | 0 | 0 | 0 |
| 586 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 587 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 588 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 589 | 0 | 0 | 0 | 0 | 0 | 0 | 1 | 0 | 0 | 0 | 0 | 0 | 0 | 0 | 0 | 0.7 | 0 | 0 | 0 | 0 | 0 |
| 590 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 591 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 592 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 593 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 594 | 0 | 0 | 0 | 0 | 0 | 0 | 0 | 0 | 0 | 1 | 1 | 0 | 0 | 0 | 0 | 0 | 0 | 0 | 0 | 0.7 | 0 |
| 595 | 0 | 0 | 0 | 0 | 0 | 0 | 0 | 0 | 0 | 0.7 | 1 | 0 | 0 | 0 | 0 | 0 | 0 | 0 | 0 | 1 | 0 |
| 596 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 597 | 1 | 0 | 0 | 0 | 0 | 0 | 0 | 0.7 | 0 | 0 | 0 | 0 | 0 | 0 | 0 | 0 | 0 | 0 | 0 | 0 | 0 |
| 598 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 599 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 600 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 601 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 602 | 1 | 0 | 0 | 0 | 0 | 0 | 0 | 0 | 0 | 0 | 0 | 0 | 0 | 0 | 1 | 0 | 0 | 0 | 0 | 0.7 | 0 |
| 603 | 1 | 0 | 1 | 0 | 0 | 0.7 | 1 | 1 | 0 | 0 | 0 | 0 | 0 | 0 | 0 | 1 | 0 | 0 | 0 | 0 | 0 |
| 604 | 0.7 | 1 | 0.7 | 0 | 0 | 1 | 1 | 0 | 0 | 0 | 0 | 1 | 0 | 0 | 0 | 0 | 0 | 0 | 0 | 0 | 0 |
| 605 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 606 | 1 | 1 | 1 | 1 | 0 | 0 | 0 | 1 | 0 | 0 | 0 | 0 | 0.7 | 0 | 0 | 1 | 1 | 0 | 0 | 0 | 0 |
| 607 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 608 | 0.7 | 1 | 0 | 0 | 0 | 0 | 1 | 1 | 0 | 0 | 0 | 0 | 0 | 0 | 0 | 1 | 0 | 0 | 0 | 0 | 0 |
| 609 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 610 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 611 | 0 | 0.7 | 0 | 0 | 0 | 1 | 0 | 0 | 0 | 0 | 0 | 0 | 0 | 0 | 0 | 0 | 0 | 0 | 0 | 0 | 0 |
| 612 | 1 | 0 | 0 | 0 | 0 | 0 | 0.7 | 0 | 0 | 0 | 0 | 0 | 0 | 0 | 0 | 0 | 0 | 0 | 0 | 0 | 0 |
| 613 | 0 | 0 | 0 | 0 | 0 | 1 | 0.7 | 0 | 0 | 0 | 0 | 0 | 0 | 0 | 0 | 0 | 0 | 0 | 0 | 0 | 0 |
| 614 | 0 | 0 | 0 | 0 | 0 | 0 | 0 | 0 | 0 | 0.7 | 0 | 0 | 1 | 0 | 0 | 0 | 0 | 0 | 0 | 1 | 0 |
| 615 | 1 | 0.7 | 0 | 0 | 0 | 1 | 1 | 1 | 0 | 0 | 0 | 1 | 0 | 0 | 0 | 0 | 0 | 0 | 0 | 0 | 0 |
| 616 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 617 | 0 | 0 | 0 | 0 | 0 | 0 | 0 | 0 | 0 | 1 | 1 | 0 | 0.7 | 0 | 0 | 0 | 0 | 0 | 0 | 0 | 0 |
| 618 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 619 | 0 | 0 | 0 | 0 | 0 | 1 | 0.7 | 0 | 0 | 0 | 0 | 0 | 0 | 0 | 0 | 0 | 0 | 0 | 0 | 0 | 0 |
| 620 | 0 | 0 | 0 | 0 | 0 | 0 | 0 | 0 | 0 | 0.7 | 1 | 0 | 0 | 0 | 0 | 0 | 0 | 0 | 0 | 0 | 0 |
| 621 | 0 | 0 | 0 | 0 | 0 | 0 | 0 | 0 | 0 | 1 | 1 | 0 | 0.7 | 0 | 0 | 0 | 0 | 0 | 0 | 0 | 0 |
| 622 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 623 | 0 | 0.7 | 0 | 0 | 0 | 0 | 1 | 0 | 0.7 | 0 | 0 | 1 | 0 | 0 | 0 | 0 | 0 | 0 | 0 | 0 | 0 |
| 624 | 0 | 0 | 0 | 0 | 0 | 0 | 0 | 0 | 1 | 0 | 0 | 0 | 0 | 0 | 0 | 0 | 0 | 0 | 0.7 | 0 | 0 |
| 625 | 0 | 0 | 1 | 0 | 0 | 0.7 | 0 | 1 | 0 | 0 | 0 | 0 | 0 | 0 | 0 | 0 | 0 | 0 | 0 | 0 | 0 |
| 626 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 627 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 628 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 629 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 630 | 1 | 0 | 0 | 0 | 0 | 0 | 0 | 0 | 0 | 0 | 0 | 0 | 0.7 | 1 | 0 | 0 | 0 | 0 | 0 | 1 | 0 |
| 631 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 632 | 0 | 0 | 0 | 0 | 0 | 0 | 0 | 0 | 0 | 0 | 1 | 0 | 0 | 0 | 0 | 0 | 0 | 0.7 | 0 | 0 | 0 |
| 633 | 0 | 0 | 0 | 0 | 0 | 0 | 0 | 0 | 0 | 1 | 1 | 0 | 0 | 0 | 0 | 0 | 0 | 0 | 0 | 0.7 | 0 |
| 634 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 635 | 1 | 0 | 0 | 0 | 0 | 0 | 0 | 0 | 0 | 0 | 1 | 0 | 0 | 0 | 0 | 0.7 | 0 | 0 | 0 | 1 | 0 |
| 636 | 0 | 0 | 0 | 0 | 0 | 0.7 | 0 | 0 | 0.7 | 0 | 0 | 0 | 0 | 0 | 0 | 0 | 0 | 0 | 0 | 0 | 0 |
| 637 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 638 | 0 | 0 | 1 | 0.7 | 0 | 0 | 1 | 0 | 0 | 0 | 0 | 0 | 0 | 0 | 0 | 0 | 0 | 0 | 0 | 0 | 0 |
| 639 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 640 | 0 | 0.7 | 1 | 0 | 0 | 0 | 0 | 0 | 0 | 0 | 0 | 0 | 0 | 0 | 0 | 0 | 0.7 | 0 | 0 | 1 | 0 |
| 641 | 0 | 1 | 0 | 0 | 0 | 0 | 0 | 0 | 0 | 0 | 1 | 0 | 0 | 0 | 0 | 0 | 0 | 1 | 0 | 0.7 | 0 |
| 642 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 643 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 644 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 645 | 0 | 0 | 0 | 0 | 0 | 0 | 0 | 0 | 0 | 1 | 0.7 | 0 | 0 | 0 | 0 | 0 | 0 | 0 | 0 | 0 | 0 |
| 646 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 647 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 648 | 0 | 0 | 0 | 0 | 0 | 0 | 0 | 0 | 0 | 0 | 0 | 0 | 0 | 0 | 0 | 0 | 0 | 1 | 1 | 0.7 | 0 |
| 649 | 0 | 0 | 0 | 0 | 0 | 0 | 0 | 0 | 0 | 1 | 1 | 0 | 0 | 0 | 0 | 0 | 0 | 0 | 0 | 0.7 | 0 |
| 650 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 651 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 652 | 0 | 1 | 0 | 0 | 0 | 1 | 0 | 0.7 | 1 | 0 | 0 | 1 | 0 | 0 | 0 | 0 | 1 | 0 | 0 | 0 | 0 |
| 653 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 654 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 655 | 1 | 0 | 0 | 0 | 0 | 0 | 0 | 0 | 0 | 1 | 0 | 0 | 0 | 0 | 0 | 0 | 0 | 0 | 0 | 0.7 | 0 |
| 656 | 0 | 0 | 0 | 0 | 0 | 0 | 0 | 0 | 0 | 0 | 0 | 0 | 0 | 0.7 | 0 | 0 | 0 | 1 | 0 | 1 | 0 |
| 657 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 658 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 659 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 660 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 661 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 662 | 0 | 0 | 0 | 0 | 0 | 0 | 0 | 0 | 0 | 0 | 0 | 0 | 0 | 0.7 | 0 | 0 | 0 | 0 | 1 | 0 | 0 |
| 663 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 664 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 665 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 666 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 667 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 668 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 669 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 670 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 671 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 672 | 0.7 | 0 | 0 | 0 | 0 | 0 | 0 | 0 | 0 | 0 | 0 | 0 | 0 | 0 | 0 | 0 | 0 | 0 | 0 | 1 | 0 |
| 673 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 674 | 1 | 0 | 0 | 0 | 0 | 0 | 0 | 0 | 0 | 0 | 0 | 0 | 0 | 0 | 0 | 0.7 | 1 | 0 | 0 | 0 | 0 |
| 675 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 676 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 677 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 678 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 679 | 0 | 0 | 0 | 0 | 0 | 0 | 0 | 0 | 0 | 0 | 0 | 0 | 0 | 0 | 0 | 0 | 1 | 0 | 0 | 0.7 | 0 |
| 680 | 0 | 0 | 0 | 0 | 0 | 0 | 0 | 0 | 0 | 0 | 0 | 0 | 0 | 0.7 | 0 | 0 | 0 | 1 | 0 | 0 | 0 |
| 681 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 682 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 683 | 0 | 0.7 | 0 | 0 | 1 | 0 | 0 | 0 | 0 | 0 | 0 | 0 | 0 | 0 | 0 | 0 | 0 | 0 | 0 | 0 | 0 |
| 684 | 0 | 0 | 0 | 0 | 0 | 0 | 0 | 0 | 0.7 | 0 | 0 | 0 | 0 | 0 | 0 | 0 | 0 | 0 | 1 | 0 | 0 |
| 685 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 686 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 687 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 688 | 1 | 0 | 0 | 0 | 0 | 0 | 0 | 0 | 0 | 0 | 0.7 | 0 | 0 | 0 | 0 | 0 | 0 | 0 | 0 | 0 | 0 |
| 689 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 690 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 691 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 692 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 693 | 0 | 0 | 0 | 0.7 | 0 | 0 | 0 | 0 | 1 | 0 | 0 | 0 | 0 | 0 | 0 | 0 | 0 | 0 | 0 | 0 | 0 |
| 694 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 695 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 696 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 697 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 698 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 699 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 700 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 701 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 702 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 703 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 704 | 0 | 0 | 0 | 0.7 | 0 | 1 | 0 | 0 | 1 | 0 | 0 | 0 | 0 | 0 | 0 | 0 | 0 | 0 | 0 | 0 | 0 |
| 705 | 0 | 0 | 0 | 0 | 0 | 0 | 0 | 1 | 0 | 0 | 0 | 0 | 0 | 0 | 0.7 | 0 | 0 | 0 | 0 | 0 | 0 |
| 706 | 0 | 0 | 0 | 1 | 0 | 0 | 0.7 | 0 | 0 | 0 | 0 | 0 | 0 | 0 | 0 | 0 | 0 | 0 | 0 | 0 | 0 |
| 707 | 0.7 | 0 | 0 | 0 | 0 | 1 | 1 | 0 | 0 | 0 | 0 | 0 | 0 | 0 | 0 | 0 | 0 | 0 | 0 | 0 | 0 |
| 708 | 1 | 0 | 0 | 0 | 0 | 0 | 0 | 0 | 0 | 0 | 0 | 0 | 0 | 0 | 0 | 0 | 0.7 | 0 | 0 | 0.7 | 0 |
| 709 | 0.7 | 0 | 0 | 0 | 0 | 0 | 0 | 0 | 0 | 0 | 1 | 0 | 0 | 0 | 0 | 0 | 0 | 0 | 0 | 0 | 0 |
| 710 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 711 | 0 | 1 | 0 | 0 | 0 | 0 | 0 | 0 | 0 | 1 | 1 | 0.7 | 0 | 0 | 0 | 0 | 0 | 0 | 0 | 0 | 0 |
| 712 | 0 | 0 | 0 | 0 | 0 | 0 | 0 | 0 | 0 | 1 | 0.7 | 0 | 1 | 0 | 0 | 0 | 0 | 0 | 0 | 0 | 0 |
| 713 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 714 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 715 | 0 | 0 | 0 | 0 | 0 | 0 | 0 | 0 | 0 | 0 | 0 | 0 | 0 | 0.7 | 0 | 0 | 0 | 1 | 0 | 0 | 0 |
| 716 | 0 | 0 | 0 | 0 | 0 | 0 | 0 | 0 | 0 | 0 | 0.7 | 0 | 0 | 1 | 0 | 0 | 0 | 0 | 0 | 0 | 0 |
| 717 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 718 | 0.7 | 1 | 0 | 0 | 0 | 0 | 0 | 0 | 1 | 0 | 0 | 1 | 0 | 0 | 0 | 0 | 0 | 0 | 0 | 0 | 0 |
| 719 | 0.7 | 0 | 0 | 0 | 1 | 0 | 0 | 0 | 0 | 0 | 0 | 0 | 0 | 0 | 0 | 0 | 0 | 0 | 0 | 0 | 0 |
| 720 | 1 | 0.7 | 0 | 0 | 0 | 0 | 0 | 0 | 0 | 0 | 0 | 1 | 0 | 0 | 0 | 0 | 0 | 0 | 0 | 0 | 0 |
| 721 | 1 | 0 | 1 | 0 | 0 | 0 | 0.7 | 0 | 0 | 0 | 0 | 0 | 0 | 0 | 0 | 0 | 0 | 0 | 0 | 0 | 0 |
| 722 | 0 | 0.7 | 1 | 1 | 0 | 0 | 1 | 0 | 0 | 0 | 0 | 0 | 0 | 0 | 0 | 0 | 0 | 0 | 0 | 0 | 0 |
| 723 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 724 | 0 | 0 | 0 | 1 | 0 | 0 | 0 | 1 | 0 | 0 | 0 | 0.7 | 0 | 0 | 0 | 1 | 1 | 0 | 0 | 0 | 0 |
| 725 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 726 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 727 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 728 | 0 | 1 | 0 | 0 | 0 | 0 | 0.7 | 0 | 0 | 0 | 0 | 1 | 0 | 0 | 0 | 0 | 0 | 0 | 0 | 0 | 0 |
| 729 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 730 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 731 | 0 | 1 | 0 | 0.7 | 0 | 0.7 | 0 | 0 | 0 | 0 | 0 | 0 | 0 | 0 | 0 | 0 | 0 | 0 | 0 | 0 | 0 |
| 732 | 0.7 | 0 | 0 | 0 | 0 | 0 | 0 | 1 | 0 | 0 | 0 | 0 | 0 | 0 | 0 | 0 | 0 | 0 | 0 | 0 | 0 |
| 733 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 734 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 735 | 0.7 | 0 | 0 | 0 | 0 | 1 | 1 | 0 | 0 | 0 | 0 | 1 | 0 | 0 | 0 | 0 | 0 | 0 | 0 | 0 | 0 |
| 736 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 737 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 738 | 0 | 1 | 0 | 0 | 0 | 0 | 1 | 0 | 0 | 0 | 0 | 0 | 0 | 0 | 0 | 0.7 | 0 | 0 | 1 | 0 | 0 |
| 739 | 1 | 0 | 0 | 0 | 0 | 0 | 0.7 | 0 | 0 | 0 | 0 | 0 | 0 | 0 | 0 | 1 | 0 | 0 | 0 | 0 | 0 |
| 740 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 741 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 742 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 743 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 744 | 0 | 0 | 0 | 0 | 0 | 0 | 0.7 | 0 | 0 | 0 | 0 | 1 | 0 | 0 | 0 | 0 | 0 | 0 | 0 | 0 | 0 |
| 745 | 0 | 0.7 | 0 | 0 | 0 | 0 | 0 | 0 | 0 | 0 | 1 | 0 | 0 | 0 | 0 | 0 | 0 | 0 | 0 | 0 | 0 |
| 746 | 0 | 0 | 0 | 0 | 0 | 0 | 0 | 0 | 0 | 0 | 0 | 0 | 0 | 0 | 0 | 0 | 0 | 1 | 0.7 | 0 | 0 |
| 747 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 748 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 749 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 750 | 0 | 0 | 0 | 0 | 0 | 0 | 0 | 0 | 0 | 0 | 0.7 | 0 | 1 | 0 | 0 | 0 | 0 | 1 | 0 | 0 | 0 |
| 751 | 1 | 0 | 0 | 0 | 0 | 0 | 0 | 0 | 0 | 0 | 1 | 0 | 0.7 | 0 | 0 | 0 | 0 | 0 | 0 | 0 | 0 |
| 752 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 753 | 0 | 0 | 0 | 0 | 0 | 0 | 0 | 0 | 0 | 0.7 | 1 | 0 | 0 | 0 | 0 | 0 | 0 | 0 | 0 | 0 | 0 |
| 754 | 0.7 | 0 | 0 | 0 | 0 | 0 | 0 | 0 | 0 | 1 | 1 | 0 | 1 | 0 | 0 | 1 | 0 | 0 | 0 | 0 | 0 |
| 755 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 756 | 1 | 0 | 0 | 0 | 0.7 | 0 | 0 | 0 | 0 | 1 | 0 | 0 | 0 | 0 | 0 | 0 | 0 | 0 | 0 | 1 | 0 |
| 757 | 1 | 0 | 0 | 0 | 0 | 0 | 0 | 0 | 0 | 1 | 0 | 0 | 1 | 0.7 | 0 | 0 | 0 | 0 | 1 | 0 | 0 |
| 758 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 759 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 760 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 761 | 0 | 0.7 | 0 | 0 | 0 | 0 | 0 | 0 | 0 | 0 | 0 | 1 | 0 | 0 | 0 | 0 | 0 | 0 | 0 | 0 | 0 |
| 762 | 0 | 0 | 0 | 0 | 0 | 0 | 0 | 0 | 0 | 0 | 0 | 0 | 0 | 0.7 | 0 | 0 | 0 | 0 | 1 | 0 | 0 |
| 763 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 764 | 0 | 0 | 0 | 0 | 0 | 0 | 0 | 0 | 0 | 0 | 0.7 | 0 | 0 | 0 | 0 | 1 | 1 | 0 | 0 | 0 | 0 |
| 765 | 0 | 0 | 1 | 0 | 0 | 0 | 0 | 0 | 0 | 0 | 0 | 1 | 0 | 0 | 0 | 0.7 | 0 | 0 | 0 | 0 | 0 |
| 766 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 767 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 768 | 0 | 1 | 0 | 0 | 0 | 0 | 0 | 0 | 1 | 0 | 0 | 0 | 0 | 0 | 0 | 0.7 | 0 | 0 | 0 | 0 | 0 |
| 769 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 770 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 771 | 0 | 0 | 0 | 0 | 0 | 0 | 0 | 0 | 0 | 0 | 0 | 0 | 0.7 | 0 | 0 | 0 | 1 | 0 | 0 | 0 | 0 |
| 772 | 0 | 1 | 0 | 0 | 0 | 0 | 0.7 | 0 | 0 | 0 | 0 | 0 | 0 | 0 | 0 | 0 | 0 | 0 | 0 | 0 | 0 |
| 773 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 774 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 775 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 776 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 777 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 778 | 0 | 0 | 0 | 0 | 0 | 0 | 0 | 0 | 0 | 1 | 0 | 0 | 0 | 0.7 | 0 | 0 | 0 | 0 | 0 | 0 | 0 |
| 779 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 780 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 781 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 782 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 783 | 0 | 0 | 0 | 0 | 0 | 0 | 0 | 0 | 0 | 0 | 0 | 0 | 0 | 1 | 0 | 0 | 0 | 0.7 | 1 | 0 | 0 |
| 784 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 785 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 786 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 787 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 788 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 789 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 790 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 791 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 792 | 0 | 0 | 0 | 0 | 0 | 0 | 0 | 0 | 0 | 0 | 1 | 0 | 0.7 | 0 | 0 | 0 | 0 | 0 | 0 | 0 | 0 |
| 793 | 0 | 0 | 0 | 0 | 0 | 0 | 0.7 | 0 | 0 | 0 | 0 | 0 | 0 | 0 | 0 | 0 | 0 | 0 | 0 | 0 | 0 |

### Example 19: Improved Activity and Expression of the SUSA from Thermosynechococcus elongatus by Computational Design

A homology model of the sucrose synthase SUSA from Thermosynechococcus elongatus was built and used as input to computational designs to improve pA21841. Computational designs were conducted to improve the stability and expression of the SuSy. forty-seven computational designs were chosen for experimental validation (SEQ ID NOs: 1181-1227). Expression plasmids for the computational designs were built as in Example 4. Each SuSy variant was expressed and purified as in Example 5. Each SuSy variant was assayed in a one-pot reaction with the B12GT, pA29798. The purified B12GT and SuSy were reacted with 100 mg/mL RA50, 250 mM Sucrose, and 0.5 mM ADP in 50 mM pH 6 phosphate buffer and 50 mM NaCl for 24 hours at 60° C. Product rebaudiosides were monitored by LCMS similar to Example 3. Several variants showed improved expression and/or Reb D conversion compared to the parent pA21841. The top designs showed up to a 2.2-fold improvement in yield or a 5.4-fold improvement in expression (34.6% conversion, 6.8 uM purified protein; Table 25).

TABLE 25
Computational Designs of pA21841

| | | Purified |
|---|---|---|
| ID | % Conversion | Conc (uM) |
| pA34126 | 75.2 | 11.7 |
| pA34103 | 73 | 9.5 |
| pA34101 | 64.3 | 9.6 |
| pA34127 | 59.9 | 9.2 |
| pA34102 | 57.7 | 8.8 |
| pA34112 | 57.2 | 4.5 |
| pA34128 | 56.2 | 8.6 |
| pA34116 | 53.4 | 13.2 |
| pA34118 | 47.9 | 17.1 |
| pA34115 | 47.5 | 3.6 |
| pA34099 | 47 | 20.6 |
| pA34117 | 46.2 | 7.2 |
| pA34131 | 45.8 | 7.1 |
| pA34125 | 45.4 | 18.3 |
| pA34121 | 45.1 | 8.3 |
| pA34134 | 43.7 | 13.3 |
| pA34086 | 42.2 | 14 |
| pA34111 | 42 | 2.2 |
| pA34113 | 40.5 | 6 |
| pA34135 | 40.3 | 13.7 |
| pA34132 | 39.6 | 9.8 |
| pA34087 | 38.2 | 5.8 |
| pA34106 | 36.8 | 14.1 |
| pA34094 | 34.4 | 14.6 |
| pA34129 | 32.4 | 10.6 |
| pA34119 | 32.1 | 23.7 |
| pA34130 | 31.7 | 5.8 |
| pA34092 | 30.7 | 10.9 |
| pA34107 | 30.2 | 15.1 |
| pA34096 | 29.7 | 2.6 |
| pA34091 | 28.5 | 13.7 |
| pA34110 | 26.6 | 25.6 |
| pA34089 | 23.5 | 26 |
| pA34088 | 22.4 | 28.6 |
| pA34122 | 22.1 | 7.8 |
| pA34109 | 21.9 | 28.4 |
| pA34120 | 21.6 | 12.2 |
| pA34097 | 15.7 | 4.2 |
| pA34133 | 15.3 | 22.9 |
| pA34095 | 13.5 | 17.9 |
| pA34105 | 12.9 | 7.8 |
| pA34090 | 12.1 | 30.7 |
| pA34123 | 7.8 | 19 |
| pA34104 | 7.7 | 36.3 |
| pA34124 | 3.3 | 17.4 |

Computational designs were also conducted to improve the stability and expression of SUSA by redesigning and repacking buried protein cores. Thirty-eight computational designs were chosen for experimental validation (SEQ ID NOs: 1231-1267). Expression plasmids for the computational designs were built as in Example 4. Each SuSy variant was expressed and purified as in Example 5. Each SuSy variant was assayed in a one-pot reaction with the B12GT, pA29798. The purified B12GT and SuSy were reacted with 100 mg/mL RA50, 250 mM Sucrose, and 0.5 mM ADP in 50 mM pH 6 phosphate buffer, and 50 mM NaCl for 24 hours at 60° C. Product rebaudiosides were monitored by LCMS similar to Example 3. Several designed enzymes expressed well and were active for Reb A to Reb D conversion (Table 26).

TABLE 26
Computational Designs of pA21841

| | | Purified |
|---|---|---|
| ID | % Conversion | Conc (uM) |
| pA35407 | 78.3 | 10.3 |
| pA35409 | 67.9 | 11.7 |
| pA35431 | 55.3 | 10.2 |
| pA35417 | 46.1 | 7.6 |
| pA35437 | 37.5 | 12.3 |
| pA35427 | 27 | 5 |
| pA35432 | 24.8 | 8.9 |
| pA35422 | 21.2 | 7.3 |
| pA35423 | 17.4 | 4.6 |
| pA35408 | 16.8 | 3.4 |
| pA35426 | 15.3 | 4 |
| pA35412 | 15.2 | 3 |
| pA35425 | 15.1 | 5.6 |
| pA35439 | 14.1 | 3.5 |
| pA35416 | 12.9 | 8.7 |
| pA35428 | 12.5 | 4.8 |
| pA35429 | 10.2 | 5.2 |
| pA35410 | 9.3 | 2.9 |
| pA35424 | 8.6 | 5.8 |
| pA35430 | 8.4 | 5.8 |
| pA35434 | 7.5 | 8.2 |
| pA35435 | 7.4 | 5.4 |
| pA35436 | 5.8 | 4.3 |
| pA35419 | 5.5 | 3 |
| pA35440 | 5.4 | 3.1 |
| pA35443 | 5.3 | 2.6 |
| pA35444 | 5.3 | 2.4 |
| pA35438 | 5.2 | 3.4 |
| pA35441 | 5.1 | 3.7 |
| pA35413 | 4.9 | 2.9 |
| pA35418 | 4.8 | 2 |
| pA35420 | 4.7 | 1.6 |
| pA35442 | 4.5 | 2.4 |
| pA35414 | 4.4 | 5 |
| pA35411 | 4.3 | 2.4 |
| pA35433 | 4.3 | 0 |
| pA35421 | 0.2 | 10.8 |

Computational designs were also conducted to improve the stability and expression of the SuSy using coevolutionary information. Sixty-five computational designs were chosen for experimental validation (SEQ ID NOs: 1268-1332). Expression plasmids for the computational designs were built as in Example 4. Each SuSy variant was expressed and purified as in Example 5. Each SuSy variant was assayed in a one-pot reaction with the B12GT, pA29798. The purified B12GT and SuSy were reacted with 100 mg/mL RA50, 250 mM Sucrose, and 0.5 mM ADP in 50 mM pH 6 phosphate buffer and 50 mM NaCl for 24 hours at 60° C. Product rebaudiosides were monitored by LCMS similar to Example 3. Several designed enzymes expressed well and were active for Reb A to Reb D conversion (Table 27).

TABLE 27
Computational Designs of pA21841

| | | Purified |
|---|---|---|
| ID | % Conversion | Conc (uM) |
| pA35907 | 83.5 | 20.3 |
| pA35928 | 82.9 | 8.5 |
| pA35903 | 71.3 | 17.1 |
| pA35933 | 71.2 | 8.9 |
| pA35901 | 68.9 | 21.4 |
| pA35932 | 68.1 | 11.3 |
| pA35938 | 66.5 | 4.5 |
| pA35882 | 65.3 | 11.2 |
| pA35942 | 64.3 | 9.7 |
| pA35897 | 60.2 | 15.8 |
| pA35943 | 57 | 8.3 |
| pA35896 | 56.8 | 21.2 |
| pA35936 | 55.4 | 5.9 |
| pA35927 | 55 | 16.1 |
| pA35926 | 49.5 | 8.4 |
| pA35929 | 49.4 | 10.1 |
| pA35891 | 48.8 | 29.6 |
| pA35898 | 47.3 | 6.6 |
| pA35895 | 47.1 | 12.9 |
| pA35937 | 46.3 | 8 |
| pA35880 | 44.1 | 7.8 |
| pA35910 | 41.1 | 13.5 |
| pA35941 | 40.6 | 8.9 |
| pA35904 | 40.5 | 11.9 |
| pA35872 | 39.4 | 7.1 |
| pA35930 | 39.1 | 6.2 |
| pA35899 | 38.6 | 22.1 |
| pA35873 | 38.5 | 11 |
| pA35935 | 36.3 | 5.3 |
| pA35905 | 34.4 | 16.5 |
| pA35902 | 33.4 | 16.3 |
| pA35911 | 32.8 | 18 |
| pA35919 | 31.4 | 9.2 |
| pA35912 | 29.7 | 6.4 |
| pA35893 | 28.1 | 23.5 |
| pA35889 | 27.7 | 6.8 |
| pA35890 | 27.1 | 20.2 |
| pA35874 | 26.5 | 8 |
| pA35886 | 25.7 | 23.7 |
| pA35888 | 24.6 | 9.2 |
| pA35875 | 24.4 | 12.1 |
| pA35908 | 24.1 | 13.6 |
| pA35892 | 23.6 | 20.4 |
| pA35887 | 23.4 | 13.9 |
| pA35879 | 22.3 | 9.6 |
| pA35931 | 19.5 | 5.9 |
| pA35915 | 19.4 | 9.8 |
| pA35940 | 18.7 | 8.8 |
| pA35914 | 18.6 | 20.9 |
| pA35876 | 18.4 | 23 |
| pA35878 | 18 | 13.4 |
| pA35921 | 17.9 | 11.7 |
| pA35881 | 16.8 | 9.1 |
| pA35877 | 13.9 | 22.6 |
| pA35923 | 13 | 28.2 |
| pA35925 | 12.1 | 13.2 |
| pA35917 | 10.3 | 12.8 |
| pA35906 | 9.1 | 7.2 |
| pA35884 | 7.8 | 18.4 |
| pA35922 | 7.6 | 6.2 |
| pA35913 | 6.7 | 3.8 |
| pA35939 | 6.4 | 5.9 |
| pA35924 | 5.7 | 7.1 |
| pA35909 | 2.1 | 10.5 |

### Example 20: Representing Successful SuSy Designs with a PSSM

The successful SuSy designs from Example 19 were used to create a PSSM (Table 28). The PSSM is a concise way to represent the successful designs and related sequences. Sequences the have a PSSM score greater than 569.5 are considered related to the active computational designs described in Example 19. To score a sequence with the generated PSSM, it must first be aligned with the representative sequence pA21841 (Seq ID No: 888). For example, the following successful designs, pA34103, pA34119, pA34099 have the following PSSM scores: 576.7, 572.5, 577.0, while the wild-type SuSy pA21841, has a PSSM score of only 565.6.

TABLE 28
Position Specific Scoring Matrix (PSSM) of Successful SuSy Designs

| Seq | Amino Acids | | | | | | | | | | | | | | | | | | | | |
|---|---|---|---|---|---|---|---|---|---|---|---|---|---|---|---|---|---|---|---|---|---|
| Pos | A | R | N | D | C | Q | E | G | H | I | L | K | M | F | P | S | T | W | Y | V | — |
| 1 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 3 | 1 | 0 | 0 | 0 | 0.7 | 0 | 0 | 0 | 0 | 0 | 0 | 0 | 0 | 0 | 0 | 1 | 0 | 0 | 0 | 0 | 0 |
| 4 | 0 | 0 | 0 | 1 | 0 | 0 | 1 | 0 | 0 | 0 | 0 | 0 | 0 | 0 | 0 | 0 | 0 | 0 | 0 | 0.7 | 0 |
| 5 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 6 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 7 | 0 | 0 | 0 | 0 | 0 | 0 | 1 | 0 | 0 | 0 | 0 | 0.7 | 0 | 0 | 0 | 0 | 0 | 0 | 0 | 0 | 0 |
| 8 | 0.7 | 0 | 0 | 0 | 0 | 0 | 0 | 0 | 0 | 0 | 0 | 0 | 0 | 0 | 0 | 1 | 0 | 0 | 0 | 0 | 0 |
| 9 | 0 | 0 | 0 | 0 | 0 | 0 | 0 | 0 | 0 | 0 | 0 | 0 | 1 | 0 | 0 | 0 | 0 | 0 | 0 | 0.7 | 0 |
| 10 | 0 | 0 | 0 | 0 | 0 | 0 | 0 | 0 | 0 | 1 | 1 | 0 | 0 | 0 | 0 | 0 | 0 | 1 | 0 | 0.7 | 0 |
| 11 | 0 | 0 | 1 | 1 | 0 | 0 | 0.7 | 0 | 0 | 0 | 0 | 0 | 0 | 0 | 0 | 0 | 0 | 0 | 0 | 0 | 0 |
| 12 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 13 | 0 | 0 | 0 | 0.7 | 0 | 0 | 1 | 0 | 0 | 0 | 0 | 0 | 0 | 0 | 0 | 0 | 0 | 0 | 0 | 0 | 0 |
| 14 | 0 | 0 | 0 | 0 | 0 | 0 | 0.7 | 0 | 1 | 0 | 0 | 0 | 0 | 0 | 0 | 0 | 0 | 0 | 0 | 0 | 0 |
| 15 | 0 | 0.7 | 0 | 0 | 0 | 0 | 0 | 0 | 0 | 0 | 0 | 1 | 0 | 0 | 0 | 0 | 0 | 0 | 0 | 0 | 0 |
| 16 | 0.7 | 0 | 1 | 0 | 0 | 0 | 1 | 0 | 0 | 0 | 0 | 0 | 0 | 0 | 0 | 0 | 1 | 0 | 0 | 0 | 0 |
| 17 | 1 | 0 | 0 | 0.7 | 0 | 0 | 0 | 0 | 0 | 0 | 0 | 0 | 0 | 0 | 0 | 0 | 0 | 0 | 0 | 0 | 0 |
| 18 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 19 | 0 | 0.7 | 0 | 0 | 0 | 0 | 0 | 0 | 1 | 0 | 0 | 0 | 0 | 0 | 0 | 0 | 0 | 0 | 0 | 0 | 0 |
| 20 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 21 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 22 | 0 | 0 | 0 | 0 | 0 | 0 | 0 | 0 | 0 | 1 | 1 | 0 | 0 | 1 | 0 | 0.7 | 0 | 0 | 0 | 0 | 0 |
| 23 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 24 | 0 | 0 | 0 | 0 | 0 | 1 | 0 | 0 | 0 | 0.7 | 1 | 0 | 0 | 0 | 0 | 0 | 0 | 0 | 1 | 0 | 0 |
| 25 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 26 | 0 | 1 | 0 | 0 | 0 | 0.7 | 0 | 0 | 0 | 0 | 0 | 0 | 0 | 0 | 0 | 0 | 0 | 0 | 0 | 0 | 0 |
| 27 | 1 | 1 | 0 | 1 | 0 | 0 | 0 | 0 | 0 | 0 | 0.7 | 0 | 0 | 0 | 0 | 0 | 1 | 0 | 0 | 0 | 0 |
| 28 | 0 | 0 | 1 | 0 | 0 | 1 | 0 | 0.7 | 0 | 0 | 0 | 0 | 0 | 0 | 0 | 1 | 0 | 0 | 0 | 0 | 0 |
| 29 | 0 | 0 | 0 | 0 | 0 | 0 | 0.7 | 1 | 0 | 0 | 0 | 0 | 0 | 0 | 0 | 0 | 0 | 0 | 0 | 0 | 0 |
| 30 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 31 | 0 | 0.7 | 0 | 0 | 0 | 0 | 0 | 1 | 0 | 0 | 0 | 0 | 0 | 0 | 0 | 0 | 0 | 0 | 0 | 0 | 0 |
| 32 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 33 | 0 | 0 | 0 | 0 | 0 | 0 | 0 | 0 | 0 | 0 | 0.7 | 0 | 0 | 1 | 0 | 0 | 0 | 0 | 0 | 0 | 0 |
| 34 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 35 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 36 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 37 | 0 | 0 | 0 | 0.7 | 0 | 0 | 1 | 0 | 0 | 0 | 0 | 0 | 0 | 0 | 0 | 0 | 0 | 0 | 0 | 0 | 0 |
| 38 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 39 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 40 | 0 | 0 | 1 | 0.7 | 0 | 1 | 0 | 1 | 0 | 0 | 0 | 0 | 0 | 0 | 0 | 0 | 0 | 0 | 0 | 0 | 0 |
| 41 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 42 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 43 | 0.7 | 0 | 0 | 0 | 0 | 0 | 1 | 0 | 1 | 0 | 0 | 0 | 0 | 0 | 0 | 0 | 0 | 0 | 0 | 0 | 0 |
| 44 | 0 | 0 | 0 | 0.7 | 0 | 0 | 1 | 0 | 0 | 0 | 0 | 0 | 0 | 0 | 0 | 0 | 0 | 0 | 0 | 0 | 0 |
| 45 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 46 | 0 | 0 | 0 | 0 | 0.7 | 0 | 0 | 0 | 0 | 0 | 0 | 0 | 0 | 0 | 0 | 0 | 0 | 0 | 0 | 0 | 0 |
| 47 | 0 | 0.7 | 0 | 0 | 0 | 0 | 0 | 0 | 1 | 0 | 0 | 0 | 0 | 0 | 0 | 0 | 0 | 0 | 0 | 0 | 0 |
| 48 | 1 | 0 | 1 | 0.7 | 0 | 0 | 0 | 0 | 0 | 0 | 0 | 0 | 0 | 0 | 0 | 0 | 0 | 0 | 0 | 0 | 0 |
| 49 | 0 | 0 | 0 | 0 | 0 | 0.7 | 0 | 0 | 0 | 0 | 1 | 1 | 0 | 0 | 0 | 0 | 0 | 0 | 0 | 0 | 0 |
| 50 | 0 | 0 | 0 | 1 | 0 | 1 | 0.7 | 1 | 0 | 0 | 0 | 0 | 0 | 0 | 0 | 0 | 0 | 0 | 0 | 0 | 0 |
| 51 | 0 | 0.7 | 0 | 0 | 0 | 0 | 0 | 0 | 0 | 0 | 0 | 1 | 0 | 0 | 0 | 0 | 0 | 0 | 0 | 0 | 0 |
| 52 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 53 | 1 | 0 | 0 | 0 | 0 | 0 | 1 | 0 | 0 | 0 | 0 | 0 | 0 | 0 | 0 | 0 | 0 | 0 | 0 | 0.7 | 0 |
| 54 | 0 | 0 | 0 | 0 | 0 | 0 | 0 | 0 | 0 | 0 | 0 | 0 | 0 | 0 | 0.7 | 0 | 0 | 0 | 1 | 0 | 0 |
| 55 | 0 | 0 | 0 | 0 | 0 | 0 | 0 | 0 | 0 | 0 | 0 | 0 | 0 | 1 | 0.7 | 0 | 1 | 0 | 0 | 0 | 0 |
| 56 | 0 | 0 | 0 | 0 | 0 | 0 | 0 | 0 | 0 | 0 | 0 | 0 | 0 | 0 | 0.7 | 0 | 0 | 0 | 1 | 0 | 0 |
| 57 | 0 | 0 | 0 | 1 | 0 | 0 | 0 | 0 | 1 | 0 | 1 | 0 | 0 | 0 | 0 | 0.7 | 0 | 0 | 0 | 0 | 0 |
| 58 | 0 | 0 | 1 | 0 | 0 | 0 | 0.7 | 0 | 0 | 0 | 0 | 0 | 0 | 0 | 0 | 1 | 0 | 0 | 0 | 0 | 0 |
| 59 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 60 | 0 | 0.7 | 0 | 0 | 0 | 0 | 0 | 1 | 0 | 0 | 0 | 0 | 0 | 0 | 0 | 0 | 0 | 0 | 0 | 0 | 0 |
| 61 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 62 | 1 | 0 | 0 | 0 | 0 | 0 | 0 | 0 | 0 | 0 | 0 | 0 | 0 | 0 | 0 | 0.7 | 0 | 1 | 0 | 0 | 0 |
| 63 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 64 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 65 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 66 | 0 | 1 | 0 | 0 | 0 | 0 | 0 | 0 | 1 | 0 | 0 | 0 | 0 | 0.7 | 0 | 0 | 0 | 0 | 1 | 0 | 0 |
| 67 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 68 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 69 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 70 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 71 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 72 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 73 | 0 | 0 | 0 | 0 | 0 | 0 | 0 | 0 | 0 | 1 | 0 | 0 | 0 | 1 | 0 | 0 | 0 | 0 | 0 | 0.7 | 0 |
| 74 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 75 | 0 | 0 | 0.7 | 1 | 0 | 0 | 1 | 0 | 0 | 0 | 0 | 0 | 0 | 0 | 0 | 0 | 0 | 0 | 0 | 0 | 0 |
| 76 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 77 | 0 | 0 | 0 | 0 | 0 | 0 | 0 | 0 | 0 | 0 | 0 | 0 | 0 | 0 | 0 | 0.7 | 0 | 1 | 0 | 0 | 0 |
| 78 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 79 | 0 | 0 | 0 | 0 | 0.7 | 0 | 0 | 0 | 0 | 0 | 0 | 0 | 0 | 0 | 0 | 0 | 0 | 1 | 0 | 0 | 0 |
| 80 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 81 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 82 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 83 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 84 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 85 | 0 | 0.7 | 0 | 0 | 0 | 1 | 0 | 0 | 0 | 0 | 0 | 1 | 0 | 0 | 0 | 0 | 0 | 0 | 0 | 0 | 0 |
| 86 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 87 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 88 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 89 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 90 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 91 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 92 | 0 | 0 | 0 | 0 | 0.7 | 0 | 0 | 0 | 0 | 0 | 0 | 0 | 0 | 0 | 0 | 0 | 0 | 1 | 1 | 1 | 0 |
| 93 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 94 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 95 | 0 | 0 | 0 | 0 | 0 | 0 | 0 | 0 | 1 | 0 | 0.7 | 0 | 0 | 0 | 0 | 0 | 0 | 0 | 0 | 0 | 0 |
| 96 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 97 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 98 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 99 | 0 | 0 | 0 | 0 | 0 | 0 | 0 | 0 | 0 | 0 | 0.7 | 0 | 1 | 0 | 0 | 0 | 0 | 0 | 0 | 0 | 0 |
| 100 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 101 | 0 | 0 | 0 | 0 | 0 | 0 | 0 | 0 | 0 | 0.7 | 0 | 0 | 0 | 1 | 0 | 0 | 0 | 0 | 0 | 1 | 0 |
| 102 | 0 | 0 | 0 | 0 | 0 | 0 | 1 | 0 | 0 | 0 | 0 | 0 | 0 | 0 | 0 | 0 | 0 | 0 | 0 | 0.7 | 0 |
| 103 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 104 | 0 | 0 | 0 | 0 | 0 | 0 | 0 | 0 | 0 | 1 | 0 | 0 | 0.7 | 0 | 0 | 0 | 0 | 0 | 0 | 0 | 0 |
| 105 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 106 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 107 | 0 | 0 | 0 | 0 | 0 | 1 | 0 | 0 | 0 | 0 | 0 | 0 | 0 | 0 | 0.7 | 0 | 0 | 0 | 0 | 0 | 0 |
| 108 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 109 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 110 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 111 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 112 | 1 | 0 | 0 | 0 | 0 | 0 | 0 | 0 | 0 | 0 | 0.7 | 0 | 0 | 1 | 0 | 0 | 0 | 0 | 0 | 0 | 0 |
| 113 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 114 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 115 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 116 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 117 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 118 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 119 | 0 | 1 | 0 | 0 | 0 | 0 | 0 | 0 | 0.7 | 0 | 0 | 0 | 0 | 0 | 0 | 0 | 0 | 0 | 0 | 0 | 0 |
| 120 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 121 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 122 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 123 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 124 | 0 | 0 | 0 | 1 | 0 | 0 | 0.7 | 0 | 0 | 0 | 0 | 0 | 0 | 0 | 0 | 0 | 0 | 0 | 0 | 0 | 0 |
| 125 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 126 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 127 | 0 | 0 | 0 | 0 | 0 | 0 | 0 | 0 | 0 | 0 | 1 | 0 | 0 | 0 | 0 | 0 | 0 | 0 | 0 | 0.7 | 0 |
| 128 | 0 | 0 | 0 | 0 | 0 | 0 | 0 | 0 | 0 | 0 | 1 | 0 | 0 | 0.7 | 0 | 0 | 0 | 0 | 0 | 0 | 0 |
| 129 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 130 | 0 | 0 | 0 | 0 | 0 | 0 | 0 | 0 | 0 | 0.7 | 1 | 0 | 0 | 0 | 0 | 0 | 0 | 0 | 0 | 0 | 0 |
| 131 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 132 | 0 | 0 | 0 | 0 | 0 | 0 | 0 | 0 | 0 | 0 | 0 | 0 | 1 | 0 | 0 | 0 | 0 | 1 | 0 | 0.7 | 0 |
| 133 | 0 | 1 | 0 | 0 | 0 | 0.7 | 1 | 0 | 0 | 0 | 0 | 0 | 0 | 0 | 0 | 0 | 0 | 0 | 0 | 0 | 0 |
| 134 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 135 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 136 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 137 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 138 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 139 | 0 | 0 | 0 | 0 | 0 | 0 | 0 | 0 | 0 | 0 | 0 | 0 | 0 | 1 | 0 | 0.7 | 0 | 0 | 0 | 0 | 0 |
| 140 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 141 | 0 | 0 | 0 | 0 | 0 | 0 | 0 | 0 | 1 | 0.7 | 0 | 1 | 0 | 0 | 0 | 0 | 0 | 0 | 0 | 1 | 0 |
| 142 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 143 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 144 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 145 | 0.7 | 0 | 0 | 0 | 0 | 0 | 0 | 0 | 0 | 0 | 0 | 0 | 0 | 0 | 1 | 0 | 0 | 0 | 0 | 0 | 0 |
| 146 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 147 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 148 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 149 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 150 | 0 | 1 | 0 | 0 | 0 | 0 | 0 | 0 | 0 | 0 | 0 | 0.7 | 0 | 0 | 0 | 0 | 0 | 0 | 0 | 0 | 0 |
| 151 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 152 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 153 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 154 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 155 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 156 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 157 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 158 | 0 | 0 | 0 | 0 | 0 | 0 | 0 | 0 | 0 | 0 | 0 | 0 | 0 | 1 | 0 | 0 | 0 | 0 | 0.7 | 0 | 0 |
| 159 | 0 | 0 | 0 | 0 | 0 | 0 | 0 | 0 | 0 | 0 | 0.7 | 0.7 | 1 | 0 | 0 | 0 | 0 | 0 | 0 | 0 | 0 |
| 160 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 161 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 162 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 163 | 1 | 0 | 0 | 0 | 0 | 0 | 0 | 0 | 0 | 0 | 0.7 | 0 | 0 | 0 | 0 | 0 | 0 | 0 | 0 | 0 | 0 |
| 164 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 165 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 166 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 167 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 168 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 169 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 170 | 0 | 0 | 0 | 0 | 0 | 0 | 0 | 1 | 0 | 0 | 0 | 0 | 0 | 0 | 0 | 0 | 0 | 0.7 | 0 | 0 | 0 |
| 171 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 172 | 0 | 0 | 0 | 0 | 0 | 0.7 | 1 | 0 | 0 | 0 | 0 | 0 | 0 | 0 | 0 | 0 | 0 | 0 | 0 | 0 | 0 |
| 173 | 1 | 1 | 0.7 | 0 | 0 | 0 | 0 | 0 | 0 | 0 | 1 | 1 | 0 | 0 | 0 | 0 | 1 | 0 | 0 | 0 | 0 |
| 174 | 0 | 0 | 0 | 0 | 0 | 0 | 0 | 0 | 0 | 0 | 0.7 | 0 | 0 | 1 | 0 | 0 | 0 | 0 | 0 | 0 | 0 |
| 175 | 0 | 0 | 0 | 0 | 0 | 0 | 0 | 0 | 0 | 0 | 1 | 0 | 0 | 0.7 | 0 | 0 | 0 | 0 | 0 | 0 | 0 |
| 176 | 0 | 0 | 0.7 | 1 | 0 | 1 | 0 | 0 | 0 | 0 | 0 | 0 | 0 | 0 | 0 | 0 | 0 | 0 | 0 | 0 | 0 |
| 177 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 178 | 0 | 0 | 0 | 0 | 0 | 0 | 0 | 0 | 0 | 0 | 0.7 | 0 | 1 | 0 | 0 | 0 | 0 | 0 | 0 | 0 | 0 |
| 179 | 0 | 0.7 | 0 | 0 | 0 | 1 | 0 | 0 | 0 | 0 | 0 | 0 | 0 | 0 | 0 | 0 | 0 | 0 | 0 | 0 | 0 |
| 180 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 181 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 182 | 0 | 0.7 | 0 | 0 | 0 | 1 | 0 | 0 | 0 | 0 | 0 | 0 | 0 | 0 | 0 | 0 | 0 | 0 | 0 | 0 | 0 |
| 183 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 184 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 185 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 186 | 0 | 0 | 0 | 0 | 0 | 1 | 0 | 0 | 0 | 1 | 0 | 0 | 0 | 0 | 0 | 0 | 0 | 0 | 0.7 | 0 | 0 |
| 187 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 188 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 189 | 0 | 0 | 0 | 0 | 0 | 0 | 0 | 1 | 0 | 0 | 0.7 | 0 | 0 | 0 | 0 | 0 | 0 | 0 | 0 | 0 | 0 |
| 190 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 191 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 192 | 0 | 0 | 0 | 1 | 0 | 0 | 0.7 | 0 | 0 | 0 | 0 | 0 | 0 | 0 | 0 | 0 | 0 | 0 | 0 | 0 | 0 |
| 193 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 194 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 195 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 196 | 0 | 0 | 1 | 0 | 0 | 0 | 0 | 0 | 0 | 0 | 0 | 0 | 0 | 0 | 0 | 0.7 | 0 | 0 | 0 | 0 | 0 |
| 197 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 198 | 0 | 0 | 0 | 0 | 0 | 0.7 | 0 | 0 | 0 | 0 | 0 | 0 | 0 | 0 | 1 | 0 | 0 | 0 | 0 | 0 | 0 |
| 199 | 0 | 0 | 0 | 0 | 0 | 1 | 0 | 0 | 0.7 | 0 | 0 | 0 | 0 | 0 | 0 | 0 | 0 | 0 | 0 | 0 | 0 |
| 200 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 201 | 1 | 0 | 0 | 0 | 0 | 0 | 0 | 0 | 0 | 0 | 0 | 0 | 1 | 0 | 0 | 0.7 | 0 | 0 | 0 | 0 | 0 |
| 202 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 203 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 204 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 205 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 206 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 207 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 208 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 209 | 0 | 0 | 0 | 0 | 0 | 0 | 0 | 0 | 0 | 0 | 0 | 1 | 0 | 0 | 0 | 0 | 0 | 0 | 0 | 0.7 | 0 |
| 210 | 1 | 0 | 0 | 0 | 0 | 0 | 0 | 0 | 0 | 0 | 0 | 0 | 0 | 1 | 0 | 0 | 1 | 1 | 1 | 0.7 | 0 |
| 211 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 212 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 213 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 214 | 0 | 0.7 | 0 | 0 | 0 | 1 | 0 | 0 | 0 | 0 | 0 | 0 | 0 | 0 | 0 | 0 | 0 | 0 | 0 | 0 | 0 |
| 215 | 1 | 0 | 0 | 0 | 0 | 0 | 0 | 0 | 0 | 0 | 0 | 0 | 0 | 0 | 0.7 | 0 | 0 | 0 | 0 | 0 | 0 |
| 216 | 0 | 0 | 0 | 0 | 0 | 0 | 0 | 0 | 0 | 0 | 0 | 0 | 0 | 0 | 0.7 | 1 | 0 | 0 | 0 | 0 | 0 |
| 217 | 1 | 0 | 0 | 1 | 0 | 0 | 0 | 0 | 0 | 0 | 0 | 0 | 0 | 0 | 0 | 0 | 0.7 | 0 | 0 | 0 | 0 |
| 218 | 1 | 0 | 0 | 0 | 0 | 0 | 0.7 | 0 | 0 | 0 | 0 | 0 | 0 | 0 | 0 | 0 | 0 | 0 | 0 | 0 | 0 |
| 219 | 0.7 | 0 | 0 | 0 | 0 | 0 | 0 | 0 | 0 | 0 | 0 | 0 | 0 | 0 | 1 | 0 | 0 | 0 | 0 | 0 | 0 |
| 220 | 0 | 0 | 0 | 0 | 0 | 0 | 0 | 0 | 0 | 0 | 0 | 0 | 0 | 1 | 0 | 0 | 0 | 0 | 0.7 | 0 | 0 |
| 221 | 0 | 0 | 0 | 0 | 0 | 1 | 1 | 0 | 0 | 0 | 0 | 0 | 0 | 0 | 0 | 0.7 | 0 | 0 | 0 | 0 | 0 |
| 222 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 223 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 224 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 225 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 226 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 227 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 228 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 229 | 0 | 0 | 0.7 | 0 | 0 | 0 | 1 | 0 | 0 | 0 | 0 | 0 | 0 | 0 | 0 | 0 | 0 | 0 | 0 | 0 | 0 |
| 230 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 231 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 232 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 233 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 234 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 235 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 236 | 0 | 0 | 0 | 0 | 0 | 0 | 0 | 0 | 0 | 0 | 1 | 0 | 0 | 0 | 0 | 0 | 0 | 0.7 | 0 | 0 | 0 |
| 237 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 238 | 0 | 1 | 0.7 | 0 | 0 | 0 | 0 | 0 | 0 | 0 | 0 | 1 | 0 | 0 | 0 | 0 | 0 | 0 | 0 | 0 | 0 |
| 239 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 240 | 1 | 0 | 0 | 0 | 0 | 0 | 0 | 0 | 0 | 0 | 0 | 0 | 0 | 0 | 0 | 0 | 0 | 0 | 0 | 0.7 | 0 |
| 241 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 242 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 243 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 244 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 245 | 0 | 0 | 0 | 0.7 | 0 | 0 | 1 | 0 | 0 | 0 | 0 | 0 | 0 | 0 | 0 | 0 | 0 | 0 | 0 | 0 | 0 |
| 246 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 247 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 248 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 249 | 0 | 0 | 0 | 0 | 0 | 0 | 0 | 0 | 0 | 0.7 | 1 | 0 | 0 | 0 | 0 | 0 | 0 | 0 | 0 | 0 | 0 |
| 250 | 0 | 0 | 0 | 0 | 0 | 0 | 0 | 0 | 0 | 1 | 0.7 | 0 | 1 | 0 | 0 | 0 | 0 | 0 | 0 | 0 | 0 |
| 251 | 1 | 0 | 0 | 0.7 | 0 | 0 | 0 | 0 | 0 | 0 | 1 | 0 | 0 | 0 | 0 | 0 | 0 | 0 | 0 | 0 | 0 |
| 252 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 253 | 1 | 0 | 0 | 0 | 0 | 0 | 0 | 0 | 0 | 0 | 0.7 | 0 | 0 | 0 | 0 | 0 | 0 | 0 | 0 | 1 | 0 |
| 254 | 1 | 0 | 0 | 0 | 0 | 0 | 0 | 0 | 0 | 1 | 0.7 | 0 | 1 | 0 | 0 | 0 | 0 | 0 | 0 | 0 | 0 |
| 255 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 256 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 257 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 258 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 259 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 260 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 261 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 262 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 263 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 264 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 265 | 0 | 0 | 0 | 0 | 0 | 0 | 0 | 0 | 0 | 0 | 1 | 0 | 0 | 0.7 | 0 | 0 | 0 | 0 | 0 | 0 | 0 |
| 266 | 0 | 0 | 0 | 0 | 0 | 0 | 0 | 0 | 0 | 0 | 1 | 0 | 0 | 1 | 0 | 0 | 0 | 0 | 0 | 0.7 | 0 |
| 267 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 268 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 269 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 270 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 271 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 272 | 0 | 0 | 0 | 0 | 0 | 0 | 0 | 0 | 0 | 1 | 0.7 | 0 | 0 | 0 | 0 | 0 | 0 | 0 | 0 | 0 | 0 |
| 273 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 274 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 275 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 276 | 0.7 | 0 | 0 | 0 | 0 | 0 | 0 | 0 | 0 | 0 | 1 | 0 | 0 | 0 | 0 | 0 | 0 | 0 | 0 | 1 | 0 |
| 277 | 0 | 0 | 0 | 0 | 0 | 0 | 0 | 0 | 0 | 1 | 0.7 | 0 | 0 | 0 | 0 | 0 | 0 | 0 | 0 | 0 | 0 |
| 278 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 279 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 280 | 1 | 0 | 0 | 0 | 0 | 0 | 0 | 0 | 0 | 0 | 0 | 0 | 0 | 0 | 0.7 | 0 | 0 | 0 | 0 | 0 | 0 |
| 281 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 282 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 283 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 284 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 285 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 286 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 287 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 288 | 0 | 0 | 1 | 0 | 0 | 0 | 0 | 0.7 | 0 | 0 | 0 | 0 | 0 | 0 | 0 | 0 | 0 | 0 | 0 | 0 | 0 |
| 289 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 290 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 291 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 292 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 293 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 294 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 295 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 296 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 297 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 298 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 299 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 300 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 301 | 0 | 0 | 0 | 0 | 0 | 0 | 0 | 0 | 0 | 0 | 1 | 0 | 0 | 0 | 0 | 0 | 0 | 0 | 0.7 | 0 | 0 |
| 302 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 303 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 304 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 305 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 306 | 1 | 0 | 0 | 0 | 0 | 0 | 0 | 0 | 0 | 0 | 0 | 0 | 0 | 0 | 0 | 0 | 0 | 0 | 0 | 0.7 | 0 |
| 307 | 0 | 1 | 0 | 0 | 0 | 1 | 0 | 0 | 0 | 0 | 0 | 0.7 | 0 | 0 | 0 | 0 | 0 | 0 | 0 | 0 | 0 |
| 308 | 1 | 0 | 1 | 0 | 0 | 0 | 0 | 0 | 0 | 0 | 0 | 0 | 0 | 0 | 0 | 0.7 | 0 | 0 | 0 | 0 | 0 |
| 309 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 310 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 311 | 0 | 1 | 0 | 0 | 0 | 0 | 0 | 0 | 0 | 0 | 0 | 0.7 | 0 | 0 | 0 | 0 | 0 | 0 | 0 | 0 | 0 |
| 312 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 313 | 0 | 0 | 0 | 0 | 0 | 0 | 0 | 0 | 0 | 0 | 1 | 0 | 0.7 | 0 | 0 | 0 | 0 | 0 | 0 | 0 | 0 |
| 314 | 0 | 0.7 | 0 | 0 | 0 | 1 | 0 | 0 | 0 | 0 | 0 | 0 | 0 | 0 | 0 | 0 | 0 | 0 | 0 | 0 | 0 |
| 315 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 316 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 317 | 0 | 0 | 0 | 0 | 0 | 0 | 0 | 0 | 0 | 1 | 0.7 | 0 | 0 | 0 | 0 | 0 | 0 | 0 | 0 | 0 | 0 |
| 318 | 0 | 0 | 0 | 0 | 0 | 1 | 0.7 | 0 | 0 | 0 | 0 | 1 | 0 | 0 | 0 | 0 | 0 | 0 | 0 | 0 | 0 |
| 319 | 0 | 0 | 0 | 0 | 0 | 0 | 1 | 0 | 0 | 0 | 0.7 | 0 | 0 | 0 | 0 | 0 | 0 | 0 | 0 | 0 | 0 |
| 320 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 321 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 322 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 323 | 0 | 0 | 0 | 1 | 0 | 0 | 1 | 0.7 | 0 | 0 | 0 | 0 | 0 | 0 | 0 | 0 | 0 | 0 | 0 | 0 | 0 |
| 324 | 0 | 0 | 0 | 0 | 0 | 0 | 0 | 0 | 0 | 0 | 0 | 0 | 0 | 0 | 0 | 0 | 0 | 1 | 0 | 0.7 | 0 |
| 325 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 326 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 327 | 0.7 | 0 | 0 | 0 | 0 | 0 | 0 | 0 | 0 | 1 | 0 | 0 | 0 | 0 | 0 | 0 | 0 | 0 | 0 | 0 | 0 |
| 328 | 0 | 1 | 0 | 0 | 0 | 0.7 | 0 | 0 | 0 | 0 | 0 | 0 | 0 | 0 | 0 | 0 | 0 | 0 | 0 | 0 | 0 |
| 329 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 330 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 331 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 332 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 333 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 334 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 335 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 336 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 337 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 338 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 339 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 340 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 341 | 0.7 | 0 | 0 | 0 | 1 | 0 | 0 | 0 | 0 | 0 | 0 | 0 | 0 | 0 | 0 | 1 | 0 | 0 | 0 | 0 | 0 |
| 342 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 343 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 344 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 345 | 0 | 1 | 0 | 0 | 0 | 0 | 0 | 0 | 0 | 0 | 0.7 | 0 | 0 | 0 | 0 | 0 | 1 | 0 | 0 | 0 | 0 |
| 346 | 0 | 0 | 0 | 0 | 0.7 | 0 | 0 | 0 | 0 | 0 | 0 | 0 | 0 | 0 | 0 | 0 | 0 | 0 | 0 | 0 | 0 |
| 347 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 348 | 0 | 0 | 0 | 0 | 0 | 0.7 | 1 | 0 | 0 | 0 | 0 | 0 | 0 | 0 | 0 | 0 | 0 | 0 | 0 | 0 | 0 |
| 349 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 350 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 351 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 352 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 353 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 354 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 355 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 356 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 357 | 0 | 0 | 0.7 | 1 | 0 | 0 | 1 | 0 | 0 | 0 | 0 | 0 | 0 | 0 | 0 | 0 | 0 | 0 | 0 | 0 | 0 |
| 358 | 0 | 0 | 1 | 0.7 | 0 | 0 | 0 | 0 | 1 | 0 | 0 | 0 | 0 | 0 | 0 | 0 | 0 | 0 | 0 | 0 | 0 |
| 359 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 360 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 361 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 362 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 363 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 364 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 365 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 366 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 367 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 368 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 369 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 370 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 371 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 372 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 373 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 374 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 375 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 376 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 377 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 378 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 379 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 380 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 381 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 382 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 383 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 384 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 385 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 386 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 387 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 388 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 389 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 390 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 391 | 0.7 | 0 | 0 | 0 | 0 | 0 | 0 | 0 | 0 | 0 | 0 | 0 | 0 | 0 | 0 | 0 | 1 | 0 | 0 | 0 | 0 |
| 392 | 0 | 0 | 0 | 0 | 0 | 0 | 0 | 0 | 0 | 0.7 | 1 | 0 | 0 | 0 | 0 | 0 | 0 | 0 | 0 | 0 | 0 |
| 393 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 394 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 395 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 396 | 0 | 0.7 | 0 | 0 | 0 | 0 | 0 | 0 | 0 | 0 | 0 | 1 | 0 | 0 | 0 | 0 | 1 | 0 | 0 | 0 | 0 |
| 397 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 398 | 0 | 0 | 0 | 0 | 0 | 0 | 0 | 0 | 0 | 0 | 0.7 | 0 | 0 | 0 | 0 | 0 | 0 | 0 | 0 | 1 | 0 |
| 399 | 0 | 0.7 | 0 | 0 | 0 | 0 | 0 | 0 | 0 | 0 | 1 | 0 | 0 | 0 | 0 | 0 | 0 | 0 | 0 | 0 | 0 |
| 400 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 401 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 402 | 0 | 0 | 0 | 0 | 0 | 0 | 0 | 0 | 0 | 0 | 1 | 0 | 1 | 0.7 | 0 | 0 | 0 | 0 | 0 | 0 | 0 |
| 403 | 0 | 0 | 0 | 0 | 0 | 1 | 0 | 0.7 | 0 | 0 | 0 | 0 | 0 | 0 | 0 | 0 | 0 | 0 | 0 | 0 | 0 |
| 404 | 0 | 0 | 0 | 0 | 0 | 0 | 0 | 1 | 0.7 | 0 | 0 | 0 | 0 | 0 | 0 | 0 | 0 | 0 | 0 | 0 | 0 |
| 405 | 0 | 1 | 0 | 0 | 0 | 0 | 0 | 0 | 1 | 0 | 0 | 0 | 0 | 0 | 0 | 0 | 0 | 0 | 0 | 0.7 | 0 |
| 406 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 407 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 408 | 0 | 0 | 0 | 0 | 0 | 0 | 0 | 0 | 0 | 0 | 0.7 | 0 | 0 | 0 | 0 | 0 | 0 | 0 | 0 | 1 | 0 |
| 409 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 410 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 411 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 412 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 413 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 414 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 415 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 416 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 417 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 418 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 419 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 420 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 421 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 422 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 423 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 424 | 0.7 | 0 | 0 | 0 | 0 | 0 | 0 | 0 | 0 | 0 | 0 | 0 | 0 | 0 | 0 | 1 | 0 | 0 | 0 | 0 | 0 |
| 425 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 426 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 427 | 0 | 0 | 0 | 0 | 0 | 0 | 0 | 0 | 0 | 0 | 0.7 | 0 | 1 | 0 | 0 | 0 | 0 | 0 | 0 | 0 | 0 |
| 428 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 429 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 430 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 431 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 432 | 0 | 0 | 0 | 0 | 0.7 | 0 | 0 | 0 | 0 | 1 | 0 | 0 | 0 | 0 | 0 | 0 | 0 | 0 | 0 | 0 | 0 |
| 433 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 434 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 435 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 436 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 437 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 438 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 439 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 440 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 441 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 442 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 443 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 444 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 445 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 446 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 447 | 0 | 0 | 0.7 | 1 | 0 | 0 | 0 | 0 | 0 | 0 | 0 | 0 | 0 | 0 | 0 | 0 | 0 | 0 | 0 | 0 | 0 |
| 448 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 449 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 450 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 451 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 452 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 453 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 454 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 455 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 456 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 457 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 458 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 459 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 460 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 461 | 0 | 0 | 0 | 0 | 0 | 0 | 0 | 0 | 0 | 0 | 0.7 | 0 | 1 | 0 | 0 | 0 | 0 | 0 | 0 | 0 | 0 |
| 462 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 463 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 464 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 465 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 466 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 467 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 468 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 469 | 0.7 | 0 | 0 | 0 | 0 | 0 | 0 | 0 | 0 | 0 | 0 | 0 | 0 | 0 | 0 | 0 | 1 | 0 | 0 | 0 | 0 |
| 470 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 471 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 472 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 473 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 474 | 0 | 0 | 0.7 | 1 | 0 | 0 | 0 | 0 | 0 | 0 | 0 | 0 | 0 | 0 | 0 | 0 | 0 | 0 | 0 | 0 | 0 |
| 475 | 1 | 0 | 0 | 0 | 0 | 0 | 0 | 0 | 0 | 0 | 0 | 0 | 0 | 0.7 | 0 | 0 | 0 | 0 | 0 | 0 | 0 |
| 476 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 477 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 478 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 479 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 480 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 481 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 482 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 483 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 484 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 485 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 486 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 487 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 488 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 489 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 490 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 491 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 492 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 493 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 494 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 495 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 496 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 497 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 498 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 499 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 500 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 501 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 502 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 503 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 504 | 0 | 0 | 0 | 0.7 | 0 | 0 | 1 | 0 | 0 | 0 | 0 | 0 | 0 | 0 | 0 | 0 | 0 | 0 | 0 | 0 | 0 |
| 505 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 506 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 507 | 0 | 1 | 0 | 0 | 0 | 0 | 0 | 0 | 0.7 | 0 | 0 | 0 | 0 | 0 | 0 | 0 | 0 | 0 | 0 | 0 | 0 |
| 508 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 509 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 510 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 511 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 512 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 513 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 514 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 515 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 516 | 0 | 0 | 0 | 0 | 0 | 0 | 0 | 0 | 1 | 0 | 0 | 0 | 0 | 0 | 0 | 0.7 | 0 | 0 | 0 | 0 | 0 |
| 517 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 518 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 519 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 520 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 521 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 522 | 0 | 0 | 0 | 0 | 0 | 0 | 0 | 0 | 0 | 1 | 0 | 0 | 0 | 0 | 0 | 0 | 0 | 0 | 0 | 0.7 | 0 |
| 523 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 524 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 525 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 526 | 1 | 0 | 0 | 0 | 0 | 0 | 0 | 0 | 0 | 0 | 0 | 0 | 0 | 0 | 0 | 0 | 0 | 0 | 0 | 0.7 | 0 |
| 527 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 528 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 529 | 0 | 0 | 1 | 0 | 0 | 0.7 | 1 | 0 | 0 | 0 | 0 | 0 | 0 | 0 | 0 | 0 | 0 | 0 | 0 | 0 | 0 |
| 530 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 531 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 532 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 533 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 534 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 535 | 0 | 0 | 0 | 0 | 0 | 0 | 0 | 0 | 0 | 0 | 0 | 0 | 0 | 0 | 0 | 0 | 1 | 0 | 0.7 | 0 | 0 |
| 536 | 0 | 1 | 0 | 0 | 0 | 0 | 1 | 0 | 0.7 | 0 | 0 | 0 | 0 | 0 | 0 | 0 | 0 | 0 | 0 | 0 | 0 |
| 537 | 0 | 1 | 1 | 0 | 0 | 1 | 0 | 0 | 0 | 0 | 0 | 1 | 0 | 0 | 0 | 0 | 1 | 0 | 0.7 | 0 | 0 |
| 538 | 0 | 0 | 0 | 0 | 0 | 1 | 1 | 0 | 0 | 0 | 0 | 0 | 0 | 0 | 0 | 0 | 0.7 | 0 | 0 | 0 | 0 |
| 539 | 0 | 1 | 1 | 1 | 0 | 0 | 0.7 | 0 | 0 | 0 | 0 | 1 | 0 | 0 | 0 | 0 | 0 | 0 | 0 | 0 | 0 |
| 540 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 541 | 0 | 0 | 0 | 0 | 0 | 0 | 0 | 0 | 0 | 0 | 0.7 | 0 | 0 | 0 | 0 | 0 | 0 | 0 | 0 | 1 | 0 |
| 542 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 543 | 0 | 0 | 1 | 0 | 0 | 0 | 0 | 0.7 | 0 | 0 | 0 | 0 | 0 | 0 | 0 | 1 | 0 | 0 | 0 | 0 | 0 |
| 544 | 0 | 0 | 0 | 0.7 | 0 | 0 | 1 | 0 | 0 | 0 | 0 | 0 | 0 | 0 | 0 | 0 | 0 | 0 | 0 | 0 | 0 |
| 545 | 1 | 0.7 | 0 | 0 | 0 | 0 | 0 | 0 | 0 | 0 | 0 | 0 | 0 | 0 | 0 | 0 | 0 | 0 | 0 | 0 | 0 |
| 546 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 547 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 548 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 549 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 550 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 551 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 552 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 553 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 554 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 555 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 556 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 557 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 558 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 559 | 0 | 0 | 0 | 0 | 0 | 0.7 | 1 | 0 | 0 | 0 | 0 | 0 | 0 | 0 | 0 | 1 | 0 | 0 | 0 | 0 | 0 |
| 560 | 0 | 0 | 0 | 0 | 0 | 0.7 | 1 | 0 | 0 | 0 | 0 | 0 | 0 | 0 | 0 | 0 | 0 | 0 | 0 | 0 | 0 |
| 561 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 562 | 0 | 0 | 0 | 0 | 0 | 0 | 0 | 0 | 0 | 0 | 0 | 0 | 0 | 1 | 0 | 0 | 0 | 0 | 0.7 | 0 | 0 |
| 563 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 564 | 0 | 0 | 1 | 0 | 0 | 0 | 0 | 0 | 1 | 0 | 0 | 1 | 0 | 0 | 0 | 0 | 0 | 0 | 0.7 | 0 | 0 |
| 565 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 566 | 0 | 0 | 0 | 1 | 0 | 0 | 0.7 | 0 | 0 | 0 | 0 | 0 | 0 | 0 | 0 | 1 | 0 | 0 | 0 | 0 | 0 |
| 567 | 0.7 | 0 | 1 | 1 | 0 | 0 | 0 | 0 | 0 | 0 | 0 | 0 | 0 | 0 | 0 | 0 | 0 | 0 | 0 | 0 | 0 |
| 568 | 0 | 0 | 0 | 0 | 0 | 1 | 0 | 0 | 0 | 0 | 1 | 0 | 0 | 0 | 0.7 | 0 | 0 | 0 | 0 | 0 | 0 |
| 569 | 0 | 0 | 1 | 0 | 0 | 1 | 0.7 | 0 | 1 | 0 | 0 | 1 | 0 | 0 | 0 | 1 | 0 | 0 | 0 | 0 | 0 |
| 570 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 571 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 572 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 573 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 574 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 575 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 576 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 577 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 578 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 579 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 580 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 581 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 582 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 583 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 584 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 585 | 0 | 0 | 0 | 0 | 0 | 0 | 0 | 0 | 0 | 1 | 0.7 | 0 | 0 | 0 | 0 | 0 | 0 | 0 | 0 | 0 | 0 |
| 586 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 587 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 588 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 589 | 0.7 | 0 | 0 | 0 | 0 | 0 | 0 | 0 | 0 | 0 | 1 | 0 | 1 | 0 | 0 | 0 | 0 | 0 | 0 | 0 | 0 |
| 590 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 591 | 0.7 | 0 | 0 | 0 | 1 | 0 | 0 | 0 | 0 | 0 | 0 | 0 | 0 | 0 | 0 | 0 | 0 | 0 | 0 | 0 | 0 |
| 592 | 0 | 0 | 0 | 0 | 0 | 0 | 0 | 0 | 0 | 0 | 0 | 0 | 0 | 0.7 | 0 | 0 | 0 | 0 | 1 | 0 | 0 |
| 593 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 594 | 0 | 0.7 | 0 | 0 | 0 | 0 | 0 | 0 | 0 | 0 | 0 | 1 | 0 | 0 | 0 | 0 | 0 | 0 | 0 | 0 | 0 |
| 595 | 0 | 0 | 1 | 0 | 0 | 0 | 0 | 0 | 0 | 0 | 0 | 0 | 0 | 0 | 0 | 0.7 | 0 | 0 | 0 | 0 | 0 |
| 596 | 0 | 0 | 0 | 0 | 0 | 1 | 0 | 0 | 0 | 0 | 0 | 0.7 | 0 | 0 | 1 | 0 | 0 | 0 | 0 | 0 | 0 |
| 597 | 0.7 | 0 | 0 | 0 | 0 | 0 | 1 | 0 | 0 | 0 | 0 | 1 | 0 | 0 | 0 | 0 | 0 | 0 | 0 | 0 | 0 |
| 598 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 599 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 600 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 601 | 0 | 0.7 | 0 | 0 | 0 | 1 | 0 | 0 | 0 | 0 | 0 | 1 | 0 | 0 | 0 | 0 | 0 | 0 | 0 | 0 | 0 |
| 602 | 0 | 0 | 0 | 0 | 0.7 | 0 | 0 | 0 | 0 | 0 | 0 | 0 | 0 | 0 | 0 | 0 | 0 | 0 | 0 | 0 | 0 |
| 603 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 604 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 605 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 606 | 0 | 0 | 0 | 0 | 0 | 0 | 0 | 0 | 0 | 0 | 0.7 | 0 | 0 | 0 | 0 | 0 | 0 | 0 | 0 | 1 | 0 |
| 607 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 608 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 609 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 610 | 0 | 0 | 0 | 0 | 0 | 0 | 0 | 0 | 0 | 0 | 0 | 0.7 | 0 | 0 | 0 | 0 | 0 | 0 | 1 | 0 | 0 |
| 611 | 0 | 0 | 0 | 0 | 0 | 0 | 0 | 0 | 0 | 0 | 0.7 | 0 | 0 | 0 | 0 | 0 | 0 | 0 | 0 | 1 | 0 |
| 612 | 0 | 0.7 | 0 | 1 | 0 | 0 | 0 | 0 | 0 | 0 | 0 | 0 | 0 | 0 | 0 | 0 | 0 | 0 | 0 | 0 | 0 |
| 613 | 0 | 0 | 0 | 0 | 0 | 0 | 0 | 0 | 0 | 0 | 0 | 0 | 0 | 0 | 1 | 0 | 0.7 | 0 | 0 | 0 | 0 |
| 614 | 0.7 | 0 | 0 | 0 | 0 | 0 | 1 | 0 | 0 | 0 | 0 | 0 | 0 | 0 | 0 | 0 | 0 | 0 | 0 | 0 | 0 |
| 615 | 0 | 0 | 0 | 0.7 | 0 | 0 | 0 | 1 | 0 | 0 | 0 | 0 | 0 | 0 | 0 | 0 | 0 | 0 | 0 | 0 | 0 |
| 616 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 617 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 618 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 619 | 0 | 0.7 | 0 | 0 | 0 | 0 | 0 | 0 | 0 | 0 | 0 | 0 | 0 | 0 | 0 | 1 | 0 | 0 | 1 | 0 | 0 |
| 620 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 621 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 622 | 0 | 1 | 0 | 0 | 0 | 0 | 0 | 0 | 0 | 0.7 | 0 | 1 | 0 | 0 | 0 | 0 | 0 | 0 | 0 | 0 | 0 |
| 623 | 0.7 | 0 | 0 | 1 | 0 | 0 | 0 | 0 | 0 | 0 | 0 | 0 | 0 | 0 | 0 | 0 | 0 | 0 | 0 | 0 | 0 |
| 624 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 625 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 626 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 627 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 628 | 0 | 0 | 0 | 0 | 0 | 0 | 0 | 0 | 0 | 0 | 0.7 | 0 | 1 | 0 | 0 | 0 | 0 | 0 | 0 | 0 | 0 |
| 629 | 0 | 0 | 0 | 0 | 0 | 0 | 0 | 0 | 1 | 0 | 0 | 0 | 0 | 0 | 0 | 0 | 0 | 0 | 0.7 | 0 | 0 |
| 630 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 631 | 0 | 0 | 0 | 0 | 0 | 0 | 0 | 0 | 0 | 0.7 | 1 | 0 | 0 | 0 | 0 | 0 | 0 | 0 | 0 | 0 | 0 |
| 632 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 633 | 0 | 0 | 0 | 1 | 0 | 0 | 1 | 0 | 0.7 | 0 | 0 | 0 | 0 | 0 | 0 | 0 | 0 | 0 | 0 | 0 | 0 |
| 634 | 0 | 0 | 0 | 0 | 0 | 0.7 | 1 | 0 | 0 | 0 | 0 | 0 | 0 | 0 | 0 | 0 | 0 | 0 | 0 | 0 | 0 |
| 635 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 636 | 0 | 0 | 0.7 | 0 | 0 | 1 | 0 | 0 | 0 | 0 | 0 | 0 | 0 | 0 | 0 | 0 | 0 | 0 | 0 | 0 | 0 |
| 637 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 638 | 0 | 0 | 1 | 0 | 0 | 1 | 0 | 0 | 0.7 | 0 | 0 | 1 | 0 | 0 | 0 | 1 | 0 | 0 | 0 | 0 | 0 |
| 639 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 640 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 641 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 642 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 643 | 0 | 0 | 0 | 0 | 0 | 0 | 0 | 0 | 0 | 0 | 1 | 0 | 0 | 0 | 0 | 0 | 0 | 0.7 | 0 | 0 | 0 |
| 644 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 645 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 646 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 647 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 648 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 649 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 650 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 651 | 0.7 | 0 | 1 | 0 | 0 | 0 | 0 | 1 | 0 | 1 | 0 | 0 | 0 | 0 | 0 | 0 | 0 | 0 | 0 | 0 | 0 |
| 652 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 653 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 654 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 655 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 656 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 657 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 658 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 659 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 660 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 661 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 662 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 663 | 0 | 0.7 | 0 | 0 | 0 | 0 | 0 | 0 | 1 | 0 | 0 | 0 | 0 | 0 | 0 | 0 | 0 | 0 | 0 | 0 | 0 |
| 664 | 0 | 0 | 0 | 0 | 0 | 0.7 | 0 | 1 | 0 | 0 | 0 | 0 | 0 | 0 | 0 | 0 | 0 | 0 | 0 | 0 | 0 |
| 665 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 666 | 0 | 0 | 0 | 0 | 0 | 0 | 0 | 0 | 0 | 0.7 | 0 | 0 | 0 | 0 | 0 | 0 | 0 | 0 | 0 | 1 | 0 |
| 667 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 668 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 669 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 670 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 671 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 672 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 673 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 674 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 675 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 676 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 677 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 678 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 679 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 680 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 681 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 682 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 683 | 0.7 | 0 | 0 | 0 | 0 | 0 | 0 | 0 | 0 | 0 | 0 | 0 | 0 | 0 | 0 | 1 | 0 | 0 | 0 | 0 | 0 |
| 684 | 0 | 0 | 0 | 0 | 0 | 0 | 0 | 0 | 0 | 0 | 0 | 0 | 0.7 | 0 | 0 | 0 | 0 | 0 | 0 | 0 | 0 |
| 685 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 686 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 687 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 688 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 689 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 690 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 691 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 692 | 1 | 0 | 0 | 0 | 0 | 0 | 0 | 0.7 | 0 | 0 | 0 | 0 | 0 | 0 | 0 | 0 | 0 | 0 | 0 | 0 | 0 |
| 693 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 694 | 0 | 0.7 | 0 | 0 | 0 | 1 | 0 | 0 | 0 | 0 | 0 | 0 | 0 | 0 | 0 | 0 | 0 | 0 | 0 | 0 | 0 |
| 695 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 696 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 697 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 698 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 699 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 700 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 701 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 702 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 703 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 704 | 0 | 0 | 1 | 0.7 | 0 | 0 | 0 | 0 | 1 | 0 | 0 | 0 | 0 | 0 | 0 | 0 | 0 | 0 | 0 | 0 | 0 |
| 705 | 0 | 0 | 0 | 0 | 0 | 1 | 0 | 0.7 | 0 | 0 | 0 | 0 | 0 | 0 | 0 | 0 | 0 | 0 | 0 | 0 | 0 |
| 706 | 0 | 0 | 0 | 0 | 0 | 0 | 0 | 0 | 0 | 0 | 0 | 1 | 0 | 0 | 0 | 0 | 0 | 0 | 0 | 0.7 | 0 |
| 707 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 708 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 709 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 710 | 0 | 0 | 0 | 0 | 0 | 0 | 0 | 0 | 1 | 0 | 0 | 0 | 0 | 0 | 0 | 0 | 0 | 0 | 0.7 | 0 | 0 |
| 711 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 712 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 713 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 714 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 715 | 0 | 0 | 0 | 1 | 0 | 0 | 0 | 0 | 0.7 | 0 | 0 | 0 | 0 | 0 | 0 | 0 | 0 | 0 | 0 | 0 | 0 |
| 716 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 717 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 718 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 719 | 0 | 0 | 0 | 0 | 0 | 0 | 0 | 0 | 0 | 0 | 0 | 0 | 0.7 | 0 | 0 | 0 | 1 | 0 | 0 | 0 | 0 |
| 720 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 721 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 722 | 1 | 0 | 0 | 0 | 0 | 0 | 0 | 0 | 0 | 0 | 0 | 1 | 0 | 0 | 0 | 0 | 0.7 | 0 | 0 | 0 | 0 |
| 723 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 724 | 0 | 0 | 0 | 0 | 0 | 0 | 0 | 0 | 0 | 0 | 1 | 0 | 1 | 1 | 0 | 0 | 0 | 0 | 0 | 0.7 | 0 |
| 725 | 0 | 0.7 | 0 | 0 | 0 | 0 | 0 | 0 | 0 | 0 | 0 | 1 | 0 | 0 | 0 | 0 | 0 | 0 | 0 | 0 | 0 |
| 726 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 727 | 0 | 0 | 0 | 0 | 0 | 0 | 0 | 0 | 0 | 1 | 0.7 | 0 | 0 | 0 | 0 | 0 | 0 | 0 | 0 | 0 | 0 |
| 728 | 1 | 0 | 0 | 0 | 0 | 0 | 0.7 | 0 | 0 | 0 | 0 | 0 | 0 | 0 | 0 | 0 | 0 | 0 | 0 | 0 | 0 |
| 729 | 0.7 | 1 | 0 | 0 | 0 | 1 | 0 | 0 | 1 | 0 | 0 | 1 | 0 | 0 | 0 | 0 | 0 | 0 | 0 | 0 | 0 |
| 730 | 0 | 0 | 0 | 0 | 0.7 | 0 | 0 | 0 | 0 | 0 | 0 | 0 | 0 | 0 | 0 | 0 | 0 | 0 | 0 | 0 | 0 |
| 731 | 0 | 0 | 1 | 0.7 | 0 | 0 | 0 | 0 | 0 | 0 | 0 | 0 | 0 | 0 | 0 | 0 | 0 | 0 | 0 | 0 | 0 |
| 732 | 0 | 0.7 | 0 | 0 | 0 | 0 | 0 | 0 | 1 | 0 | 0 | 1 | 0 | 0 | 0 | 0 | 0 | 0 | 0 | 0 | 0 |
| 733 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 734 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 735 | 0 | 0 | 1 | 0 | 0 | 0.7 | 1 | 0 | 0 | 0 | 0 | 0 | 0 | 0 | 0 | 0 | 0 | 0 | 0 | 0 | 0 |
| 736 | 0 | 0 | 0 | 0 | 0 | 1 | 0.7 | 0 | 1 | 0 | 0 | 0 | 0 | 0 | 0 | 0 | 0 | 0 | 1 | 0 | 0 |
| 737 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 738 | 0 | 0 | 0 | 0 | 0 | 0.7 | 1 | 0 | 0 | 0 | 0 | 0 | 0 | 0 | 0 | 0 | 0 | 0 | 1 | 0 | 0 |
| 739 | 0 | 0.7 | 0 | 0 | 0 | 0 | 1 | 0 | 0 | 0 | 0 | 0 | 0 | 0 | 0 | 0 | 0 | 0 | 0 | 0 | 0 |
| 740 | 0 | 0 | 0 | 0 | 0 | 0 | 0 | 0 | 0 | 0.7 | 1 | 0 | 0 | 0 | 0 | 0 | 0 | 0 | 0 | 0 | 0 |
| 741 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 742 | 0 | 0 | 0 | 0 | 0 | 1 | 1 | 0 | 0 | 0 | 0 | 0.7 | 0 | 0 | 0 | 0 | 0 | 0 | 0 | 0 | 0 |
| 743 | 0.7 | 0 | 0 | 0 | 0 | 0 | 0 | 0 | 0 | 0 | 0 | 1 | 0 | 0 | 0 | 0 | 0 | 0 | 0 | 0 | 0 |
| 744 | 1 | 0 | 0 | 0 | 0 | 0 | 0 | 0.7 | 0 | 0 | 0 | 0 | 0 | 0 | 0 | 1 | 0 | 0 | 0 | 0 | 0 |
| 745 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 746 | 0 | 0 | 0 | 0 | 0 | 1 | 0.7 | 0 | 0 | 0 | 0 | 0 | 0 | 0 | 0 | 0 | 0 | 0 | 0 | 0 | 0 |
| 747 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 748 | 0 | 0 | 0 | 0 | 0 | 0 | 0 | 0 | 0 | 0 | 0 | 0 | 0 | 0 | 0 | 0 | 0 | 0 | 0 | 0.7 | 0 |
| 749 | 0 | 1 | 0 | 0 | 0 | 0 | 0 | 0 | 0 | 0 | 0 | 0 | 0 | 0 | 0 | 0 | 0 | 0 | 0.7 | 0 | 0 |
| 750 | 0 | 0 | 0 | 0 | 0 | 0 | 1 | 0 | 0 | 0 | 0 | 0 | 0 | 0 | 0 | 0.7 | 0 | 0 | 0 | 0 | 0 |
| 751 | 0 | 0 | 1 | 0 | 0 | 0 | 0 | 0 | 0 | 0 | 0 | 1 | 0 | 0 | 0 | 0 | 0.7 | 0 | 0 | 0 | 0 |
| 752 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 753 | 0 | 0 | 0 | 0 | 0 | 0 | 0 | 0 | 0 | 0 | 0 | 0 | 0 | 0 | 0 | 0 | 0.7 | 0 | 0 | 0 | 0 |
| 754 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 755 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 756 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 757 | 0 | 0 | 0 | 0 | 0 | 0 | 0 | 0 | 0.7 | 0 | 0 | 0 | 0 | 1 | 0 | 0 | 0 | 0 | 1 | 0 | 0 |
| 758 | 1 | 0 | 0 | 0 | 0.7 | 0 | 0 | 0 | 0 | 0 | 0 | 0 | 0 | 0 | 0 | 0 | 1 | 0 | 0 | 0 | 0 |
| 759 | 0 | 0 | 0 | 0 | 0 | 0 | 1 | 0 | 0 | 0 | 0 | 1 | 0 | 0 | 0 | 1 | 0.7 | 0 | 0 | 0 | 0 |
| 760 | 0 | 0.7 | 0 | 0 | 0 | 0 | 0 | 0 | 0 | 0 | 0 | 1 | 0 | 0 | 0 | 0 | 0 | 0 | 0 | 0 | 0 |
| 761 | 0 | 0 | 0 | 0 | 0 | 0 | 0 | 0 | 0 | 1 | 0.7 | 0 | 1 | 0 | 0 | 0 | 0 | 0 | 0 | 0 | 0 |
| 762 | 0 | 0 | 0 | 0 | 0 | 0 | 0 | 0 | 0 | 0 | 0.7 | 0 | 1 | 0 | 0 | 0 | 0 | 0 | 0 | 0 | 0 |
| 763 | 0 | 0 | 0 | 0 | 0 | 0 | 0 | 0 | 0 | 0 | 0 | 0 | 0 | 0 | 0 | 0.7 | 1 | 0 | 0 | 0 | 0 |
| 764 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 765 | 0.7 | 0 | 0 | 0 | 0 | 0 | 0 | 0 | 0 | 0 | 0 | 0 | 0 | 0 | 0 | 1 | 0 | 0 | 0 | 0 | 0 |
| 766 | 0 | 1 | 0 | 0 | 0 | 0 | 0 | 0 | 0 | 0 | 0 | 0.7 | 0 | 0 | 0 | 0 | 0 | 0 | 0 | 0 | 0 |
| 767 | 0 | 0 | 0 | 0 | 0 | 0 | 0 | 0 | 0 | 0.7 | 0 | 0 | 0 | 0 | 0 | 0 | 0 | 0 | 0 | 0 | 0 |
| 768 | 0 | 0 | 0 | 0 | 0 | 0 | 0 | 0 | 0 | 0 | 0 | 0 | 1 | 0 | 0 | 0 | 0 | 0 | 0.7 | 0 | 0 |
| 769 | 0 | 0 | 0 | 0 | 0 | 0 | 0 | 0.7 | 0 | 0 | 0 | 0 | 0 | 0 | 0 | 0 | 0 | 0 | 0 | 0 | 0 |
| 770 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 771 | 0 | 0 | 0 | 0 | 0 | 0 | 0 | 0 | 0 | 0 | 0 | 0 | 0 | 0 | 0 | 0 | 0 | 0.7 | 0 | 0 | 0 |
| 772 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 773 | 0 | 0 | 0 | 0 | 0 | 0 | 0 | 0 | 0 | 0 | 0 | 0 | 0 | 0.7 | 0 | 0 | 0 | 0 | 0 | 0 | 0 |
| 774 | 0 | 0 | 0 | 0 | 0 | 0 | 0 | 0 | 0 | 1 | 0 | 0 | 1 | 0 | 0 | 0.7 | 1 | 0 | 0 | 1 | 0 |
| 775 | 0 | 0 | 0 | 0 | 0 | 0 | 0 | 0 | 0 | 0 | 0 | 0 | 0 | 0 | 0 | 0.7 | 0 | 0 | 0 | 0 | 0 |
| 776 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 777 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 778 | 0 | 0 | 0.7 | 0 | 0 | 0 | 0 | 0 | 0 | 0 | 0 | 0 | 0 | 0 | 0 | 0 | 0 | 0 | 0 | 0 | 0 |
| 779 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 780 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 781 | 0 | 0 | 0 | 0.7 | 0 | 0 | 0 | 0 | 0 | 0 | 0 | 0 | 0 | 0 | 0 | 0 | 0 | 0 | 0 | 0 | 0 |
| 782 | 0 | 0 | 0 | 0 | 0 | 0 | 0 | 0 | 0 | 0 | 1 | 0 | 0.7 | 0 | 0 | 0 | 0 | 0 | 0 | 0 | 0 |
| 783 | 0 | 0 | 0 | 0 | 0 | 0 | 0 | 0 | 0 | 0 | 1 | 0 | 0.7 | 0 | 0 | 0 | 0 | 1 | 0 | 0 | 0 |
| 784 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 785 | 0 | 0 | 0 | 0 | 0 | 0 | 0 | 0 | 0 | 0 | 0 | 0 | 0 | 0 | 0 | 0 | 0 | 0 | 0.7 | 0 | 0 |
| 786 | 0 | 0 | 0 | 0 | 0 | 0 | 0 | 0 | 0 | 1 | 1 | 0 | 0.7 | 0 | 0 | 0 | 0 | 0 | 0 | 0 | 0 |
| 787 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 788 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 789 | 0 | 0 | 0 | 0 | 0 | 0 | 0 | 0 | 0 | 0 | 0.7 | 0 | 1 | 0 | 0 | 0 | 0 | 0 | 0 | 0 | 0 |
| 790 | 0 | 0 | 0 | 0 | 0 | 0 | 0 | 0 | 0 | 0 | 0 | 0 | 0 | 0.7 | 0 | 0 | 0 | 0 | 1 | 0 | 0 |
| 791 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 792 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 793 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 794 | 0 | 0 | 0 | 0 | 0 | 0 | 0 | 0 | 0 | 0 | 0 | 0 | 0 | 1 | 0 | 0 | 0 | 0 | 0.7 | 0 | 0 |
| 795 | 0 | 0 | 0 | 0 | 0 | 0 | 0 | 0 | 0 | 0 | 0 | 0.7 | 0 | 0 | 0 | 0 | 0 | 0 | 0 | 0 | 0 |
| 796 | 0 | 0 | 0 | 0 | 0 | 0 | 0 | 0 | 0 | 0 | 0 | 0 | 0 | 0 | 0.7 | 0 | 0 | 0 | 0 | 0 | 0 |
| 797 | 0 | 0.7 | 0 | 0 | 0 | 0 | 0 | 0 | 0 | 0 | 0 | 0 | 0 | 0 | 0 | 0 | 0 | 0 | 0 | 0 | 0 |
| 798 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 799 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 800 | 0.7 | 1 | 0 | 0 | 0 | 1 | 0 | 0 | 0 | 0 | 0 | 1 | 0 | 0 | 0 | 0 | 0 | 0 | 0 | 0 | 0 |
| 801 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 802 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 803 | 0.7 | 0 | 0 | 0 | 0 | 0 | 0 | 0 | 0 | 0 | 0 | 0 | 0 | 0 | 0 | 0 | 0 | 0 | 0 | 0 | 0 |
| 804 | 0 | 0 | 0 | 0 | 0 | 0 | 0.7 | 0 | 0 | 0 | 0 | 0 | 0 | 0 | 0 | 0 | 0 | 0 | 0 | 0 | 0 |
| 805 | 0 | 0 | 0 | 0 | 0 | 0 | 0 | 0 | 0.7 | 0 | 0 | 0 | 0 | 0 | 0 | 0 | 0 | 0 | 0 | 0 | 0 |
| 806 | 0 | 0 | 0 | 0 | 0 | 0 | 0 | 0 | 0 | 0 | 0.7 | 0 | 0 | 0 | 0 | 0 | 0 | 0 | 0 | 0 | 0 |
| 807 | 0 | 0 | 0 | 0 | 0 | 0.7 | 0 | 0 | 0 | 0 | 0 | 0 | 0 | 0 | 0 | 0 | 0 | 0 | 0 | 0 | 0 |
| 808 | 0 | 0.7 | 0 | 0 | 0 | 0 | 0 | 0 | 0 | 0 | 0 | 0 | 0 | 0 | 0 | 0 | 0 | 0 | 0 | 0 | 0 |

### Example 21: Scaled Up One-Pot Reaction of pA21841 and pA29798

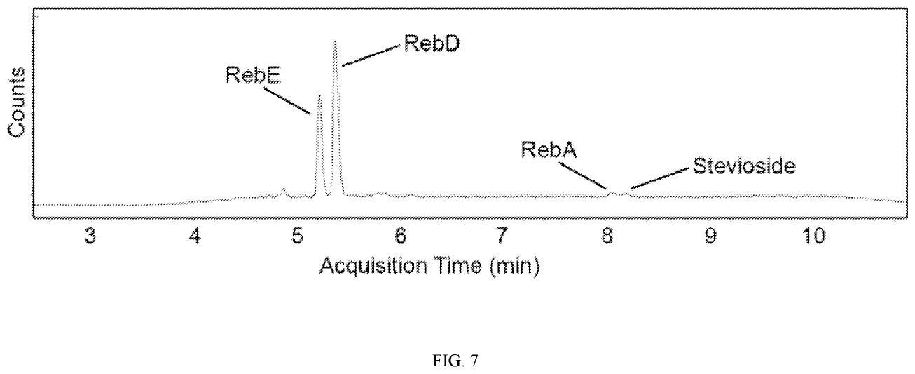

E. coli microorganisms containing either the SuSy, pA21841, or the B12GT, pA29798, were expressed in 1 L and 10 L fermenters. The cells were collected and lysed by French press. The expressed protein was purified by immobilized metal affinity chromatography (IMAC) and dialyzed into desalt buffer (20 mM KPO4 pH6, 50 mM NaCl). A one-pot reaction to convert Reb A and stevioside to Reb D and Reb E, respectively, was carried out. pA21841 and pA29798 were reacted with 100 mg/ml RA50, 250 mM Sucrose, and 0.5 mM ADP in 50 mM KPO4 pH6 and 50 mM NaCl. In total, ten 20 mL 1pot reactions were conducted. The reactions were lyophilized and the combined reaction product was analyzed for rebaudioside content by liquid chromatography-mass spectrometry (LCMS) using an Agilent 6545 QTOF mass spectrometer (column: 150×2.1 mm Phenomenex C18-PS). Full conversion of Reb A to Reb D and stevioside to Reb E was observed (FIG. 7; Table 29).

TABLE 29
Rebaudioside Content of pA21841 and
pA29798 One-Pot Reaction Product

| | Retention | | % |
|---|---|---|---|
| Rebaudioside | Time (min) | Area | (SG Only) |
| Unknown (mass: 1127) | 4.6 | 28703 | 0.3 |
| Reb E | 5.2 | 3768926 | 37.5 |
| Reb D | 5.4 | 6011185 | 59.8 |
| Reb A | 8.1 | 164560 | 1.6 |
| Stevioside | 8.2 | 86265 | 0.9 |

### Example 22: Scaled Up One-Pot Reaction of pA21841 and pA29646

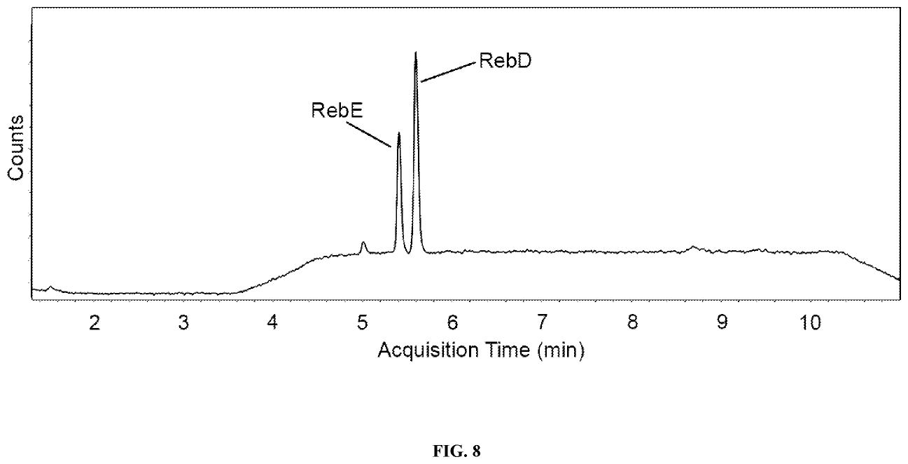

E. coli microorganisms containing either the SuSy, pA21841, or the B12GT, pA29646, were expressed in 10 L fermenters. The cells were collected and lysed by French press. The expressed protein was purified by immobilized metal affinity chromatography (IMAC) and dialyzed into desalt buffer (20 mM KPO4 pH6, 50 mM NaCl). A one-pot reaction to convert Reb A and stevioside to Reb D and Reb E, respectively, was carried out. pA21841 and pA29646 were reacted with 100 mg/ml RA50, 250 mM Sucrose, and 0.5 mM ADP in 50 mM KPO4 pH6 and 50 mM NaCl. In total, ten 20 mL 1pot reactions were conducted. The reactions were lyophilized and the combined reaction product was analyzed for rebaudioside content by liquid chromatography-mass spectrometry (LCMS) using an Agilent 6545 QTOF mass spectrometer (column: 150×2.1 mm phenomenex C18-PS). Full conversion of Reb A to Reb D and stevioside to Reb E was observed (FIG. 8; Table 30).

TABLE 30
Rebaudioside Content of pA21841 and
pA29646 One-Pot Reaction Product

| | Retention | | % |
|---|---|---|---|
| | Time (min) | Area | (SG Only) |
| Unknown (mass: 1127) | 5 | 2075 | 0 |
| Reb E | 5.4 | 954516 | 32 |
| Reb D | 5.6 | 1658236 | 65 |
| Reb A | 8.7 | 49223 | 2 |
| Stevioside | 8.8 | 22057 | 1 |

### Example 23: Pichia pastoris Expression of Designed B12GTs and SuSys

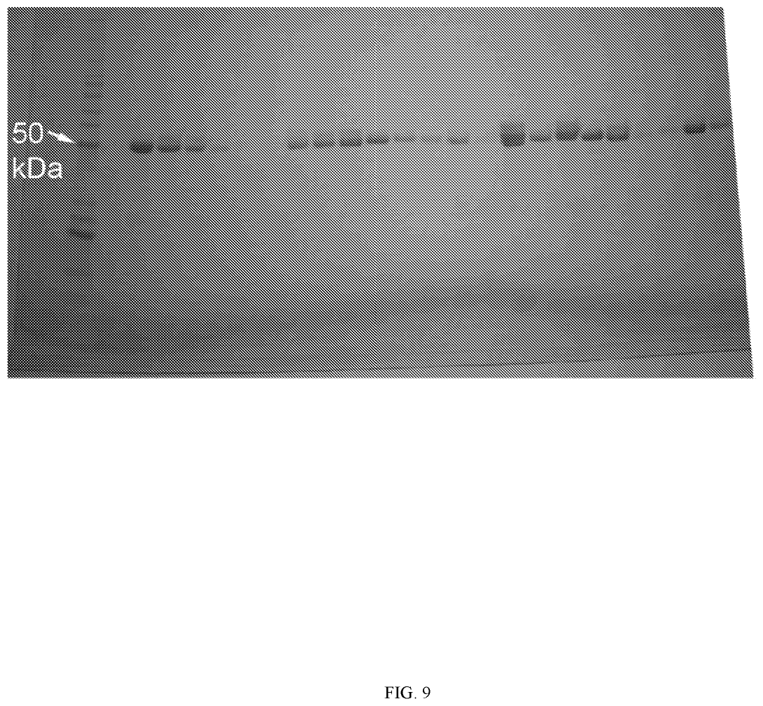

Polynucleotides optimized for Pichia pastoris expression of top designed B12GT (from Examples 12, 13 and 15) and SuSys (from Examples 17 and 19) were synthesized (Twist Bioscience) and inserted into a Pichia shuttle vector. The vectors were transformed into a commercially available Pichia pastoris strain (ATCC). The transformed microorganisms were grown in BMGY (buffered glycerol complex) media and protein expression was induced by feeding of methanol. The Pichia cells were lysed with Y-PER (Yeast Protein Extraction Reagent; Thermo Scientific) and the expressed proteins were purified by immobilized metal affinity chromatography (IMAC) and desalted into desalt buffer (20 mM KPO4 pH6, 50 mM NaCl). The designed B12GTs and SuSys solubly expressed and were catalytically active. FIG. 9 shows an SDS-PAGE gel of designed B12GTs purified from Pichia pastoris expression.

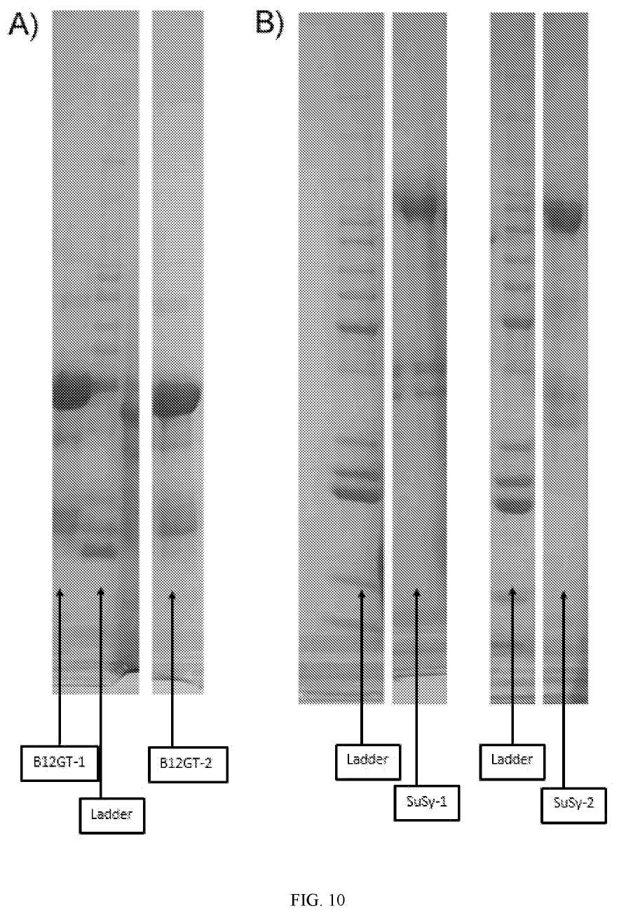

Two designed B12GTs and two designed SuSys were also expressed in 1 L fermentations. The Pichia microorganisms were grown with glycerol as the main carbon source for ˜24 hours, and then were fed methanol for ˜72 hours to express the desired B12GT or SUSY. The cells were collected and lysed by French press. The expressed protein was purified by immobilized metal affinity chromatography (IMAC) and dialyzed into desalt buffer (20 mM KPO4 pH6, 50 mM NaCl). FIG. 11A shows an SDS-PAGE gel of two designed B12GTs, pA29798 (left, B12GT-1) and pA32946 (right, B12GT-2), purified from 1 L Pichia pastoris fermentations. FIG. 10B shows SDS-PAGE gels of two designed SuSys, pA34103 (left, SuSy-1) and pA32691 (right, SuSy-2), purified from 1 L Pichia pastoris fermentations. All four enzymes successfully expressed in the fermentations and had the desired activity.

1-11. (canceled)

12. An engineered sucrose synthase polypeptide that comprises an amino acid sequence that is at least 60% identical to an amino acid sequence selected from the group consisting of SEQ ID NOs: 925-1180, wherein the amino acid sequence comprises H at position 425, R at position 567, K at position 572, and E at position 663, numbered according to SEQ ID NO: 885.

13-16. (canceled)

17. An engineered sucrose synthase polypeptide that has a score greater than 556 when scored by the position-specific scoring matrix (PSSM) shown in Table 24.

18. A polypeptide having the sequence of
MIEXLXXXLXXXXXXXXXXLRXXXXXXRXXXXXXDLXXXXXXFXXXXXX
XXXXXXXXXXXXXXXXQEAXXXXPWXXXAXRXRXXXWXYXRXHXEXLXV
EEXXXXEXLXXKEXLVXXXXEGXAVXXXDXXDXXXXXQXXKDESTIGXG
XXHLNRHLXGRXWXDXXXGXXXXXXXLXXHXXXXXXLXLXXXXXXFDXL
RXXXQYLGXXPXXXPXXXXXXXXXXXGFEPGXGXTXXRXRXTXRLLXDX
LDSPSPXXLEXFLXRXPXIXXXXIXSXHGXFXQXXVLGXPDTGGQVVXI
LDQXRALEXEXRXRLXXQGXDXEPXIXXXTRLIPXXXGTTCDQRLEPXX
GXXXXXILRXPFRXEXGXXXPXWISRFXXWPYLERXXXDXEXEXXAELG
XRPDXIIGNYSDGXLXAXXXXXKXGXTQXNXAHALEKXKYXXSDLXWXX
XEXXXHFXCQFTADXIAMNAADXIXTSTYQEIAGXDXXVGQYESXXXXT
XPGLYRXXXGXDVFDXXFNIXSPGADXXXYFXYXXXEXRXXXLXPEIEX
XXXXXXXXXXXRGVLXDXXKPXXXXXXRXDRIKNXXGXXEXXGXXXRLR
XLANLXXXXGHXDXXXSXDXEEXXXXXRXHXXXDXXXLDGQXRXXGXXL
XKXXVGEXYRXXADXRGXXXQPALXEAFGLTVIEXMXSGLPXXATXXGG
PXEIIEXGVSGFHIDPNXXXXXXEXXADXXEXXXXXPXYWEXISXXALX
RVXXRYTWXXXAERXXTXXRXXGFWXXVXXREXQVXXRYLQMXRHLQXR
PLAHAVPXE
wherein residue 4 is A or R or E or S or V
wherein residue 6 is R or D or E
wherein residue 7 is Q or E
wherein residue 8 is Q or F
wherein residue 10 is A or R or Q or H or L or K or S
wherein residue 11 is D or Q or E or S
wherein residue 12 is N or H or S
wherein residue 13 is R or P
wherein residue 14 is R or N or D or E
wherein residue 15 is A or D or Q or E or S or T
wherein residue 16 is L or W or V
wherein residue 17 is R or H or Y
wherein residue 18 is A or R or L
wherein residue 19 is L or F
wherein residue 22 is R or H
wherein residue 23 is L or Y
wherein residue 24 is L or V
wherein residue 25 is A or G
wherein residue 26 is Q or L or S
wherein residue 27 is Q or G
wherein residue 29 is D or G or P or T
wherein residue 30 is I or L or M or F or S
wherein residue 31 is L or W
wherein residue 32 is Q or L
wherein residue 33 is R or H
wherein residue 34 is H or S or T
wherein residue 37 is Q or W
wherein residue 38 is R or D or H
wherein residue 39 is A or E
wherein residue 40 is C or L or F
wherein residue 41 is A or D or E or L or K
wherein residue 42 is A or R or N or D or E or T
wherein residue 44 is R or C or Y
wherein residue 45 is A or E
wherein residue 46 is Q or E or P
wherein residue 47 is D or Q or E or P
wherein residue 48 is D or G or P
wherein residue 49 is N or E
wherein residue 50 is E or G
wherein residue 51 is E or S or Y
wherein residue 52 is A or E or G or L or P
wherein residue 53 is E or L
wherein residue 54 is A or D or C or G or L or K or M or T
wherein residue 55 is D or G or I or S or T
wherein residue 56 is G or S
wherein residue 57 is A or P or W or V
wherein residue 58 is L or F
wherein residue 59 is A or E or G
wherein residue 60 is R or D or Q or E
wherein residue 61 is A or L or F or V
wherein residue 62 is I or V
wherein residue 63 is A or R or Q or E or K
wherein residue 64 is A or R or H or K
wherein residue 65 is A or C or T
wherein residue 69 is A or I or V
wherein residue 70 is I or L or F or V
wherein residue 71 is R or D or E or H or L
wherein residue 72 is A or D
wherein residue 75 is A or I or L or M or F or V
wherein residue 76 is C or Y or V
wherein residue 77 is L or F
wherein residue 79 is I or L or W or V
wherein residue 81 is E or L or P
wherein residue 83 is P or V
wherein residue 84 is A or G
wherein residue 85 is R or V
wherein residue 87 is R or E or Y
wherein residue 89 is L or Y or V
wherein residue 91 is I or F
wherein residue 93 is I or L or V
wherein residue 95 is D or Q or T
wherein residue 97 is A or D or E or T or V
wherein residue 101 is I or L or V
wherein residue 102 is D or S or T
wherein residue 103 is T or V
wherein residue 104 is D or E or S
wherein residue 106 is F or Y
wherein residue 108 is A or Q
wherein residue 109 is A or F
wherein residue 112 is R or Q or E
wherein residue 115 is N or D or G or K or T
wherein residue 116 is G or L
wherein residue 117 is A or N or D or E or G
wherein residue 118 is A or Q or G or H or P or S
wherein residue 121 is N or D or E or P
wherein residue 124 is L or T or W
wherein residue 125 is E or K or T
wherein residue 126 is I or W or V
wherein residue 128 is L or F or W
wherein residue 129 is E or G
wherein residue 131 is L or F
wherein residue 132 is R or N
wherein residue 133 is R or P
wherein residue 134 is N or E or G or H or S or Tor V
wherein residue 135 is F or S or V
wherein residue 137 is R or K
wherein residue 138 is L or M
wherein residue 146 is R or N or D
wherein residue 148 is L or V
wherein residue 149 is Q or E or T
wherein residue 156 is A or S
wherein residue 159 is I or L or M
wherein residue 161 is Q or G or T
wherein residue 163 is L or T
wherein residue 164 is A or G or K
wherein residue 165 is A or R or D or Q or E or G or K
wherein residue 167 is R or D or L or M
wherein residue 168 is D or Q or E or S or Y
wherein residue 169 is A or R or Q or K
wherein residue 170 is I or L
wherein residue 171 is I or L
wherein residue 172 is D or E
wherein residue 173 is F or W
wherein residue 175 is R or G or S
wherein residue 176 is L or V
wherein residue 178 is R or Q
wherein residue 179 is H or I or L or Y
wherein residue 180 is R or D
wherein residue 181 is N or G
wherein residue 182 is R or Q
wherein residue 183 is N or Q or H
wherein residue 185 is G or M
wherein residue 187 is N or S
wherein residue 188 is N or D or E
wherein residue 189 is R or G
wherein residue 190 is N or I or M or F
wherein residue 191 is R or Q or K or T
wherein residue 192 is D or S
wherein residue 195 is A or E or G or S
wherein residue 198 is R or Q
wherein residue 199 is A or T or V
wherein residue 200 is E or I or L or M or V
wherein residue 205 is R or G or K or T
wherein residue 206 is Q or L
wherein residue 208 is A or R or D or E or P
wherein residue 209 is D or E
wherein residue 210 is A or T
wherein residue 212 is L or W
wherein residue 213 is A or E or S
wherein residue 214 is D or E
wherein residue 215 is L or F or V
wherein residue 216 is A or R or Q or E or G
wherein residue 217 is E or H or P
wherein residue 218 is A or R or D or E or K or T
wherein residue 219 is L or M
wherein residue 220 is R or Q
wherein residue 221 is R or E
wherein residue 222 is R or L or M or W
wherein residue 228 is L or W
wherein residue 230 is R or N or D
wherein residue 232 is A or V
wherein residue 233 is A or Q or E or G
wherein residue 235 is I or M or V
wherein residue 237 is D or E
wherein residue 239 is L or M
wherein residue 243 is M or V
wherein residue 245 is I or L
wherein residue 252 is A or R or E or G or S
wherein residue 253 is A or N or T
wherein residue 256 is R or E or K or S or T
wherein residue 259 is A or D or G
wherein residue 261 is I or V
wherein residue 263 is L or M
wherein residue 265 is F or S
wherein residue 266 is N or S
wherein residue 267 is I or L or V
wherein residue 268 is A or I or L or T or V
wherein residue 270 is I or L or V
wherein residue 272 is I or P or V
wherein residue 275 is W or Y
wherein residue 277 is A or G
wherein residue 279 is A or D or S
wherein residue 280 is N or K
wherein residue 284 is R or L or Y
wherein residue 293 is F or W or Y
wherein residue 298 is A or V
wherein residue 303 is R or K
wherein residue 305 is I or L or M
wherein residue 307 is R or N or D or Q or E or K
wherein residue 310 is A or R or H or Y
wherein residue 311 is Q or E
wherein residue 314 is L or V
wherein residue 316 is I or V
wherein residue 319 is R or Q
wherein residue 321 is I or L
wherein residue 322 is I or V
wherein residue 323 is A or I or L or V
wherein residue 329 is D or E
wherein residue 330 is A or S
wherein residue 331 is R or D or E or G or K
wherein residue 342 is I or V
wherein residue 343 is H or S or V
wherein residue 345 is A or T
wherein residue 346 is R or E
wherein residue 347 is N or H or Y
wherein residue 348 is A or V
wherein residue 349 is R or Q or H or W
wherein residue 353 is I or V
wherein residue 357 is N or Y
wherein residue 359 is D or S
wherein residue 361 is R or N or E or T
wherein residue 362 is I or V
wherein residue 363 is H or I or L or V
wherein residue 365 is Q or H
wherein residue 371 is R or E or K
wherein residue 372 is I or V
wherein residue 379 is F or W or Y
wherein residue 380 is A or V
wherein residue 381 is R or Q or E or L or S
wherein residue 383 is A or L or V
wherein residue 385 is R or K
wherein residue 387 is I or L or V
wherein residue 388 is L or K
wherein residue 393 is G or S
wherein residue 397 is A or L or V
wherein residue 406 is N or G
wherein residue 408 is I or V
wherein residue 410 is S or T
wherein residue 411 is I or L
wherein residue 412 is I or L or M
wherein residue 413 is A or S
wherein residue 414 is Q or E
wherein residue 416 is L or W
wherein residue 418 is I or V
wherein residue 421 is C or I or M or T or V
wherein residue 423 is I or F
wherein residue 430 is S or T
wherein residue 433 is L or P
wherein residue 434 is D or G or Y
wherein residue 438 is H or Y
wherein residue 440 is R or K or P
wherein residue 441 is R or D or L or K
wherein residue 442 is N or H or F or Y
wherein residue 444 is A or D or Q or E or P
wherein residue 445 is D or Q or K
wherein residue 446 is H or Y
wherein residue 449 is A or S
wherein residue 456 is L or W
wherein residue 464 is A or I or F or V
wherein residue 466 is I or V
wherein residue 476 is N or T
wherein residue 478 is R or N or D or H
wherein residue 479 is E or S
wherein residue 486 is H or Y
wherein residue 487 is A or Q or G or S or T
wherein residue 488 is A or D or H or S
wherein residue 489 is F or Y
wherein residue 491 is L or M
wherein residue 497 is I or V
wherein residue 498 is E or I or V
wherein residue 499 is N or H
wherein residue 501 is I or V
wherein residue 506 is P or S
wherein residue 507 is R or K
wherein residue 511 is I or V
wherein residue 517 is A or P
wherein residue 518 is R or D or E or S
wherein residue 519 is I or T or V
wherein residue 522 is P or S
wherein residue 524 is A or S or T
wherein residue 525 is R or D or E
wherein residue 526 is H or K or T
wherein residue 528 is R or E or K
wherein residue 530 is L or F
wherein residue 531 is S or T
wherein residue 532 is G or S
wherein residue 534 is H or W
wherein residue 539 is R or E or K or S
wherein residue 540 is I or L or M
wherein residue 541 is I or L or W or V
wherein residue 542 is F or Y
wherein residue 543 is G or S
wherein residue 544 is R or D or G or P
wherein residue 545 is D or E or P
wherein residue 546 is D or Q or E or P or T
wherein residue 547 is R or G
wherein residue 548 is A or G or P
wherein residue 549 is D or E or P
wherein residue 550 is A or H or I
wherein residue 555 is A or E or K or S
wherein residue 557 is R or P
wherein residue 558 is D or Q or S
wherein residue 561 is I or L or V
wherein residue 562 is I or L
wherein residue 563 is L or F
wherein residue 564 is S or T
wherein residue 565 is I or M or V
wherein residue 566 is A or M or S
wherein residue 568 is L or M
wherein residue 574 is Q or I or L or M
wherein residue 575 is S or T
wherein residue 577 is L or W
wherein residue 578 is A or L or M or V
wherein residue 580 is I or L or W
wherein residue 581 is F or Y or V
wherein residue 583 is A or R
wherein residue 584 is N or S
wherein residue 585 is A or N or E or P or S
wherein residue 589 is E or S
wherein residue 594 is I or L or V
wherein residue 595 is I or L or V
wherein residue 596 is I or V
wherein residue 597 is A or G
wherein residue 600 is I or V
wherein residue 602 is A or P or V
wherein residue 603 is A or N or Q or E or G or S
wherein residue 604 is A or R or N or Q or E or K
wherein residue 606 is A or R or N or D or G or M or S or T
wherein residue 608 is A or R or E or G or H or S
wherein residue 611 is R or Q
wherein residue 612 is A or E
wherein residue 613 is Q or E
wherein residue 614 is I or M or V
wherein residue 615 is A or R or Q or E or G or K
wherein residue 617 is I or L or M
wherein residue 619 is Q or E
wherein residue 620 is I or L
wherein residue 621 is I or L or M
wherein residue 623 is R or E or H or K
wherein residue 624 is H or Y
wherein residue 625 is N or Q or G
wherein residue 630 is A or M or F or V
wherein residue 632 is L or W
wherein residue 633 is I or L or V
wherein residue 635 is A or L or S or V
wherein residue 636 is Q or H
wherein residue 638 is N or D or E
wherein residue 640 is R or N or T or V
wherein residue 641 is R or L or W or V
wherein residue 645 is I or L
wherein residue 648 is W or Y or V
wherein residue 649 is I or L or V
wherein residue 652 is R or Q or G or H or K or T
wherein residue 655 is A or I or V
wherein residue 656 is F or W or V
wherein residue 657 is I or V
wherein residue 662 is F or Y
wherein residue 672 is A or V
wherein residue 674 is A or S or T
wherein residue 679 is T or V
wherein residue 680 is F or W
wherein residue 683 is R or C
wherein residue 684 is H or Y
wherein residue 688 is A or L
wherein residue 693 is D or H
wherein residue 704 is D or Q or H
wherein residue 705 is G or P
wherein residue 706 is D or E
wherein residue 707 is A or Q or E
wherein residue 708 is A or T or V
wherein residue 709 is A or L
wherein residue 711 is R or I or L or K
wherein residue 712 is I or L or M
wherein residue 715 is F or W
wherein residue 716 is L or F
wherein residue 718 is A or R or H or K
wherein residue 719 is A or C
wherein residue 720 is A or R or K
wherein residue 721 is A or N or E
wherein residue 722 is R or N or D or E
wherein residue 724 is D or G or K or S or T
wherein residue 728 is R or E or K
wherein residue 731 is R or D or Q
wherein residue 732 is A or G
wherein residue 735 is A or Q or E or K
wherein residue 738 is R or E or S or Y
wherein residue 739 is A or E or S
wherein residue 744 is E or K
wherein residue 745 is R or L
wherein residue 746 is W or Y
wherein residue 750 is L or M or W
wherein residue 751 is A or L or M
wherein residue 753 is I or L
wherein residue 754 is A or I or L or M or S or V
wherein residue 756 is A or C or I or V
wherein residue 757 is A or I or M or F or W or Y
wherein residue 761 is R or K
wherein residue 762 is F or Y
wherein residue 764 is L or S or T
wherein residue 765 is N or K or S
wherein residue 768 is R or H or S
wherein residue 771 is M or T
wherein residue 772 is R or E
wherein residue 778 is I or F
wherein residue 783 is F or W or Y
wherein residue 792 is L or M (SEQ ID NO: 1229)

19. (canceled)

20. (canceled)

21. (canceled)

22. A method for transferring a sugar moiety to a substrate steviol glycoside, the method comprising contacting a β-1,2-glycosyltransferase (B12GT) and a sucrose synthase with one or more steviol glycosides, a non-UDP nucleotide diphosphate, and sucrose.

23. The method of claim 22, wherein the B12GT polypeptide is an engineered B12GT that comprises an amino acid sequence that is at least 60% identical to an amino acid sequence selected from the group consisting of SEQ ID NOs: 1-882 and 1333-1466.

24. The method of claim 22, wherein the sucrose synthase polypeptide is an engineered sucrose synthase that comprises an amino acid sequence that is at least 60% identical to an amino acid sequence selected from the group consisting of SEQ ID NOs: 925-1180, wherein the amino acid sequence comprises H at position 425, R at position 567, K at position 572, and E at position 663, numbered according to SEQ ID NO: 885.

25. (canceled)

26. The method of claim 22, wherein the B12GT polypeptide is an engineered B12GT polypeptide that has a score greater than 266.7 when scored by the position-specific scoring matrix (PSSM) shown in Table 17.

27. The method of claim 22, wherein the sucrose synthase is an engineered sucrose synthase polypeptide that has a score greater than 556 when scored by the position-specific scoring matrix (PSSM) shown in Table 24.

28. (canceled)

29. (canceled)

30. (canceled)

31. The method of claim 22, wherein the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof.

32. The method of claim 22, wherein the substrate steviol glycoside is a mixture of stevioside and rebaudioside A.

33. The method of claim 22, wherein the target steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof.

34. The method of claim 22, wherein the target steviol glycoside is a mixture of rebaudioside E and rebaudioside D.

35. The method of claim 22, wherein the target steviol glycoside is rebaudioside D.

36. The method of claim 22, wherein the target steviol glycoside is rebaudioside E.

37. The method of claim 22, wherein the non-UDP nucleotide diphosphate is ADP, GDP, CDP, or TDP.

38. The method of claim 22, wherein the non-UDP nucleotide diphosphate is ADP.

39. A polynucleotide encoding a polypeptide of claim 12.

40. A host microorganism heterologously expressing the polynucleotide of claim 39.

41. The engineered sucrose synthase polypeptide of claim 12, wherein the amino acid sequence selected from the group is SEQ ID NO: 1132.

42. The method of claim 24, wherein the amino acid sequence selected from the group is SEQ ID NO: 1132.
